(19) United States
(12) Patent Application Publication
(10) Pub. No.: US 2026/0125374 A1
(43) Pub. Date: May 7, 2026

(54) PI3K-ALPHA INHIBITORS AND METHODS OF MAKING AND USING THE SAME

(71) Applicant: Relay Therapeutics, Inc., Cambridge, MA (US)

(72) Inventors: André Lescarbeau, Cambridge, MA (US); Alessandro Boezio, Cambridge, MA (US); Surendra P. Singh, Cambridge, MA (US); Qinglin Che, Cambridge, MA (US); Siyi Jiang, Shanghai (CN); Hongyan He, Shanghai (CN); Qiuxiang Zhou, Shanghai (CN); Jiajia Zhou, Shanghai (CN); Yuan Lin, Shanghai (CN); Wei Gu, Shanghai (CN); Min Lu, Shanghai (CN); Yunfei Zhou, Shanghai (CN); Xijian Gong, Shanghai (CN); Jiahui Chen, Shanghai (CN); Xiaohong Wang, Shanghai (CN); Changbo Yin, Shanghai (CN)

(21) Appl. No.: 18/706,511

(22) PCT Filed: Nov. 3, 2022
(86) PCT No.: PCT/US2022/079221

§ 371 Date: May 1, 2024

Related U.S. Application Data
(60) Provisional application No. 63/263,474, filed on Nov. 3, 2021.

(30) Foreign Application Priority Data
Nov. 3, 2021 (WO) PCT/CN2021/128533

(51) Int. Cl. C07D 471/04; A61K 31/437; A61K 45/06

(52) U.S. Cl. CPC C07D 471/04; A61K 31/437; A61K 45/06

(57) ABSTRACT

The present disclosure relates to PI3Ka inhibitors, the crystalline forms, salts, and cocrystals thereof, and the compositions and methods of making and use thereof.

### CROSS-REFERENCE TO RELATED APPLICATIONS

This application claims the benefit of U.S. Provisional Application No. 63/263,474, filed Nov. 3, 2021, and International (PCT) Patent Application No. PCT/CN2021/128533, filed Nov. 3, 2021; the entirety of each of which is hereby incorporated by reference.

### BACKGROUND

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃), which, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997); Katso et al., Annu. Rev. Cell Dev. Biol. 17:615-675 (2001)).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP₂ and PIP₃ recruit Aid, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)).

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Ka would be of particular value in the treatment of proliferative disease and other disorders. While multiple inhibitors of PI3Ks have been developed (for example, taselisib, alpelisib, buparlisib and others), these molecules inhibit multiple Class TA PI3K isoforms. Inhibitors that are active against multiple Class TA PI3K isoforms are known as “pan-PI3K” inhibitors. A major hurdle for the clinical development of existing PI3K inhibitors has been the inability to achieve the required level of target inhibition in tumors while avoiding toxicity in cancer patients. Pan-PI3K inhibitors share certain target-related toxicities including diarrhea, rash, fatigue, and hyperglycemia. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (Hanker et al., Cancer Discovery (2019) PMID: 30837161. Therefore, selective inhibitors of PI3Kα may increase the therapeutic window, enabling sufficient target inhibition in the tumor while avoiding dose-limiting toxicity in cancer patients.

### SUMMARY OF THE INVENTION

This disclosure is generally directed to the compounds of formulae I-III and solvates thereof, and crystalline forms thereof.

In some embodiments, the present disclosure provides a compound of Formula (I):

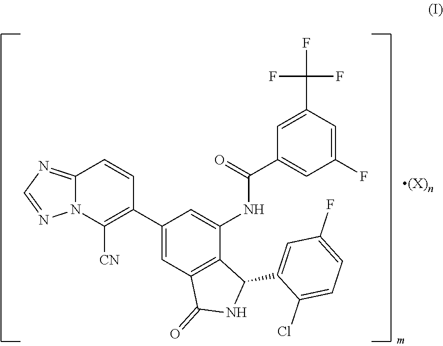

or a solvate thereof, wherein each of X, m, and n is independently as defined and described in embodiments herein. In some embodiments, a compound of Formula (I), or a solvate thereof, is a crystalline form as described herein.

In another aspect, provided herein is a compound of Formula (II):

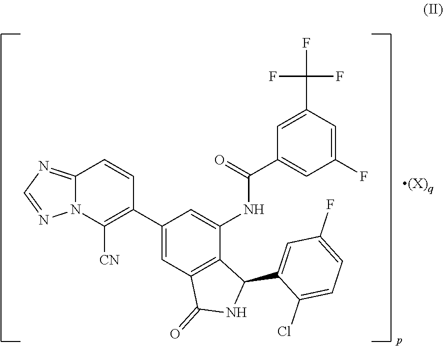

or a solvate thereof, wherein each o X, p, and q is independently as defined and described in embodiments herein. In some embodiments, a compound of Formula (II), or a solvate thereof, is a crystalline form as described herein.

In another aspect, provided herein is a compound of Formula (III):

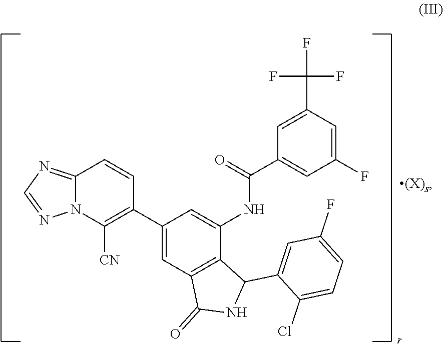

or a solvate thereof, wherein each of X, r, and s is independently as defined and described in embodiments herein. In some embodiments, a compound of Formula (III), or a solvate thereof, is a crystalline form as described herein.

In one aspect, provided herein is a compound of Formula (IV-1)

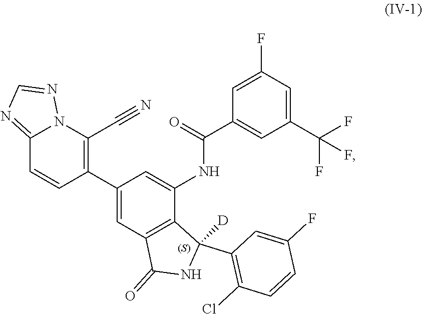

or a pharmaceutically acceptable salt thereof.

In another aspect, provided herein is a compound of Formula (IV-2)

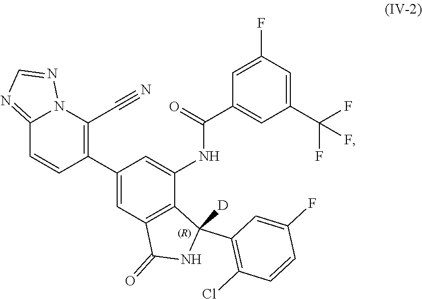

or a pharmaceutically acceptable salt thereof.

In another aspect, provided herein is a method comprising deuteration of compound III-1 followed by a purification step to separate the enantiomers, thereby forming compounds IV-1 and IV-2:

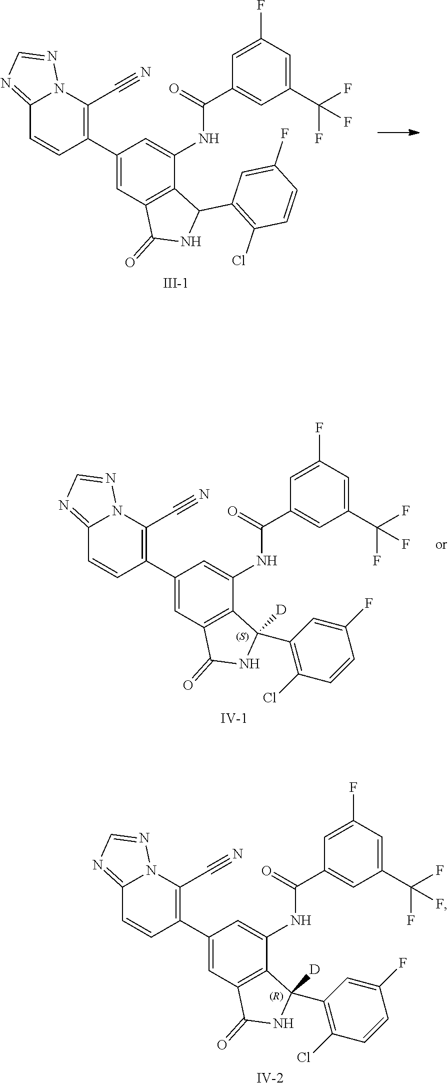

for example, as described in Example 3-A.

In another aspect, provided herein is a method for preparing compounds I-1 and II-1 by subjecting compound III-1 to a SMB separation:

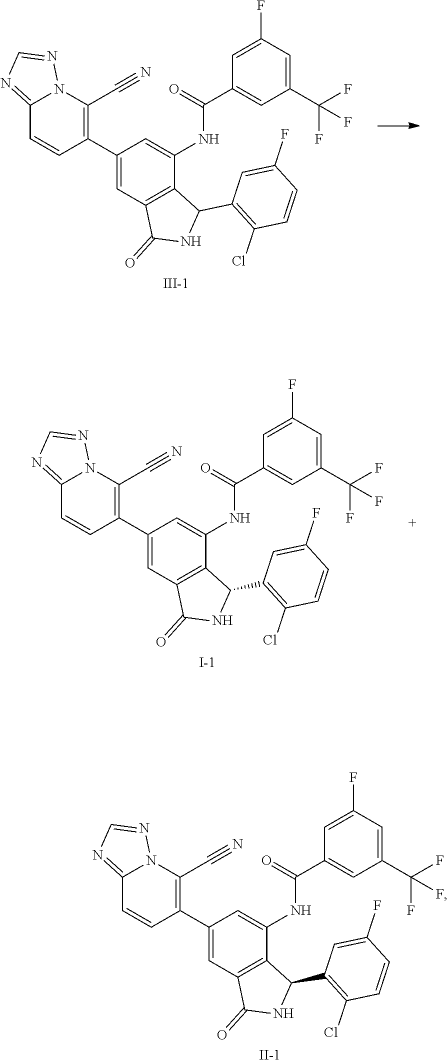

for example, as described in Example 1-A.

In another aspect, provided herein is a method for preparing compound III-1 by a racemization of compound II-1:

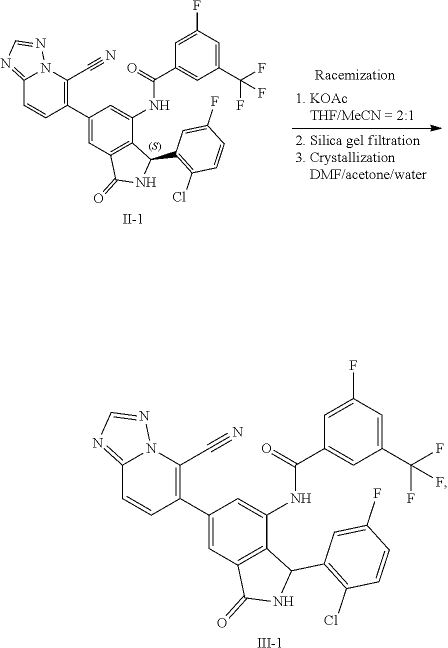

for example, as described in Example 2-A.

In another aspect, provided herein is a pharmaceutical composition comprising a compound or a pharmaceutically acceptable salt thereof, or a solvate thereof, or a crystalline form, as described herein, and a pharmaceutically acceptable excipient. In another aspect, provided herein is a pharmaceutical composition comprising a compound or a pharmaceutically acceptable salt thereof, as described herein, and a pharmaceutically acceptable excipient.

In another aspect, provided herein is a method of using a compound or a solvate thereof, or a crystalline form, or a pharmaceutical composition thereof, as described herein, for inhibiting PI3Kα activity and for treating a disorder, disease, and/or condition as described herein. In another aspect, provided herein is a method of using a compound or a pharmaceutically acceptable salt thereof, or a pharmaceutical composition thereof, as described herein, for inhibiting PI3Kα activity and for treating a disorder, disease, and/or condition as described herein.

### BRIEF DESCRIPTION OF THE DRAWINGS

FIG. TA depicts an XRPD pattern of I-1 Form A.

FIG. 1B depicts a DSC thermogram of I-1 Form A (heating rate: 10° C./min).

FIG. 1C depicts a DSC thermogram of I-1 Form A (heating rate: 2° C./min).

FIG. 1D depicts a DSC thermogram of I-1 Form A (heating rate: 2° C./min).

FIG. 1E depicts a TGA thermogram of I-1 Form A.

FIG. 2A depicts an XRPD pattern of I-1 Form B.

FIG. 2B depicts a DSC thermogram of I-1 Form B.

FIG. 2C depicts a TGA thermogram of I-1 Form B.

FIG. 3A depicts an XRPD pattern of I-1 Form C.

FIG. 3B depicts a DSC thermogram of I-1 Form C.

FIG. 3C depicts a TGA thermogram of I-1 Form C.

FIG. 4A depicts an XRPD pattern of III-1 Form A.

FIG. 4B depicts a DSC thermogram of III-1 Form A.

FIG. 4C depicts a TGA thermogram of III-1 Form A.

FIG. 5A depicts an XRPD pattern of III-1 Form B.

FIG. 5B depicts a DSC thermogram of III-1 Form B.

FIG. 6A depicts an XRPD pattern of III-1 Form C.

FIG. 6B depicts a DSC thermogram of III-1 Form C.

FIG. 6C depicts a TGA thermogram of III-1 Form C.

FIG. 7A depicts an XRPD pattern of III-1 Form D.

FIG. 7B depicts a DSC thermogram of III-1 Form D.

FIG. 7C depicts a TGA thermogram of III-1 Form D.

FIG. 8 depicts an XRPD pattern of III-1 Form E.

FIG. 9A depicts an XRPD pattern of III-1 Form F.

FIG. 9B depicts a DSC thermogram of III-1 Form F.

FIG. 10 depicts an XRPD pattern of II-1 Form A.

FIG. 11 depicts an XRPD pattern of II-1 Form B.

FIG. 12 depicts an XRPD pattern of II-1 Form C.

FIG. 13 depicts an XRPD overlay of solids obtained from competitive equilibration experiments at 25° C. with I-1 Form A and Form C. The patterns from top to bottom are: Compound I-1 Form A in EA/heptane; Compound I-1 Form A in MeOH/DCM; Compound I-1 Form A in THF/MTBE; Compound I-1 Form A in THF/heptane; Compound I-1 Form C; and Compound I-1 Form A.

FIG. 14 depicts an XRPD overlay of solids obtained from CE1-THF/heptane (2:3, v/v) at 25° C. with I-1 Form A and Form C. The patterns from top to bottom are: Compound I-1 Form A in THF/heptane; Compound I-1 Form C; and Compound I-1 Form A.

FIG. 15 depicts an XRPD overlay of solids obtained from CE2-THF/MTBE (1:4, v/v) at 25° C. with Form A and Form C. The patterns from top to bottom are: Compound I-1 Form A in THF/MTBE; Compound I-1 Form C; and Compound I-1 Form A.

FIG. 16 depicts an XRPD overlay of solids obtained from CE3-MeOH/DCM (1:2, v/v) at 25° C. with Form A and Form C. The patterns from top to bottom are: Compound I-1 Form A in MeOH/DCM; Compound I-1 Form C; and Compound I-1 Form A.

FIG. 17 depicts an XRPD overlay of solids obtained from CE4-EA/heptane (1:1, v/v) at 25° C. with Form A and Form C. The patterns from top to bottom are: Compound I-1 Form A in EA/heptane; Compound I-1 Form C; and Compound I-1 Form A.

FIG. 18 depicts an XRPD overlay of solids obtained from CE5-MeOH/DCM (1:2, v/v) at 25° C. and CE3-MeOH/DCM (1:2, v/v) at 25° C.

FIG. 19 depicts an XRPD overlay of solids obtained from competitive experiments of CE6, CE7 and CE8 at 25° C. The patterns from top to bottom are: Compound I-1 Form A in THF/ACN; Compound I-1 Form A in THF/MTBE; Compound I-1 Form A in 1, 4-dioxane; Compound I-1 Form C; and Compound I-1 Form A.

FIG. 20 depicts an XRPD overlay of solids obtained from behavior under compression experiments. The patterns from top to bottom are: I-1 Form A compressed for 5 minutes at 1OMPa, 5 MPa and 2 MPa, and I-1 Form A.

FIG. 21 depicts an XRPD overlay of solids obtained from grinding simulation experiments. The patterns from top to bottom are: I-1 Form A ground manually with a mortar and pestle for 5, 3 and 1 min, and I-1 Form A.

FIG. 22 depicts an XRPD overlay of solids obtained from granulation simulation experiments.

FIG. 23 depicts an XRPD overlay of Form A after heating to different temperature by DSC at 2° C./min. The patterns from top to bottom are: Form A heated at 300° C., 270° C., and 260° C., and I-1 Form A.

FIG. 24 depicts an DSC overlay of Form A after heating to different temperature by DSC at 2° C./min. The patterns from top to bottom are: Form A heated at 260° C., 270° C., and 300° C.

FIG. 25 depicts an XRPD overlay of Form A heating to 260° C. at 2° C./min by DSC. The patterns from top to bottom are: Form A after being heated, and I-1 Form A.

FIG. 26 depicts an XRPD overlay of Form A heating to 260° C. and 270° C. at 2° C./min by DSC. The patterns from top to bottom are: Form A heated to 270° C. and 260° C., and I-1 Form A.

FIG. 27A depicts an XRPD pattern of I-2 Form A.

FIG. 27B depicts a DSC thermogram of I-2 Form A.

FIG. 27C depicts a TGA thermogram of I-2 Form A.

FIG. 28 depicts an XRPD pattern of II-2 Form A.

FIG. 29A depicts an XRPD pattern of III-2 Form A.

FIG. 29B depicts a DSC thermogram of III-2 Form A.

FIG. 29C depicts a TGA thermogram of III-2 Form A.

FIG. 30A depicts an XRPD pattern of I-3 Form A.

FIG. 30B depicts a DSC thermogram of I-3 Form A.

FIG. 30C depicts a TGA thermogram of I-3 Form A.

FIG. 31A depicts an XRPD pattern of I-4 Form A.

FIG. 31B depicts a DSC thermogram of I-4 Form A.

FIG. 31C depicts a TGA thermogram of I-4 Form A.

FIG. 32A depicts an XRPD pattern of I-5 Form A.

FIG. 32B depicts a DSC thermogram of I-5 Form A.

FIG. 32C depicts a DSC thermogram of I-5 Form A.

FIG. 32D depicts a TGA thermogram of I-5 Form A.

FIG. 33 depicts an XRPD pattern of I-5 Form B.

FIG. 34A depicts an XRPD pattern of III-6 Form A.

FIG. 34B depicts a DSC thermogram of III-6 Form A.

FIG. 34C depicts a DSC thermogram of III-6 Form A.

FIG. 35A depicts an XRPD overlay of samples from VH-XRPD experiment of I-3 Form A.

FIG. 35B depicts an XRPD overlay of I-3 Form B and samples from VH-XRPD experiment of I-3 Form A.

FIG. 35C depicts an XRPD overlay of I-3 Form A in different humidity chamber after 1 week.

FIG. 35D depicts an XRPD overlay of solids from VH-XRPD experiments and different humidity chamber of I-3 Form A.

### DETAILED DESCRIPTION

### General Description of Certain Embodiments of the Invention

It has been found that compounds of formulae:

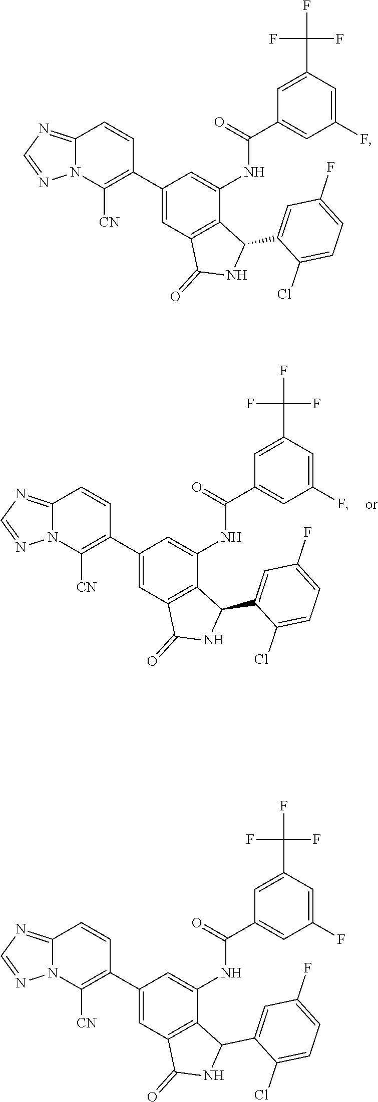 [SMILES: N#Cc1c(-c2cc(NC(=O)c3cc(F)cc(C(F)(F)F)c3)c3c(c2)C(=O)N[C@H]3c2cc(F)ccc2Cl)ccc2ncnn12 ; N#Cc1c(-c2cc(NC(=O)c3cc(F)cc(C(F)(F)F)c3)c3c(c2)C(=O)N[C@@H]3c2cc(F)ccc2Cl)ccc2ncnn12 ; N#Cc1c(-c2cc(NC(=O)c3cc(F)cc(C(F)(F)F)c3)c3c(c2)C(=O)NC3c2cc(F)ccc2Cl)ccc2ncnn12]

PI3Kα inhibitors and useful for treating disorders, diseases, and/or conditions, for example, the “PI3Kα-mediated” disorders, diseases, and/or conditions as described herein. It would be desirable to provide solid forms of the compounds (e.g., as a freebase, or a salt, or a solvate) that imparts characteristics such as improved aqueous solubility, stability, and ease of formulation. It would be desirable to provide deuterated analogues of the compounds, that imparts characteristics such as improved aqueous solubility, stability, and ease of formulation.

### Compound of Formula (I)

In some embodiments, provided herein is a compound of Formula (I)

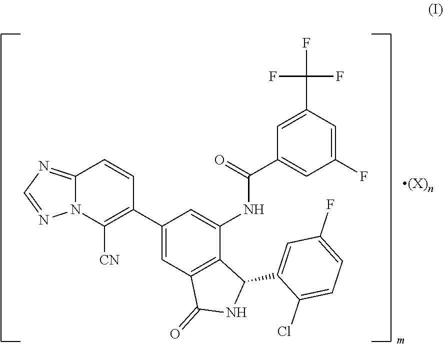

or a solvate thereof;
wherein:
m is 1, 2, 3, 4, 5, 6, 7, 8, or 9;
n is 0, 0.5, 1, 1.5, 2, 2.5, or 3; and
X is hydrochloric acid, p-toluene sulfonic acid, methane sulfonic acid, naphthalene-1,5-disulfonic acid, or 2-naphthalene sulfonic acid.

It will be appreciated by one of ordinary skill in the art that the acid moiety indicated as “X” and (R)-N-(3-(2-chloro-5-fluorophenyl)-6-(5-cyano-[1,2,4]triazolo[1,5-a]pyridin-6-yl)-1-oxoisoindolin-4-yl)-3-fluoro-5-(trifluoromethyl)benzamide are ionically bonded to form a compound of Formula (I). It will also be appreciated that when n is 0, X is absent, indicating that the compound of Formula (I) exists as a “free base,” i.e., “free form.”

It is contemplated that a compound of Formula (I) can exist in a variety of physical forms. For example, a compound of Formula (I) can be in solution, suspension, or in solid form. In certain embodiments, a compound of Formula (I) is in solid form. When a compound of Formula (I) is in solid form, said compound may be amorphous, crystalline, or a mixture thereof. Exemplary solid forms are described in more detail below.

In some embodiments, a compound of Formula (I) is anhydrate. In some embodiments, a compound of Formula (I) may be in a hydrate form. In some embodiments, a compound of Formula (I) may be in a hemi-hydrate form.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9.

In some embodiments, n is 0. In some embodiments, n is 1. In some embodiments, n is 2. In some embodiments, n is 3. In some embodiments, n is 0.5. In some embodiments, n is 1.5. In some embodiments, n is 2.5.

In some embodiments, X is hydrochloric acid. In some embodiments, X is p-toluene sulfonic acid. In some embodiments, X is methane sulfonic acid. In some embodiments, X is naphthalene-1,5-disulfonic acid. In some embodiments, X is 2-naphthalene sulfonic acid.

In some embodiments, the present invention provides a form of compound I substantially free of impurities. As used herein, the term “substantially free of impurities” means that the compound contains no significant amount of extraneous matter. Such extraneous matter may include different forms of compound I, residual solvents, or any other impurities that may result from the preparation of, and/or isolation of, compound I.

In some embodiments, a compound of Formula (I), or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a compound of Formula (I), or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, a compound of Formula (I), or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a compound of Formula (I), or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound of Formula (I) is also meant to include all tautomeric forms. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ³C- or ¹⁴C-enriched carbon are within the scope of this invention.

### Compound I-1

In some embodiments, a compound of Formula (I) is compound I-1, which is a free base (or “free form”),

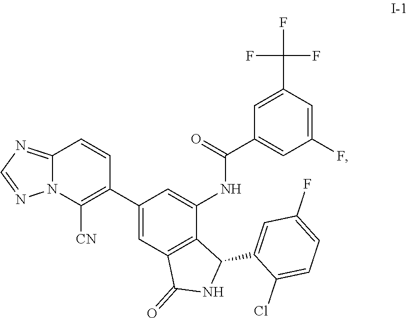 [SMILES: N#Cc1c(-c2cc(NC(=O)c3cc(F)cc(C(F)(F)F)c3)c3c(c2)C(=O)N[C@H]3c2cc(F)ccc2Cl)ccc2ncnn12]

or a solvate thereof.

In some embodiments compound I-1 is an amorphous solid. In some embodiments, Compound I-1 is a crystalline solid. In some embodiments, Compound I-1 is a mixture of amorphous solid form and crystalline solid form.

In some embodiments, the present invention provides a form of compound I-1 substantially free of impurities. As used herein, the term “substantially free of impurities” means that the compound contains no significant amount of extraneous matter. Such extraneous matter may include different forms of compound I-1, residual solvents, or any other impurities that may result from the preparation of, and/or isolation of, compound I-1.

In some embodiments, compound I-1, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound I-1, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound I-1, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound I-1, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound I-1 is also meant to include all tautomeric forms of compound I-1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In other embodiments, compound I-1 is a crystalline solid substantially free of amorphous compound I-1. As used herein, the term “substantially free of amorphous compound I-1” means that the compound contains no significant amount of amorphous compound I-1. In certain embodiments, at least about 95% by weight of crystalline compound I-1 is present. In certain embodiments, at least about 99% by weight of crystalline compound I-1 is present.

It has been found that compound I-1 can exist in a variety of solid forms. Exemplary such forms include polymorphs such as those described herein.

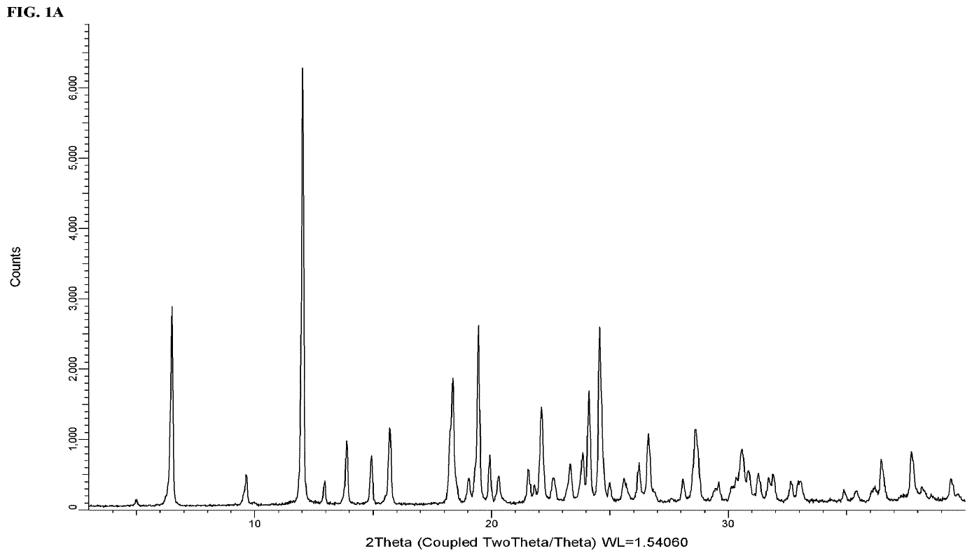

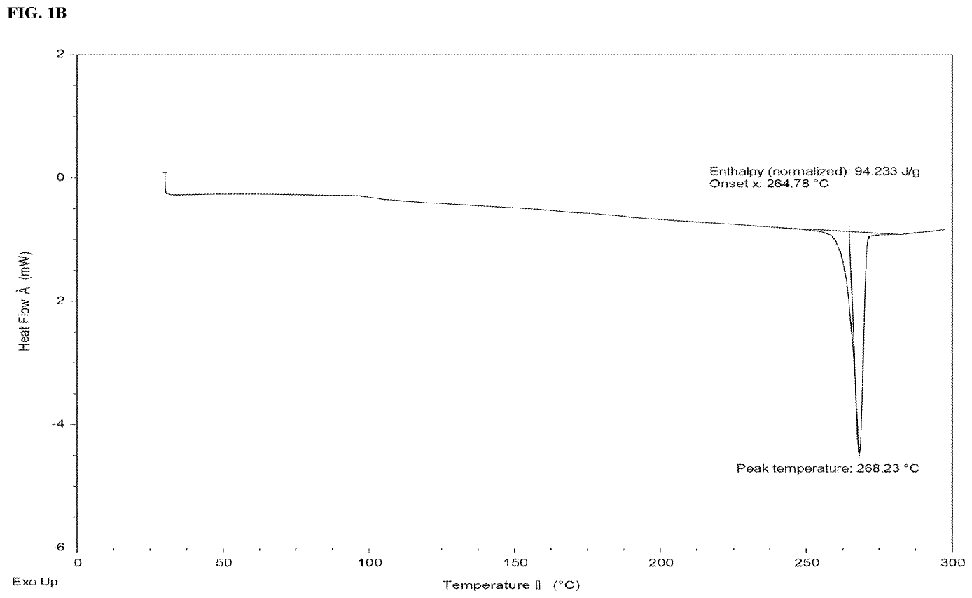

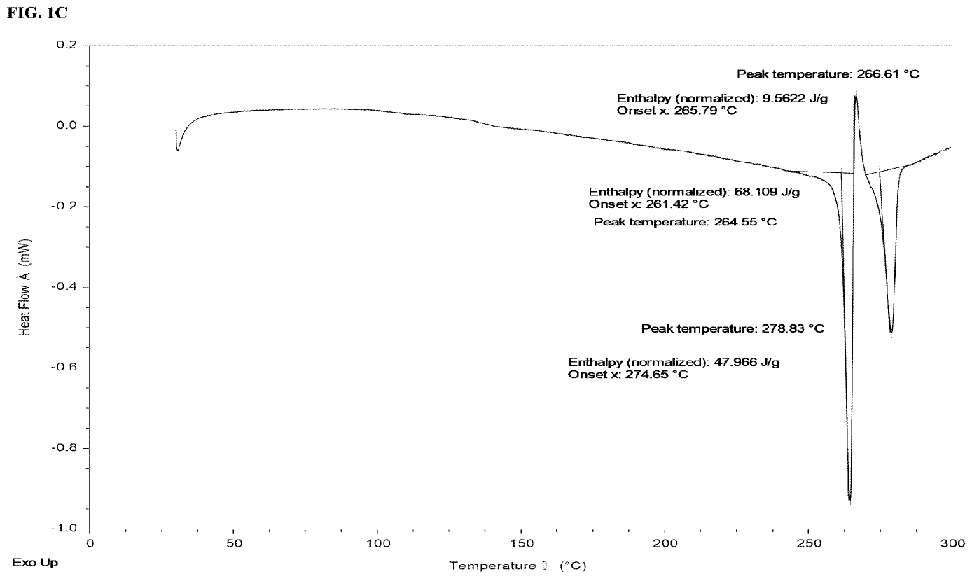

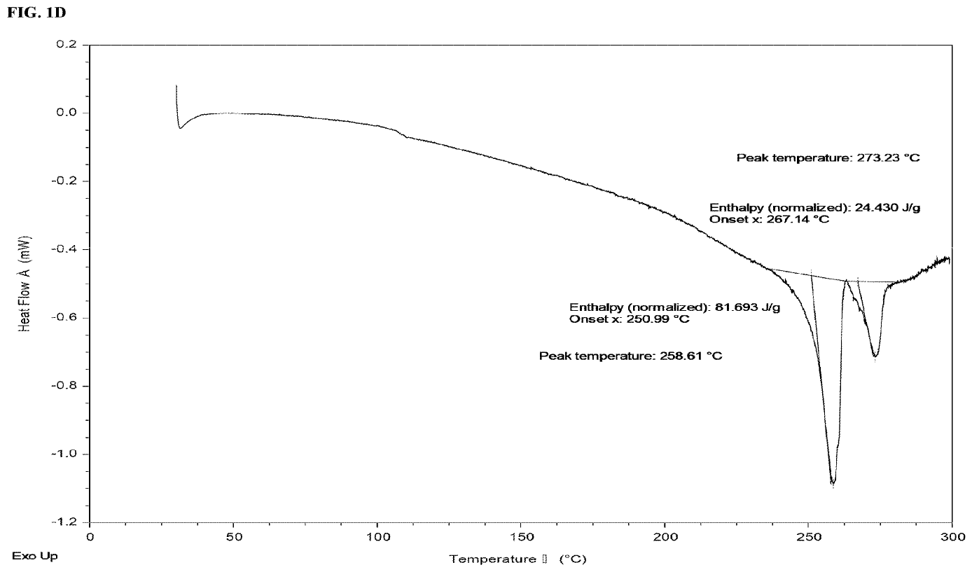

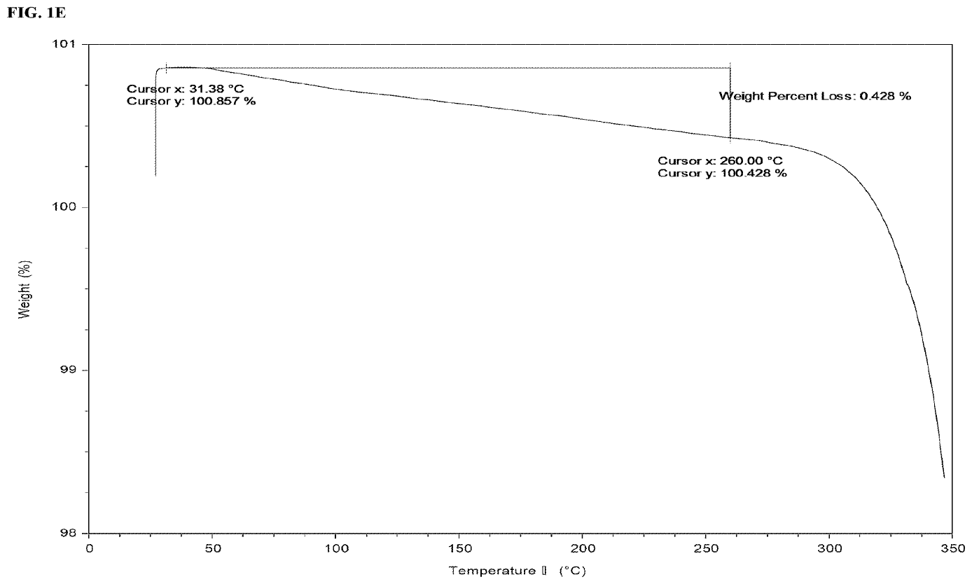

In some embodiments, the solid crystalline form of Compound I-1 is Form A. In some embodiments, Form A of Compound I-1 may be characterized by a powder X-ray diffraction pattern with at least two characteristic peaks, in degrees 2θ, each selected from the group consisting of about 12.0 2θ, about 6.5 2θ and about 19.5 2θ. In some embodiments, Form A of Compound I-1 may be characterized by a powder X-ray diffraction pattern with at least two characteristic peaks, in degrees 2θ, each selected from the group consisting of about 12.0 2θ, about 6.5 2θ, about 19.5 2θ, about 24.6 2θ, about 18.4 2θ, about 24.1 2θ and about 22.1 2θ. In some embodiments, Form A of Compound I-1 may be characterized by a powder X-ray diffraction pattern with at least three characteristic peaks, in degrees 2θ, each selected from the group consisting of about 12.0 2θ, about 6.5 2θ, about 19.5 2θ, about 24.6 2θ, about 18.4 2θ, about 24.1 2θ and about 22.1 2θ. In some embodiments, Form A of Compound I-1 may be characterized by a powder X-ray diffraction pattern with at least four characteristic peaks, in degrees 2θ, each selected from the group consisting of about 12.0 2θ, about 6.5 2θ, about 19.5 2θ, about 24.6 2θ, about 18.4 2θ, about 24.1 2θ and about 22.1 2θ. In some embodiments, Form A of Compound I-1 may be characterized by a powder X-ray diffraction pattern with at least five characteristic peaks, in degrees 2θ, each selected from the group consisting of about 12.0 2θ, about 6.5 2θ, about 19.5 2θ, about 24.6 2θ, about 18.4 2θ, about 24.1 2θ and about 22.1 2θ. In some embodiments, Form A of Compound I-1 may be characterized by a powder X-ray diffraction pattern with at least six characteristic peaks, in degrees 2θ, each selected from the group consisting of about 12.0 2θ, about 6.5 2θ, about 19.5 2θ, about 24.6 2θ, about 18.4 2θ, about 24.1 2θ and about 22.1 2θ. In some embodiments, Form A of Compound I-1 may be characterized by a powder X-ray diffraction pattern comprising characteristic peaks at about 12.0 2θ, about 6.5 2θ, about 19.5 2θ, about 24.6 2θ, about 18.4 2θ, about 24.1 2θ and about 22.1 2θ. In some embodiments, Form A of Compound I-1 has a X-Ray diffraction pattern substantially similar to that depicted in FIG. 1A. In some embodiments, Form A of Compound I-1 may be characterized by a powder X-ray diffraction pattern with at least two characteristic peaks, in degrees 2θ, each selected from the group consisting of the peaks listed in Table 1.1. In some embodiments, Form A of Compound I-1 may be characterized by a powder X-ray diffraction pattern with at least three characteristic peaks, in degrees 2θ, each selected from the group consisting of the peaks listed in Table 1.1. In some embodiments, Form A of Compound I-1 may be characterized by a powder X-ray diffraction pattern with at least four characteristic peaks, in degrees 2θ, each selected from the group consisting of the peaks listed in Table 1.1. In some embodiments, Form A of Compound I-1 may be characterized by a powder X-ray diffraction pattern with at least five characteristic peaks, in degrees 2θ, each selected from the group consisting of the peaks listed in Table 1.1. In some embodiments, Form A of Compound I-1 may be characterized by a powder X-ray diffraction pattern with at least six characteristic peaks, in degrees 2θ, each selected from the group consisting of the peaks listed in Table 1.1. In some embodiments, Form A of Compound I-1 may be characterized by a powder X-ray diffraction pattern with at least seven characteristic peaks, in degrees 2θ, each selected from the group consisting of the peaks listed in Table 1.1. Table 1.1 I-1 Form A XRPD peak listing (the angle 20 is within ±0.2).

TABLE 1.1
I-1 Form A XRPD peak listing (the angle 2θ is within ± 0.2).

| Index | Angle | d Value | Rel. Intensity |
|---|---|---|---|
| 1 | 9.973 | 8.86192 | 0.50% |
| 2 | 5.013 | 17.61308 | 1.20% |
| 3 | 35.414 | 2.53266 | 2.40% |
| 4 | 26.834 | 3.31978 | 2.60% |
| 5 | 34.896 | 2.56902 | 2.60% |
| 6 | 29.473 | 3.02827 | 2.70% |
| 7 | 38.18 | 2.35527 | 2.90% |
| 8 | 36.189 | 2.48017 | 3.20% |
| 9 | 21.809 | 4.07203 | 3.30% |
| 10 | 24.995 | 3.55959 | 3.80% |
| 11 | 29.597 | 3.01577 | 4.20% |
| 12 | 32.648 | 2.74063 | 4.50% |
| 13 | 33.052 | 2.70807 | 4.60% |
| 14 | 28.093 | 3.17375 | 4.90% |
| 15 | 25.612 | 3.47529 | 5.00% |
| 16 | 39.415 | 2.2843 | 5.00% |
| 17 | 12.96 | 6.82564 | 5.00% |
| 18 | 30.351 | 2.94256 | 5.10% |
| 19 | 22.622 | 3.92733 | 5.10% |
| 20 | 31.709 | 2.81956 | 5.20% |
| 21 | 19.047 | 4.65567 | 5.40% |
| 22 | 20.309 | 4.36912 | 6.00% |
| 23 | 31.9 | 2.80311 | 6.00% |
| 24 | 31.277 | 2.85757 | 6.10% |
| 25 | 9.659 | 9.14935 | 6.60% |
| 26 | 30.854 | 2.89575 | 6.70% |
| 27 | 21.573 | 4.11603 | 7.10% |
| 28 | 23.331 | 3.80957 | 8.10% |
| 29 | 26.23 | 3.39475 | 8.50% |
| 30 | 36.472 | 2.46158 | 9.30% |
| 31 | 23.855 | 3.72715 | 10.50% |
| 32 | 19.933 | 4.45064 | 10.70% |
| 33 | 14.936 | 5.92659 | 10.80% |
| 34 | 37.741 | 2.38165 | 10.80% |
| 35 | 30.582 | 2.92088 | 11.70% |
| 36 | 13.888 | 6.37134 | 14.50% |
| 37 | 26.633 | 3.34438 | 15.30% |
| 38 | 28.62 | 3.11653 | 16.40% |
| 39 | 15.711 | 5.63611 | 17.40% |
| 40 | 22.116 | 4.01604 | 21.10% |
| 41 | 24.12 | 3.68674 | 24.90% |
| 42 | 18.364 | 4.82721 | 28.70% |
| 43 | 24.576 | 3.61941 | 39.30% |
| 44 | 19.454 | 4.55925 | 40.70% |
| 45 | 6.51 | 13.56587 | 44.90% |
| 46 | 12.032 | 7.34974 | 100.00% |

As used herein, the term “about” in the context of peaks at degrees 2θ means that a peak can be the given 2θ value±0.2, or the given 2θ value±0.1, or the given value. For example, a peak of “about 12.0 2θ” means a peak can be 11.8 2θ, 11.9 2θ, 12.0 2θ, 12.1 2θ, or 12.2 2θ.

In some embodiments, Form A of Compound I-1 has a differential scanning calorimetry (DSC) pattern substantially similar to that depicted in FIG. 1B. In some embodiments, Form A of Compound I-1 has a differential scanning calorimetry (DSC) pattern substantially similar to that depicted in FIG. 1C. In some embodiments, Form A of Compound I-1 has a differential scanning calorimetry (DSC) pattern substantially similar to that depicted in FIG. 1D. In some embodiments, Form A of Compound I-1 has a thermogravimetric analysis (TGA) pattern substantially similar to that depicted in FIG. 1E. In some embodiments, Form A of Compound I-1 can be characterized by substantial similarity to two or more of these figures simultaneously.

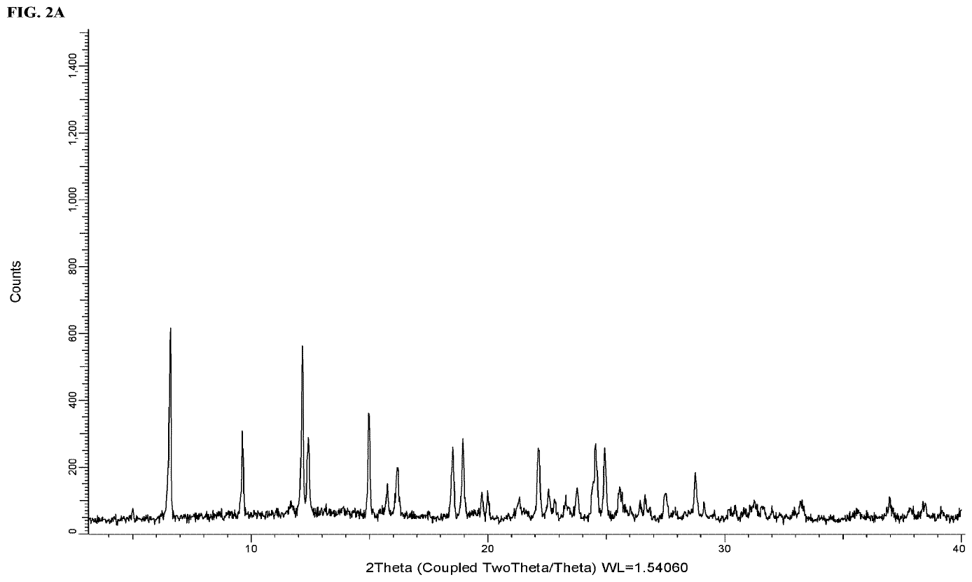

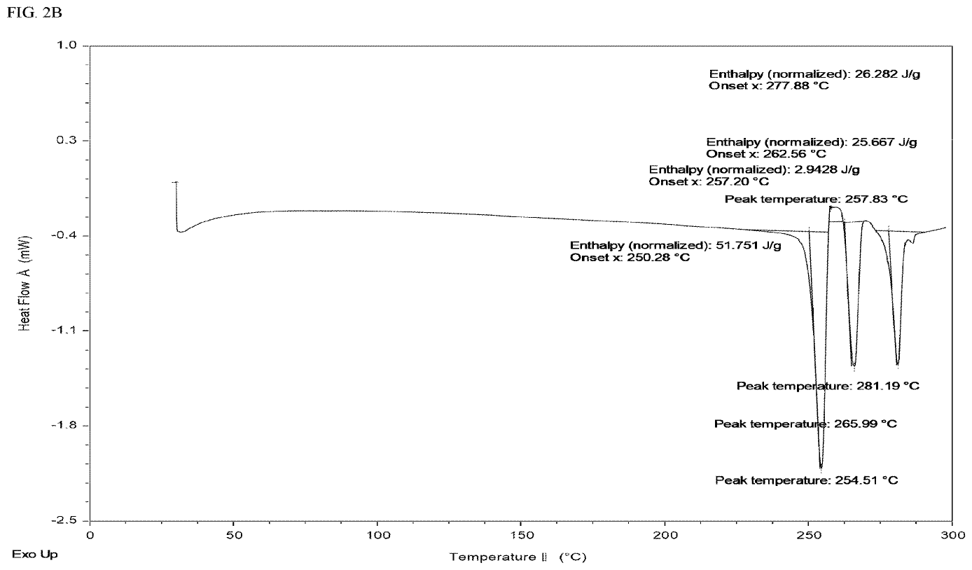

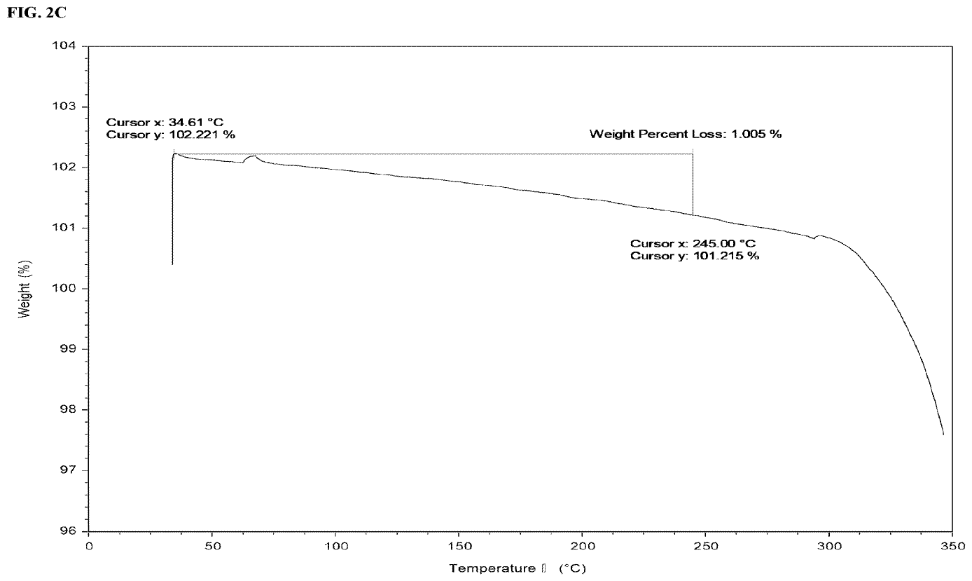

In some embodiments, the solid crystalline form of Compound I-1 is Form B. In some embodiments, Form B of Compound I-1 may be characterized by a powder X-ray diffraction pattern with at least two characteristic peaks, in degrees 2θ, each selected from the group consisting of about 6.6 2θ, about 12.2 2θ and about 15.0 2θ. In some embodiments, Form B of Compound I-1 may be characterized by a powder X-ray diffraction pattern with at least two characteristic peaks, in degrees 2θ, each selected from the group consisting of about 6.6 2θ, about 12.2 2θ, about 15.0 2θ, about 9.6 2θ, about 19.0 2θ, about 12.4 2θ and about 24.6 2θ. In some embodiments, Form B of Compound I-1 may be characterized by a powder X-ray diffraction pattern with at least three characteristic peaks, in degrees 2θ, each selected from the group consisting of about 6.6 2θ, about 12.2 2θ, about 15.0 2θ, about 9.6 2θ, about 19.0 2θ, about 12.4 2θ and about 24.6 2θ. In some embodiments, Form B of Compound I-1 may be characterized by a powder X-ray diffraction pattern with at least four characteristic peaks, in degrees 2θ, each selected from the group consisting of about 6.6 2θ, about 12.2 2θ, about 15.0 2θ, about 9.6 2θ, about 19.0 2θ, about 12.4 2θ and about 24.6 2θ. In some embodiments, Form B of Compound I-1 may be characterized by a powder X-ray diffraction pattern with at least five characteristic peaks, in degrees 2θ, each selected from the group consisting of about 6.6 2θ, about 12.2 2θ, about 15.0 2θ, about 9.6 2θ, about 19.0 2θ, about 12.4 2θ and about 24.6 2θ. In some embodiments, Form B of Compound I-1 may be characterized by a powder X-ray diffraction pattern with at least six characteristic peaks, in degrees 2θ, each selected from the group consisting of about 6.6 2θ, about 12.2 2θ, about 15.0 2θ, about 9.6 2θ, about 19.0 2θ, about 12.4 2θ and about 24.6 2θ. In some embodiments, Form B of Compound I-1 may be characterized by a powder X-ray diffraction pattern comprising characteristic peaks at about 6.6 2θ, about 12.2 2θ, about 15.0 2θ, about 9.6 2θ, about 19.0 2θ, about 12.4 2θ and about 24.6 2θ. In some embodiments, Form B of Compound I-1 has a X-Ray diffraction pattern substantially similar to that depicted in FIG. 2A. In some embodiments, Form B of Compound I-1 may be characterized by a powder X-ray diffraction pattern with at least two characteristic peaks, in degrees 2θ, each selected from the group consisting of the peaks listed in Table 1.2. In some embodiments, Form B of Compound I-1 may be characterized by a powder X-ray diffraction pattern with at least three characteristic peaks, in degrees 2θ, each selected from the group consisting of the peaks listed in Table 1.2. In some embodiments, Form B of Compound I-1 may be characterized by a powder X-ray diffraction pattern with at least four characteristic peaks, in degrees 2θ, each selected from the group consisting of the peaks listed in Table 1.2. In some embodiments, Form B of Compound I-1 may be characterized by a powder X-ray diffraction pattern with at least five characteristic peaks, in degrees 2θ, each selected from the group consisting of the peaks listed in Table 1.2. In some embodiments, Form B of Compound I-1 may be characterized by a powder X-ray diffraction pattern with at least six characteristic peaks, in degrees 2θ, each selected from the group consisting of the peaks listed in Table 1.2. In some embodiments, Form B of Compound I-1 may be characterized by a powder X-ray diffraction pattern with at least seven characteristic peaks, in degrees 2θ, each selected from the group consisting of the peaks listed in Table 1.2.

TABLE 1.2
I-1 Form B XRPD peak listing (the angle 2θ is within ± 0.2).

| Index | Angle | d Value | Rel. Intensity |
|---|---|---|---|
| 1 | 37.843 | 2.37548 | 4.70% |
| 2 | 31.598 | 2.82926 | 6.00% |
| 3 | 26.845 | 3.31846 | 6.20% |
| 4 | 26.027 | 3.42085 | 6.30% |
| 5 | 32.95 | 2.71619 | 6.60% |
| 6 | 32.011 | 2.79372 | 7.00% |
| 7 | 30.453 | 2.93298 | 7.20% |
| 8 | 11.703 | 7.5558 | 7.30% |
| 9 | 38.48 | 2.33763 | 7.70% |
| 10 | 26.659 | 3.34118 | 8.30% |
| 11 | 29.137 | 3.06233 | 8.50% |
| 12 | 33.252 | 2.69222 | 9.10% |
| 13 | 26.435 | 3.36888 | 9.20% |
| 14 | 22.831 | 3.89194 | 9.50% |
| 15 | 31.256 | 2.85946 | 9.70% |
| 16 | 36.984 | 2.42864 | 10.20% |
| 17 | 21.334 | 4.1616 | 10.70% |
| 18 | 23.302 | 3.81427 | 11.10% |
| 19 | 22.582 | 3.93431 | 12.90% |
| 20 | 19.762 | 4.48879 | 13.70% |
| 21 | 27.505 | 3.24021 | 13.70% |
| 22 | 20.005 | 4.4349 | 14.40% |
| 23 | 25.567 | 3.48134 | 15.40% |
| 24 | 23.776 | 3.73932 | 16.00% |
| 25 | 15.755 | 5.62023 | 16.50% |
| 26 | 28.774 | 3.10022 | 22.00% |
| 27 | 16.187 | 5.47123 | 25.40% |
| 28 | 18.522 | 4.78656 | 36.40% |
| 29 | 22.145 | 4.01085 | 36.60% |
| 30 | 24.946 | 3.56654 | 36.60% |
| 31 | 24.567 | 3.6207 | 39.10% |
| 32 | 12.42 | 7.12084 | 40.10% |
| 33 | 18.955 | 4.6781 | 41.90% |
| 34 | 9.647 | 9.16076 | 42.30% |
| 35 | 14.982 | 5.90848 | 53.60% |
| 36 | 12.17 | 7.26695 | 87.10% |
| 37 | 6.59 | 13.40144 | 100.00% |

In some embodiments, Form B of Compound I-1 has a differential scanning calorimetry (DSC) pattern substantially similar to that depicted in FIG. 2B. In some embodiments, Form B of Compound I-1 has a thermogravimetric analysis (TGA) pattern substantially similar to that depicted in FIG. 2C. In some embodiments, Form B of Compound I-1 can be characterized by substantial similarity to two or more of these figures simultaneously.

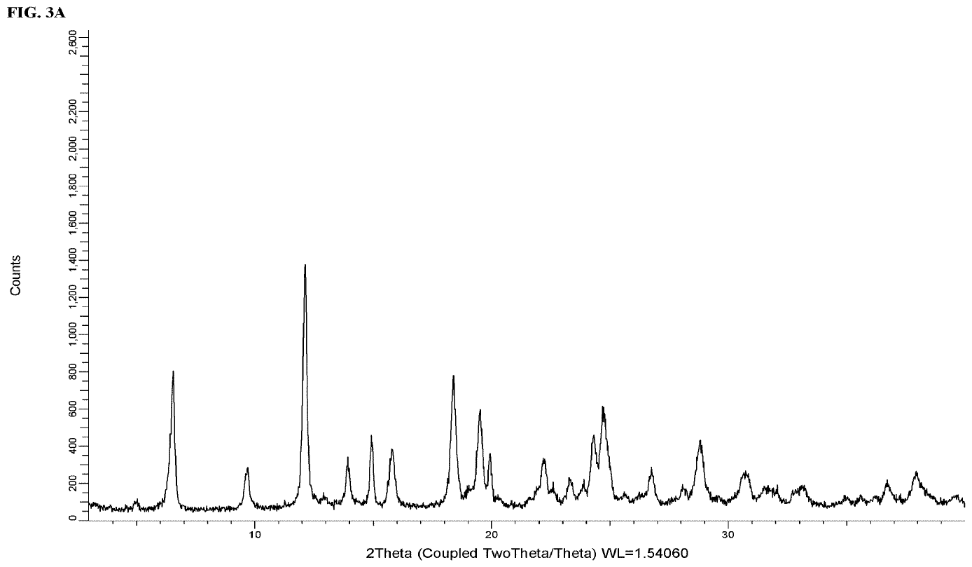

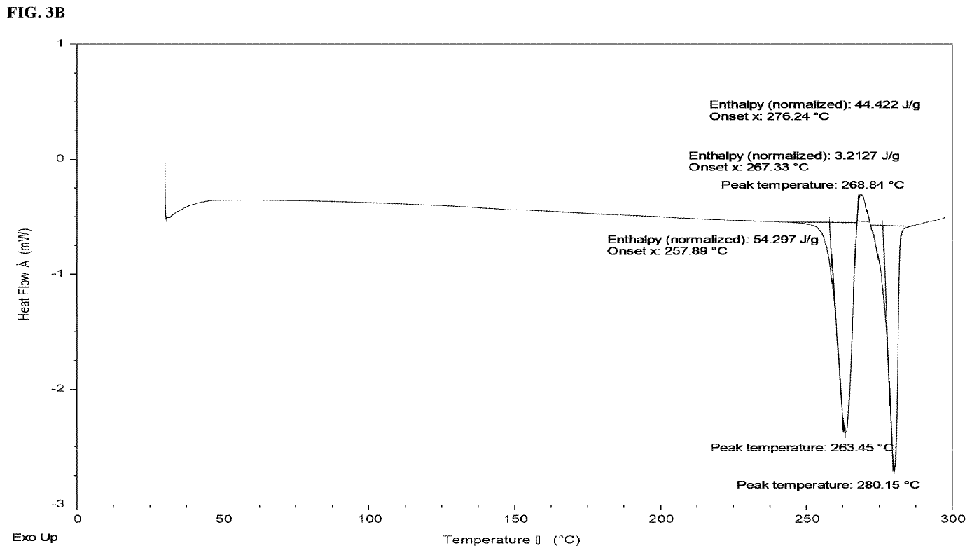

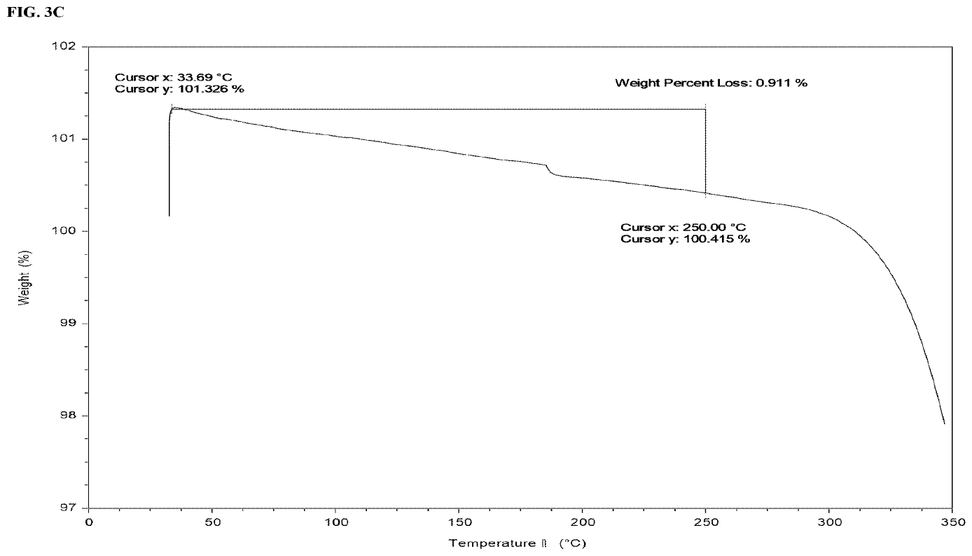

In some embodiments, the solid crystalline form of Compound I-1 is Form C. In some embodiments, Form C of Compound I-1 may be characterized by a powder X-ray diffraction pattern with at least two characteristic peaks, in degrees 2θ, each selected from the group consisting of about 12.1 2θ, about 6.6 2θ and about 18.4 2θ. In some embodiments, Form C of Compound I-1 may be characterized by a powder X-ray diffraction pattern with at least two characteristic peaks, in degrees 2θ, each selected from the group consisting of about 12.1 2θ, about 6.6 2θ, about 18.4 2θ, about 19.5 2θ, about 24.7 2θ, about 14.9 2θ and about 24.3 2θ. In some embodiments, Form C of Compound I-1 may be characterized by a powder X-ray diffraction pattern with at least three characteristic peaks, in degrees 2θ, each selected from the group consisting of about 12.1 2θ, about 6.6 2θ, about 18.4 2θ, about 19.5 2θ, about 24.7 2θ, about 14.9 2θ and about 24.3 2θ. In some embodiments, Form C of Compound I-1 may be characterized by a powder X-ray diffraction pattern with at least four characteristic peaks, in degrees 2θ, each selected from the group consisting of about 12.1 2θ, about 6.6 2θ, about 18.4 2θ, about 19.5 2θ, about 24.7 2θ, about 14.9 2θ and about 24.3 2θ. In some embodiments, Form C of Compound I-1 may be characterized by a powder X-ray diffraction pattern with at least five characteristic peaks, in degrees 2θ, each selected from the group consisting of about 12.1 2θ, about 6.6 2θ, about 18.4 2θ, about 19.5 2θ, about 24.7 2θ, about 14.9 2θ and about 24.3 2θ. In some embodiments, Form C of Compound I-1 may be characterized by a powder X-ray diffraction pattern with at least six characteristic peaks, in degrees 2θ, each selected from the group consisting of about 12.1 2θ, about 6.6 2θ, about 18.4 2θ, about 19.5 2θ, about 24.7 2θ, about 14.9 2θ and about 24.3 2θ. In some embodiments, Form C of Compound I-1 may be characterized by a powder X-ray diffraction pattern comprising characteristic peaks at about 12.1 2θ, about 6.6 2θ, about 18.4 2θ, about 19.5 2θ, about 24.7 2θ, about 14.9 2θ and about 24.3 2θ. In some embodiments, Form C of Compound I-1 has a X-Ray diffraction pattern substantially similar to that depicted in FIG. 3A. In some embodiments, Form C of Compound I-1 may be characterized by a powder X-ray diffraction pattern with at least two characteristic peaks, in degrees 2θ, each selected from the group consisting of the peaks listed in Table 1.3. In some embodiments, Form C of Compound I-1 may be characterized by a powder X-ray diffraction pattern with at least three characteristic peaks, in degrees 2θ, each selected from the group consisting of the peaks listed in Table 1.3. In some embodiments, Form C of Compound I-1 may be characterized by a powder X-ray diffraction pattern with at least four characteristic peaks, in degrees 2θ, each selected from the group consisting of the peaks listed in Table 1.3. In some embodiments, Form C of Compound I-1 may be characterized by a powder X-ray diffraction pattern with at least five characteristic peaks, in degrees 2θ, each selected from the group consisting of the peaks listed in Table 1.3. In some embodiments, Form C of Compound I-1 may be characterized by a powder X-ray diffraction pattern with at least six characteristic peaks, in degrees 2θ, each selected from the group consisting of the peaks listed in Table 1.3. In some embodiments, Form C of Compound I-1 may be characterized by a powder X-ray diffraction pattern with at least seven characteristic peaks, in degrees 2θ, each selected from the group consisting of the peaks listed in Table 1.3.

TABLE 1.3
I-1 Form C XRPD peak listing (the angle 2θ is within ± 0.2).

| Index | Angle | d Value | Rel. Intensity |
|---|---|---|---|
| 1 | 39.655 | 2.27101 | 1.90% |
| 2 | 21.657 | 4.10012 | 2.40% |
| 3 | 25.626 | 3.4734 | 2.40% |
| 4 | 26.316 | 3.38384 | 2.90% |
| 5 | 35.598 | 2.51994 | 3.20% |
| 6 | 5.026 | 17.5687 | 3.70% |
| 7 | 12.92 | 6.84629 | 3.90% |
| 8 | 20.281 | 4.37506 | 4.00% |
| 9 | 32.088 | 2.78718 | 4.10% |
| 10 | 32.839 | 2.72511 | 5.30% |
| 11 | 31.586 | 2.8303 | 5.50% |
| 12 | 28.087 | 3.17443 | 6.00% |
| 13 | 23.883 | 3.72289 | 6.20% |
| 14 | 34.929 | 2.56671 | 6.20% |
| 15 | 22.605 | 3.93032 | 7.00% |
| 16 | 33.127 | 2.70204 | 7.00% |
| 17 | 23.326 | 3.81049 | 8.20% |
| 18 | 36.711 | 2.44609 | 8.70% |
| 19 | 30.615 | 2.91781 | 11.10% |
| 20 | 30.762 | 2.90417 | 11.10% |
| 21 | 37.966 | 2.36805 | 11.20% |
| 22 | 26.763 | 3.32844 | 14.20% |
| 23 | 9.683 | 9.1272 | 16.80% |
| 24 | 13.922 | 6.35612 | 18.00% |
| 25 | 22.187 | 4.00345 | 18.40% |
| 26 | 19.938 | 4.44972 | 21.10% |
| 27 | 15.807 | 5.60214 | 23.30% |
| 28 | 28.81 | 3.09636 | 25.70% |
| 29 | 24.318 | 3.65721 | 26.90% |
| 30 | 14.935 | 5.92706 | 28.30% |
| 31 | 24.739 | 3.59598 | 38.10% |
| 32 | 19.512 | 4.54571 | 38.60% |
| 33 | 18.395 | 4.81919 | 53.30% |
| 34 | 6.551 | 13.4819 | 56.80% |
| 35 | 12.123 | 7.29477 | 100.00% |

In some embodiments, Form C of Compound I-1 has a differential scanning calorimetry (DSC) pattern substantially similar to that depicted in FIG. 3B. In some embodiments, Form C of Compound I-1 has a thermogravimetric analysis (TGA) pattern substantially similar to that depicted in FIG. 3C. In some embodiments, Form C of Compound I-1 can be characterized by substantial similarity to two or more of these figures simultaneously.

### Compound I-2

In some embodiments, a compound of Formula (I) is Compound I-2:

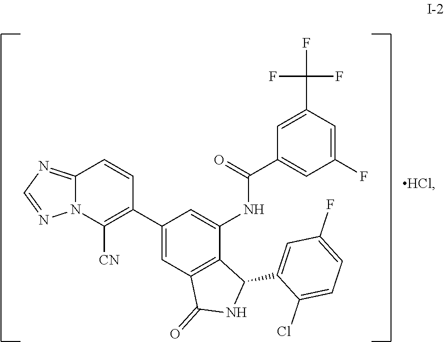

or a solvate thereof.

In some embodiments, Compound I-2 is an anhydrous solid.

In some embodiments, Compound I-2 is an amorphous solid. In other embodiments, Compound I-2 is a crystalline solid. In some embodiments, Compound I-2 is a mixture of amorphous solid form and crystalline solid form.

In some embodiments, the present invention provides a form of compound I-2 substantially free of impurities. As used herein, the term “substantially free of impurities” means that the compound contains no significant amount of extraneous matter. Such extraneous matter may include different forms of compound I-2, residual solvents, or any other impurities that may result from the preparation of, and/or isolation of, compound I-2.

In some embodiments, compound I-2, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound I-2, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound I-2, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound I-2, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound I-2 is also meant to include all tautomeric forms of compound I-2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound I-2 is a crystalline solid. In other embodiments, compound I-2 is a crystalline solid substantially free of amorphous compound I-2. As used herein, the term “substantially free of amorphous compound I-2” means that the compound contains no significant amount of amorphous compound I-2. In certain embodiments, at least about 95% by weight of crystalline compound I-2 is present. In still other embodiments of the invention, at least about 99% by weight of crystalline compound I-2 is present.

It has been found that compound I-2 can exist in a variety of solid forms. Exemplary such forms include polymorphs such as those described herein.

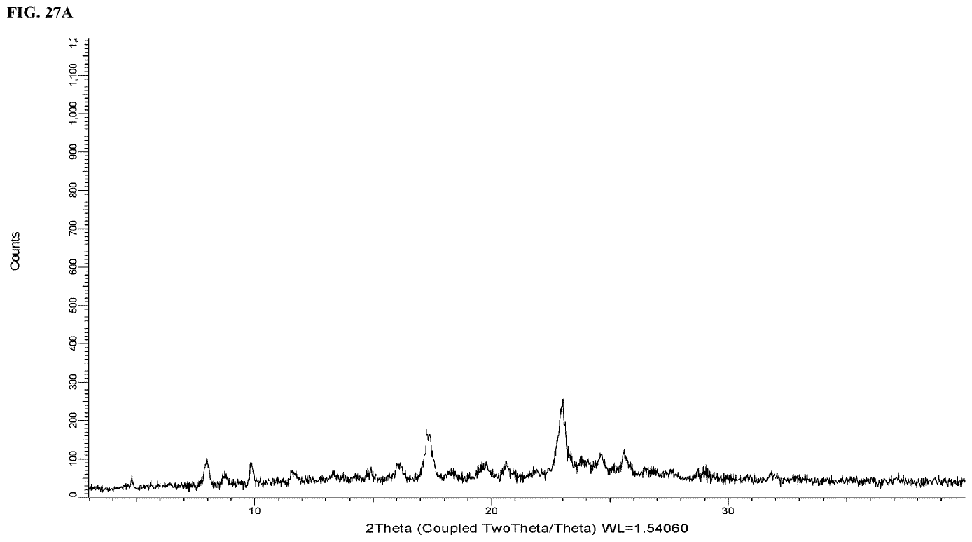

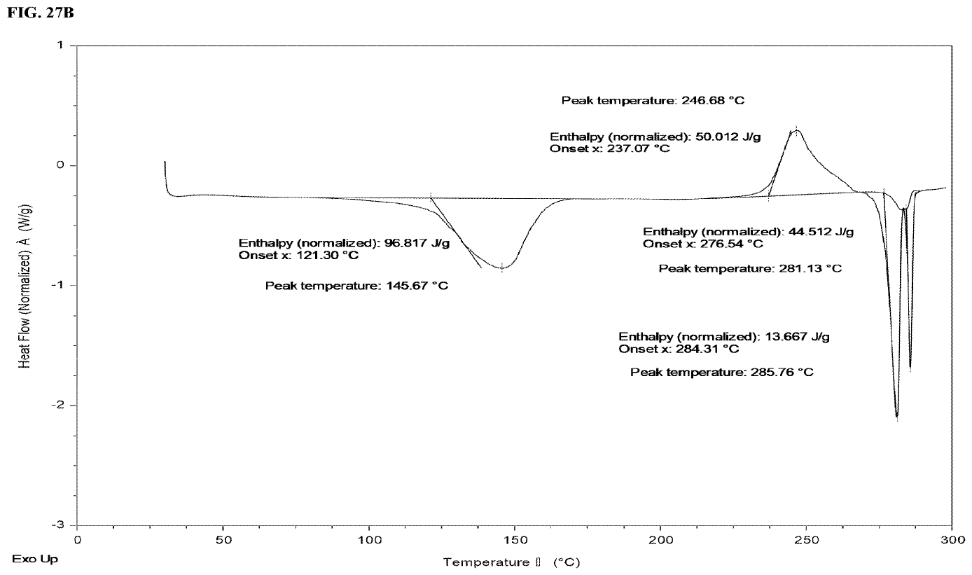

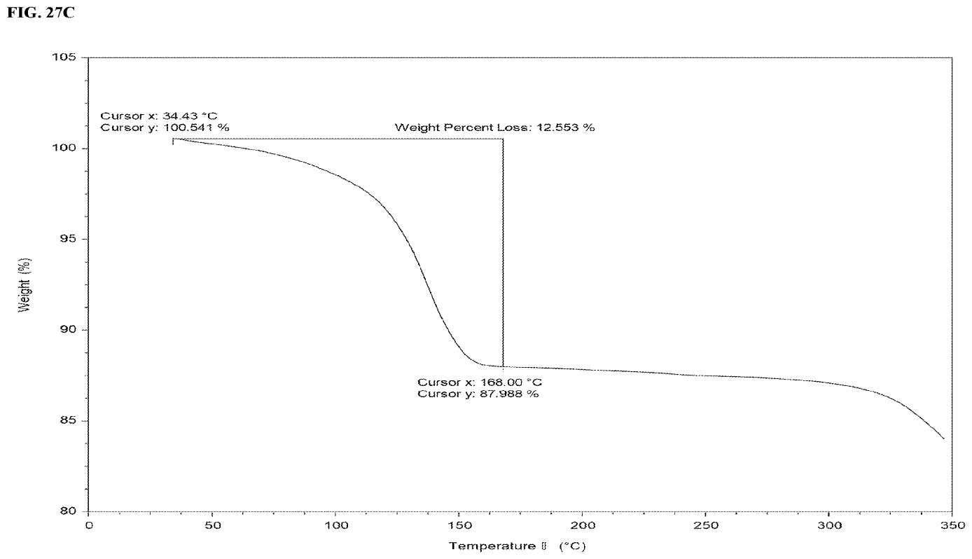

In some embodiments, the solid crystalline form of Compound I-2 is Form A. In some embodiments, Form A of Compound I-2 has a X-Ray diffraction pattern substantially similar to that depicted in FIG. 27A.

In some embodiments, Form A of Compound I-2 has a differential scanning calorimetry (DSC) pattern substantially similar to that depicted in FIG. 27B. In some embodiments, Form A of Compound I-2 has a thermogravimetric analysis (TGA) pattern substantially similar to that depicted in FIG. 27C. In some embodiments, Form A of Compound I-2 can be characterized by substantial similarity to two or more of these figures simultaneously.

### Compound I-3

In some embodiments, a compound of Formula (I) is Compound I-3:

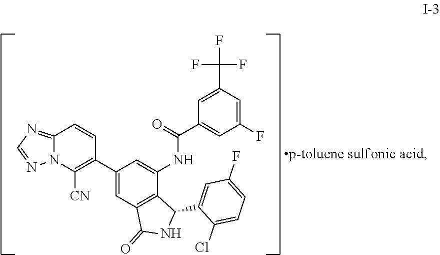

or a solvate thereof

In some embodiments, Compound I-3 is an anhydrous solid.

In some embodiments, Compound I-3 is an amorphous solid. In other embodiments, Compound I-3 is a crystalline solid. In some embodiments, Compound I-3 is a mixture of amorphous solid form and crystalline solid form.

In some embodiments, the present invention provides a form of compound I-3 substantially free of impurities. As used herein, the term “substantially free of impurities” means that the compound contains no significant amount of extraneous matter. Such extraneous matter may include different forms of compound I-3, residual solvents, or any other impurities that may result from the preparation of, and/or isolation of, compound I-3.

In some embodiments, compound I-3, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound I-3, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound I-3, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound I-3, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound I-3 is also meant to include all tautomeric forms of compound I-3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound I-3 is a crystalline solid. In other embodiments, compound I-3 is a crystalline solid substantially free of amorphous compound I-3. As used herein, the term “substantially free of amorphous compound I-3” means that the compound contains no significant amount of amorphous compound I-3. In certain embodiments, at least about 95% by weight of crystalline compound I-3 is present. In still other embodiments of the invention, at least about 99% by weight of crystalline compound I-3 is present.

It has been found that compound I-3 can exist in a variety of solid forms. Exemplary such forms include polymorphs such as those described herein.

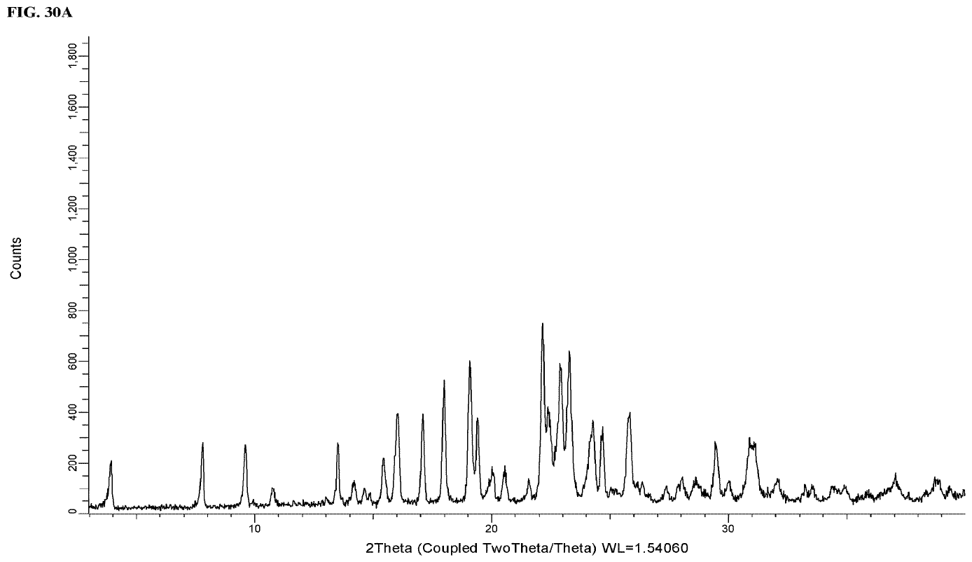

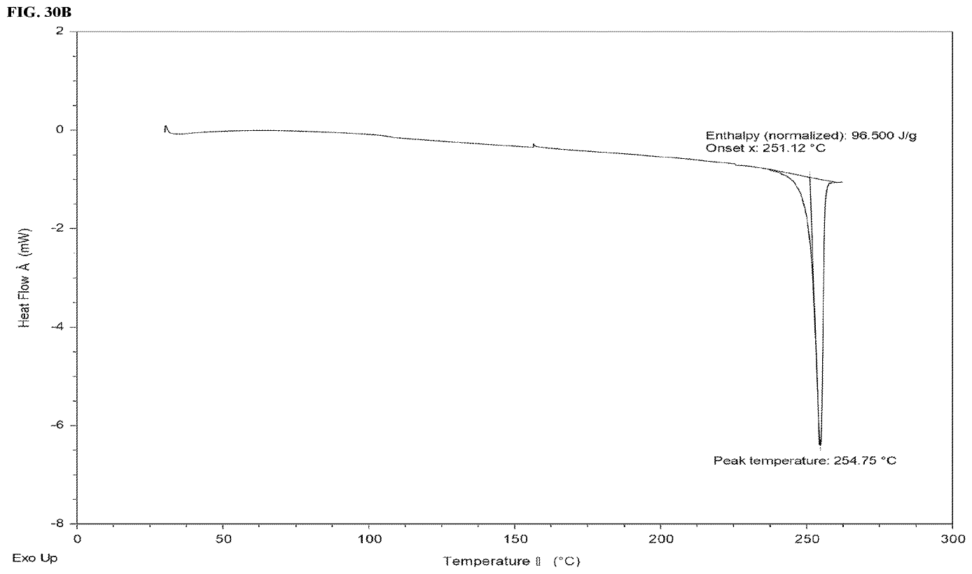

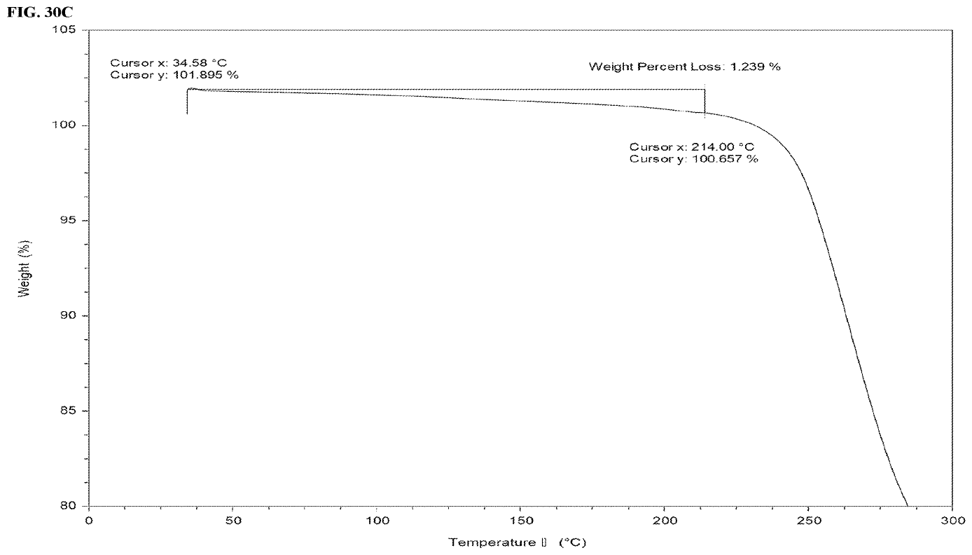

In some embodiments, the solid crystalline form of Compound I-3 is Form A. In some embodiments, Form A of Compound I-3 has a X-Ray diffraction pattern substantially similar to that depicted in FIG. 30A.

In some embodiments, Form A of Compound I-3 has a differential scanning calorimetry (DSC) pattern substantially similar to that depicted in FIG. 30B. In some embodiments, Form A of Compound I-3 has a thermogravimetric analysis (TGA) pattern substantially similar to that depicted in FIG. 30C. In some embodiments, Form A of Compound I-3 can be characterized by substantial similarity to two or more of these figures simultaneously.

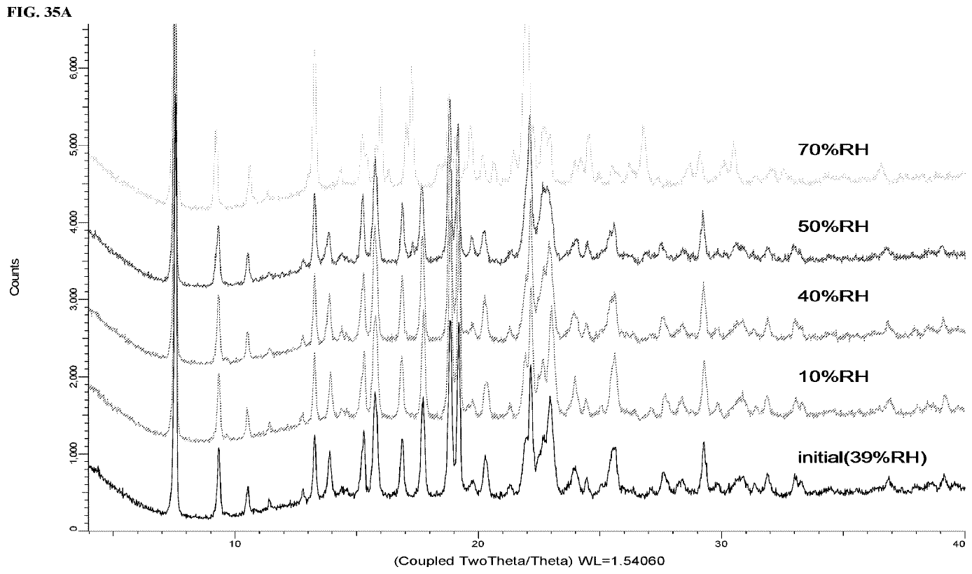

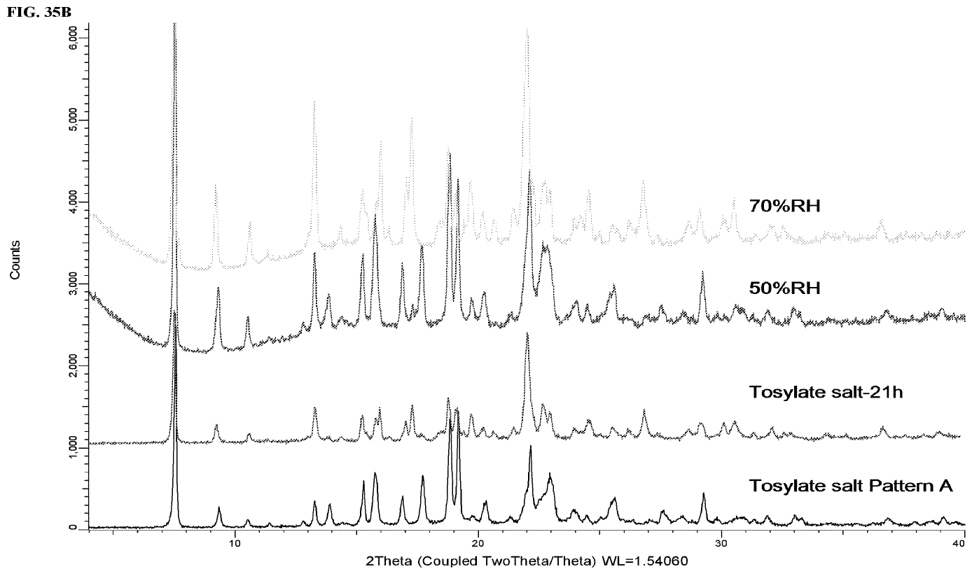

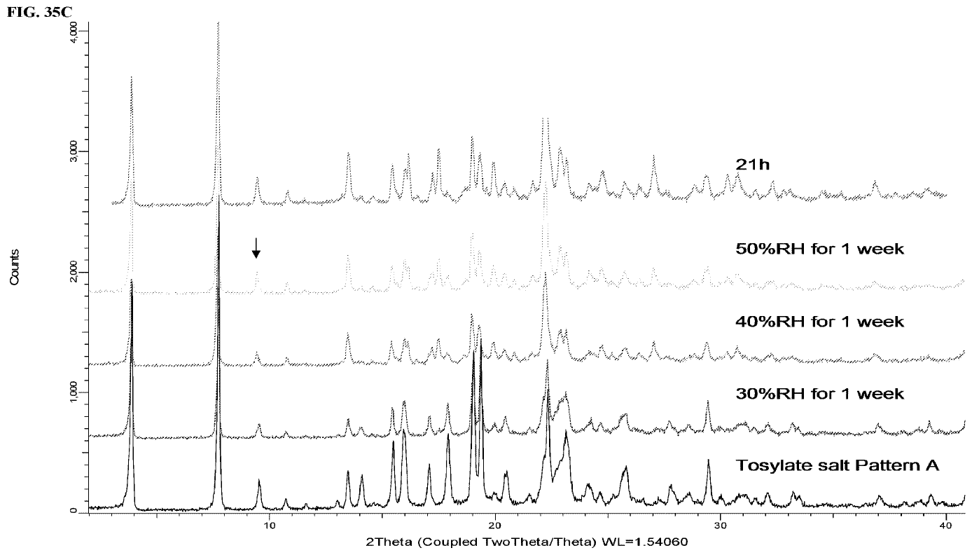

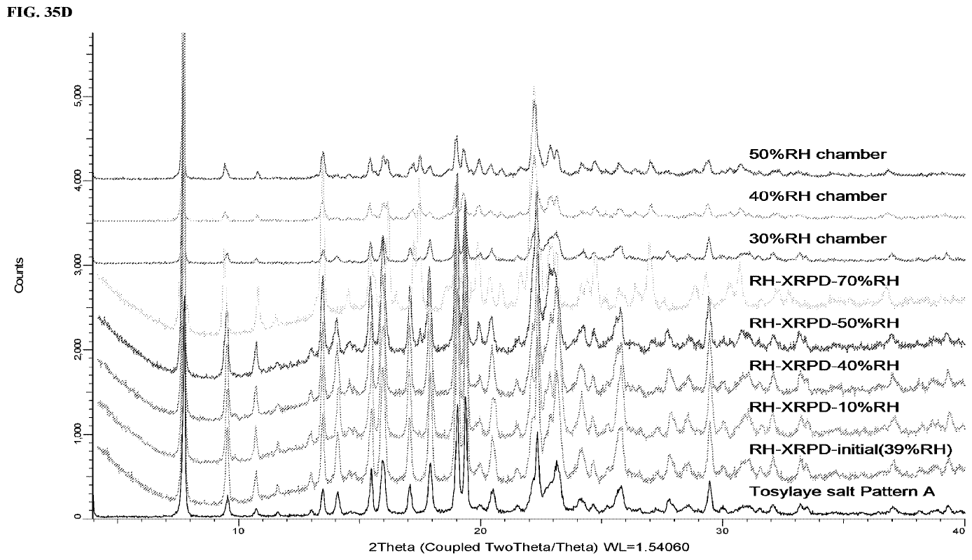

In some embodiments, the solid crystalline form of Compound I-3 is Form B. In some embodiments, Form B of Compound I-3 has a X-Ray diffraction pattern substantially similar to that depicted in FIG. 35A. In some embodiments, Form B of Compound I-3 has a X-Ray diffraction pattern substantially similar to that depicted in FIG. 35B. In some embodiments, Form B of Compound I-3 has a X-Ray diffraction pattern substantially similar to that depicted in FIG. 35C. In some embodiments, Form B of Compound I-3 has a X-Ray diffraction pattern substantially similar to that depicted in FIG. 35D. In some embodiments, Form B of Compound I-3 can be characterized by substantial similarity to two or more of these figures simultaneously.

### Compound I-4

In some embodiments, a compound of Formula (I) is Compound I-4:

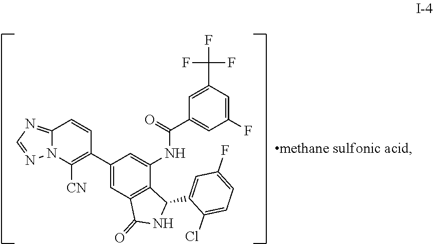

or a solvate thereof.

In some embodiments, Compound I-4 is an anhydrous solid.

In some embodiments, Compound I-4 is an amorphous solid. In other embodiments, Compound I-4 is a crystalline solid. In some embodiments, Compound I-4 is a mixture of amorphous solid form and crystalline solid form.

In some embodiments, the present invention provides a form of compound I-4 substantially free of impurities. As used herein, the term “substantially free of impurities” means that the compound contains no significant amount of extraneous matter. Such extraneous matter may include different forms of compound I-4, residual solvents, or any other impurities that may result from the preparation of, and/or isolation of, compound I-4.

In some embodiments, compound I-4, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound I-4, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound I-4, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound I-4, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound I-4 is also meant to include all tautomeric forms of compound I-4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound I-4 is a crystalline solid. In other embodiments, compound I-4 is a crystalline solid substantially free of amorphous compound I-4. As used herein, the term “substantially free of amorphous compound I-4” means that the compound contains no significant amount of amorphous compound I-4. In certain embodiments, at least about 95% by weight of crystalline compound I-4 is present. In still other embodiments of the invention, at least about 99% by weight of crystalline compound I-4 is present.

It has been found that compound I-4 can exist in a variety of solid forms. Exemplary such forms include polymorphs such as those described herein.

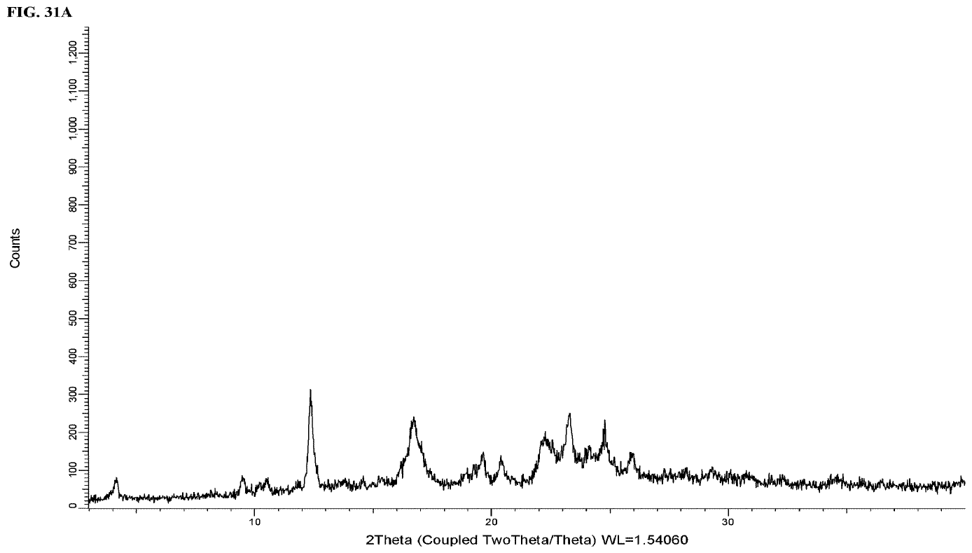

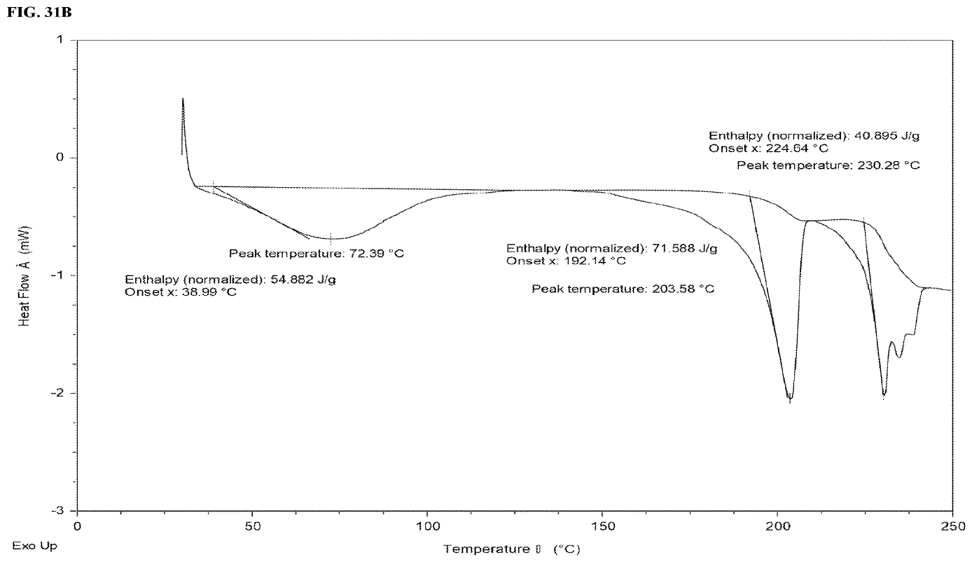

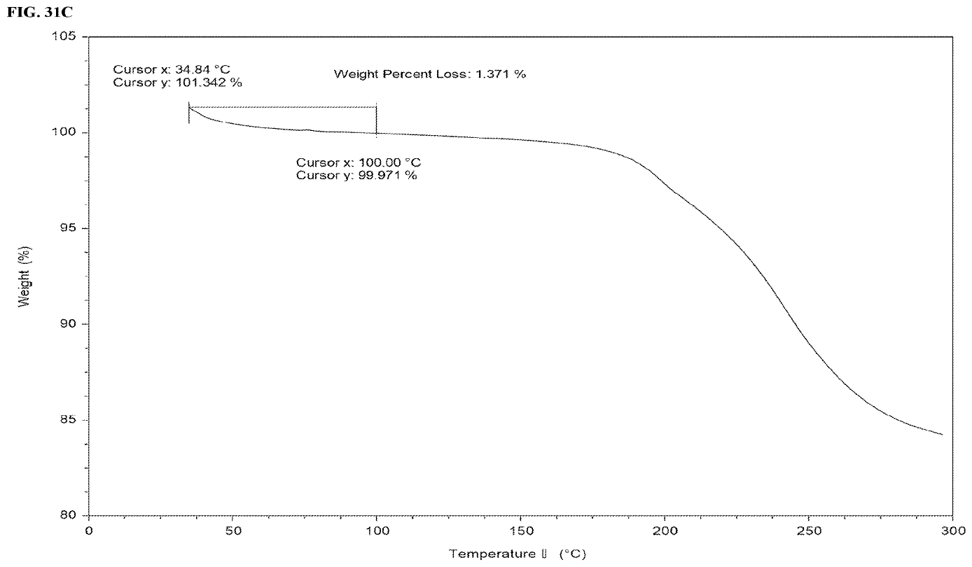

In some embodiments, the solid crystalline form of Compound I-4 is Form A. In some embodiments, Form A of Compound I-4 has a X-Ray diffraction pattern substantially similar to that depicted in FIG. 31A.

In some embodiments, Form A of Compound I-4 has a differential scanning calorimetry (DSC) pattern substantially similar to that depicted in FIG. 31B. In some embodiments, Form A of Compound I-4 has a thermogravimetric analysis (TGA) pattern substantially similar to that depicted in FIG. 31C. In some embodiments, Form A of Compound I-4 can be characterized by substantial similarity to two or more of these figures simultaneously.

### Compound I-5

In some embodiments, a compound of Formula (I) is Compound I-5:

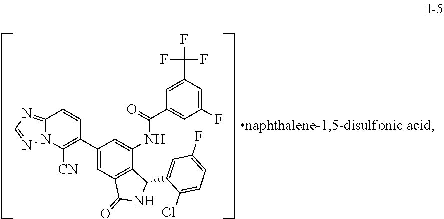

or a solvate thereof

In some embodiments, Compound I-5 is an anhydrous solid.

In some embodiments, Compound I-5 is an amorphous solid. In other embodiments, Compound I-5 is a crystalline solid. In some embodiments, Compound I-5 is a mixture of amorphous solid form and crystalline solid form.

In some embodiments, the present invention provides a form of compound I-5 substantially free of impurities. As used herein, the term “substantially free of impurities” means that the compound contains no significant amount of extraneous matter. Such extraneous matter may include different forms of compound I-5, residual solvents, or any other impurities that may result from the preparation of, and/or isolation of, compound I-5.

In some embodiments, compound I-5, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound I-5, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound I-5, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound I-5, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound I-5 is also meant to include all tautomeric forms of compound I-5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound I-5 is a crystalline solid. In other embodiments, compound I-5 is a crystalline solid substantially free of amorphous compound I-5. As used herein, the term “substantially free of amorphous compound I-5” means that the compound contains no significant amount of amorphous compound I-5. In certain embodiments, at least about 95% by weight of crystalline compound I-5 is present. In still other embodiments of the invention, at least about 99% by weight of crystalline compound I-5 is present.

It has been found that compound I-5 can exist in a variety of solid forms. Exemplary such forms include polymorphs such as those described herein.

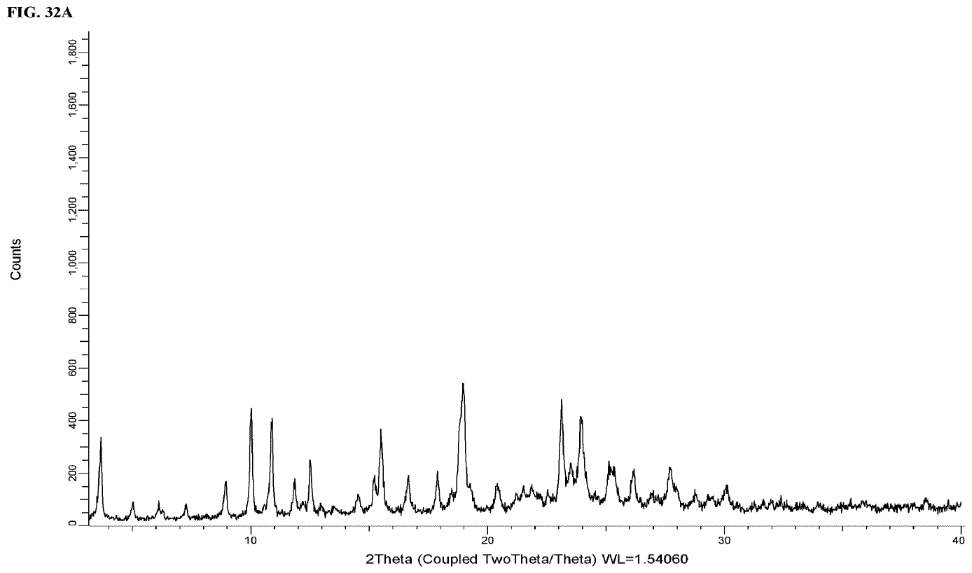

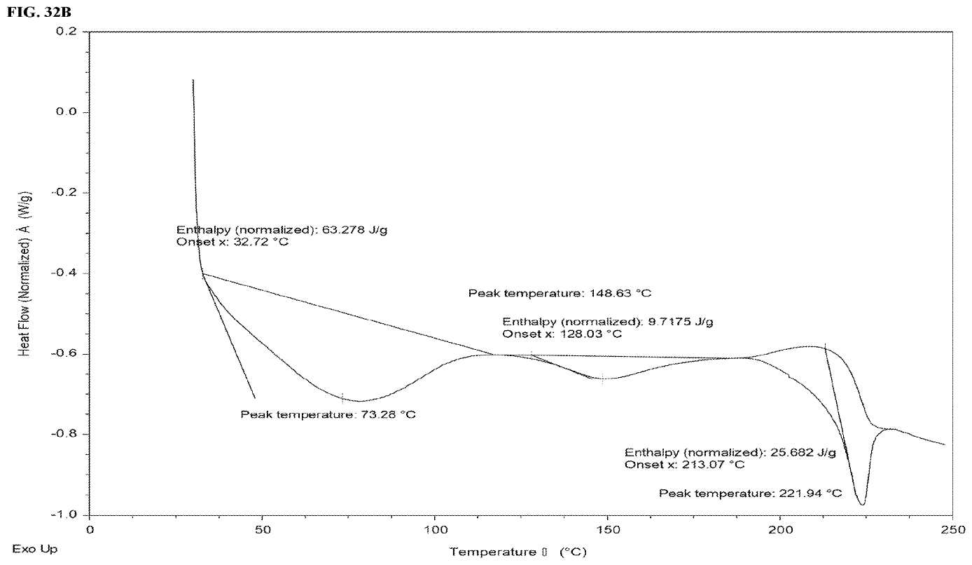

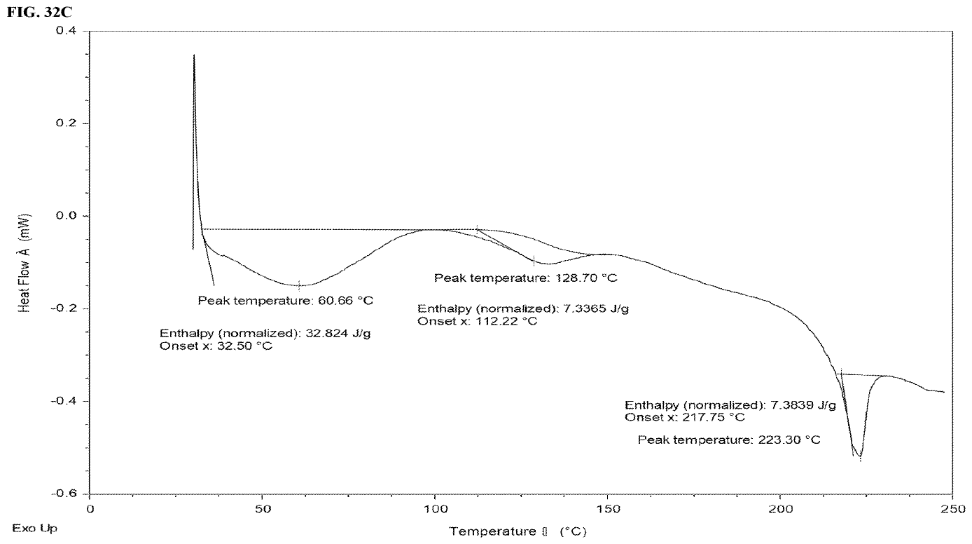

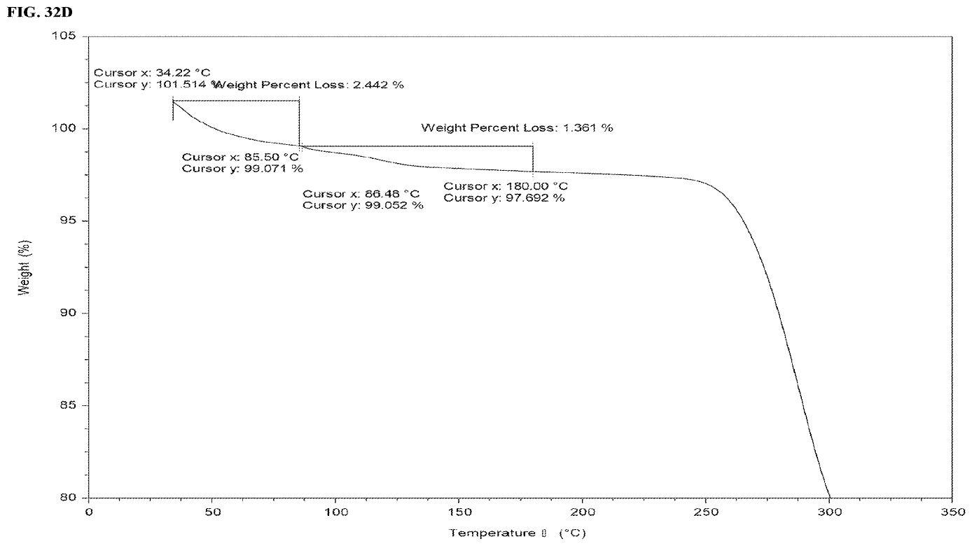

In some embodiments, the solid crystalline form of Compound I-5 is Form A. In some embodiments, Form A of Compound I-5 has a X-Ray diffraction pattern substantially similar to that depicted in FIG. 32A.

In some embodiments, Form A of Compound I-5 has a differential scanning calorimetry (DSC) pattern substantially similar to that depicted in FIG. 32B. In some embodiments, Form A of Compound I-5 has a differential scanning calorimetry (DSC) pattern substantially similar to that depicted in FIG. 32C. In some embodiments, Form A of Compound I-5 has a thermogravimetric analysis (TGA) pattern substantially similar to that depicted in FIG. 32D. In some embodiments, Form A of Compound I-5 can be characterized by substantial similarity to two or more of these figures simultaneously.

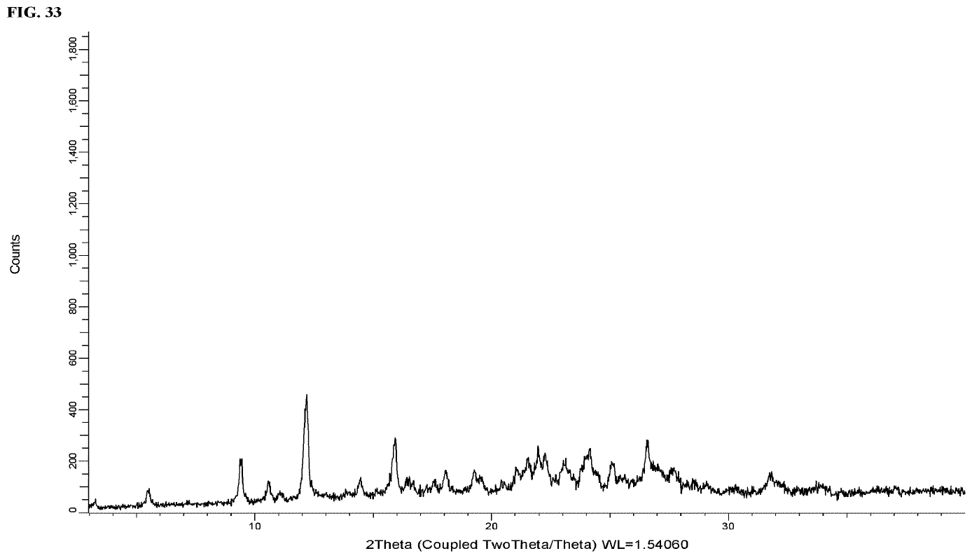

In some embodiments, the solid crystalline form of Compound I-5 is Form B. In some embodiments, Form B of Compound I-5 has a X-Ray diffraction pattern substantially similar to that depicted in FIG. 33.

### Compound I-6

In some embodiments, a compound of Formula (I) is Compound I-6:

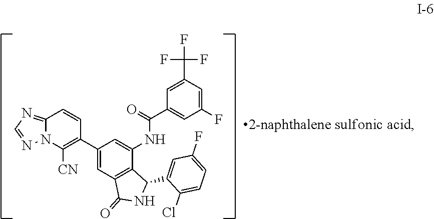

or a solvate thereof.

In some embodiments, Compound I-6 is an anhydrous solid.

In some embodiments, Compound I-6 is an amorphous solid. In other embodiments, Compound I-6 is a crystalline solid. In some embodiments, Compound I-6 is a mixture of amorphous solid form and crystalline solid form.

In some embodiments, the present invention provides a form of compound I-6 substantially free of impurities. As used herein, the term “substantially free of impurities” means that the compound contains no significant amount of extraneous matter. Such extraneous matter may include different forms of compound I-6, residual solvents, or any other impurities that may result from the preparation of, and/or isolation of, compound I-6.

In some embodiments, compound I-6, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound I-6, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound I-6, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound I-6, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound I-6 is also meant to include all tautomeric forms of compound I-6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound I-6 is a crystalline solid. In other embodiments, compound I-6 is a crystalline solid substantially free of amorphous compound I-6. As used herein, the term “substantially free of amorphous compound I-6” means that the compound contains no significant amount of amorphous compound I-6. In certain embodiments, at least about 95% by weight of crystalline compound I-6 is present. In still other embodiments of the invention, at least about 99% by weight of crystalline compound I-6 is present.

### Compound of Formula (II)

In some embodiments, provided herein is a compound of Formula (II):

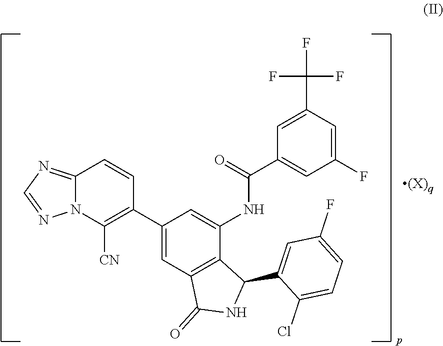

or a solvate thereof,
wherein:
p is 1, 2, 3, 4, 5, 6, 7, 8, or 9;
q is 0, 0.5, 1, 1.5, 2, 2.5, or 3; and
X is hydrochloric acid, p-toluene sulfonic acid, methane sulfonic acid, naphthalene-1,5-disulfonic acid, or 2-naphthalene sulfonic acid.

It will be appreciated by one of ordinary skill in the art that the acid moiety indicated as “X” and (S)-N-(3-(2-chloro-5-fluorophenyl)-6-(5-cyano-[1,2,4]triazolo[1,5-a]pyridin-6-yl)-1-oxoisoindolin-4-yl)-3-fluoro-5-(trifluoromethyl)benzamide are ionically bonded to form a compound of Formula (II). It will also be appreciated that when q is 0, X is absent, indicating that the compound of Formula (II) exists as a “free base,” i.e., “free form.”

It is contemplated that a compound of Formula (II) can exist in a variety of physical forms. For example, a compound of Formula (II) can be in solution, suspension, or in solid form. In certain embodiments, a compound of Formula (II) is in solid form. When a compound of Formula (II) is in solid form, said compound may be amorphous, crystalline, or a mixture thereof. Exemplary solid forms are described in more detail below.

In some embodiments, a compound of Formula (II) is anhydrate. In some embodiments, a compound of Formula (II) may be in a hydrate form. In some embodiments, a compound of Formula (II) may be in a hemi-hydrate form.

In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9.

In some embodiments, q is 0. In some embodiments, q is 1. In some embodiments, q is 2. In some embodiments, q is 3. In some embodiments, q is 0.5. In some embodiments, q is 1.5. In some embodiments, q is 2.5.

In some embodiments, X is hydrochloric acid. In some embodiments, X is p-toluene sulfonic acid. In some embodiments, X is methane sulfonic acid. In some embodiments, X is naphthalene-1,5-disulfonic acid. In some embodiments, X is 2-naphthalene sulfonic acid.

In some embodiments, the present invention provides a form of compound (II) substantially free of impurities. As used herein, the term “substantially free of impurities” means that the compound contains no significant amount of extraneous matter. Such extraneous matter may include different forms of compound (II), residual solvents, or any other impurities that may result from the preparation of, and/or isolation of, compound (II).

In some embodiments, a compound of Formula (II), or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a compound of Formula (II), or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, a compound of Formula (II), or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a compound of Formula (II), or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound of Formula (II) is also meant to include all tautomeric forms. Additionally, structures depicted here are also meant to include compounds that differ only in the presence of one or more isotopically enriched atoms. For example, compounds having the present structure except for the replacement of hydrogen by deuterium or tritium, or the replacement of a carbon by a 13C- or ¹⁴C-enriched carbon are within the scope of this invention.

### Compound II-1

In some embodiments, a compound of Formula (II) is compound II-1, which is a free base (or “free form”),

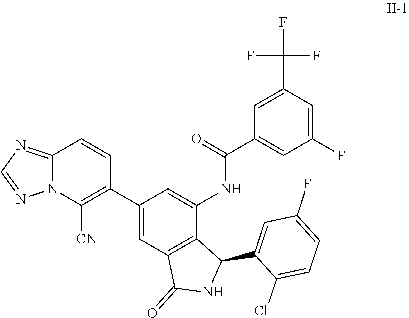 [SMILES: N#Cc1c(-c2cc(NC(=O)c3cc(F)cc(C(F)(F)F)c3)c3c(c2)C(=O)N[C@@H]3c2cc(F)ccc2Cl)ccc2ncnn12]

or a solvate thereof

In some embodiments compound (II-1) is an amorphous solid. In some embodiments, Compound (II-1) is a crystalline solid. In some embodiments, Compound (II-1) is a mixture of amorphous solid form and crystalline solid form.

In some embodiments, the present invention provides a form of compound II-1 substantially free of impurities. As used herein, the term “substantially free of impurities” means that the compound contains no significant amount of extraneous matter. Such extraneous matter may include different forms of compound II-1, residual solvents, or any other impurities that may result from the preparation of, and/or isolation of, compound II-1.

In some embodiments, compound II-1, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound II-1, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound II-1, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound II-1, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound II-1 is also meant to include all tautomeric forms of compound II-1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In other embodiments, compound (II-1) is a crystalline solid substantially free of amorphous compound (II-1). As used herein, the term “substantially free of amorphous compound (II-1)” means that the compound contains no significant amount of amorphous compound (II-1). In certain embodiments, at least about 95% by weight of crystalline compound (II-1) is present. In certain embodiments, at least about 99% by weight of crystalline compound (II-1) is present.

It has been found that compound (II-1) can exist in a variety of solid forms. Exemplary such forms include polymorphs such as those described herein.

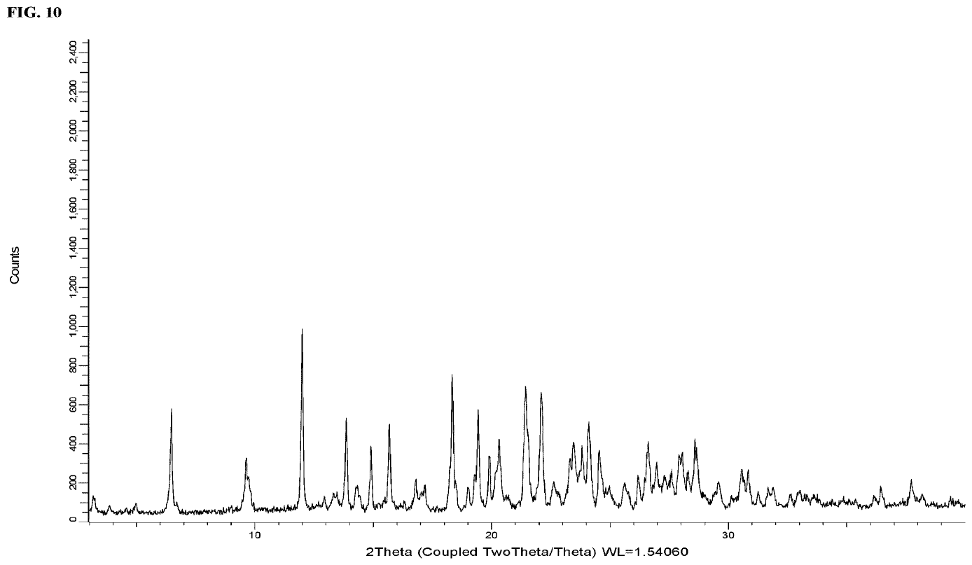

In some embodiments, the solid crystalline form of Compound (II-1) is Form A. In some embodiments, Form A of Compound (II-1) has a X-Ray diffraction pattern substantially similar to that depicted in FIG. 10.

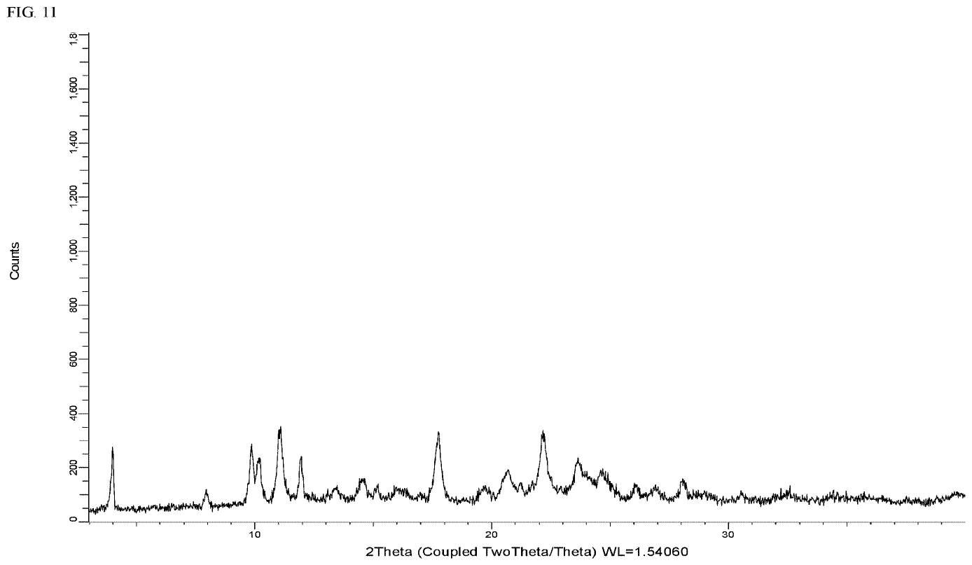

In some embodiments, the solid crystalline form of Compound (II-1) is Form B. In some embodiments, Form B of Compound (II-1) has a X-Ray diffraction pattern substantially similar to that depicted in FIG. 11.

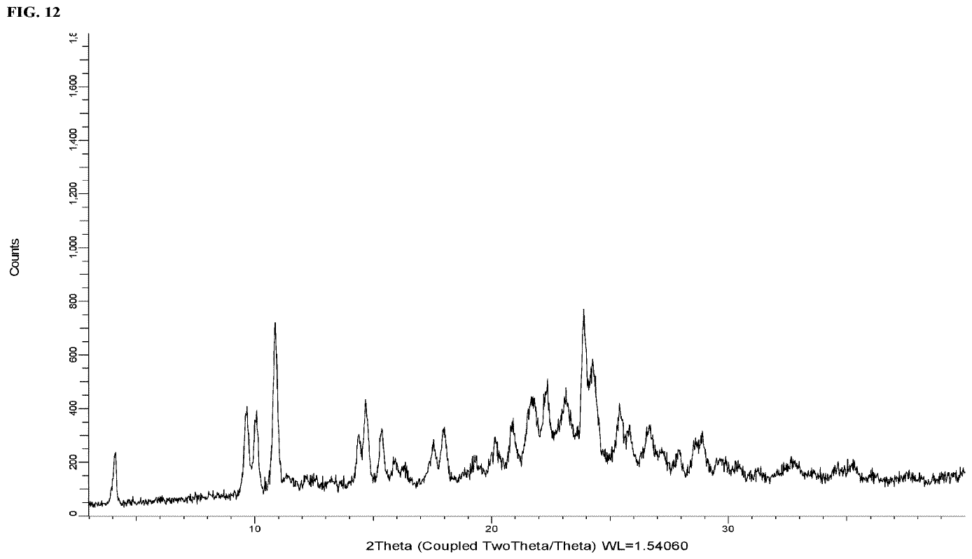

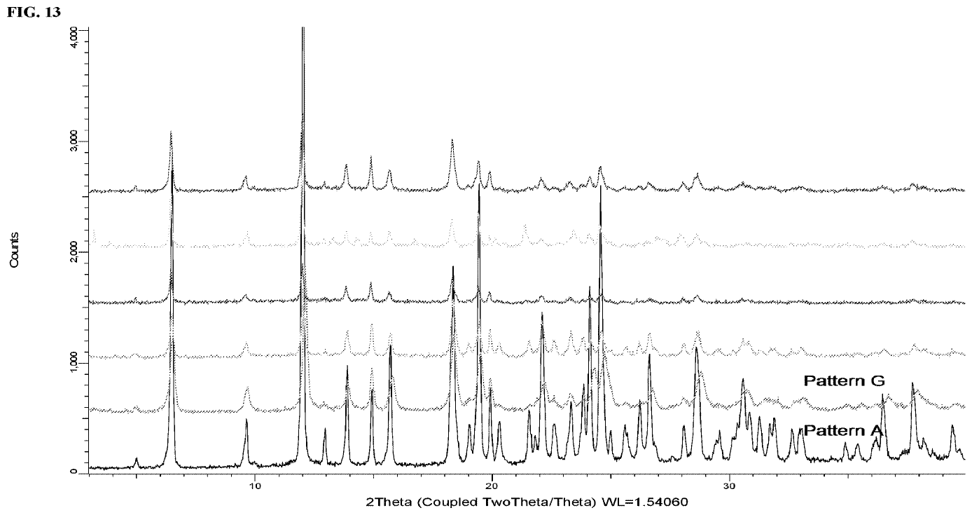

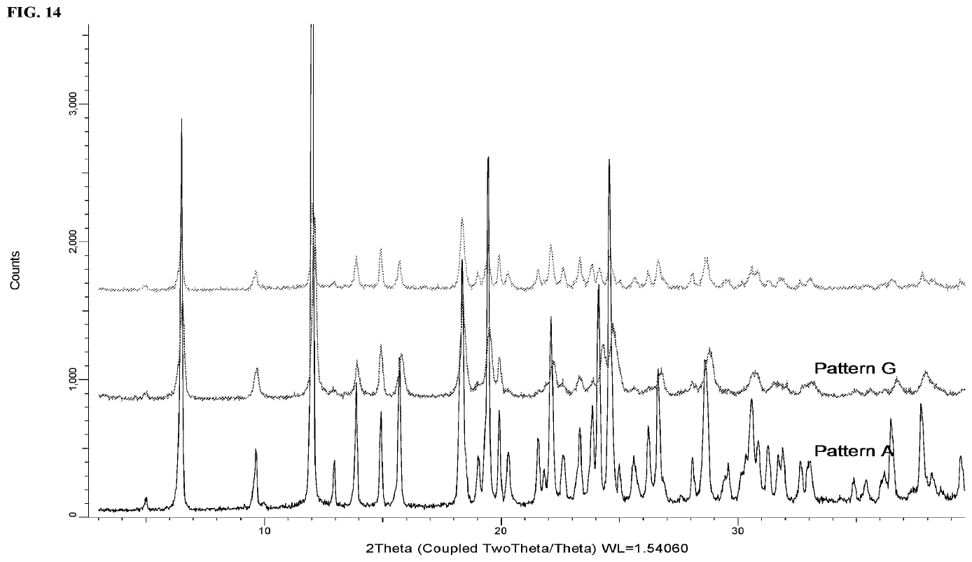

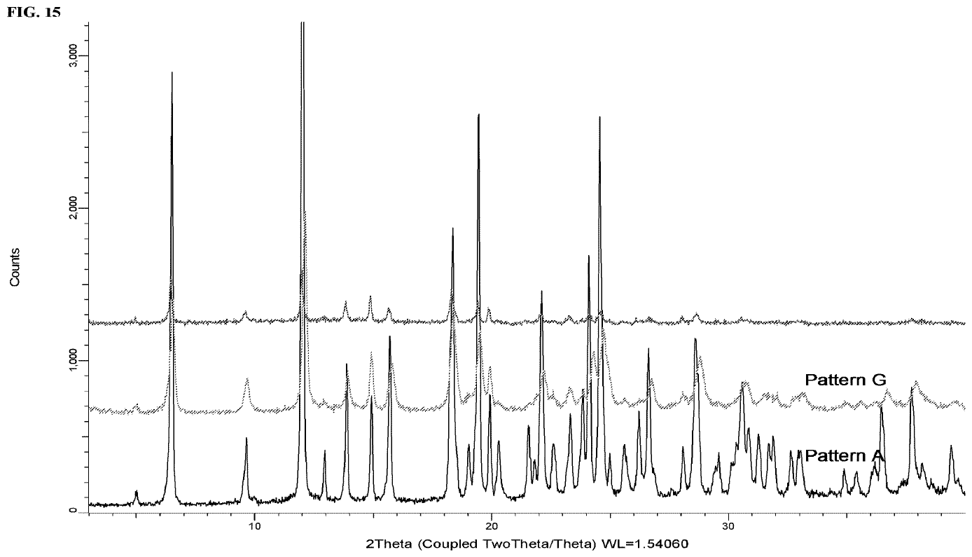

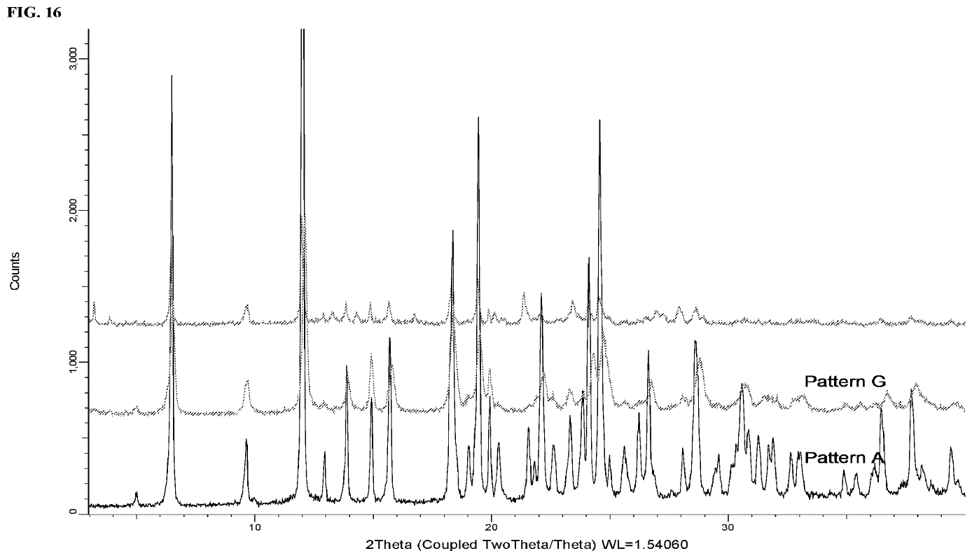

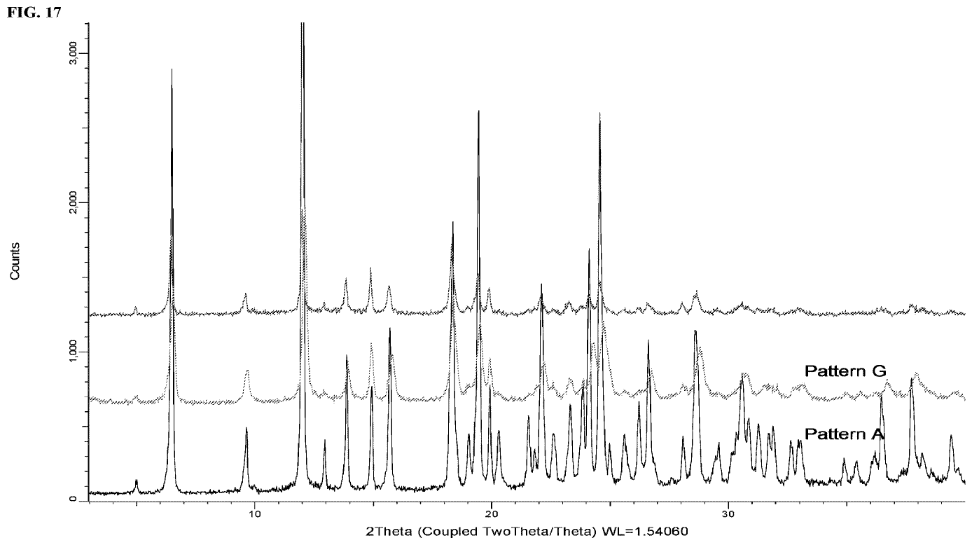

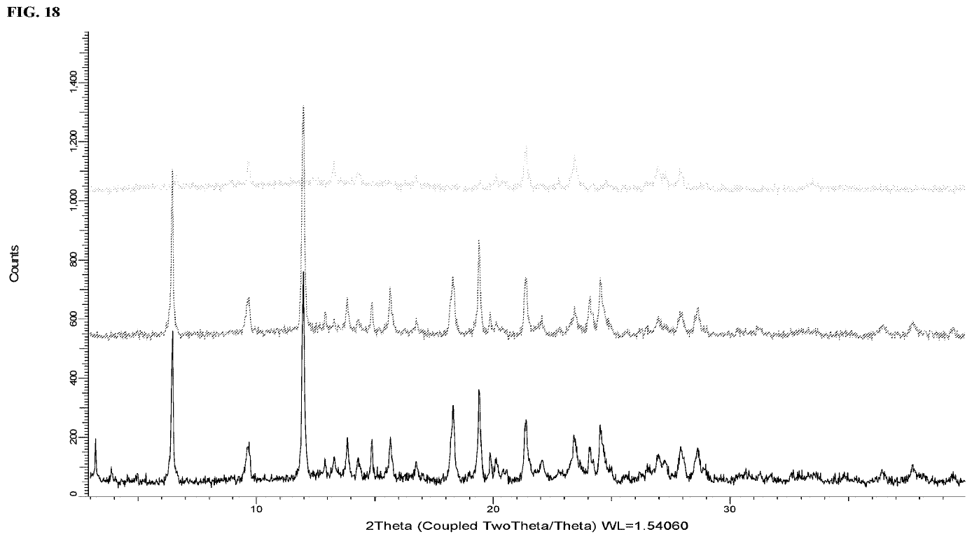

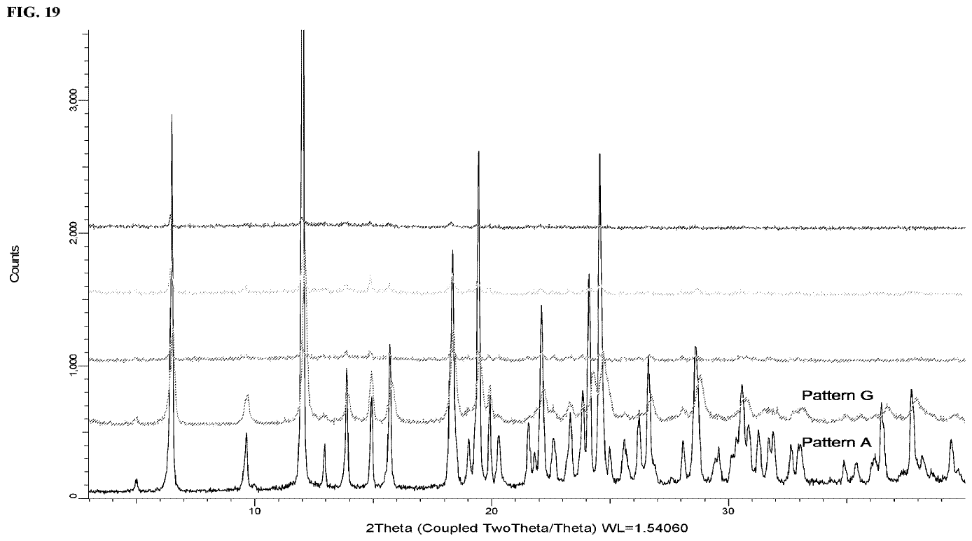

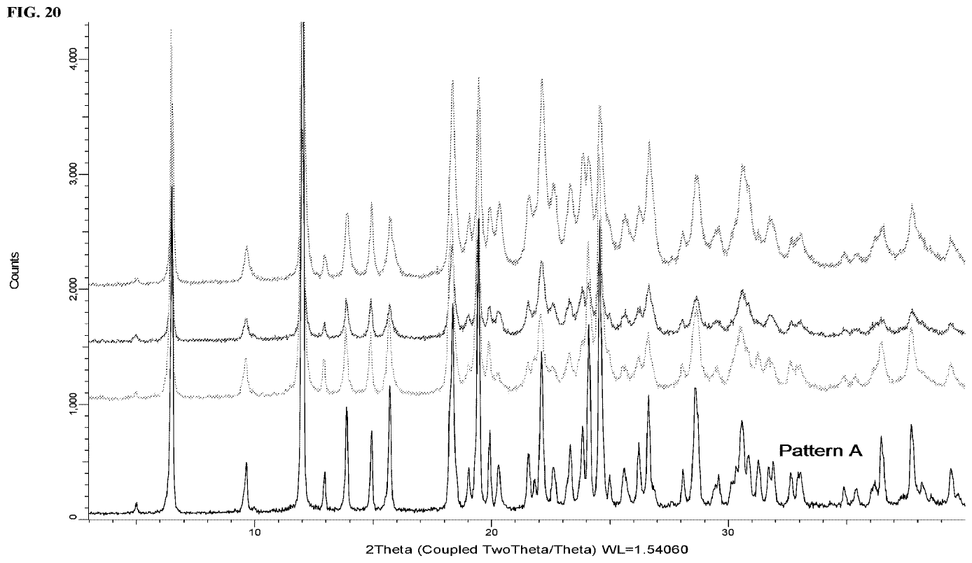

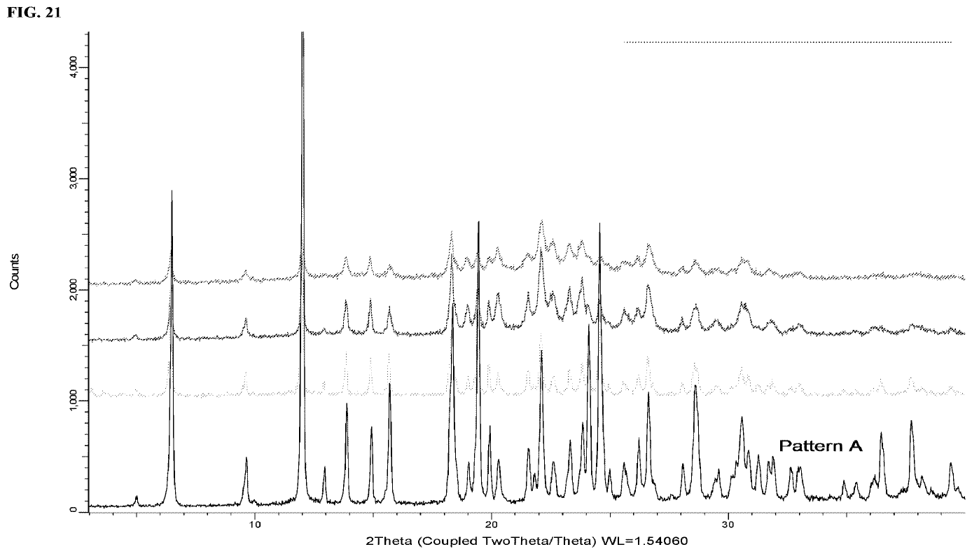

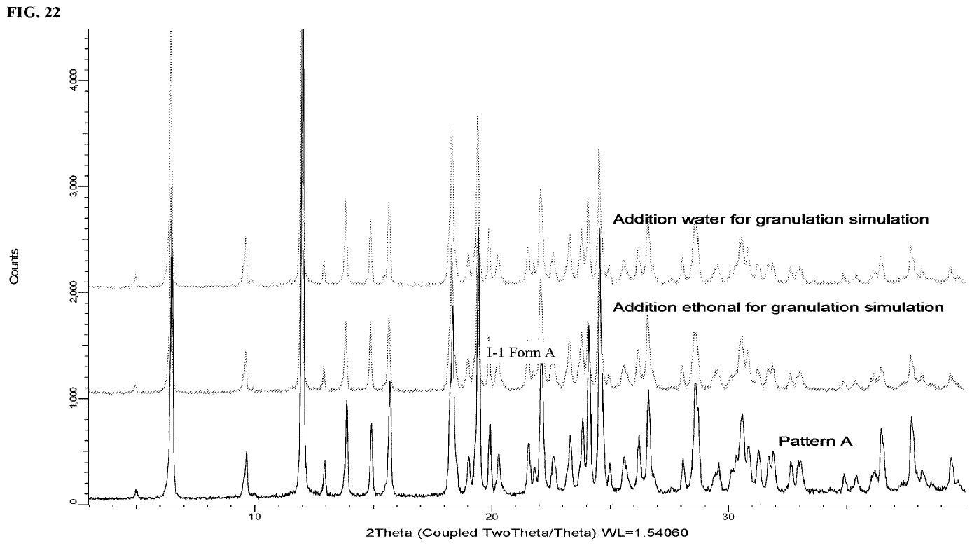

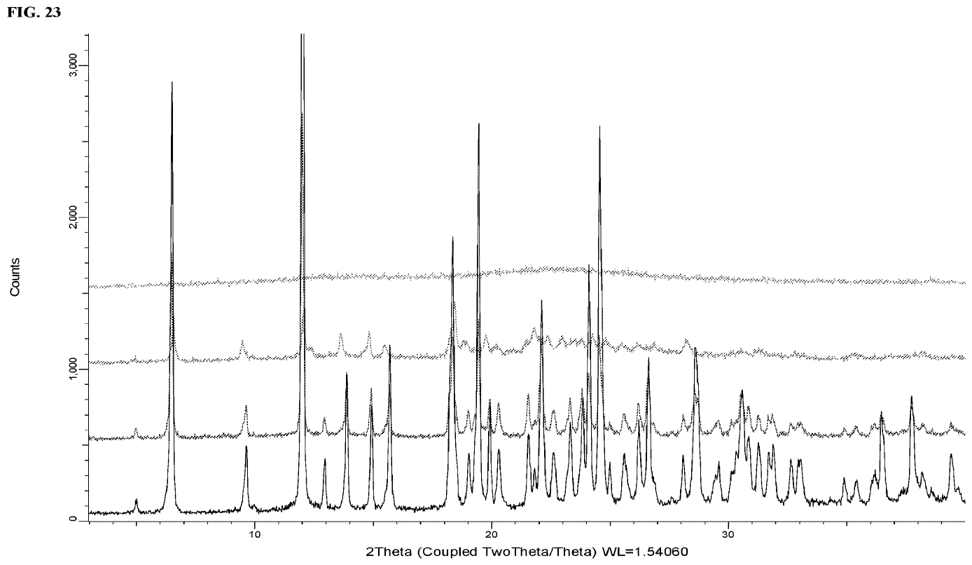

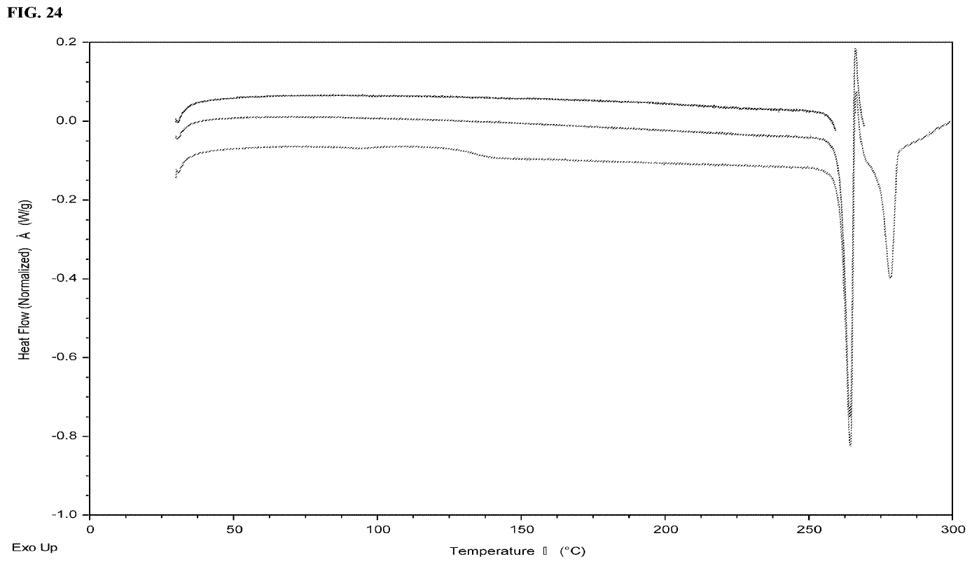

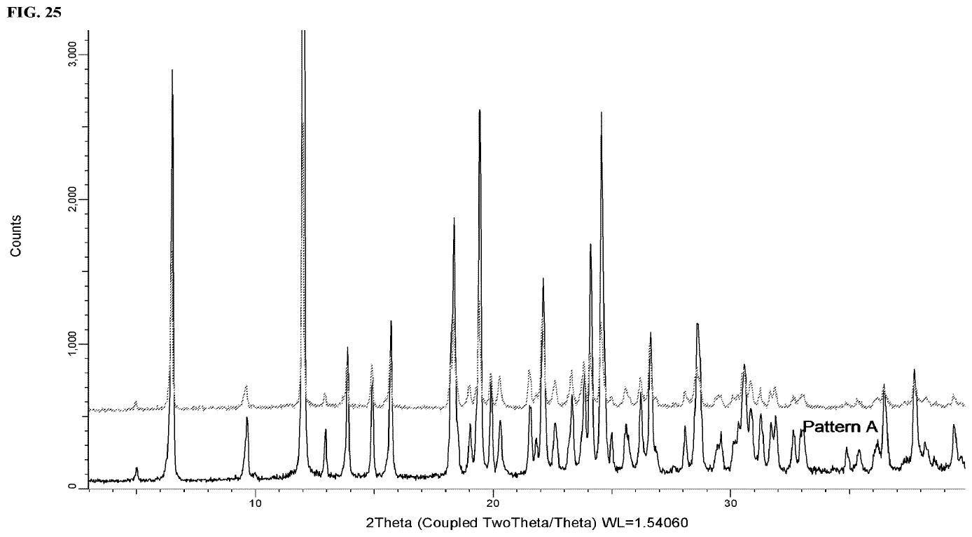

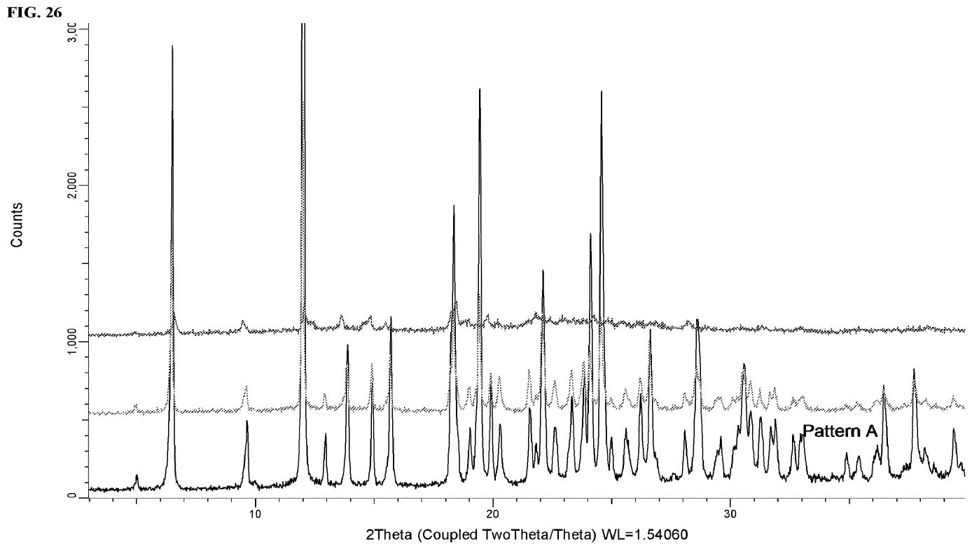

In some embodiments, the solid crystalline form of Compound (II-1) is Form C. In some embodiments, Form C of Compound (II-1) has a X-Ray diffraction pattern substantially similar to that depicted in FIG. 12.

### Compound II-2

In some embodiments, a compound of Formula (II) is Compound II-2:

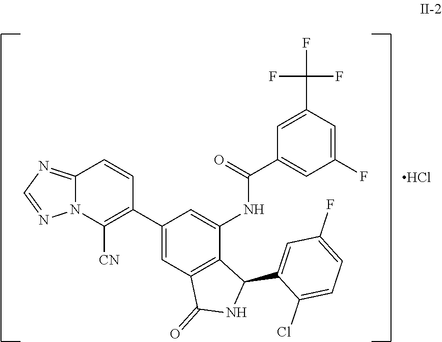

or a solvate thereof.

In some embodiments, Compound II-2 is an anhydrous solid.

In some embodiments, Compound II-2 is an amorphous solid. In other embodiments, Compound II-2 is a crystalline solid. In some embodiments, Compound II-2 is a mixture of amorphous solid form and crystalline solid form.

In some embodiments, the present invention provides a form of compound II-2 substantially free of impurities. As used herein, the term “substantially free of impurities” means that the compound contains no significant amount of extraneous matter. Such extraneous matter may include different forms of compound II-2, residual solvents, or any other impurities that may result from the preparation of, and/or isolation of, compound II-2.

In some embodiments, compound II-2, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound II-2, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound II-2, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound II-2, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound II-2 is also meant to include all tautomeric forms of compound II-2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound II-2 is a crystalline solid. In other embodiments, compound II-2 is a crystalline solid substantially free of amorphous compound II-2. As used herein, the term “substantially free of amorphous compound II-2” means that the compound contains no significant amount of amorphous compound II-2. In certain embodiments, at least about 95% by weight of crystalline compound II-2 is present. In still other embodiments of the invention, at least about 99% by weight of crystalline compound II-2 is present.

### Compound II-3

In some embodiments, a compound of Formula (II) is Compound II-3:

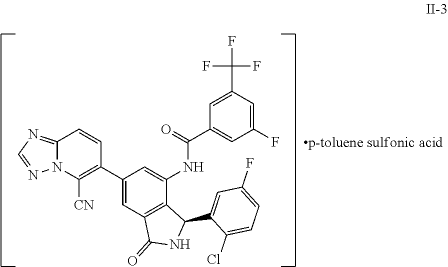

or a solvate thereof.

In some embodiments, Compound II-3 is an anhydrous solid.

In some embodiments, Compound II-3 is an amorphous solid. In other embodiments, Compound II-3 is a crystalline solid. In some embodiments, Compound II-3 is a mixture of amorphous solid form and crystalline solid form.

In some embodiments, the present invention provides a form of compound II-3 substantially free of impurities. As used herein, the term “substantially free of impurities” means that the compound contains no significant amount of extraneous matter. Such extraneous matter may include different forms of compound II-3, residual solvents, or any other impurities that may result from the preparation of, and/or isolation of, compound II-3.

In some embodiments, compound II-3, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound II-3, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound II-3, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound II-3, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound II-3 is also meant to include all tautomeric forms of compound II-3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound II-3 is a crystalline solid. In other embodiments, compound II-3 is a crystalline solid substantially free of amorphous compound II-3. As used herein, the term “substantially free of amorphous compound II-3” means that the compound contains no significant amount of amorphous compound II-3. In certain embodiments, at least about 95% by weight of crystalline compound II-3 is present. In still other embodiments of the invention, at least about 99% by weight of crystalline compound II-3 is present.

### Compound II-4

In some embodiments, a compound of Formula (II) is Compound II-4:

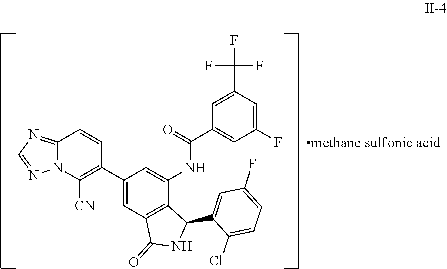

or a solvate thereof.

In some embodiments, Compound II-4 is an anhydrous solid.

In some embodiments, Compound II-4 is an amorphous solid. In other embodiments, Compound II-4 is a crystalline solid. In some embodiments, Compound II-4 is a mixture of amorphous solid form and crystalline solid form.

In some embodiments, the present invention provides a form of compound II-4 substantially free of impurities. As used herein, the term “substantially free of impurities” means that the compound contains no significant amount of extraneous matter. Such extraneous matter may include different forms of compound II-4, residual solvents, or any other impurities that may result from the preparation of, and/or isolation of, compound II-4.

In some embodiments, compound II-4, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound II-4, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound II-4, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound II-4, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound II-4 is also meant to include all tautomeric forms of compound II-4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound II-4 is a crystalline solid. In other embodiments, compound II-4 is a crystalline solid substantially free of amorphous compound II-4. As used herein, the term “substantially free of amorphous compound II-4” means that the compound contains no significant amount of amorphous compound II-4. In certain embodiments, at least about 95% by weight of crystalline compound II-4 is present. In still other embodiments of the invention, at least about 99% by weight of crystalline compound II-4 is present.

### Compound II-5

In some embodiments, a compound of Formula (II) is Compound II-5:

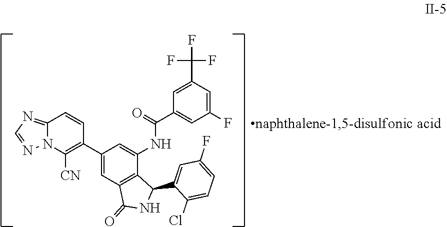

or a solvate thereof

In some embodiments, Compound II-5 is an anhydrous solid.

In some embodiments, Compound II-5 is an amorphous solid. In other embodiments, Compound II-5 is a crystalline solid. In some embodiments, Compound II-5 is a mixture of amorphous solid form and crystalline solid form.

In some embodiments, the present invention provides a form of compound II-5 substantially free of impurities. As used herein, the term “substantially free of impurities” means that the compound contains no significant amount of extraneous matter. Such extraneous matter may include different forms of compound II-5, residual solvents, or any other impurities that may result from the preparation of, and/or isolation of, compound II-5.

In some embodiments, compound II-5, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound II-5, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound II-5, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound II-5, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound II-5 is also meant to include all tautomeric forms of compound II-5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound II-5 is a crystalline solid. In other embodiments, compound II-5 is a crystalline solid substantially free of amorphous compound II-5. As used herein, the term “substantially free of amorphous compound II-5” means that the compound contains no significant amount of amorphous compound II-5. In certain embodiments, at least about 95% by weight of crystalline compound II-5 is present. In still other embodiments of the invention, at least about 99% by weight of crystalline compound II-5 is present.

### Compound II-6

In some embodiments, a compound of Formula (II) is Compound II-6:

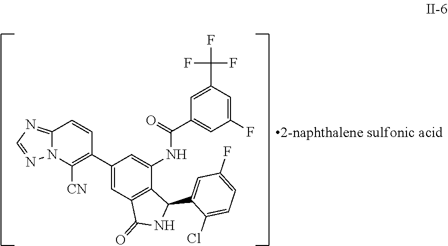

or a solvate thereof.

In some embodiments, Compound II-6 is an anhydrous solid.

In some embodiments, Compound II-6 is an amorphous solid. In other embodiments, Compound II-6 is a crystalline solid. In some embodiments, Compound II-6 is a mixture of amorphous solid form and crystalline solid form.

In some embodiments, the present invention provides a form of compound II-6 substantially free of impurities. As used herein, the term “substantially free of impurities” means that the compound contains no significant amount of extraneous matter. Such extraneous matter may include different forms of compound II-6, residual solvents, or any other impurities that may result from the preparation of, and/or isolation of, compound II-6.

In some embodiments, compound II-6, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound II-6, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound II-6, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound II-6, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound II-6 is also meant to include all tautomeric forms of compound II-6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound II-6 is a crystalline solid. In other embodiments, compound II-6 is a crystalline solid substantially free of amorphous compound II-6. As used herein, the term “substantially free of amorphous compound II-6” means that the compound contains no significant amount of amorphous compound II-6. In certain embodiments, at least about 95% by weight of crystalline compound II-6 is present. In still other embodiments of the invention, at least about 99% by weight of crystalline compound II-6 is present.

### Compound of Formula (III)

In some embodiments, provided herein is a compound of Formula (III)

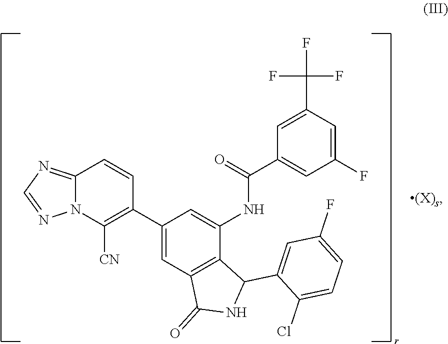

or a solvate thereof,
wherein:
r is 1, 2, 3, 4, 5, 6, 7, 8, or 9;
s is 0, 0.5, 1, 1.5, 2, 2.5, or 3; and
X is hydrochloric acid, p-toluene sulfonic acid, methane sulfonic acid, naphthalene-1,5-disulfonic acid, or 2-naphthalene sulfonic acid.

It will be appreciated by one of ordinary skill in the art that the acid moiety indicated as “X” and N-(3-(2-chloro-5-fluorophenyl)-6-(5-cyano-[1,2,4]triazolo[1,5-a]pyridin-6-yl)-1-oxoisoindolin-4-yl)-3-fluoro-5-(trifluoromethyl)benzamide are ionically bonded to form a compound of Formula (III). It will also be appreciated that when n is 0, X is absent, indicating that the compound of Formula (III) exists as a “free base,” i.e., “free form.”

It is contemplated that a compound of Formula (III) can exist in a variety of physical forms. For example, a compound of Formula (III) can be in solution, suspension, or in solid form. In certain embodiments, a compound of Formula (III) is in solid form. When a compound of Formula (III) is in solid form, said compound may be amorphous, crystalline, or a mixture thereof. Exemplary solid forms are described in more detail below.

In some embodiments, a compound of Formula (III) is anhydrate. In some embodiments, a compound of Formula (III) may be in a hydrate form. In some embodiments, a compound of Formula (III) may be in a hemi-hydrate form.

In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9.

In some embodiments, s is 0. In some embodiments, s is 1. In some embodiments, s is 2. In some embodiments, s is 3. In some embodiments, s is 0.5. In some embodiments, s is 1.5. In some embodiments, s is 2.5.

In some embodiments, X is hydrochloric acid. In some embodiments, X is p-toluene sulfonic acid. In some embodiments, X is methane sulfonic acid. In some embodiments, X is naphthalene-1,5-disulfonic acid. In some embodiments, X is 2-naphthalene sulfonic acid.

In some embodiments, the present invention provides a form of compound (III) substantially free of impurities. As used herein, the term “substantially free of impurities” means that the compound contains no significant amount of extraneous matter. Such extraneous matter may include different forms of compound (III), residual solvents, or any other impurities that may result from the preparation of, and/or isolation of, compound (III).

In some embodiments, a compound of Formula (III), or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a compound of Formula (III), or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, a compound of Formula (III), or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a compound of Formula (III), or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound of Formula (III) is also meant to include all tautomeric forms. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

### Compound III-1

In some embodiments, a compound of Formula (III) is compound III-1, which is a free base (or “free form”),

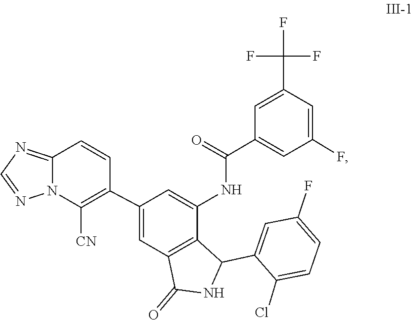 [SMILES: N#Cc1c(-c2cc(NC(=O)c3cc(F)cc(C(F)(F)F)c3)c3c(c2)C(=O)NC3c2cc(F)ccc2Cl)ccc2ncnn12]

or a solvate thereof.

In some embodiments compound (III-1) is an amorphous solid. In some embodiments, Compound (III-1) is a crystalline solid. In some embodiments, Compound (III-1) is a mixture of amorphous solid form and crystalline solid form.

In some embodiments, the present invention provides a form of compound III-1 substantially free of impurities. As used herein, the term “substantially free of impurities” means that the compound contains no significant amount of extraneous matter. Such extraneous matter may include different forms of compound III-1, residual solvents, or any other impurities that may result from the preparation of, and/or isolation of, compound III-1.

In some embodiments, compound III-1, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound III-1, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound III-1, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound III-1, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound III-1 is also meant to include all tautomeric forms of compound III-1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In other embodiments, compound (III-1) is a crystalline solid substantially free of amorphous compound (III-1). As used herein, the term “substantially free of amorphous compound (III-1)” means that the compound contains no significant amount of amorphous compound (III-1). In certain embodiments, at least about 95% by weight of crystalline compound (III-1) is present. In certain embodiments, at least about 99% by weight of crystalline compound (III-1) is present.

It has been found that compound (III-1) can exist in a variety of solid forms. Exemplary such forms include polymorphs such as those described herein.

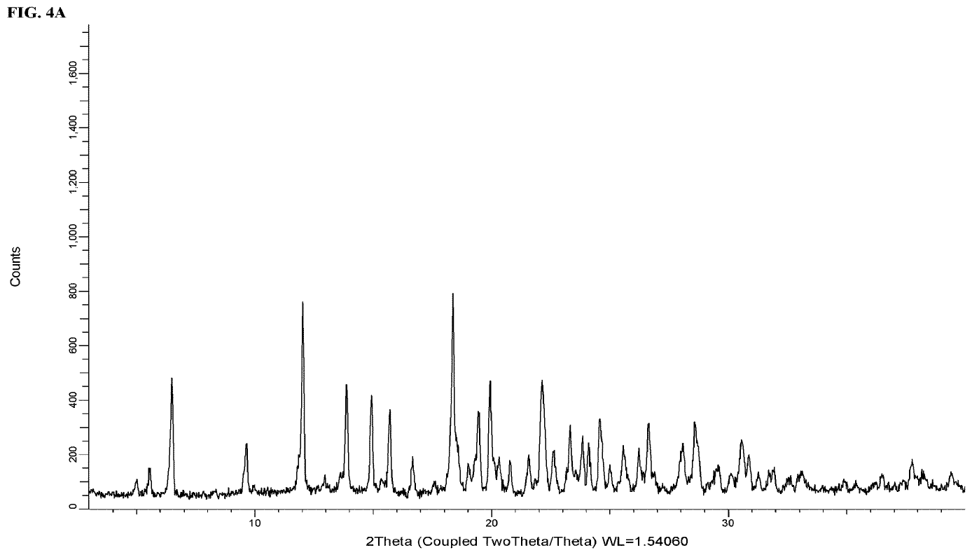

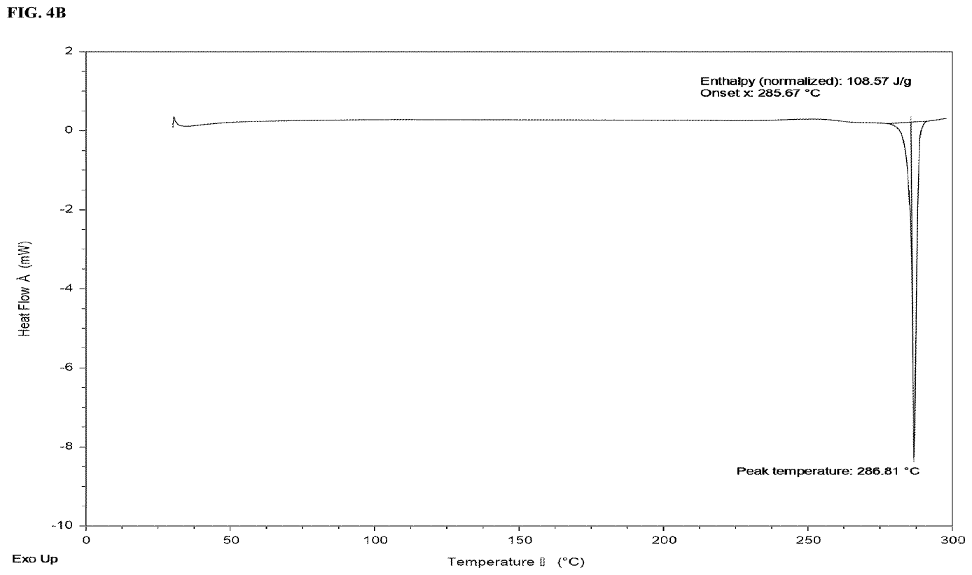

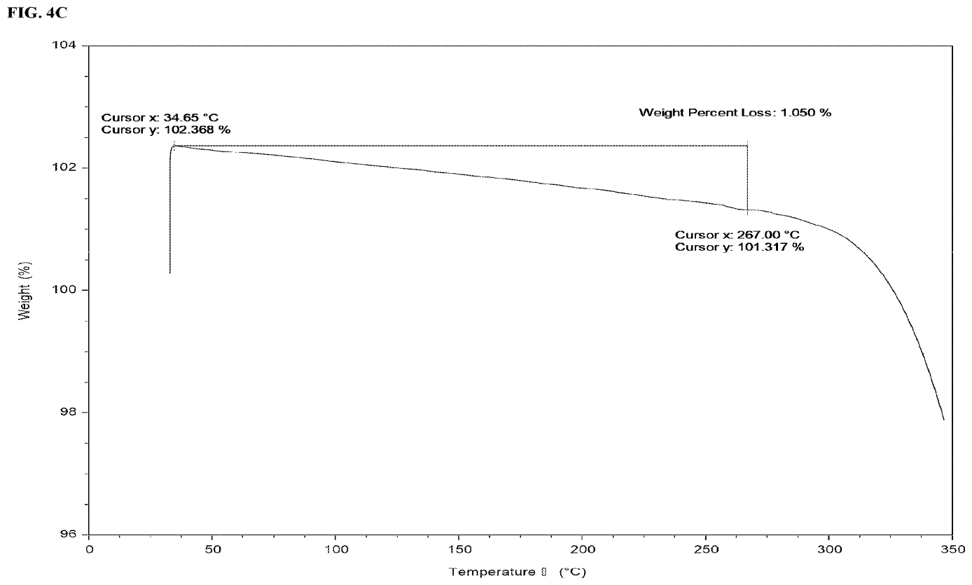

In some embodiments, the solid crystalline form of Compound (III-1) is Form A. In some embodiments, Form A of Compound (III-1) may be characterized by a powder X-ray diffraction pattern with at least two characteristic peaks, in degrees 2θ, each selected from the group consisting of about 18.4 2θ, about 12.0 2θ and about 6.5 2θ. In some embodiments, Form A of Compound (III-1) may be characterized by a powder X-ray diffraction pattern with at least two characteristic peaks, in degrees 2θ, each selected from the group consisting of about 18.4 2θ, about 12.0 2θ, about 6.5 2θ, about 22.1 2θ, about 19.9 2θ, about 13.9 2θ and about 14.9 2θ. In some embodiments, Form A of Compound (III-1) may be characterized by a powder X-ray diffraction pattern with at least three characteristic peaks, in degrees 2θ, each selected from the group consisting of about 18.4 2θ, about 12.0 2θ, about 6.5 2θ, about 22.1 2θ, about 19.9 2θ, about 13.9 2θ and about 14.9 2θ. In some embodiments, Form A of Compound (III-1) may be characterized by a powder X-ray diffraction pattern with at least four characteristic peaks, in degrees 2θ, each selected from the group consisting of about 18.4 2θ, about 12.0 2θ, about 6.5 2θ, about 22.1 2θ, about 19.9 2θ, about 13.9 2θ and about 14.9 2θ. In some embodiments, Form A of Compound (III-1) may be characterized by a powder X-ray diffraction pattern with at least five characteristic peaks, in degrees 2θ, each selected from the group consisting of about 18.4 2θ, about 12.0 2θ, about 6.5 2θ, about 22.1 2θ, about 19.9 2θ, about 13.9 2θ and about 14.9 2θ. In some embodiments, Form A of Compound (III-1) may be characterized by a powder X-ray diffraction pattern with at least six characteristic peaks, in degrees 2θ, each selected from the group consisting of about 18.4 2θ, about 12.0 2θ, about 6.5 2θ, about 22.1 2θ, about 19.9 2θ, about 13.9 2θ and about 14.9 2θ. In some embodiments, Form A of Compound (III-1) may be characterized by a powder X-ray diffraction pattern comprising characteristic peaks at about 18.4 2θ, about 12.0 2θ, about 6.5 2θ, about 22.1 2θ, about 19.9 2θ, about 13.9 2θ and about 14.9 2θ. In some embodiments, Form A of Compound (III-1) has a X-Ray diffraction pattern substantially similar to that depicted in FIG. 4A. In some embodiments, Form A of Compound (III-1) may be characterized by a powder X-ray diffraction pattern with at least two characteristic peaks, in degrees 2θ, each selected from the group consisting of the peaks listed in Table 3.1. In some embodiments, Form A of Compound (III-1) may be characterized by a powder X-ray diffraction pattern with at least three characteristic peaks, in degrees 2θ, each selected from the group consisting of the peaks listed in Table 3.1. In some embodiments, Form A of Compound (III-1) may be characterized by a powder X-ray diffraction pattern with at least four characteristic peaks, in degrees 2θ, each selected from the group consisting of the peaks listed in Table 3.1. In some embodiments, Form A of Compound (III-1) may be characterized by a powder X-ray diffraction pattern with at least five characteristic peaks, in degrees 2θ, each selected from the group consisting of the peaks listed in Table 3.1. In some embodiments, Form A of Compound (III-1) may be characterized by a powder X-ray diffraction pattern with at least six characteristic peaks, in degrees 2θ, each selected from the group consisting of the peaks listed in Table 3.1. In some embodiments, Form A of Compound (III-1) may be characterized by a powder X-ray diffraction pattern with at least seven characteristic peaks, in degrees 2θ, each selected from the group consisting of the peaks listed in Table 3.1.

TABLE 3.1
III-1 Form A XRPD peak listing (the angle 2θ is within ± 0.2).

| Index | Angle | d Value | Rel. Intensity |
|---|---|---|---|
| 1 | 35.338 | 2.53791 | 1.50% |
| 2 | 37.411 | 2.40188 | 4.00% |
| 3 | 17.59 | 5.03807 | 4.60% |
| 4 | 38.221 | 2.35287 | 5.00% |
| 5 | 34.898 | 2.56886 | 5.10% |
| 6 | 15.356 | 5.76543 | 6.10% |
| 7 | 5 | 17.6598 | 6.60% |
| 8 | 32.58 | 2.74614 | 6.80% |
| 9 | 32.972 | 2.7144 | 7.30% |
| 10 | 39.428 | 2.28353 | 7.30% |
| 11 | 12.963 | 6.82386 | 7.80% |
| 12 | 36.482 | 2.46091 | 8.10% |
| 13 | 13.628 | 6.49225 | 8.40% |
| 14 | 30.114 | 2.96521 | 8.50% |
| 15 | 31.278 | 2.85742 | 8.70% |
| 16 | 31.934 | 2.80025 | 10.60% |
| 17 | 25.009 | 3.55771 | 11.30% |
| 18 | 5.551 | 15.9081 | 12.10% |
| 19 | 29.575 | 3.01805 | 12.50% |
| 20 | 19.026 | 4.66089 | 13.50% |
| 21 | 37.767 | 2.38006 | 14.80% |
| 22 | 20.79 | 4.26924 | 16.10% |
| 23 | 30.871 | 2.89423 | 17.70% |
| 24 | 20.318 | 4.36717 | 17.90% |
| 25 | 21.577 | 4.11521 | 18.00% |
| 26 | 16.664 | 5.31564 | 19.20% |
| 27 | 22.622 | 3.92749 | 19.90% |
| 28 | 26.23 | 3.3948 | 21.00% |
| 29 | 25.581 | 3.47943 | 21.80% |
| 30 | 24.108 | 3.68853 | 22.80% |
| 31 | 28.078 | 3.17541 | 24.20% |
| 32 | 23.843 | 3.72902 | 24.80% |
| 33 | 30.573 | 2.92177 | 25.30% |
| 34 | 9.645 | 9.16234 | 25.40% |
| 35 | 18.473 | 4.79908 | 30.30% |
| 36 | 23.325 | 3.81066 | 33.10% |
| 37 | 28.6 | 3.11867 | 33.50% |
| 38 | 26.628 | 3.34491 | 34.10% |
| 39 | 24.582 | 3.61846 | 35.60% |
| 40 | 19.462 | 4.55743 | 39.10% |
| 41 | 15.707 | 5.6374 | 41.70% |
| 42 | 14.926 | 5.93049 | 48.60% |
| 43 | 13.879 | 6.37538 | 54.10% |
| 44 | 19.943 | 4.44849 | 56.00% |
| 45 | 22.141 | 4.01164 | 57.30% |
| 46 | 6.501 | 13.5857 | 57.70% |
| 47 | 12.026 | 7.35361 | 96.00% |
| 48 | 18.369 | 4.82596 | 100.00% |

In some embodiments, Form A of Compound (III-1) has a differential scanning calorimetry (DSC) pattern substantially similar to that depicted in FIG. 4B. In some embodiments, Form A of Compound (III-1) has a thermogravimetric analysis (TGA) pattern substantially similar to that depicted in FIG. 4C. In some embodiments, Form A of Compound (III-1) can be characterized by substantial similarity to two or more of these figures simultaneously.

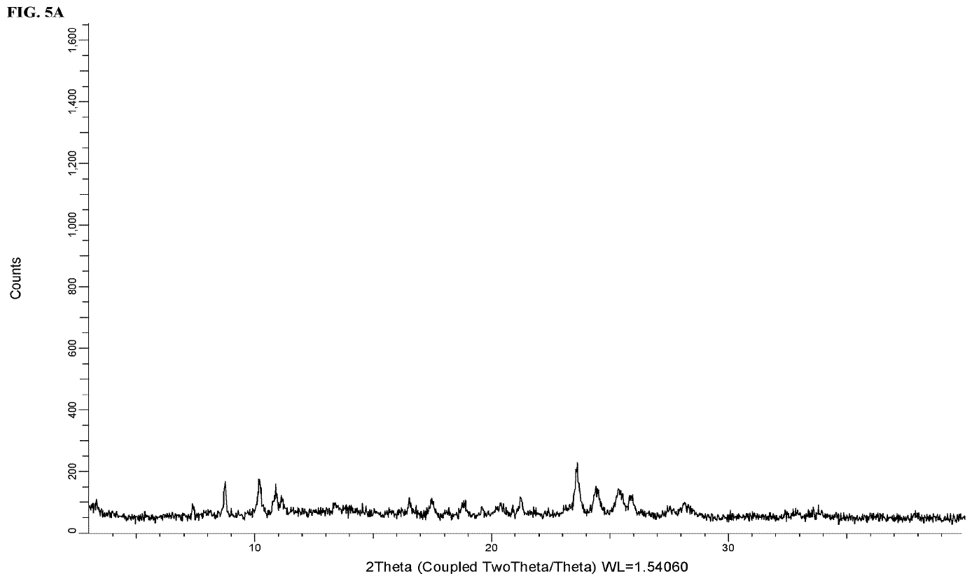

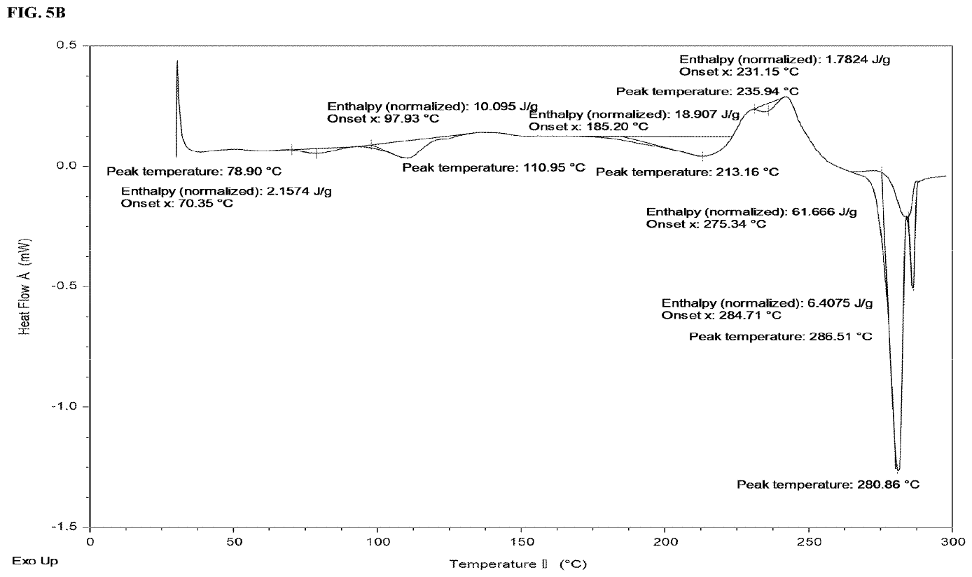

In some embodiments, the solid crystalline form of Compound (III-1) is Form B. In some embodiments, Form B of Compound (III-1) may be characterized by a powder X-ray diffraction pattern with at least two characteristic peaks, in degrees 2θ, each selected from the group consisting of about 23.6 2θ, about 10.2 2θ and about 8.7 2θ. In some embodiments, Form B of Compound (III-1) may be characterized by a powder X-ray diffraction pattern with at least two characteristic peaks, in degrees 2θ, each selected from the group consisting of about 23.6 2θ, about 10.2 2θ, about 8.7 2θ, about 24.4 2θ, about 25.4 2θ, about 10.9 2θ and about 21.2 2θ. In some embodiments, Form B of Compound (III-1) may be characterized by a powder X-ray diffraction pattern with at least three characteristic peaks, in degrees 2θ, each selected from the group consisting of about 23.6 2θ, about 10.2 2θ, about 8.7 2θ, about 24.4 2θ, about 25.4 2θ, about 10.9 2θ and about 21.2 2θ. In some embodiments, Form B of Compound (III-1) may be characterized by a powder X-ray diffraction pattern with at least four characteristic peaks, in degrees 2θ, each selected from the group consisting of about 23.6 2θ, about 10.2 2θ, about 8.7 2θ, about 24.4 2θ, about 25.4 2θ, about 10.9 2θ and about 21.2 2θ. In some embodiments, Form B of Compound (III-1) may be characterized by a powder X-ray diffraction pattern with at least five characteristic peaks, in degrees 2θ, each selected from the group consisting of about 23.6 2θ, about 10.2 2θ, about 8.7 2θ, about 24.4 2θ, about 25.4 2θ, about 10.9 2θ and about 21.2 2θ. In some embodiments, Form B of Compound (III-1) may be characterized by a powder X-ray diffraction pattern with at least six characteristic peaks, in degrees 2θ, each selected from the group consisting of about 23.6 2θ, about 10.2 2θ, about 8.7 2θ, about 24.4 2θ, about 25.4 2θ, about 10.9 2θ and about 21.2 2θ. In some embodiments, Form B of Compound (III-1) may be characterized by a powder X-ray diffraction pattern comprising characteristic peaks at about 23.6 2θ, about 10.2 2θ, about 8.7 2θ, about 24.4 2θ, about 25.4 2θ, about 10.9 2θ and about 21.2 2θ. In some embodiments, Form B of Compound (III-1) has a X-Ray diffraction pattern substantially similar to that depicted in FIG. 5A. In some embodiments, Form B of Compound (III-1) may be characterized by a powder X-ray diffraction pattern with at least two characteristic peaks, in degrees 2θ, each selected from the group consisting of the peaks listed in Table 3.2. In some embodiments, Form B of Compound (III-1) may be characterized by a powder X-ray diffraction pattern with at least three characteristic peaks, in degrees 2θ, each selected from the group consisting of the peaks listed in Table 3.2. In some embodiments, Form B of Compound (III-1) may be characterized by a powder X-ray diffraction pattern with at least four characteristic peaks, in degrees 2θ, each selected from the group consisting of the peaks listed in Table 3.2. In some embodiments, Form B of Compound (III-1) may be characterized by a powder X-ray diffraction pattern with at least five characteristic peaks, in degrees 2θ, each selected from the group consisting of the peaks listed in Table 3.2. In some embodiments, Form B of Compound (III-1) may be characterized by a powder X-ray diffraction pattern with at least six characteristic peaks, in degrees 2θ, each selected from the group consisting of the peaks listed in Table 3.2. In some embodiments, Form B of Compound (III-1) may be characterized by a powder X-ray diffraction pattern with at least seven characteristic peaks, in degrees 2θ, each selected from the group consisting of the peaks listed in Table 3.2.

TABLE 3.2
III-1 Form B XRPD peak listing (the angle 2θ is within ± 0.2).

| Index | Angle | d Value | Rel. Intensity |
|---|---|---|---|
| 1 | 19.626 | 4.51962 | 16.90% |
| 2 | 7.393 | 11.9478 | 21.50% |
| 3 | 20.413 | 4.34708 | 24.10% |
| 4 | 11.141 | 7.93525 | 32.40% |
| 5 | 18.837 | 4.70723 | 32.80% |
| 6 | 17.494 | 5.06547 | 34.80% |
| 7 | 25.858 | 3.44282 | 35.20% |
| 8 | 28.159 | 3.16646 | 37.80% |
| 9 | 16.528 | 5.35908 | 38.10% |
| 10 | 21.227 | 4.1822 | 41.80% |
| 11 | 10.895 | 8.11376 | 57.00% |
| 12 | 25.383 | 3.50609 | 57.20% |
| 13 | 24.423 | 3.64174 | 60.50% |
| 14 | 8.743 | 10.1058 | 72.80% |
| 15 | 10.207 | 8.65947 | 78.30% |
| 16 | 23.604 | 3.76618 | 100.00% |

In some embodiments, Form B of Compound (III-1) has a differential scanning calorimetry (DSC) pattern substantially similar to that depicted in FIG. 5B.

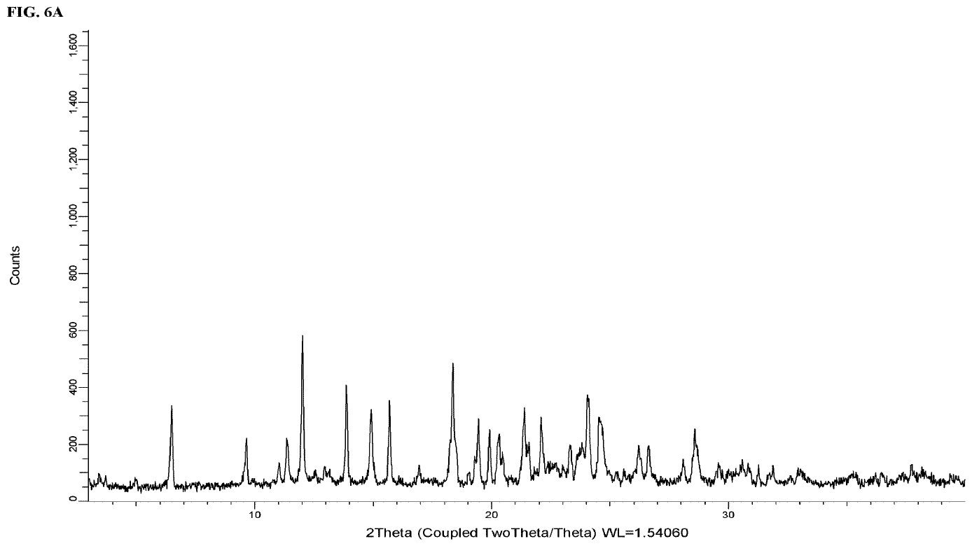

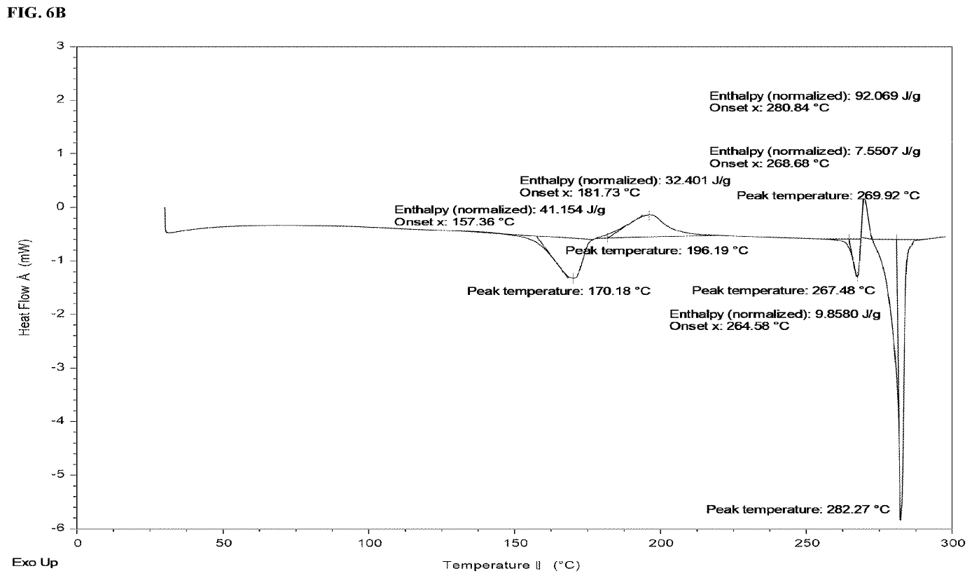

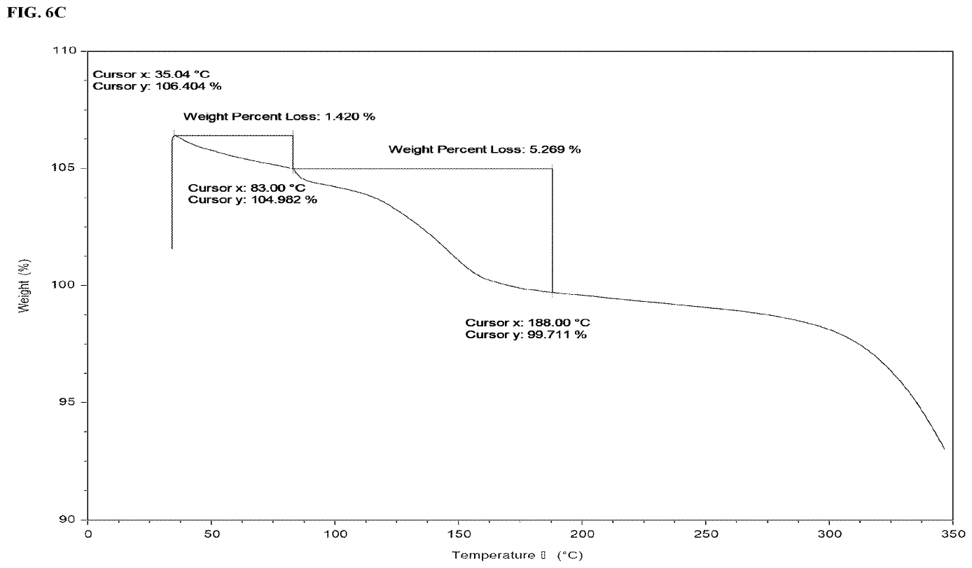

In some embodiments, the solid crystalline form of Compound (III-1) is Form C. In some embodiments, Form C of Compound (III-1) may be characterized by a powder X-ray diffraction pattern with at least two characteristic peaks, in degrees 2θ, each selected from the group consisting of about 12.0 2θ, about 18.4 2θ and about 13.9 2θ. In some embodiments, Form C of Compound (III-1) may be characterized by a powder X-ray diffraction pattern with at least two characteristic peaks, in degrees 2θ, each selected from the group consisting of about 12.0 2θ, about 18.4 2θ, about 13.9 2θ, about 6.5 2θ, about 24.1 2θ, about 15.7 2θ and about 21.4 2θ. In some embodiments, Form C of Compound (III-1) may be characterized by a powder X-ray diffraction pattern with at least three characteristic peaks, in degrees 2θ, each selected from the group consisting of about 12.0 2θ, about 18.4 2θ, about 13.9 2θ, about 6.5 2θ, about 24.1 2θ, about 15.7 2θ and about 21.4 2θ. In some embodiments, Form C of Compound (III-1) may be characterized by a powder X-ray diffraction pattern with at least four characteristic peaks, in degrees 2θ, each selected from the group consisting of about 12.0 2θ, about 18.4 2θ, about 13.9 2θ, about 6.5 2θ, about 24.1 2θ, about 15.7 2θ and about 21.4 2θ. In some embodiments, Form C of Compound (III-1) may be characterized by a powder X-ray diffraction pattern with at least five characteristic peaks, in degrees 2θ, each selected from the group consisting of about 12.0 2θ, about 18.4 2θ, about 13.9 2θ, about 6.5 2θ, about 24.1 2θ, about 15.7 2θ and about 21.4 2θ. In some embodiments, Form C of Compound (III-1) may be characterized by a powder X-ray diffraction pattern with at least six characteristic peaks, in degrees 2θ, each selected from the group consisting of about 12.0 2θ, about 18.4 2θ, about 13.9 2θ, about 6.5 2θ, about 24.1 2θ, about 15.7 2θ and about 21.4 2θ. In some embodiments, Form C of Compound (III-1) may be characterized by a powder X-ray diffraction pattern comprising characteristic peaks at about 12.0 2θ, about 18.4 2θ, about 13.9 2θ, about 6.5 2θ, about 24.1 2θ, about 15.7 2θ and about 21.4 2θ. In some embodiments, Form C of Compound (III-1) has a X-Ray diffraction pattern substantially similar to that depicted in FIG. 6A. In some embodiments, Form C of Compound (III-1) may be characterized by a powder X-ray diffraction pattern with at least two characteristic peaks, in degrees 2θ, each selected from the group consisting of the peaks listed in Table 3.3. In some embodiments, Form C of Compound (III-1) may be characterized by a powder X-ray diffraction pattern with at least three characteristic peaks, in degrees 2θ, each selected from the group consisting of the peaks listed in Table 3.3. In some embodiments, Form C of Compound (III-1) may be characterized by a powder X-ray diffraction pattern with at least four characteristic peaks, in degrees 2θ, each selected from the group consisting of the peaks listed in Table 3.3. In some embodiments, Form C of Compound (ITT-1) may be characterized by a powder X-ray diffraction pattern with at least five characteristic peaks, in degrees 2θ, each selected from the group consisting of the peaks listed in Table 3.3. In some embodiments, Form C of Compound (ITT-1) may be characterized by a powder X-ray diffraction pattern with at least six characteristic peaks, in degrees 2θ, each selected from the group consisting of the peaks listed in Table 3.3. In some embodiments, Form C of Compound (ITT-1) may be characterized by a powder X-ray diffraction pattern with at least seven characteristic peaks, in degrees 2θ, each selected from the group consisting of the peaks listed in Table 3.3.

TABLE 3.3
III-1 Form C XRPD peak listing (the angle 2θ is within ± 0.2).

| Index | Angle | d Value | Rel. Intensity |
|---|---|---|---|
| 1 | 17.638 | 5.02447 | 4.50% |
| 2 | 4.99 | 17.6946 | 5.60% |
| 3 | 32.605 | 2.74414 | 5.60% |
| 4 | 35.249 | 2.54411 | 5.60% |
| 5 | 25.29 | 3.51885 | 6.30% |
| 6 | 31.715 | 2.81905 | 6.40% |
| 7 | 3.702 | 23.8473 | 6.50% |
| 8 | 13.14 | 6.73242 | 7.00% |
| 9 | 23.023 | 3.85997 | 7.00% |
| 10 | 36.208 | 2.4789 | 7.40% |
| 11 | 19.041 | 4.65715 | 7.90% |
| 12 | 36.46 | 2.46233 | 8.00% |
| 13 | 12.567 | 7.03831 | 8.10% |
| 14 | 3.434 | 25.7095 | 8.30% |
| 15 | 30 | 2.97617 | 8.70% |
| 16 | 25.596 | 3.47739 | 9.30% |
| 17 | 33.052 | 2.70805 | 10.40% |
| 18 | 29.63 | 3.01252 | 11.20% |
| 19 | 30.853 | 2.89585 | 11.20% |
| 20 | 12.955 | 6.82806 | 11.40% |
| 21 | 31.268 | 2.85834 | 11.50% |
| 22 | 37.739 | 2.38177 | 12.40% |
| 23 | 31.885 | 2.80444 | 12.50% |
| 24 | 16.941 | 5.2296 | 14.00% |
| 25 | 28.099 | 3.17305 | 14.30% |
| 26 | 11.035 | 8.01143 | 14.70% |
| 27 | 30.586 | 2.92052 | 15.50% |
| 28 | 19.306 | 4.59384 | 16.80% |
| 29 | 20.472 | 4.33477 | 21.30% |
| 30 | 23.331 | 3.80963 | 22.60% |
| 31 | 21.561 | 4.11829 | 24.70% |
| 32 | 23.827 | 3.73145 | 24.70% |
| 33 | 26.62 | 3.34588 | 25.60% |
| 34 | 26.212 | 3.39711 | 25.80% |
| 35 | 11.376 | 7.77233 | 30.30% |
| 36 | 9.653 | 9.15503 | 32.20% |
| 37 | 20.308 | 4.36946 | 33.00% |
| 38 | 19.918 | 4.45405 | 36.40% |
| 39 | 28.6 | 3.11869 | 37.10% |
| 40 | 22.106 | 4.01786 | 43.10% |
| 41 | 24.562 | 3.62145 | 43.60% |
| 42 | 19.446 | 4.56114 | 46.10% |
| 43 | 14.92 | 5.93293 | 49.60% |
| 44 | 21.384 | 4.15193 | 52.30% |
| 45 | 15.696 | 5.64143 | 57.20% |
| 46 | 24.075 | 3.69356 | 57.70% |
| 47 | 6.494 | 13.5988 | 58.60% |
| 48 | 13.878 | 6.37616 | 67.20% |
| 49 | 18.369 | 4.82605 | 86.10% |
| 50 | 12.02 | 7.35722 | 100.00% |

In some embodiments, Form C of Compound (III-1) has a differential scanning calorimetry (DSC) pattern substantially similar to that depicted in FIG. 6B. In some embodiments, Form C of Compound (III-1) has a thermogravimetric analysis (TGA) pattern substantially similar to that depicted in FIG. 6C. In some embodiments, Form C of Compound (III-1) can be characterized by substantial similarity to two or more of these figures simultaneously.

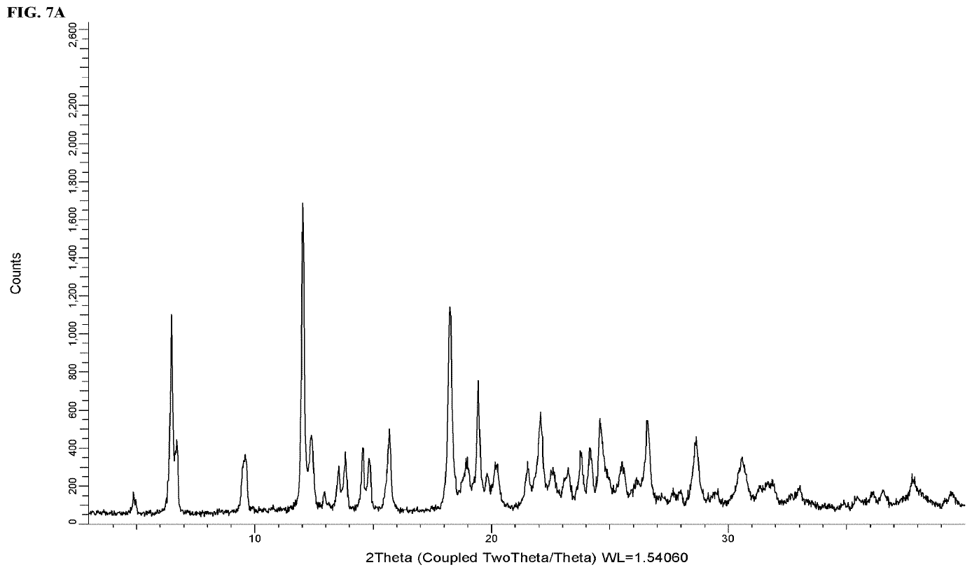

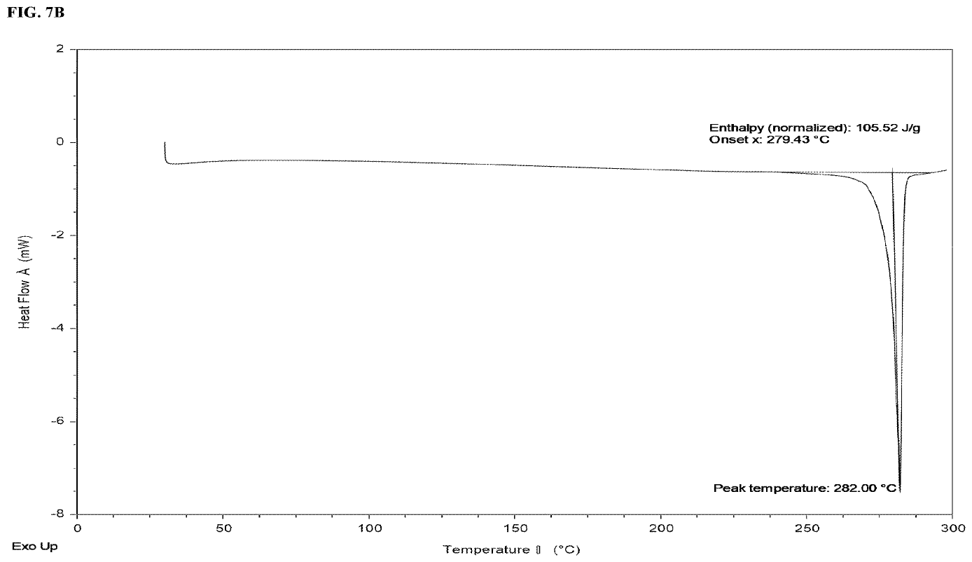

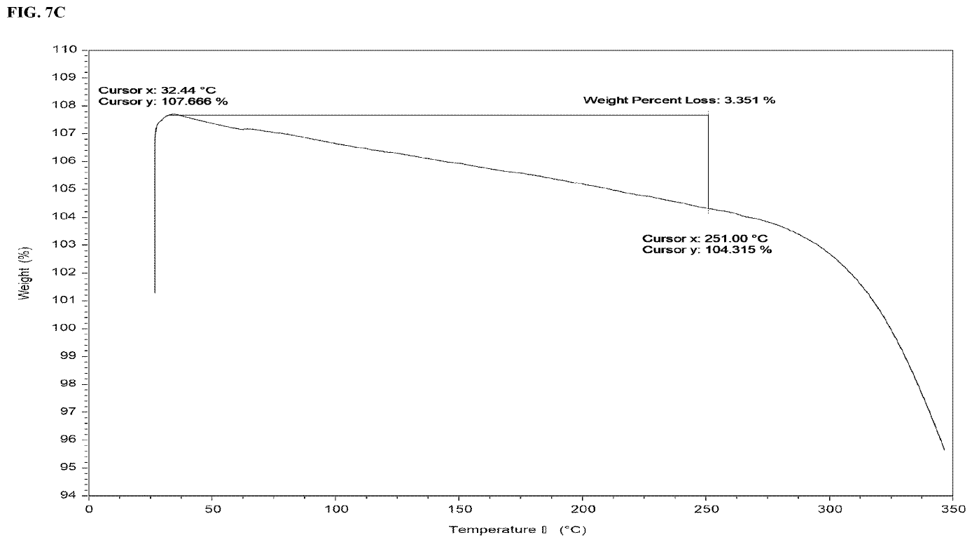

In some embodiments, the solid crystalline form of Compound (III-1) is Form D. In some embodiments, Form D of Compound (ITT-1) may be characterized by a powder X-ray diffraction pattern with at least two characteristic peaks, in degrees 2θ, each selected from the group consisting of about 12.0 2θ, about 18.3 2θ and about 6.5 2θ. In some embodiments, Form D of Compound (ITT-1) may be characterized by a powder X-ray diffraction pattern with at least two characteristic peaks, in degrees 2θ, each selected from the group consisting of about 12.0 2θ, about 18.3 2θ, about 6.5 2θ, about 19.4 2θ, about 22.1 2θ, about 15.7 2θ and about 26.6 2θ. In some embodiments, Form D of Compound (ITT-1) may be characterized by a powder X-ray diffraction pattern with at least three characteristic peaks, in degrees 2θ, each selected from the group consisting of about 12.0 2θ, about 18.3 2θ, about 6.5 2θ, about 19.4 2θ, about 22.1 2θ, about 15.7 2θ and about 26.6 2θ. In some embodiments, Form D of Compound (ITT-1) may be characterized by a powder X-ray diffraction pattern with at least four characteristic peaks, in degrees 2θ, each selected from the group consisting of about 12.0 2θ, about 18.3 2θ, about 6.5 2θ, about 19.4 2θ, about 22.1 2θ, about 15.7 2θ and about 26.6 2θ. In some embodiments, Form D of Compound (III-1) may be characterized by a powder X-ray diffraction pattern with at least five characteristic peaks, in degrees 2θ, each selected from the group consisting of about 12.0 2θ, about 18.3 2θ, about 6.5 2θ, about 19.4 2θ, about 22.1 2θ, about 15.7 2θ and about 26.6 2θ. In some embodiments, Form D of Compound (III-1) may be characterized by a powder X-ray diffraction pattern with at least six characteristic peaks, in degrees 2θ, each selected from the group consisting of about 12.0 2θ, about 18.3 2θ, about 6.5 2θ, about 19.4 2θ, about 22.1 2θ, about 15.7 2θ and about 26.6 2θ. In some embodiments, Form D of Compound (III-1) may be characterized by a powder X-ray diffraction pattern comprising characteristic peaks at about 12.0 2θ, about 18.3 2θ, about 6.5 2θ, about 19.4 2θ, about 22.1 2θ, about 15.7 2θ and about 26.6 2θ. In some embodiments, Form D of Compound (III-1) has a X-Ray diffraction pattern substantially similar to that depicted in FIG. 7A. In some embodiments, Form D of Compound (III-1) may be characterized by a powder X-ray diffraction pattern with at least two characteristic peaks, in degrees 2θ, each selected from the group consisting of the peaks listed in Table 3.4. In some embodiments, Form D of Compound (III-1) may be characterized by a powder X-ray diffraction pattern with at least three characteristic peaks, in degrees 2θ, each selected from the group consisting of the peaks listed in Table 3.4. In some embodiments, Form D of Compound (III-1) may be characterized by a powder X-ray diffraction pattern with at least four characteristic peaks, in degrees 2θ, each selected from the group consisting of the peaks listed in Table 3.4. In some embodiments, Form D of Compound (III-1) may be characterized by a powder X-ray diffraction pattern with at least five characteristic peaks, in degrees 2θ, each selected from the group consisting of the peaks listed in Table 3.4. In some embodiments, Form D of Compound (III-1) may be characterized by a powder X-ray diffraction pattern with at least six characteristic peaks, in degrees 2θ, each selected from the group consisting of the peaks listed in Table 3.4. In some embodiments, Form D of Compound (III-1) may be characterized by a powder X-ray diffraction pattern with at least seven characteristic peaks, in degrees 2θ, each selected from the group consisting of the peaks listed in Table 3.4.

TABLE 3.4
III-1 Form D XRPD peak listing (the angle 2θ is within ± 0.2).

| Index | Angle | d Value | Rel. Intensity |
|---|---|---|---|
| 1 | 34.902 | 2.56858 | 2.00% |
| 2 | 29.513 | 3.02417 | 2.60% |
| 3 | 32.695 | 2.73674 | 2.80% |
| 4 | 35.469 | 2.52884 | 3.20% |
| 5 | 35.469 | 2.52884 | 3.20% |
| 6 | 27.974 | 3.18694 | 3.30% |
| 7 | 36.106 | 2.48564 | 3.80% |
| 8 | 39.455 | 2.28206 | 3.80% |
| 9 | 27.679 | 3.22031 | 4.40% |
| 10 | 31.342 | 2.8518 | 4.70% |
| 11 | 36.547 | 2.45666 | 4.70% |
| 12 | 26.177 | 3.4016 | 4.90% |
| 13 | 12.953 | 6.82937 | 5.10% |
| 14 | 33.021 | 2.71048 | 5.70% |
| 15 | 23.113 | 3.84509 | 6.20% |
| 16 | 23.09 | 3.8489 | 6.50% |
| 17 | 4.885 | 18.0756 | 6.80% |
| 18 | 31.871 | 2.80562 | 7.30% |
| 19 | 37.757 | 2.38069 | 8.50% |
| 20 | 22.606 | 3.93023 | 10.40% |
| 21 | 19.828 | 4.47415 | 10.50% |
| 22 | 25.528 | 3.48655 | 11.40% |
| 23 | 20.202 | 4.39204 | 12.70% |
| 24 | 13.548 | 6.5304 | 12.80% |
| 25 | 21.535 | 4.12308 | 13.70% |
| 26 | 23.788 | 3.73745 | 13.90% |
| 27 | 30.589 | 2.92025 | 14.50% |
| 28 | 24.169 | 3.67947 | 16.00% |
| 29 | 14.845 | 5.96258 | 16.40% |
| 30 | 18.995 | 4.66842 | 16.50% |
| 31 | 13.838 | 6.3942 | 17.30% |
| 32 | 9.604 | 9.20126 | 18.60% |
| 33 | 14.568 | 6.07548 | 20.00% |
| 34 | 28.656 | 3.11272 | 21.50% |
| 35 | 12.394 | 7.13563 | 23.90% |
| 36 | 6.699 | 13.185 | 24.30% |
| 37 | 24.599 | 3.61603 | 24.90% |
| 38 | 26.589 | 3.34973 | 25.50% |
| 39 | 15.693 | 5.64229 | 26.00% |
| 40 | 22.077 | 4.02306 | 29.70% |
| 41 | 19.447 | 4.5609 | 41.30% |
| 42 | 6.491 | 13.6064 | 65.10% |
| 43 | 18.253 | 4.85635 | 66.00% |
| 44 | 12.038 | 7.34586 | 100.00% |

In some embodiments, Form D of Compound (III-1) has a differential scanning calorimetry (DSC) pattern substantially similar to that depicted in FIG. 7B. In some embodiments, Form D of Compound (III-1) has a thermogravimetric analysis (TGA) pattern substantially similar to that depicted in FIG. 7C. In some embodiments, Form D of Compound (III-1) can be characterized by substantial similarity to two or more of these figures simultaneously.

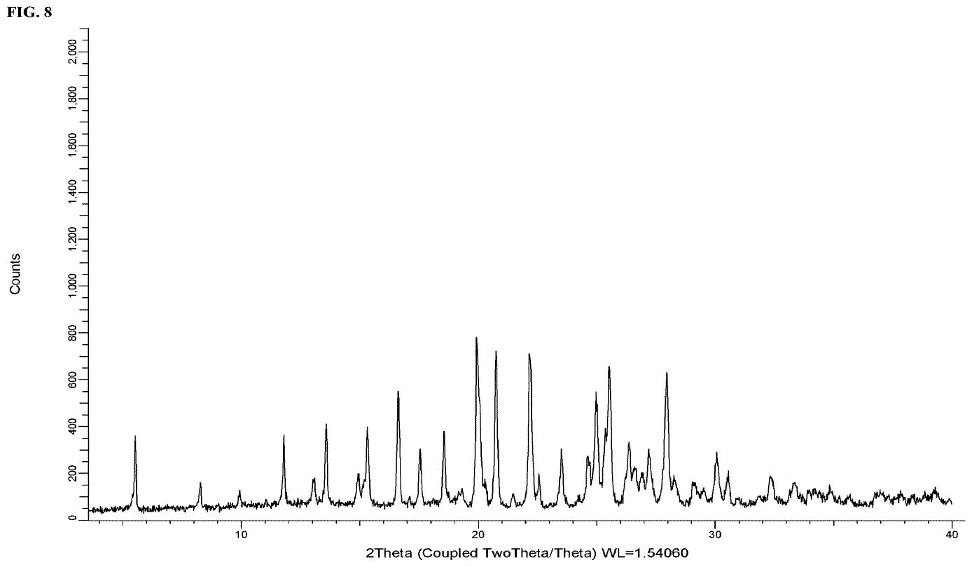

In some embodiments, the solid crystalline form of Compound (III-1) is Form E. In some embodiments, Form E of Compound (III-1) may be characterized by a powder X-ray diffraction pattern with at least two characteristic peaks, in degrees 2θ, each selected from the group consisting of about 20.8 2θ, about 22.2 2θ and about 20.0 2θ. In some embodiments, Form E of Compound (III-1) may be characterized by a powder X-ray diffraction pattern with at least two characteristic peaks, in degrees 2θ, each selected from the group consisting of about 20.8 2θ, about 22.2 2θ, about 20.0 2θ, about 25.5 2θ, about 28.0 2θ, about 16.6 2θ and about 25.0 2θ. In some embodiments, Form E of Compound (III-1) may be characterized by a powder X-ray diffraction pattern with at least three characteristic peaks, in degrees 2θ, each selected from the group consisting of about 20.8 2θ, about 22.2 2θ, about 20.0 2θ, about 25.5 2θ, about 28.0 2θ, about 16.6 2θ and about 25.0 2θ. In some embodiments, Form E of Compound (III-1) may be characterized by a powder X-ray diffraction pattern with at least four characteristic peaks, in degrees 2θ, each selected from the group consisting of about 20.8 2θ, about 22.2 2θ, about 20.0 2θ, about 25.5 2θ, about 28.0 2θ, about 16.6 2θ and about 25.0 2θ. In some embodiments, Form E of Compound (III-1) may be characterized by a powder X-ray diffraction pattern with at least five characteristic peaks, in degrees 2θ, each selected from the group consisting of about 20.8 2θ, about 22.2 2θ, about 20.0 2θ, about 25.5 2θ, about 28.0 2θ, about 16.6 2θ and about 25.0 2θ. In some embodiments, Form E of Compound (III-1) may be characterized by a powder X-ray diffraction pattern with at least six characteristic peaks, in degrees 2θ, each selected from the group consisting of about 20.8 2θ, about 22.2 2θ, about 20.0 2θ, about 25.5 2θ, about 28.0 2θ, about 16.6 2θ and about 25.0 2θ. In some embodiments, Form E of Compound (III-1) may be characterized by a powder X-ray diffraction pattern comprising characteristic peaks at about 20.8 2θ, about 22.2 2θ, about 20.0 2θ, about 25.5 2θ, about 28.0 2θ, about 16.6 2θ and about 25.0 2θ. In some embodiments, Form E of Compound (III-1) has a X-Ray diffraction pattern substantially similar to that depicted in FIG. 8. In some embodiments, Form E of Compound (III-1) may be characterized by a powder X-ray diffraction pattern with at least two characteristic peaks, in degrees 2θ, each selected from the group consisting of the peaks listed in Table 3.5. In some embodiments, Form E of Compound (III-1) may be characterized by a powder X-ray diffraction pattern with at least three characteristic peaks, in degrees 2θ, each selected from the group consisting of the peaks listed in Table 3.5. In some embodiments, Form E of Compound (ITT-1) may be characterized by a powder X-ray diffraction pattern with at least four characteristic peaks, in degrees 2θ, each selected from the group consisting of the peaks listed in Table 3.5. In some embodiments, Form E of Compound (ITT-1) may be characterized by a powder X-ray diffraction pattern with at least five characteristic peaks, in degrees 2θ, each selected from the group consisting of the peaks listed in Table 3.5. In some embodiments, Form E of Compound (ITT-1) may be characterized by a powder X-ray diffraction pattern with at least six characteristic peaks, in degrees 2θ, each selected from the group consisting of the peaks listed in Table 3.5. In some embodiments, Form E of Compound (ITT-1) may be characterized by a powder X-ray diffraction pattern with at least seven characteristic peaks, in degrees 2θ, each selected from the group consisting of the peaks listed in Table 3.5.

TABLE 3.5
III-1 Form E XRPD peak listing (the angle 2θ is within ± 0.2).

| Index | Angle | d Value | Rel. Intensity |
|---|---|---|---|
| 1 | 30.954 | 2.88664 | 3.90% |
| 2 | 11.042 | 8.0061 | 4.10% |
| 3 | 31.88 | 2.80488 | 4.40% |
| 4 | 35.67 | 2.51504 | 5.40% |
| 5 | 21.473 | 4.13489 | 6.50% |
| 6 | 36.84 | 2.43781 | 7.30% |
| 7 | 33.979 | 2.63626 | 7.90% |
| 8 | 37.834 | 2.37602 | 8.00% |
| 9 | 34.861 | 2.57156 | 9.50% |
| 10 | 39.293 | 2.29106 | 9.70% |
| 11 | 29.518 | 3.02372 | 10.20% |
| 12 | 9.934 | 8.89648 | 10.80% |
| 13 | 19.319 | 4.59072 | 10.90% |
| 14 | 33.349 | 2.68455 | 12.10% |
| 15 | 29.132 | 3.06284 | 12.30% |
| 16 | 20.296 | 4.37198 | 13.50% |
| 17 | 13.084 | 6.76115 | 16.00% |
| 18 | 28.282 | 3.15299 | 16.40% |
| 19 | 8.27 | 10.6834 | 17.30% |
| 20 | 32.337 | 2.76625 | 17.70% |
| 21 | 30.547 | 2.92412 | 18.00% |
| 22 | 14.94 | 5.92515 | 18.10% |
| 23 | 26.94 | 3.30697 | 18.40% |
| 24 | 26.628 | 3.34489 | 20.80% |
| 25 | 22.568 | 3.9366 | 21.20% |
| 26 | 24.652 | 3.60848 | 30.40% |
| 27 | 30.072 | 2.96921 | 33.00% |
| 28 | 27.211 | 3.27461 | 34.10% |
| 29 | 17.545 | 5.05076 | 36.10% |
| 30 | 23.518 | 3.77976 | 36.40% |
| 31 | 26.376 | 3.37631 | 37.70% |
| 32 | 11.801 | 7.49331 | 45.00% |
| 33 | 15.329 | 5.77558 | 46.70% |
| 34 | 5.529 | 15.9712 | 47.60% |
| 35 | 18.567 | 4.77494 | 47.90% |
| 36 | 13.592 | 6.50947 | 48.20% |
| 37 | 24.991 | 3.56017 | 67.10% |
| 38 | 16.631 | 5.32618 | 73.80% |
| 39 | 27.96 | 3.18854 | 84.90% |
| 40 | 25.539 | 3.48509 | 88.80% |
| 41 | 19.994 | 4.43731 | 90.00% |
| 42 | 22.189 | 4.0031 | 98.70% |
| 43 | 20.762 | 4.27496 | 100.00% |

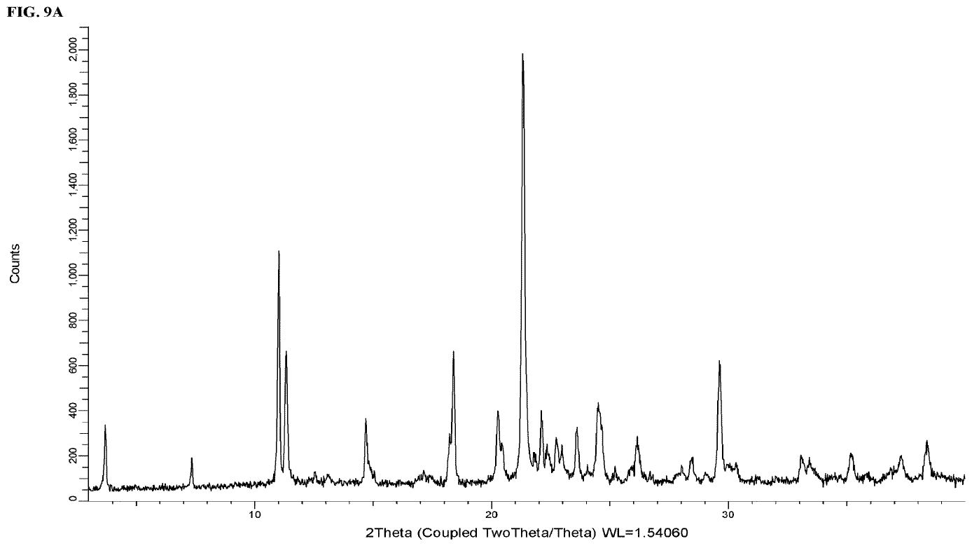

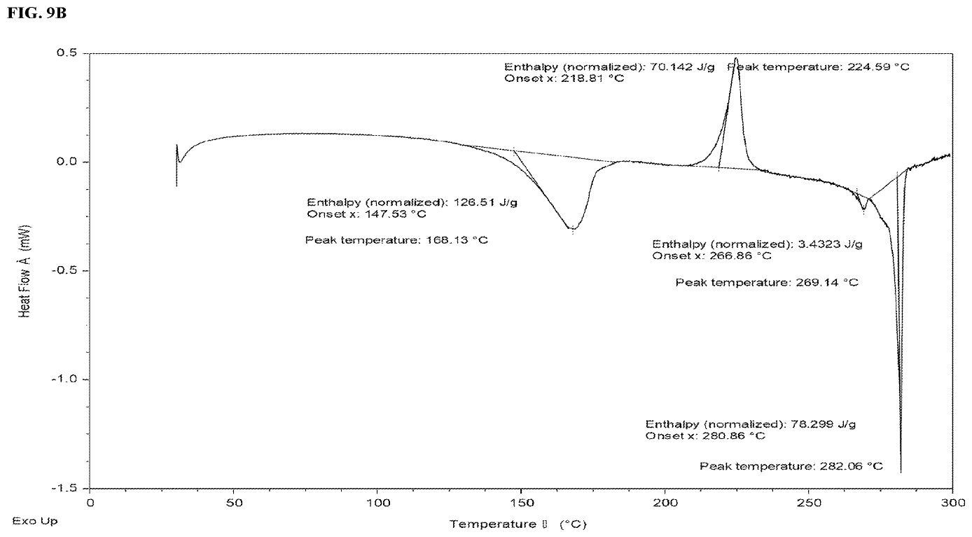

In some embodiments, the solid crystalline form of Compound (III-1) is Form F. In some embodiments, Form F of Compound (III-1) may be characterized by a powder X-ray diffraction pattern with at least two characteristic peaks, in degrees 2θ, each selected from the group consisting of about 21.3 2θ, about 11.0 2θ and about 11.3 2θ. In some embodiments, Form F of Compound (III-1) may be characterized by a powder X-ray diffraction pattern with at least two characteristic peaks, in degrees 2θ, each selected from the group consisting of about 21.3 2θ, about 11.0 2θ, about 11.3 2θ, about 18.4 2θ, about 29.6 2θ, about 24.5 2θ and about 20.3 2θ. In some embodiments, Form F of Compound (III-1) may be characterized by a powder X-ray diffraction pattern with at least three characteristic peaks, in degrees 2θ, each selected from the group consisting of about 21.3 2θ, about 11.0 2θ, about 11.3 2θ, about 18.4 2θ, about 29.6 2θ, about 24.5 2θ and about 20.3 2θ. In some embodiments, Form F of Compound (III-1) may be characterized by a powder X-ray diffraction pattern with at least four characteristic peaks, in degrees 2θ, each selected from the group consisting of about 21.3 2θ, about 11.0 2θ, about 11.3 2θ, about 18.4 2θ, about 29.6 2θ, about 24.5 2θ and about 20.3 2θ. In some embodiments, Form F of Compound (III-1) may be characterized by a powder X-ray diffraction pattern with at least five characteristic peaks, in degrees 2θ, each selected from the group consisting of about 21.3 2θ, about 11.0 2θ, about 11.3 2θ, about 18.4 2θ, about 29.6 2θ, about 24.5 2θ and about 20.3 2θ. In some embodiments, Form F of Compound (III-1) may be characterized by a powder X-ray diffraction pattern with at least six characteristic peaks, in degrees 2θ, each selected from the group consisting of about 21.3 2θ, about 11.0 2θ, about 11.3 2θ, about 18.4 2θ, about 29.6 2θ, about 24.5 2θ and about 20.3 2θ. In some embodiments, Form F of Compound (III-1) may be characterized by a powder X-ray diffraction pattern comprising characteristic peaks at about 21.3 2θ, about 11.0 2θ, about 11.3 2θ, about 18.4 2θ, about 29.6 2θ, about 24.5 2θ and about 20.3 2θ. In some embodiments, Form F of Compound (III-1) has a X-Ray diffraction pattern substantially similar to that depicted in FIG. 9A. In some embodiments, Form F of Compound (III-1) may be characterized by a powder X-ray diffraction pattern with at least two characteristic peaks, in degrees 2θ, each selected from the group consisting of the peaks listed in Table 3.6. In some embodiments, Form F of Compound (III-1) may be characterized by a powder X-ray diffraction pattern with at least three characteristic peaks, in degrees 2θ, each selected from the group consisting of the peaks listed in Table 3.6. In some embodiments, Form F of Compound (III-1) may be characterized by a powder X-ray diffraction pattern with at least four characteristic peaks, in degrees 2θ, each selected from the group consisting of the peaks listed in Table 3.6. In some embodiments, Form F of Compound (III-1) may be characterized by a powder X-ray diffraction pattern with at least five characteristic peaks, in degrees 2θ, each selected from the group consisting of the peaks listed in Table 3.6. In some embodiments, Form F of Compound (III-1) may be characterized by a powder X-ray diffraction pattern with at least six characteristic peaks, in degrees 2θ, each selected from the group consisting of the peaks listed in Table 3.6. In some embodiments, Form F of Compound (III-1) may be characterized by a powder X-ray diffraction pattern with at least seven characteristic peaks, in degrees 2θ, each selected from the group consisting of the peaks listed in Table 3.6.

TABLE 3.6
III-1 Form F XRPD peak listing (the angle 2θ is within ± 0.2).

| Index | Angle | d Value | Rel. Intensity |
|---|---|---|---|
| 1 | 17.415 | 5.08832 | 1.40% |
| 2 | 29.047 | 3.07166 | 1.50% |
| 3 | 35.835 | 2.50382 | 1.60% |
| 4 | 13.108 | 6.74859 | 1.80% |
| 5 | 26.694 | 3.33688 | 2.40% |
| 6 | 12.557 | 7.04354 | 2.80% |
| 7 | 17.147 | 5.16712 | 2.80% |
| 8 | 24.059 | 3.69606 | 2.80% |
| 9 | 25.894 | 3.43801 | 2.90% |
| 10 | 25.231 | 3.52684 | 3.20% |
| 11 | 28.036 | 3.18005 | 3.40% |
| 12 | 30.328 | 2.94475 | 4.10% |
| 13 | 21.798 | 4.07399 | 4.80% |
| 14 | 33.428 | 2.67839 | 4.80% |
| 15 | 28.456 | 3.13412 | 5.20% |
| 16 | 37.303 | 2.40862 | 5.90% |
| 17 | 33.071 | 2.7065 | 6.10% |
| 18 | 35.182 | 2.54877 | 6.10% |
| 19 | 22.973 | 3.86818 | 6.60% |
| 20 | 7.351 | 12.0166 | 6.90% |
| 21 | 22.346 | 3.9753 | 7.50% |
| 22 | 20.413 | 4.34708 | 7.60% |
| 23 | 22.755 | 3.90471 | 8.00% |
| 24 | 18.209 | 4.86798 | 8.30% |
| 25 | 38.398 | 2.34243 | 8.50% |
| 26 | 26.157 | 3.40413 | 10.60% |
| 27 | 23.603 | 3.76632 | 11.30% |
| 28 | 22.114 | 4.0164 | 13.90% |
| 29 | 3.699 | 23.8651 | 14.80% |
| 30 | 14.703 | 6.01997 | 14.80% |
| 31 | 20.282 | 4.37486 | 16.40% |
| 32 | 24.526 | 3.62671 | 17.70% |
| 33 | 29.647 | 3.01082 | 26.10% |
| 34 | 18.399 | 4.81808 | 31.30% |
| 35 | 11.335 | 7.79984 | 31.70% |
| 36 | 11.025 | 8.01881 | 54.50% |
| 37 | 21.336 | 4.1611 | 100.00% |

In some embodiments, Form F of Compound (III-1) has a differential scanning calorimetry (DSC) pattern substantially similar to that depicted in FIG. 9B.

### Compound III-2

In some embodiments, a compound of Formula (III) is Compound III-2:

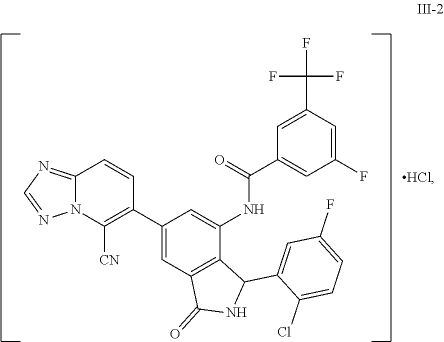

or a solvate thereof.

In some embodiments, Compound III-2 is an anhydrous solid.

In some embodiments, Compound III-2 is an amorphous solid. In other embodiments, Compound III-2 is a crystalline solid. In some embodiments, Compound III-2 is a mixture of amorphous solid form and crystalline solid form.

In some embodiments, the present invention provides a form of compound II-2 substantially free of impurities. As used herein, the term “substantially free of impurities” means that the compound contains no significant amount of extraneous matter. Such extraneous matter may include different forms of compound III-2, residual solvents, or any other impurities that may result from the preparation of, and/or isolation of, compound III-2.

In some embodiments, compound III-2, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound III-2, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound III-2, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound III-2, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound III-2 is also meant to include all tautomeric forms of compound III-2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound III-2 is a crystalline solid. In other embodiments, compound III-2 is a crystalline solid substantially free of amorphous compound III-2. As used herein, the term “substantially free of amorphous compound III-2” means that the compound contains no significant amount of amorphous compound III-2. In certain embodiments, at least about 95% by weight of crystalline compound III-2 is present. In still other embodiments of the invention, at least about 99% by weight of crystalline compound III-2 is present.

It has been found that compound III-2 can exist in a variety of solid forms. Exemplary such forms include polymorphs such as those described herein.

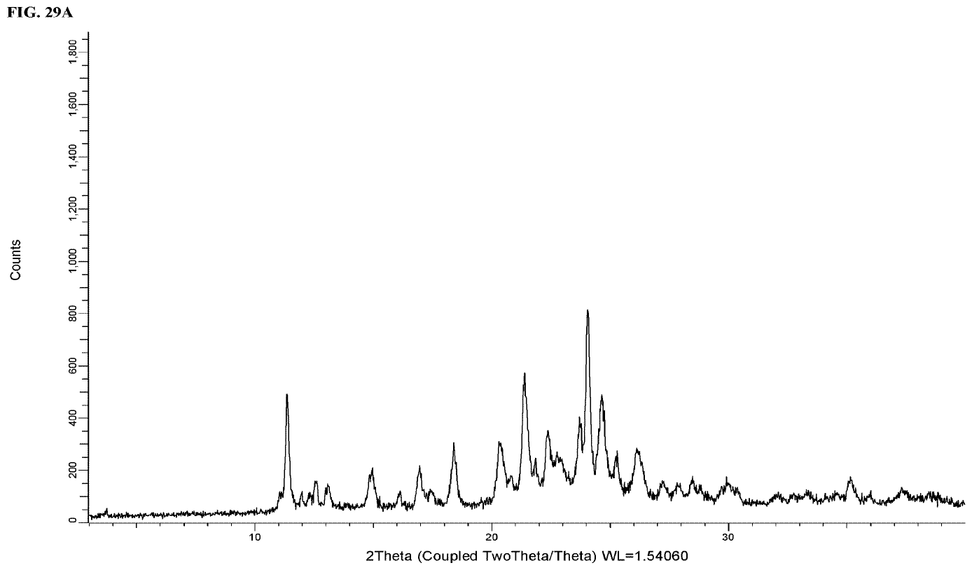

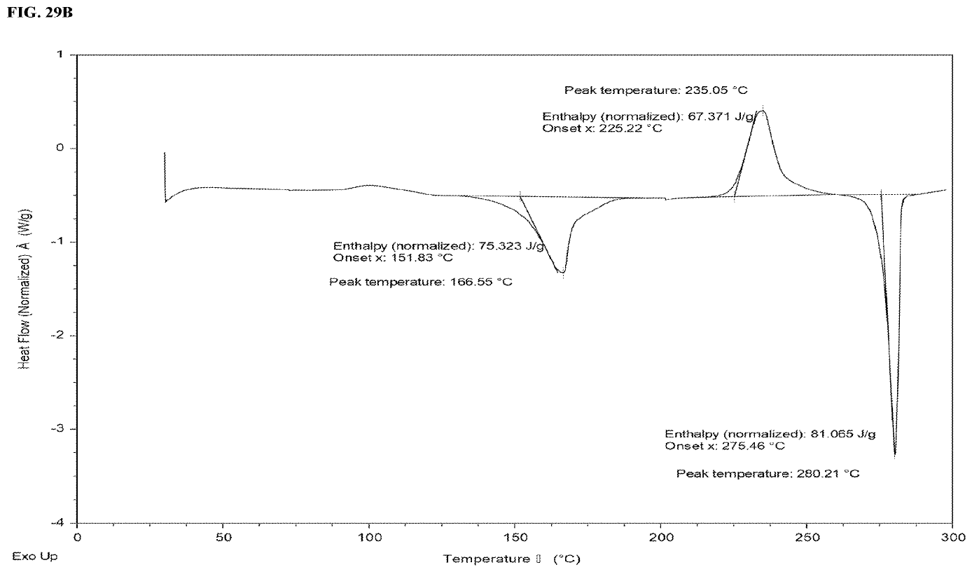

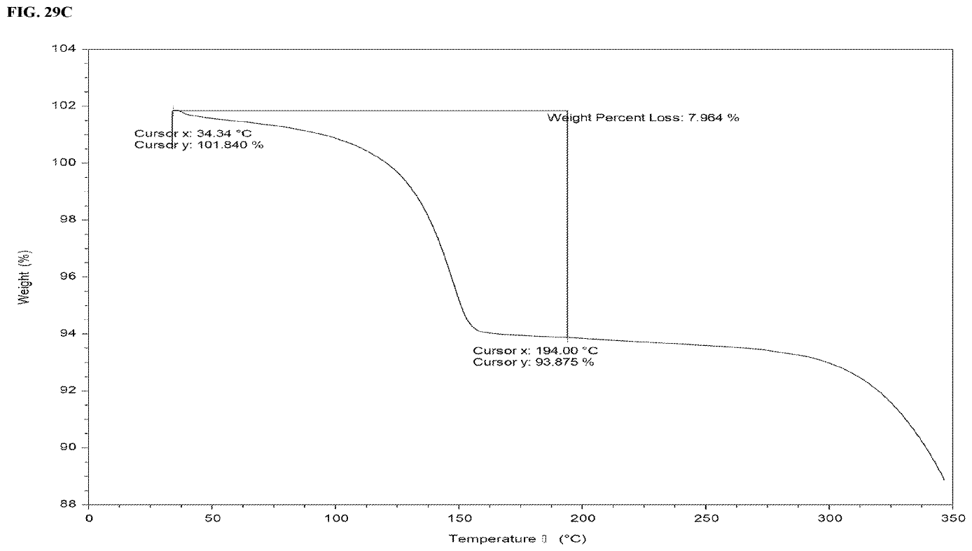

In some embodiments, the solid crystalline form of Compound III-2 is Form A. In some embodiments, Form A of Compound III-2 has a X-ray diffraction (XRPD) pattern substantially similar to that depicted in FIG. 29A.

In some embodiments, Form A of Compound III-2 has a differential scanning calorimetry (DSC) pattern substantially similar to that depicted in FIG. 29B. In some embodiments, Form A of Compound III-2 has a thermogravimetric analysis (TGA) pattern substantially similar to that depicted in FIG. 29C. In some embodiments, Form A of Compound III-2 can be characterized by substantial similarity to two or more of these figures simultaneously.

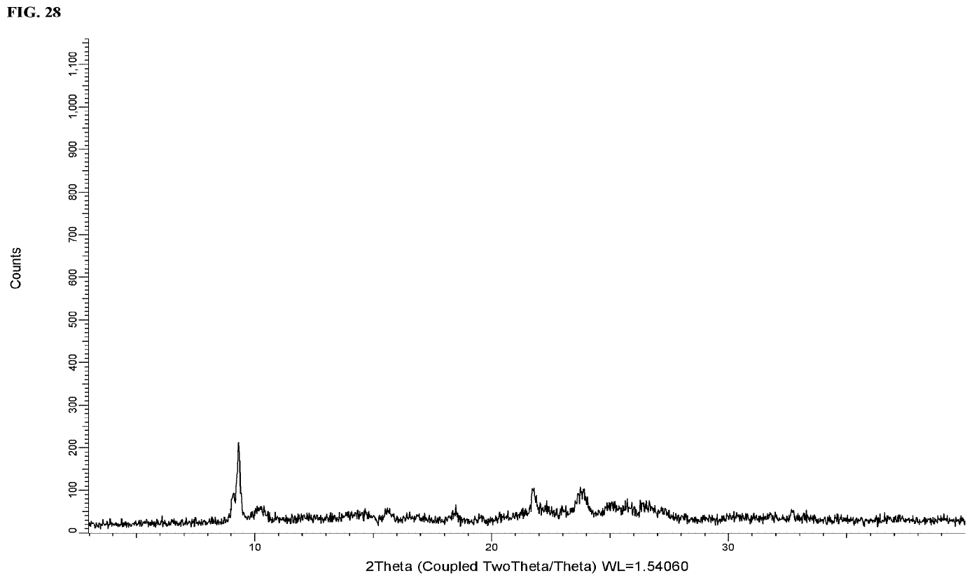

In some embodiments, the solid crystalline form of Compound III-2 is Form B. In some embodiments, Form B of Compound III-2 has a X-Ray diffraction pattern substantially similar to that depicted in FIG. 28.

### Compound III-3

In some embodiments, a compound of Formula (III) is Compound III-3:

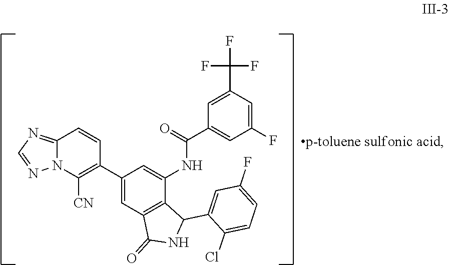

or a solvate thereof

In some embodiments, Compound III-3 is an anhydrous solid.

In some embodiments, Compound III-3 is an amorphous solid. In other embodiments, Compound III-3 is a crystalline solid. In some embodiments, Compound III-3 is a mixture of amorphous solid form and crystalline solid form.

In some embodiments, the present invention provides a form of compound III-3 substantially free of impurities. As used herein, the term “substantially free of impurities” means that the compound contains no significant amount of extraneous matter. Such extraneous matter may include different forms of compound III-3, residual solvents, or any other impurities that may result from the preparation of, and/or isolation of, compound III-3.

In some embodiments, compound III-3, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound III-3, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound III-3, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound III-3, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound III-3 is also meant to include all tautomeric forms of compound III-3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound III-3 is a crystalline solid. In other embodiments, compound III-3 is a crystalline solid substantially free of amorphous compound III-3. As used herein, the term “substantially free of amorphous compound III-3” means that the compound contains no significant amount of amorphous compound III-3. In certain embodiments, at least about 95% by weight of crystalline compound III-3 is present. In still other embodiments of the invention, at least about 99% by weight of crystalline compound III-3 is present.

### Compound III-4

In some embodiments, a compound of Formula (I) is Compound III-4:

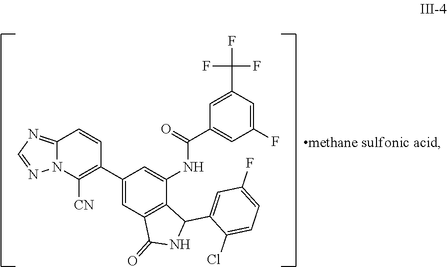

or a solvate thereof.

In some embodiments, Compound III-4 is an anhydrous solid.

In some embodiments, Compound III-4 is an amorphous solid. In other embodiments, Compound III-4 is a crystalline solid. In some embodiments, Compound III-4 is a mixture of amorphous solid form and crystalline solid form.

In some embodiments, the present invention provides a form of compound III-4 substantially free of impurities. As used herein, the term “substantially free of impurities” means that the compound contains no significant amount of extraneous matter. Such extraneous matter may include different forms of compound III-4, residual solvents, or any other impurities that may result from the preparation of, and/or isolation of, compound III-4.

In some embodiments, compound III-4, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound III-4, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound III-4, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound III-4, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound III-4 is also meant to include all tautomeric forms of compound III-4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound III-4 is a crystalline solid. In other embodiments, compound III-4 is a crystalline solid substantially free of amorphous compound III-4. As used herein, the term “substantially free of amorphous compound III-4” means that the compound contains no significant amount of amorphous compound III-4. In certain embodiments, at least about 95% by weight of crystalline compound III-4 is present. In still other embodiments of the invention, at least about 99% by weight of crystalline compound III-4 is present.

### Compound III-5

In some embodiments, a compound of Formula (I) is Compound III-5:

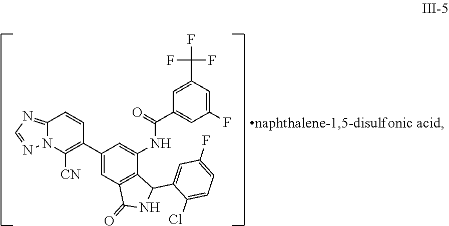

or a solvate thereof.

In some embodiments, Compound III-5 is an anhydrous solid.

In some embodiments, Compound III-5 is an amorphous solid. In other embodiments, Compound III-5 is a crystalline solid. In some embodiments, Compound III-5 is a mixture of amorphous solid form and crystalline solid form.

In some embodiments, the present invention provides a form of compound III-5 substantially free of impurities. As used herein, the term “substantially free of impurities” means that the compound contains no significant amount of extraneous matter. Such extraneous matter may include different forms of compound III-5, residual solvents, or any other impurities that may result from the preparation of, and/or isolation of, compound III-5.

In some embodiments, compound III-5, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound III-5, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound III-5, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound III-5, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound III-5 is also meant to include all tautomeric forms of compound III-5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound III-5 is a crystalline solid. In other embodiments, compound III-5 is a crystalline solid substantially free of amorphous compound III-5. As used herein, the term “substantially free of amorphous compound III-5” means that the compound contains no significant amount of amorphous compound III-5. In certain embodiments, at least about 95% by weight of crystalline compound III-5 is present. In still other embodiments of the invention, at least about 99% by weight of crystalline compound III-5 is present.

### Compound III-6

In some embodiments, a compound of Formula (III) is Compound III-6:

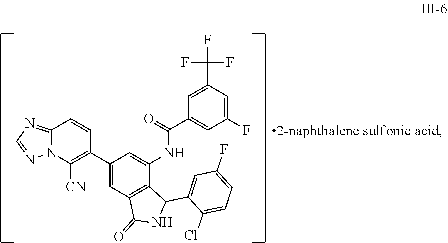

or a solvate thereof.

In some embodiments, Compound III-6 is an anhydrous solid.

In some embodiments, Compound III-6 is an amorphous solid. In other embodiments, Compound III-6 is a crystalline solid. In some embodiments, Compound III-6 is a mixture of amorphous solid form and crystalline solid form.

In some embodiments, the present invention provides a form of compound III-6 substantially free of impurities. As used herein, the term “substantially free of impurities” means that the compound contains no significant amount of extraneous matter. Such extraneous matter may include different forms of compound III-6, residual solvents, or any other impurities that may result from the preparation of, and/or isolation of, compound III-6.

In some embodiments, compound III-6, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, compound III-6, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, an impurity is selected from those as described in the examples herein.

In some embodiments, compound III-6, or a solvate thereof, or a crystalline form thereof, is present in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, compound III-6, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, an impurity is selected from those as described in the examples herein. In some embodiments, a HPLC method is selected from the HPLC methods as described in Examples herein.

The structure depicted for compound III-6 is also meant to include all tautomeric forms of compound III-6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound III-6 is a crystalline solid. In other embodiments, compound III-6 is a crystalline solid substantially free of amorphous compound III-6. As used herein, the term “substantially free of amorphous compound III-6” means that the compound contains no significant amount of amorphous compound III-6. In certain embodiments, at least about 95% by weight of crystalline compound III-6 is present. In still other embodiments of the invention, at least about 99% by weight of crystalline compound III-6 is present.

It has been found that compound III-6 can exist in a variety of solid forms. Exemplary such forms include polymorphs such as those described herein.

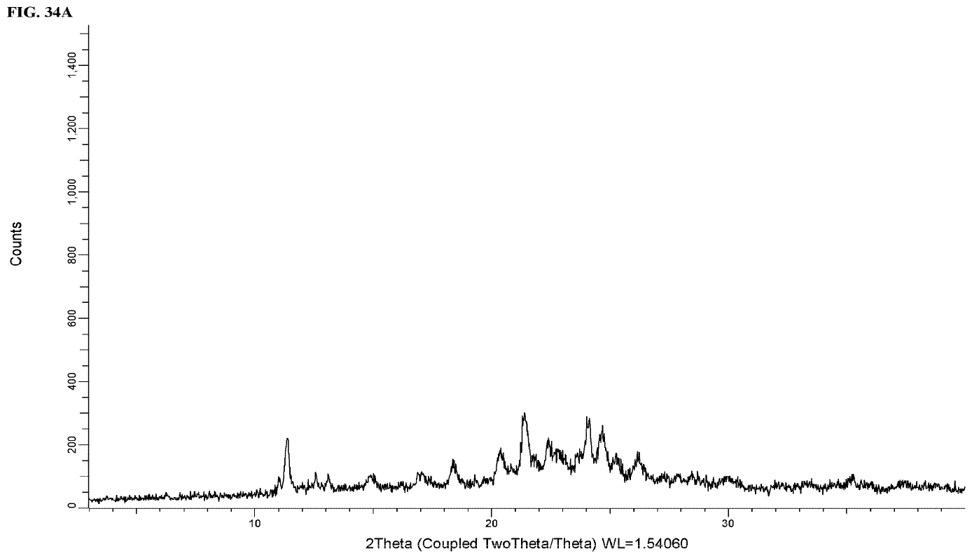

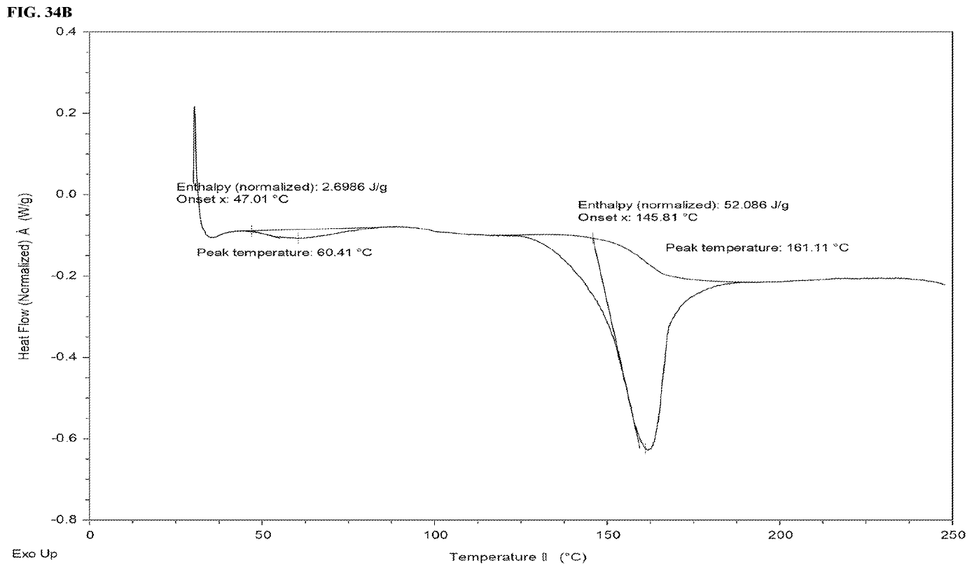

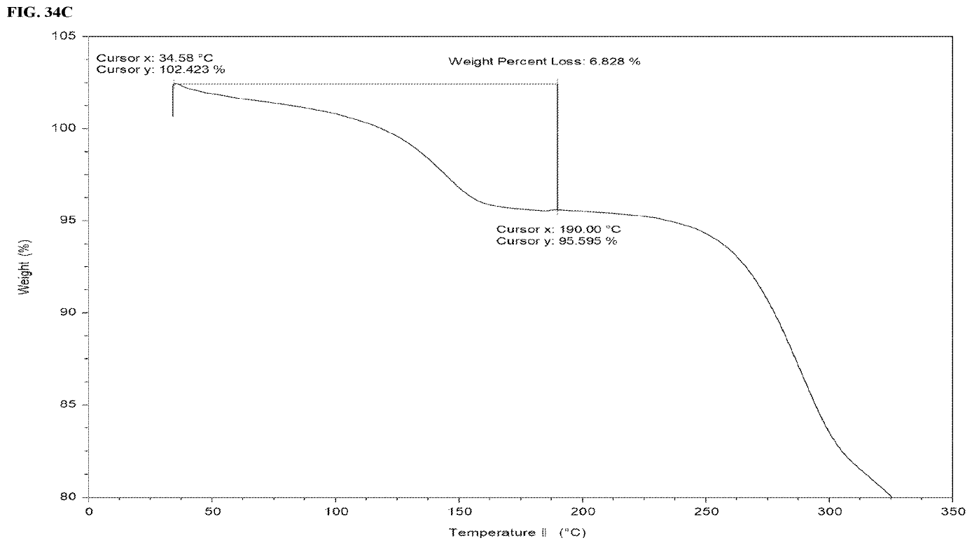

In some embodiments, the solid crystalline form of Compound III-6 is Form A. In some embodiments, Form A of Compound III-6 has a X-Ray diffraction pattern substantially similar to that depicted in FIG. 34A.

In some embodiments, Form A of Compound III-6 has a differential scanning calorimetry (DSC) pattern substantially similar to that depicted in FIG. 34B. In some embodiments, Form A of Compound III-6 has a thermogravimetric analysis (TGA) pattern substantially similar to that depicted in FIG. 34C. In some embodiments, Form A of Compound III-6 can be characterized by substantial similarity to two or more of these figures simultaneously.

### Compound of Formula (IV-1) and (IV-2)

In some embodiments, provided herein is a compound of Formula (IV-1)

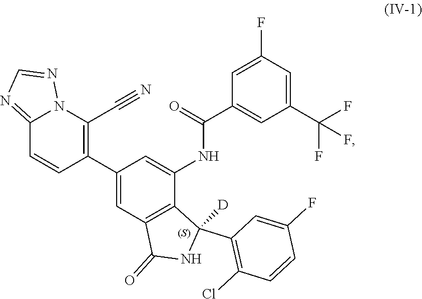

or a pharmaceutically acceptable salt thereof.

In some embodiments, provided herein is a compound of Formula (IV-2)

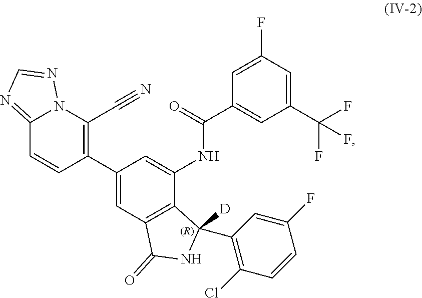

or a pharmaceutically acceptable salt thereof.

### Compositions

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, topical, buccal, ocular, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral, subcutaneous or intravenous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments, pharmaceutically acceptable compositions can contain a disclosed compound and/or a pharmaceutically acceptable salt thereof at a concentration ranging from about 0.01 to about 2.0 wt %, such as 0.01 to about 1 wt % or about 0.05 to about 0.5 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

For preparing solid compositions such as tablets, the principal active ingredient may be mixed with a pharmaceutical carrier, e.g., conventional tableting ingredients such as com starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient. Often, the pharmaceutically acceptable carrier is chemically inert toward the active compounds and is non-toxic under the conditions of use. Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modem Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

In some embodiments, the compounds of the disclosure are formulated into pharmaceutical compositions for administration to subjects in a biologically compatible form suitable for administration in vivo. According to another aspect, the present disclosure provides a pharmaceutical composition comprising a disclosed compound in admixture with a pharmaceutically acceptable diluent and/or carrier. The pharmaceutically-acceptable carrier is “acceptable” in the sense of being compatible with the other ingredients of the composition and not deleterious to the recipient thereof. The pharmaceutically-acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

When administered to a subject, the disclosed compound and pharmaceutically acceptable carriers can be sterile. Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain minor amounts of wetting or emulsifying agents, or pH buffering agents.

The pharmaceutical formulations of the present disclosure are prepared by methods well-known in the pharmaceutical arts. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

Additionally, the compounds and/or compositions of the present disclosure are administered to a human or animal subject by known procedures including oral administration, sublingual or buccal administration. In some embodiments, the compound and/or composition is administered orally.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

For oral administration, a formulation of the compounds of the disclosure may be presented in dosage forms such as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants. For example, crystalline forms provided herein may be milled to obtain a particular particle size, and in at least some embodiments, such crystalline forms may remain substantially stable upon milling.

For example, provided herein is a composition suitable for subcutaneous administration, comprising a suspension of the disclosed crystalline form. Subcutaneous administration can be advantageous over intravenous administration, which typically requires a doctor visit, and can be more painful and invasive. A typical dose of the crystalline compound, when administered to a patient, may be about 1 mg to about 8 mg of compound. In an embodiment, disclosed herein is a pharmaceutically acceptable composition formed from a disclosed crystalline form, e.g. by mixing a crystalline form with an excipient and/or a solvent.

In an embodiment, provided herein is a composition comprising a disclosed crystalline form suitable for subcutaneous administration at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.001 mg/kg to about 4 mg/kg, from about 0.1 mg/kg to about 10 mg/kg, from about 1 mg/kg to about 25 mg/kg, of subject body weight, administered daily, one or more times a day, every other day, every third or fourth day, every week, every two weeks, every three weeks, or every four weeks. In certain embodiments, the desired dosage may be delivered using multiple administrations (e.g., two, three, four, five, six, seven, eight, nine, or ten administrations). In certain embodiments, administration may occur once, twice, or thrice weekly.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a weight loss target, is achieved. A treatment regimen can include a corrective phase, during which dose sufficient to provide reduction of weight is administered, and can be followed by a maintenance phase, during which a e.g. lower dose sufficient to weight gain is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain body weight in subjects whose body weight has been previously controlled by other means, including diet and exercise, bariatric procedures such as bypass or banding surgeries, or treatments employing other pharmacological agents.

In certain embodiments, provided herein is a pharmaceutical composition comprising a crystalline form of compound I, II, or III, or a solvate thereof, as described herein. In certain embodiments, provided herein is a pharmaceutical composition comprising a crystalline form of compound I-1, including, for example, Form A, Form B, or Form C, or a solvate thereof, as described herein. In certain embodiments, provided herein is a pharmaceutical composition comprising a crystalline form of compound III-1, including, for example, Form A, Form B, Form C, Form D, Form E, or Form F, or a solvate thereof, as described herein. In certain embodiments, provided herein is a pharmaceutical composition comprising compound of Formula IV-1 or IV-2, or a pharmaceutically acceptable salt thereof, as described herein. In certain embodiments, a pharmaceutical composition provided herein comprises one or more pharmaceutically acceptable excipient, as described herein.

### Kits

In one embodiment, a kit for treating or mitigating a contemplated disease of disorder is provided. For example, a disclosed kit comprises a disclosed crystalline compound, e.g. a crystalline form of a compound of Formula (I), disposed in a first container. In some embodiments, a kit may further include a pharmaceutically acceptable excipient, disposed in a second container. Such contemplated kits may include written instructions describing preparation of a pharmaceutical composition suitable for administration to a patient from the crystalline form. For example, the written instructions may describe preparing a pharmaceutically acceptable form for patient administration by mixing an excipient and a crystalline compound disclosed herein. Disclosed kits may further comprise written instructions describing how to administer the resulting composition to the patient.

In one embodiment, a kit for treating or mitigating a contemplated disease of disorder is provided. For example, a disclosed kit comprises a compound as described herein, disposed in a first container. In some embodiments, a kit may further include a pharmaceutically acceptable excipient, disposed in a second container. Such contemplated kits may include written instructions describing preparation of a pharmaceutical composition suitable for administration to a patient from a disclosed compound. For example, the written instructions may describe preparing a pharmaceutically acceptable form for patient administration by mixing an excipient and a compound disclosed herein. Disclosed kits may further comprise written instructions describing how to administer the resulting composition to the patient.

### Processes

In some embodiments, a process for preparing a disclosed, crystalline form of a compound of Formula (I) is contemplated herein, comprising: a) preparing a solution of a compound of Formula (I); b) adjusting the temperature so that solid crystalline form of a compound of Formula (I) precipitates out of the solution; and c) isolating the solid crystalline form. In some embodiments, the step of preparing a solution of a compound of Formula (I) comprises mixing a solution of compound I-1 with an solution of acid X, wherein X is as defined and described in embodiments herein. In some embodiments, a solution of a compound of Formula (I) comprises a solvent selected from Methanol, Ethanol, Acetone, Methyl ethyl ketone, Ethyl acetate, Isopropyl acetate, Acetonitrile, t-Butyl methyl ether, Dichloromethane, Tetrahydrofuran, 1,4-Dioxane, Benzyl alcohol, 2-MeTHF, IPAc, and MtBE. In some embodiments, a solution of a compound of Formula (I) comprises a solvent selected from those as described in the examples herein.

In some embodiments, a process for preparing a disclosed, crystalline form of a compound of Formula (II) is contemplated herein, comprising: a) preparing a solution of a compound of Formula (II); b) adjusting the temperature so that solid crystalline form of a compound of Formula (II) precipitates out of the solution; and c) isolating the solid crystalline form. In some embodiments, the step of preparing a solution of a compound of Formula (II) comprises mixing a solution of compound II-1 with an solution of acid X, wherein X is as defined and described in embodiments herein. In some embodiments, a solution of a compound of Formula (II) comprises a solvent selected from Methanol, Ethanol, Acetone, Methyl ethyl ketone, Ethyl acetate, Isopropyl acetate, Acetonitrile, t-Butyl methyl ether, Dichloromethane, Tetrahydrofuran, 1,4-Dioxane, Benzyl alcohol, 2-MeTHF, IPAc, and MtBE. In some embodiments, a solution of a compound of Formula (II) comprises a solvent selected from those as described in the examples herein.

In some embodiments, a process for preparing a disclosed, crystalline form of a compound of Formula (III) is contemplated herein, comprising: a) preparing a solution of a compound of Formula (III); b) adjusting the temperature so that solid crystalline form of a compound of Formula (III) precipitates out of the solution; and c) isolating the solid crystalline form. In some embodiments, the step of preparing a solution of a compound of Formula (III) comprises mixing a solution of compound III-1 with an solution of acid X, wherein X is as defined and described in embodiments herein. In some embodiments, a solution of a compound of Formula (III) comprises a solvent selected from Methanol, Ethanol, Acetone, Methyl ethyl ketone, Ethyl acetate, Isopropyl acetate, Acetonitrile, t-Butyl methyl ether, Dichloromethane, Tetrahydrofuran, 1,4-Dioxane, Benzyl alcohol, 2-MeTHF, IPAc, and MtBE. In some embodiments, a solution of a compound of Formula (III) comprises a solvent selected from those as described in the examples herein.

In some embodiments, a solution of a compound of Formulae (I), (II), or (III) comprises a solvent selected from MeOH, EtOH, acetone, IPAc, MtBE, acetonitrile, EtOAc, IPA, THF, heptane, 1,4 dioxane, DMF, and water. In some embodiments, a solution of a compound of Formulae (I), (II), or (III) comprises a solvent of acetone/heptane (1:2, v/v). In some embodiments, a solution of a compound of Formulae (I), (II), or (III) comprises a solvent of acetone/MTBE (1:4, v/v). In some embodiments, a solution of a compound of Formulae (I), (II), or (III) comprises a solvent of THF/heptane(2:3, v/v). In some embodiments, a solution of a compound of Formulae (I), (II), or (III) comprises a solvent of Ethyl acetate/heptane(1:1, v/v). In some embodiments, a solution of a compound of Formulae (I), (II), or (III) comprises a solvent of THF/MTBE(1:4, v/v). In some embodiments, a solution of a compound of Formulae (I), (II), or (III) comprises a solvent of THF/ACN(2:1, v/v). In some embodiments, a solution of a compound of Formulae (I), (II), or (III) comprises a solvent of EtOH/water(50:50, v/v). In some embodiments, a solution of a compound of Formulae (I), (II), or (III) comprises a solvent of ACN/water(80:20, v/v). In some embodiments, a solution of a compound of Formulae (I), (II), or (III) comprises a solvent of THF/water (85:15, v/v). In some embodiments, a solution of a compound of Formulae (I), (II), or (III) comprises a solvent of acetone/water (60:40, v/v). In some embodiments, a solution of a compound of Formulae (I), (II), or (III) comprises a solvent of THF/heptane (2:3, v/v). In some embodiments, a solution of a compound of Formulae (I), (II), or (III) comprises a solvent of THF/MTBE (1:4, v/v). In some embodiments, a solution of a compound of Formulae (I), (II), or (III) comprises a solvent of MeOH/MTBE (1:4, v/v). In some embodiments, a solution of a compound of Formulae (I), (II), or (III) comprises a solvent of DMF/Acetone water.

In some embodiments, heating the solution comprises heating the solution to about 50° C. In some embodiments, adjusting the temperature comprises cooling the solution to about 25° C.

In other embodiments, a disclosed process further comprises a purification step to separate the enantiomers of compound III-1, thereby forming compound I-1:

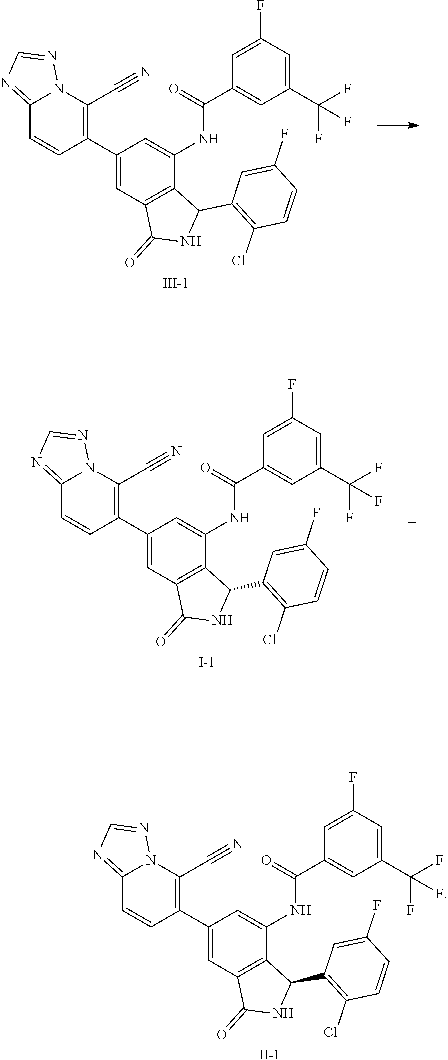

In other embodiments, a disclosed process further comprises a purification step to separate the enantiomers of compound III-1 by subjecting compound III-1 to a SMB separation, for example, as described in Example 1-A, thereby forming compounds I-1 and II-1:

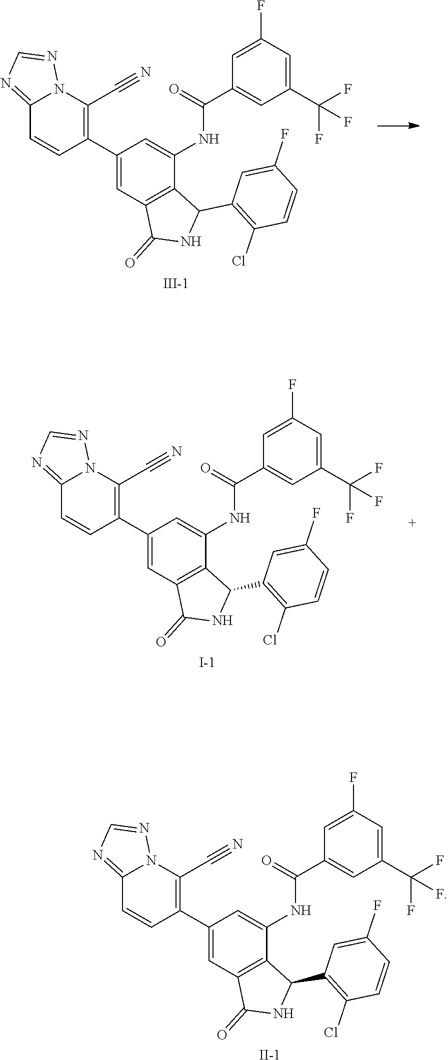

In other embodiments, a disclosed process further comprises a racemization of II-1, thereby forming mixture of I-1 and II-1 (or III-1):

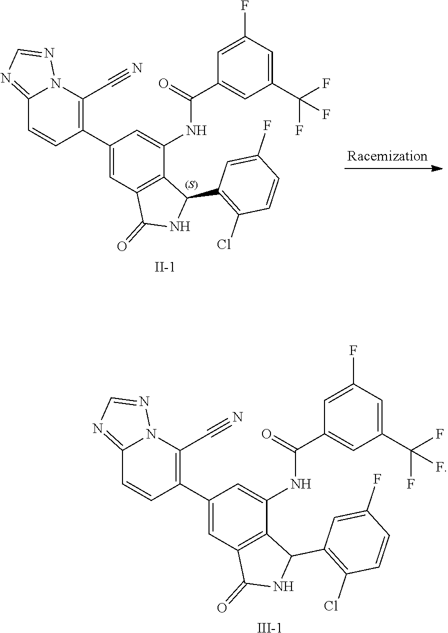

In other embodiments, a disclosed process further comprises a racemization of compound II-1, thereby forming compound III-1 (mixture of I-1 and II-1):

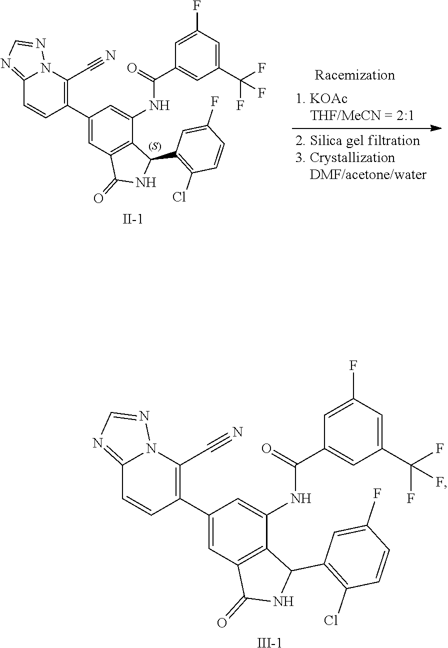

for example, as described in Example 2-A.

In other embodiments, a disclosed process further comprises a step of coupling compound 2 with compound 3, thereby forming compound III-1:

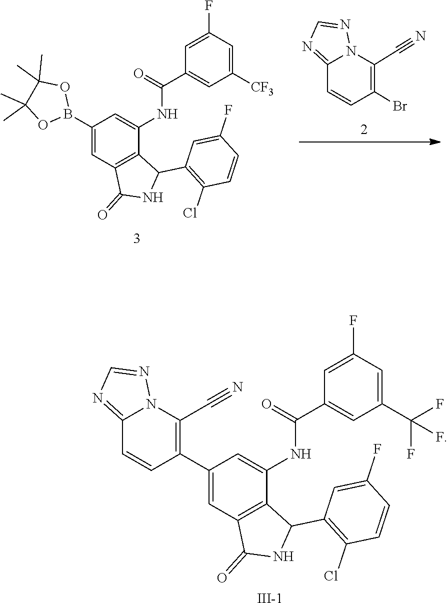

In other embodiments, a disclosed process further comprises a step of converting compound 4 to compound 3:

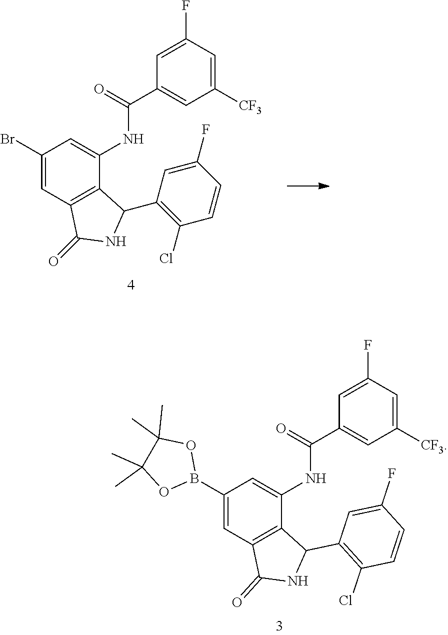 [SMILES: O=C(Nc1cc(Br)cc2c1C(c1cc(F)ccc1Cl)NC2=O)c1cc(F)cc(C(F)(F)F)c1 ; CC1(C)OB(c2cc(NC(=O)c3cc(F)cc(C(F)(F)F)c3)c3c(c2)C(=O)NC3c2cc(F)ccc2Cl)OC1(C)C]

In other embodiments, a disclosed process further comprises the step of converting compound 5 to compound 4:

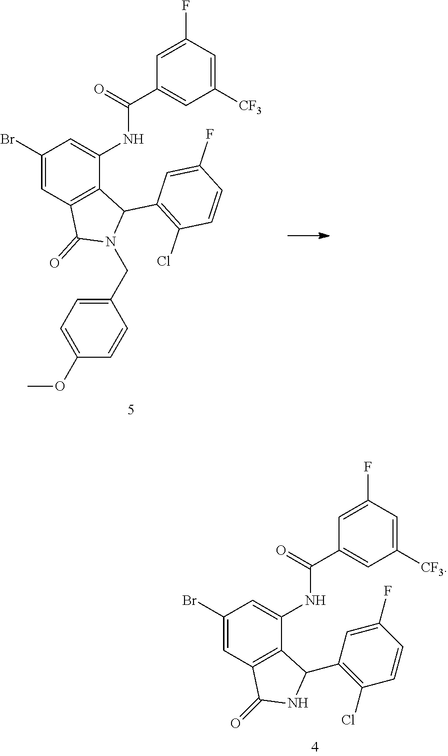 [SMILES: COc1ccc(CN2C(=O)c3cc(Br)cc(NC(=O)c4cc(F)cc(C(F)(F)F)c4)c3C2c2cc(F)ccc2Cl)cc1 ; O=C(Nc1cc(Br)cc2c1C(c1cc(F)ccc1Cl)NC2=O)c1cc(F)cc(C(F)(F)F)c1]

In other embodiments, a disclosed process further comprises the step of coupling compound 6 with compound 7, thereby forming compound 5:

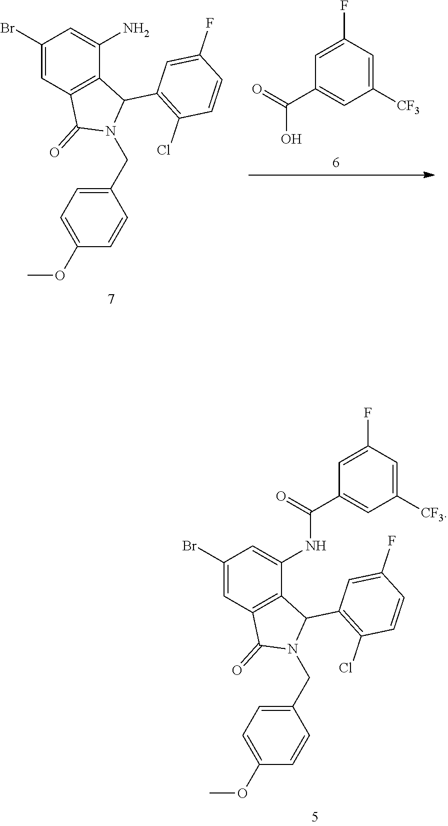

In some embodiments, a disclosed process comprises deuteration of compound III-1 followed by a purification step to separate the enantiomers, thereby forming compounds IV-1 and IV-2:

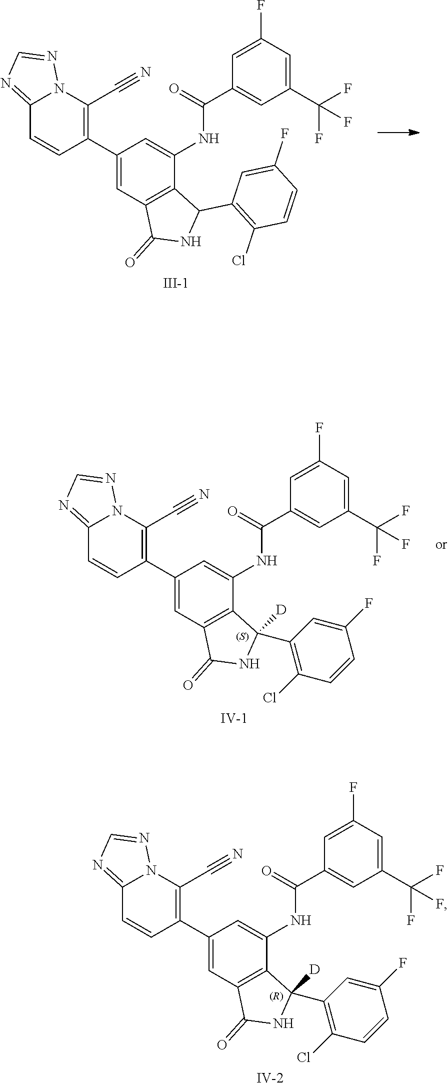

for example, as described in Example 3-A.

### Methods

Compounds and compositions described herein are generally useful for the inhibition of a kinase or a mutant thereof. In some embodiments, the kinase inhibited by the compounds and compositions described herein is a phosphatidylinositol 3-kinase (PI3K). In some embodiments, the kinase inhibited by the compounds and compositions described herein is one or more of a PI3Kα, PI3Kδ, and PI3Kγ. In some embodiments, the kinase inhibited by the compounds and compositions described herein is a PI3Kα. In some embodiments, the kinase inhibited by the compounds and compositions described herein is a PI3Kα containing at least one of the following mutations: E542X, E545X, Q546X, H1047X, and G1049X, wherein X is any amino acid besides its wildtype. In some embodiments, the kinase inhibited by the compounds and compositions described herein is a PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the kinase inhibited by the compounds and compositions described herein is a PI3Kα containing at least one of the following mutations: E542K, E545K, and H1047R. In some embodiments, the kinase inhibited by the compounds and compositions described herein is a PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the kinase inhibited by the compounds and compositions described herein is a PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

Compounds or compositions of the disclosure can be useful in applications that benefit from inhibition of PI3K enzymes. For example, PI3K inhibitors of the present invention are useful for the treatment of cellular proliferative diseases generally. Compounds or compositions of the disclosure can be useful in applications that benefit from inhibition of PI3Kα enzymes. For example, PI3Kα inhibitors of the present invention are useful for the treatment of cellular proliferative diseases generally.

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

### Treatment of Disorders

Provided compounds are inhibitors of PI3Kα and are therefore useful for treating one or more disorders associated with activity of PI3Kα or mutants thereof. Thus, in certain embodiments, the present invention provides a method of treating a PI3Kα-mediated disorder in a subject, comprising administering a therapeutically effective amount of a compound of the present invention, or a pharmaceutically acceptable salt thereof, or a pharmaceutically acceptable composition of either of the foregoing, to a subject in need thereof. In certain embodiments, the present invention provides a method of treating a PI3Kα-mediated disorder in a subject comprising administering a therapeutically effective amount of a compound of the present invention, or a pharmaceutically acceptable composition thereof, to a subject in need thereof. In some embodiments, the subject has a mutant PI3Kα. In some embodiments, the subject has PI3Kα containing at least one of the following mutations: H1047R, E542K, and E545K. In some embodiments, the subject has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the subject has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the subject has PI3Kα containing at least one of the following mutations: E81K, R88Q, R93Q, R93W, G106R, G106V, R108H, K111N, K111E, GT18D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M1043I, M1043V, N1044K, H1047R, H1047L, H1047Y, G1049R, G1049S, I1058F, A1066V, and N1068fs*4.

As used herein, the term “PI3Kα-mediated” disorders, diseases, and/or conditions means any disease or other deleterious condition in which PI3Kα or a mutant thereof is known to play a role. Accordingly, another embodiment of the present invention relates to treating or lessening the severity of one or more diseases in which PI3Kα, or a mutant thereof, is known to play a role. Such PI3Kα-mediated disorders include, but are not limited to, cellular proliferative disorders (e.g. cancer). In some embodiments, the PI3Kα-mediated disorder is a disorder mediated by a mutant PI3Kα. In some embodiments, the PI3Kα-mediated disorder is a disorder mediated by a PI3Kα containing at least one of the following mutations: H1047R, E542K, and E545K. In some embodiments, the subject has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the subject has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the subject has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the present invention provides a method for treating a cellular proliferative disease, said method comprising administering to a patient in need thereof a therapeutically effective amount of a compound of the present invention, or a pharmaceutically acceptable salt thereof, or a pharmaceutically acceptable composition of either of the foregoing. In some embodiments, the present invention provides a method for treating a cellular proliferative disease, said method comprising administering to a patient in need thereof, a therapeutically effective amount of a compound of the present invention, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof, and (iii) administering said provided compound in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K. In some embodiments, the subject has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the subject has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the subject has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the method of treatment comprises the steps of: i) identifying a subject in need of such treatment; (ii) providing a composition comprising a disclosed compound, or a pharmaceutically acceptable salt thereof, and (iii) administering said composition in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K. In some embodiments, the subject has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the subject has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the subject has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

Another aspect of the invention provides a compound according to the definitions herein, or a pharmaceutically acceptable salt thereof, or a pharmaceutical composition of either of the foregoing, for use in the treatment of a disorder described herein. Another aspect of the invention provides the use of a compound according to the definitions herein, or a pharmaceutically acceptable salt thereof, or a pharmaceutical composition of either of the foregoing, for the treatment of a disorder described herein. Similarly, the invention provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the solid tumor has PI3Kα containing at least one of the following mutations: E542X, E545X, Q546X, H1047X, and G1049X, wherein X is any amino acid besides its wildtype. In some embodiments, the solid tumor has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the solid tumor has PI3Kα containing at least one of the following mutations: H1047R, E542K, and E545K. In some embodiments, the solid tumor has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the solid tumor has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is selected from breast, brain, cervix, endometrium, esophagus, lymph node, kidney, large intestine, liver, lung, ovary, pancreas, penis, prostate, skin, small intestine, stomach, thyroid, head and neck, thymus, and bladder. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+ breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cancer is ovarian cancer. In some embodiments, the ovarian cancer is clear cell ovarian cancer.

In some embodiments, the cancer is squamous cell carcinoma. In some embodiments, the cancer is squamous cell carcinoma of the head and neck.

In some embodiments, the cancer is cervical cancer.

In some embodiments, the cellular proliferative disease has mutant PI3Kα. In some embodiments, the cancer has mutant PI3Kα. In some embodiments, the breast cancer has mutant PI3Kα. In some embodiments, the ovarian cancer has mutant PI3Kα. In some embodiments, the clear cell ovarian cancer has mutant PI3Kα.

In some embodiments, the cellular proliferative disease has PI3Kα containing at least one of the following mutations: H1047R, E542K, and E545K. In some embodiments, the cellular proliferative disease has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the cellular proliferative disease has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the cellular proliferative disease has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the cancer has PI3Kα containing at least one of the following mutations: H1047R, E542K, and E545K. In some embodiments, the cancer has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the cancer has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the cancer has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the breast cancer has PI3Kα containing at least one of the following mutations: H1047R, E542K, and E545K. In some embodiments, the breast cancer has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the breast cancer has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the breast cancer has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the ovarian cancer has PI3Kα containing at least one of the following mutations: H1047R, E542K, and E545K. In some embodiments, the ovarian cancer has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the ovarian cancer has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the ovarian cancer has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the clear cell ovarian cancer has PI3Kα containing at least one of the following mutations: H1047R, E542K, and E545K. In some embodiments, the clear cell ovarian cancer has PI3Kα containing at least one of the following mutations: E542K, E542Q, E545A, E545G, E545K, E545Q, Q546E, Q546K, Q546L, Q546P, Q546R, H1047R, H1047L, H1047Y, G1049R, and G1049S. In some embodiments, the clear cell ovarian cancer has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the clear cell ovarian cancer has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of PI3Kα, somatic mutation of PIK3CA, germline mutations or somatic mutation of PTEN, or mutations and translocation of p85a that serve to up-regulate the p85-p110 complex. In some embodiments, the cellular proliferative disease displays overexpression or amplification of PI3Kα. In some embodiments, the cellular proliferative disease displays somatic mutation of PIK3CA. In some embodiments, the cellular proliferative disease displays germline mutations or somatic mutation of PTEN. In some embodiments, the cellular proliferative disease displays mutations and translocation of p85a that serve to up-regulate the p85-p110 complex.

### Additional Disorders

In some embodiments, the PI3Kα-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the PI3Kα-mediated disorder is polycythemia vera, essential thrombocythemia, or myelofibrosis with myeloid metaplasia. In some embodiments, the PI3Kα-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the PI3Kα-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, or scleroderma. In some embodiments, the PI3Kα-mediated disorder is vitiligo, hypersensitivity angiitis, urticaria, bullous pemphigoid, lupus erythematosus, pemphisus, epidermolysis bullosa acquisita, or autoimmune haematogical disorders (e.g. haemolytic anaemia, aplastic anaemia, pure red cell anaemia and idiopathic thrombocytopenia). In some embodiments, the PI3Kα-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g. ulcerative colitis and Crohn's disease).

In some embodiments, the PI3Kα-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the PI3Kα-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the PI3Kα-mediated disorder is retinopathy, such as diabetic retinopathy or hyperbaric oxygen-induced retinopathy, and conditions characterized by elevated intraocular pressure or secretion of ocular aqueous humor, such as glaucoma.

### Routes of Administration and Dosage Forms

The compounds and compositions, according to the methods of the present invention, may be administered using any amount and any route of administration effective for treating or lessening the severity of the disorder (e.g. a proliferative disorder). The exact amount required will vary from subject to subject, depending on the species, age, and general condition of the subject, the severity of the infection, the particular agent, its mode of administration, and the like. Compounds of the invention are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg. In some embodiments the therapeutically effective dosage is in the range of about 1 to 2,000 mg, about 250 to 2,000 mg, about 250 to 1,500 mg, about 250 to 1,000 mg, about 250 to 750 mg, about 250 to 500 mg, about 500 to 2,000 mg, about 500 to 1,500 mg, about 500 to 1,000 mg, about 500 to 750 mg, about 750 to 2,000 mg, about 750 to 1,500 mg, about 750 to 1,000 mg, about 1,000 to 2,000 mg, about 1,000 to 1,500 mg, or about 1,500 to 2,000 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the invention relates to a method of inhibiting protein kinase activity in a biological sample comprising the step of contacting said biological sample with a compound of this invention, or a composition comprising said compound. According to another embodiment, the invention relates to a method of inhibiting activity of a PI3K, or a mutant thereof, in a biological sample comprising the step of contacting said biological sample with a compound of this invention, or a composition comprising said compound. According to another embodiment, the invention relates to a method of inhibiting activity of PI3Kα, or a mutant thereof, in a biological sample comprising the step of contacting said biological sample with a compound of this invention, or a composition comprising said compound. In some embodiments, the PI3Kα is a mutant PI3Kα. In some embodiments, the PI3Kα contains at least one of the following mutations: H1047R, E542K, and E545K. In some embodiments, the PI3Kα contains at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the PI3Kα contains at least one of the following mutations: E81K, R88Q, R93Q, R93W, GT06R, GT06V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M1043I, M1043V, N1044K, H1047R, H1047L, H1047Y, G1049R, G1049S, I1058F, A1066V, and N1068fs*4.

In another embodiment, the invention provides a method of selectively inhibiting PI3Kα over one or both of PI3Kδ and PI3Kγ. In some embodiments, a compound of the present invention is more than 5-fold selective over PI3Kδ and PI3Kγ. In some embodiments, a compound of the present invention is more than 10-fold selective over PI3Kδ and PI3Kγ. In some embodiments, a compound of the present invention is more than 50-fold selective over PI3Kδ and PI3Kγ. In some embodiments, a compound of the present invention is more than 100-fold selective over PI3Kδ and PI3Kγ. In some embodiments, a compound of the present invention is more than 200-fold selective over PI3Kδ and PI3Kγ. In some embodiments, the PI3Kα is a mutant PI3Kα. In some embodiments, the PI3Kα contains at least one of the following mutations: H1047R, E542K, and E545K. In some embodiments, the PI3Kα contains at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the PI3Kα contains at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

In another embodiment, the invention provides a method of selectively inhibiting a mutant PI3Kα over a wild-type PI3Kα. In some embodiments, a compound of the present invention is more than 5-fold selective for mutant PI3Kα over wild-type PI3Kα. In some embodiments, a compound of the present invention is more than 10-fold selective for mutant PI3Kα over wild-type PI3Kα. In some embodiments, a compound of the present invention is more than 50-fold selective for mutant PI3Kα over wild-type PI3Kα. In some embodiments, a compound of the present invention is more than 100-fold selective for mutant PI3Kα over wild-type PI3Kα. In some embodiments, a compound of the present invention is more than 200-fold selective for mutant PI3Kα over wild-type PI3Kα. In some embodiments, the mutant PI3Kα contains at least one of the following mutations: H1047R, E542K, and E545K. In some embodiments, the mutant PI3Kα contains at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the mutant PI3Kα contains at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of inhibiting protein kinase activity in a patient comprising the step of administering to said patient a compound of the present invention, or a composition comprising said compound.

According to another embodiment, the invention relates to a method of inhibiting activity of a PI3K, or a mutant thereof, in a patient comprising the step of administering to said patient a compound of the present invention, or a composition comprising said compound. In some embodiments, the invention relates to a method of inhibiting activity of PI3Kα, or a mutant thereof, in a patient comprising the step of administering to said patient a compound of the present invention, or a composition comprising said compound. In some embodiments, the PI3Kα is a mutant PI3Kα. In some embodiments, the PI3Kα contains at least one of the following mutations: H1047R, E542K, and E545K. In some embodiments, the PI3Kα contains at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the PI3Kα contains at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M1043I, M1043V, N1044K, H1047R, H1047L, H1047Y, G1049R, G1049S, I1058F, A1066V, and N1068fs*4.

According to another embodiment, the present invention provides a method for treating a disorder mediated by a PI3K, or a mutant thereof, in a patient in need thereof, comprising the step of administering to said patient a compound according to the present invention or pharmaceutically acceptable composition thereof. In some embodiments, the present invention provides a method for treating a disorder mediated by PI3Kα, or a mutant thereof, in a patient in need thereof, comprising the step of administering to said patient a compound according to the present invention or pharmaceutically acceptable composition thereof. In some embodiments, the PI3Kα is a mutant PI3Kα. In some embodiments, the PI3Kα contains at least one of the following mutations: H1047R, E542K, and E545K. In some embodiments, the PI3Kα contains at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the PI3Kα contains at least one of the following mutations: E81K, R88Q, R93Q, R93W, GT06R, GT06V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M1043I, M1043V, N1044K, H1047R, H1047L, H1047Y, G1049R, G1049S, I1058F, A1066V, and N1068fs*4.

According to another embodiment, the present invention provides a method of inhibiting signaling activity of PI3Kα, or a mutant thereof, in a subject, comprising administering a therapeutically effective amount of a compound according to the present invention, or a pharmaceutically acceptable composition thereof, to a subject in need thereof. In some embodiments, the present invention provides a method of inhibiting PI3Kα signaling activity in a subject, comprising administering a therapeutically effective amount of a compound according to the present invention, or a pharmaceutically acceptable composition thereof, to a subject in need thereof. In some embodiments, the PI3Kα is a mutant PI3Kα. In some embodiments, the PI3Kα contains at least one of the following mutations: H1047R, E542K, and E545K. In some embodiments, the subject has a mutant PI3Kα. In some embodiments, the subject has PI3Kα containing at least one of the following mutations: H1047R, E542K, and E545K. In some embodiments, the subject has PI3Kα containing at least one of the following mutations: E81X, R88X, R93X, G106X, R108X, K111X, G118X, A222X, V344X, N345X, G364X, E365X, C420X, E453X, P539X, E542X, E545X, Q546X, D549X, F667X, H701X, M1004X, Y1021X, T1025X, M1040X, M1043X, N1044X, H1047X, G1049X, I1058X, A1066X, and N1068X, wherein X is any amino acid besides its wildtype. In some embodiments, the subject has PI3Kα containing at least one of the following mutations: E81K, R88Q, R93Q, R93W, G106R, G106V, R108H, K111N, K111E, G118D, A222V, V344A, N345K, G364R, E365K, C420R, E453A, E453K, P539R, E542K, E542Q, E545A, E545G, E545K, E545Q, Q546E, Q546K, Q546L, Q546P, Q546R, D549N, F667L, H701P, M1004I, Y1021C, T1025A, T1025N, M1040L, M10431, M1043V, N1044K, H1047R, H1047L, H1047Y, G1049R, G1049S, I1058F, A1066V, and N1068fs*4.

The compounds described herein can also inhibit PI3Kα function through incorporation into agents that catalyze the destruction of PI3Kα. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of PI3Kα to the E3 ligase will thus result in the destruction of the PI3Kα protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions of this invention. As used herein, additional therapeutic agents that are normally administered to treat a particular disease, or condition, are known as “appropriate for the disease, or condition, being treated.”

Additionally, PI3K serves as a second messenger node that integrates parallel signaling pathways, and evidence is emerging that the combination of a PI3K inhibitor with inhibitors of other pathways will be useful in treating cancer and cellular proliferative diseases.

Accordingly, in certain embodiments, the method of treatment comprises administering the compound or composition of the invention in combination with one or more additional therapeutic agents. In certain other embodiments, the methods of treatment comprise administering the compound or composition of the invention as the only therapeutic agent.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI13K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)). Accordingly, in certain embodiments, the method of treatment comprises administering the compound or composition of the invention in combination with trastuzumab. In certain embodiments, the cancer is a human breast cancer that overexpresses Her-2/neu-ErbB2.

A variety of human malignancies express activating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway.

For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Accordingly, in certain embodiments, the method of treatment comprises administering the compound or composition of the invention in combination with an inhibitor of Her1/EGFR. In certain embodiments, the method of treatment comprises administering the compound or composition of the invention in combination with one or more of tarceva, gefitinib, and erbitux. In certain embodiments, the method of treatment comprises administering the compound or composition of the invention in combination with gefitinib. In certain embodiments, the cancer expresses activating mutations or increased levels of Her1/EGFR.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Aid also abrogates its role to arrest the cell cycle (Viglietto et al., Nat. Med. 8:1145 (2002)).

Accordingly, in certain embodiments, the method of treatment comprises administering the compound or composition of the invention in combination with a treatment for a hormone-dependent cancer. In certain embodiments, the method of treatment comprises administering the compound or composition of the invention in combination with tamoxifen. In certain embodiments, the cancer is a hormone dependent cancer, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Ab1 employs the Ras-Raf kinase pathway to elicit its effects. In addition, inhibiting more than one kinase in the same pathway provides additional protection against resistance-conferring mutations.

Accordingly, in another aspect, the compounds and compositions of the invention are used in combination with at least one additional agent selected from the group of kinase inhibitors, such as imatinib, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of P13 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In some embodiments, the one or more additional therapeutic agents is selected from antibodies, antibody-drug conjugates, kinase inhibitors, immunomodulators, and histone deacetylase inhibitors. Synergistic combinations with PIK3CA inhibitors and other therapeutic agents are described in, for example, Castel et al., Mol. Cell Oncol. (2014)1(3) e963447.

In some embodiments, the one or more additional therapeutic agent is selected from the following agents, or a pharmaceutically acceptable salt thereof: BCR-ABL inhibitors (see e.g. Ultimo et al. Oncotarget (2017) 8 (14) 23213-23227.): e.g. imatinib, inilotinib, nilotinib, dasatinib, bosutinib, ponatinib, bafetinib, danusertib, saracatinib, PF03814735; ALK inhibitors (see e.g. Yang et al. Tumour Biol. (2014) 35 (10) 9759-67): e.g. crizotinib, NVP-TAE684, ceritinib, alectinib, brigatinib, entrecinib, lorlatinib; BRAF inhibitors (see e.g. Silva et al. Mol. Cancer Res. (2014) 12, 447-463): e.g. vemurafenib, dabrafenib; FGFR inhibitors (see e.g. Packer et al. Mol. Cancer Ther. (2017) 16(4) 637-648): e.g. infigratinib, dovitinib, erdafitinib, TAS-120, pemigatinib, BLU-554, AZD4547; FLT3 inhibitors: e.g. sunitinib, midostaurin, tanutinib, sorafenib, lestaurtinib, quizartinib, and crenolanib; MEK Inhibitors (see e.g. Jokinen et al. Ther. Adv. Med. Oncol. (2015) 7(3) 170-180): e.g. trametinib, cobimetinib, binimetinib, selumetinib; ERK inhibitors: e.g. ulixertinib, MK 8353, LY 3214996; KRAS inhibitors: e.g. AMG-510, MRTX849, ARS-3248; Tyrosine kinase inhibitors (see e.g. Makhov et al. Mol. Cancer. Ther. (2012) 11(7) 1510-1517): e.g. erlotinib, linifanib, sunitinib, pazopanib; Epidermal growth factor receptor (EGFR) inhibitors (see e.g. She et al. BMC Cancer (2016) 16, 587): gefitnib, osimertinib, cetuximab, panitumumab; HER2 receptor inhibitors (see e.g. Lopez et al. Mol. Cancer Ther. (2015) 14(11) 2519-2526): e.g. trastuzumab, pertuzumab, neratinib, lapatinib, lapatinib; MET inhibitors (see e.g. Hervieu et al. Front. Mol. Biosci. (2018) 5, 86): e.g. crizotinib, cabozantinib; CD20 antibodies: e.g. rituximab, tositumomab, ofatumumab; DNA Synthesis inhibitors: e.g. capecitabine, gemcitabine, nelarabine, hydroxycarbamide; Antineoplastic agents (see e.g. Wang et al. Cell Death & Disease (2018) 9, 739): e.g. oxaliplatin, carboplatin, cisplatin; Immunomodulators: e.g. afutuzumab, lenalidomide, thalidomide, pomalidomide; CD40 inhibitors: e.g. dacetuzumab; Pro-apoptotic receptor agonists (PARAs): e.g. dulanermin; Heat Shock Protein (HSP) inhibitors (see e.g. Chen et al. Oncotarget (2014) 5 (9). 2372-2389): e.g. tanespimycin; Hedgehog antagonists (see e.g. Chaturvedi et al. Oncotarget (2018) 9 (24), 16619-16633): e.g. vismodegib; Proteasome inhibitors (see e.g. Lin et al. Int. J. Oncol. (2014) 44 (2), 557-562): e.g. bortezomib; PI3K inhibitors: e.g. pictilisib, dactolisib, alpelisib, buparlisib, taselisib, idelalisib, duvelisib, umbralisib; SHP2 inhibitors (see e.g. Sun et al. Am. J. Cancer Res. (2019) 9 (1), 149-159: e.g. SHP099, RMC-4550, RMC-4630); BCL-2 inhibitors (see e.g. Bojarczuk et al. Blood (2018) 133 (1), 70-80): e.g. venetoclax; Aromatase inhibitors (see e.g. Mayer et al. Clin. Cancer Res. (2019) 25 (10), 2975-2987): exemestane, letrozole, anastrozole, fulvestrant, tamoxifen; mTOR inhibitors (see e.g. Woo et al. Oncogenesis (2017) 6, e385): e.g. temsirolimus, ridaforolimus, everolimus, sirolimus; CTLA-4 inhibitors (see e.g. O'Donnell et al. (2018) 48, 91-103): e.g. tremelimumab, ipilimumab; PD1 inhibitors (see O'Donnell, supra): e.g. nivolumab, pembrolizumab; an immunoadhesin; Other immune checkpoint inhibitors (see e.g. Zappasodi et al. Cancer Cell (2018) 33, 581-598, where the term “immune checkpoint” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta): e.g. pidilizumab, AMP-224; PDL1 inhibitors (see e.g. O'Donnell supra): e.g. MSB0010718C; YW243.55.S70, MPDL3280A; MEDI-4736, MSB-0010718C, or MDX-1105; Histone deacetylase inhibitors (HDI, see e.g. Rahmani et al. Clin. Cancer Res. (2014) 20(18), 4849-4860): e.g. vorinostat; Androgen Receptor inhibitors (see e.g. Thomas et al. Mol. Cancer Ther. (2013) 12(11), 2342-2355): e.g. enzalutamide, abiraterone acetate, orteronel, galeterone, seviteronel, bicalutamide, flutamide; Androgens: e.g. fluoxymesterone; CDK4/6 inhibitors (see e.g. Gul et al. Am. J. Cancer Res. (2018) 8(12), 2359-2376): e.g. alvocidib, palbociclib, ribociclib, trilaciclib, abemaciclib.

In some embodiments, the one or more additional therapeutic agent is selected from the following agents: anti-FGFR antibodies; FGFR inhibitors, cytotoxic agents; Estrogen Receptor-targeted or other endocrine therapies, immune-checkpoint inhibitors, CDK inhibitors, Receptor Tyrosine Kinase inhibitors, BRAF inhibitors, MEK inhibitors, other PI3K inhibitors, SHP2 inhibitors, and SRC inhibitors. (See Katoh, Nat. Rev. Clin. Oncol. (2019), 16:105-122; Chae, et al. Oncotarget (2017), 8:16052-16074; Formisano et al., Nat. Comm. (2019), 10:1373-1386; and references cited therein.).

In some embodiments, the estrogen receptor targeted therapy is a selective estrogen receptor degrader (SERD, e.g. fulvestrant, elacestrant, giredestrant). In some embodiments, the estrogen receptor targeted therapy is an estrogen receptor degrading PROTAC (e.g. ARV-471). In some embodiments, the endocrine therapy is an aromatase inhibitor (e.g. anastrozole, letrozole, exemestane).

In some embodiments, the one or more additional therapeutic agents are inhibitors of one or more of CDK2, CDK4, and CDK6 enzymes. In some embodiments, the CDK inhibitor is a CDK2 inhibitor (e.g. PF-07104091). In some embodiments, the CDK inhibitor is a CDK4 inhibitor (e.g. PF-07220060, AU2-94). In some embodiments, the CDK inhibitor is a dual CDK4/6 inhibitor (e.g. palbociclib, abemaciclib, ribociclib, trilaciclib). In some embodiment,s the CDK inhibitor is an inhibitor of CDK2/4/6.

In some embodiments, more than one CDK inhibitor is administered together with compound of the invention. In some embodiments, the additional therapeutic agents comprise one or more CDK inhibitors and an estrogen receptor targeted therapy. In some embodiments, the additional therapeutic agent comprises a selective estrogen receptor degrader and one or more CDK inhibitors.

In some embodiments, the additional therapeutic agents comprise a CDK2 inhibitor and an estrogen receptor targeted therapy. In some embodiments, the additional therapeutic agents comprise a CDK4 inhibitor and an estrogen receptor targeted therapy. In some embodiments, the additional therapeutic agents comprise a CDK2 inhibitor, a CDK4 inhibitor, and an estrogen receptor targeted therapy. In some embodiments, the additional therapeutic agents comprise a CDK4/6 inhibitor and an estrogen receptor targeted therapy. In some embodiments, the additional therapeutic agents comprise a CDK2 inhibitor, a CDK4/6 inhibitor, and an estrogen receptor targeted therapy.

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to other classes and subclasses and species of each of these compounds, as described herein. Additional compounds of the invention were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### Examples 1-5

The compounds described herein can be prepared in a number of ways based on the teachings contained herein and synthetic procedures known in the art. The following non-limiting examples illustrate the disclosures herein.

### X-ray Powder Diffraction (XRPD)

Instrument: Bruker D8 Advance
Method 1 (about 10 min):
   Detector LYNXEYE_XE_T(T1D mode)
   Open angle 2.940
   Radiation Cu/K-Alpha1 (λ=1.5406 Å)
   X-ray generator power 40 kV, 40 mA
   Primary beam path slits Twin_Primary motorized slit 10.0 mm by sample length;
   SollerMount axial soller 2.5°
   Secondary beam path slits Detector OpticsMount soller slit 2.5°; Twin_Secondary
   motorized slit 5.2 mm
   Scan mode Continuous scan
   Scan type Locked coupled
   Step size 0.02°
   Time per step 0.3 second per step
   Scan range 2° to 400
   Sample rotation speed 15 rpm
   Sample holder Monocrystalline silicon, flat surface
Method 2 (about 4 min, for evaluation samples: bulk stability, solubility study, suspension stability study):
   Detector LYNXEYE_XE_T(1D mode)
   Open angle 2.94°
   Radiation Cu/K-Alpha1 (λ=1.5406 Å)
   X-ray generator power 40 kV, 40 mA
   Primary beam path slits Twin_Primary motorized slit 10.0 mm by sample length;
   SollerMount axial soller 2.5°
   Secondary beam path slits Detector OpticsMount soller slit 2.5°; Twin_Secondary
   motorized slit 5.2 mm
   Scan mode Continuous scan
   Scan type Locked coupled
   Step size 0.020
   Time per step 0.12 second per step
   Scan range 3° to 400
   Sample rotation speed 15 rpm
   Sample holder Monocrystalline silicon, flat surface
Method 3: (about 2 min, for samples from salt screening experiments, slow evaporation and additional of anti-solvents experiments):
   Detector LYNXEYE_XE_T(1D mode)
   Open angle 2.940
   Radiation Cu/K-Alpha1 (λ=1.5406 Å)
   X-ray generator power 40 kV, 40 mA
   Primary beam path slits Twin_Primary motorized slit 10.0 mm by sample length;
   SollerMount axial soller 2.5°
   Secondary beam path slits Detector OpticsMount soller slit 2.5°; Twin_Secondary
   motorized slit 5.2 mm
   Scan mode Continuous scan
   Scan type Locked coupled
   Step size 0.020
   Time per step 0.06 second per step
   Scan range 3° to 400
   Sample rotation speed 15 rpm
   Sample holder Monocrystalline silicon, flat surface
Various Humidity X-ray Powder Diffractometer (VH-XRPD):
   Instrument Bruker D8 Advance
   Detector LynxEye
   Open angle 3°
   Radiation Cu/K-Alpha1 (λ=1.5406 Å)
   X-ray generator power 40 kV, 40 mA
   Primary beam path slits Primary Soller slit 2.5°; divergence slit 0.6 mm
   Secondary beam path slits Secondary Soller slit 2.5°; antiscattering slit 7.100 mm;
   detector slit 10.50 mm
   Scan mode Continuous scan
   Scan type Locked coupled
   Step size 0.02°
   Time per step 0.6 second per step
   Scan range 4° to 400
   Non-ambient stage CHC Plus+ cryo & humidity chamber

### Differential Scanning Calorimetric (DSC)

Instrument TA Discovery 2500 or Q2000
Sample pan Tzero pan and Tzero hermetic lid with a pin hole of 0.7 mm in diameter
Temperature range 30 to 250° C. or before decomposition
Heating rate 10° C./min or 2° C./min
Nitrogen flow 50 mL/min
Sample mass About 1-2 mg

### Thermal Gravimetric Analysis (TGA)

Instrument Discovery 5500 or Q5000
Sample pan Aluminum, open
Start temperature Ambient condition (below 35° C.)
Final temperature 300° C. or abort next segment if weight <80% (w/w)
(The weight loss of the compound is no more than 20% (w/w))
Heating rate 10° C./min
Nitrogen flow Balance 10 mL/min; sample chamber 25 mL/min
Sample mass About 2-10 mg

### Dynamic Vapor Sorption (DVS)

Method 1 (for I-1 Form A and I-3 Form A)
   Instrument Intrinsic, Advantage or Adventure
   Total gas flow 200 sccm
   Oven temperature 25° C.
   Solvent Water
   Method Cycle: 40-0-95-0-40% RH
   Stage Step: 10%
   Equilibrium: 0.002 dm/dt (%/min)
   Minimum dm/dt stability duration: 60 min
   Maximum dm/dt stage time: 360 min
Method 2 (for I-4 Form A)
   Instrument Intrinsic, Advantage or Adventure
   Total gas flow 200 sccm
   Oven temperature 25° C.
   Solvent Water
   Method Cycle: 40-95-0-95-40% RH
   Stage Step: 10%
   Equilibrium: 0.002 dm/dt (%/min)
   Minimum dm/dt stability duration: 60 min
   Maximum dm/dt stage time: 360 min

### Karl Fischer

Instrument Mettler Toledo Coulometric KF Titrator C30
Method Coulometric

### Polarized Light Microscope (PLM)

Instrument Olympus BX53LED
Method Crossed polarizer, silicone oil added

### Nuclear Magnetic Resonance (NMR)

Instrument Bruker Avance-AV 400M (for 1H-NMR, 19F-NMR and 31P-NMR)
Bruker Avance-III 400M (for 13C-NMR)
Frequency 400 MHz
Probe 5 mm PABBO BB/19F-1HID Z-GRD Z108618/0406 (for 1H-NMR, 19F-NMR and 31P-NMR)
5 mm PABBO BB-1H/D Z-GRD Z108618/0229 (for 13C NMR)
Number of scan 8
Temperature 297.6K
Relaxation delay 1 second

### Fourier Transform Infrared Spectrum (FT-IR)

Instrument: Fourier Transform Infrared Spectroscopy (Nicolet 6700, Thermo Scientific)
No. of sample scans: 32
No. of background scans: 32
Resolution: 4
Wavelength range: 4000 to 525 cm-1
Baseline correction: Yes
Optical velocity: 0.4747
Aperture: 150
Window: Diamond

### Supercritical Fluid Chromatography (SFC)

Instrument: CAS-SH-ANA-SFC-H(Watera UPCC with PDA Detector)
Wave length: 220 nm
Column: Chiralcel OD-3 (4.6×150 mm×3 μm)
Detector: PDA
Column temperature: 35° C.
Flow rate: 2.5 mL/min
Mobile phase A: CO2
Mobile phase B: Methanol(0.05% DEA)
Diluent: ACN
Injection volume: 1.00 μL
Sample Preparation: 2 mg/mL
Needle Wash Solvent: ACN:H2O=90:10 (v/v)
Gradient: from 5% to 40% of B in 5 min and hold 40% for 2.5 min, then 5% of B for 2.5 mm

### High Performance Liquid Chromatograph (HPLC)

Instrument Agilent 1260, SHIMADZU CBM-40,
Chiral purity Wave length: 220 nm
Column: Daicel OD-RH (4.6×150 mm×5 μm)
Detector: DAD, PDA
Column temperature: 40° C.
Flow rate: 1 mL/min
Mobile phase A: 10 mM NH4OAc in water
Mobile phase B: ACN
Diluent: ACN
Injection volume: 5 μL
Sample Preparation: 2 mg/mL
Needle Wash Solvent: ACN:H2O=90:10(v/v)
Gradient: Isocratic elution

| Time (min) | Mobile Phase A (%) | Mobile Phase B (%) |
|---|---|---|
| 0 | 55 | 45 |
| 30 | 55 | 45 |

### High Performance Liquid Chromatograph (HPLC)

Instrument Agilent 1260, SHIMADZU CBM-40,
Chemical purity and solubility
Wave length: 220 nm
Column: Phenomenex Luna PFP(2), 4.6×150 mm, 3 μm
Detector: DAD, PDA
Column temperature: 40° C.
Flow rate: 1 mL/min
Mobile phase A: 0.05% TFA in water, v/v
Mobile phase B: 0.05% TFA in (MeOH:ACN=1:9), v/v, for example, mix 100 mL of
MeOH and 900 mL of ACN, accurately, transfer 0.5 mL TFA to it, mix well and degas by ultrasonic.
Diluent: ACN
Injection volume: 5 μL
Sample Preparation: 0.8 mg/mL
Needle Wash Solvent: ACN:H2O=9:1(v/v)
Gradient:

| Time (min) | Mobile Phase A (%) | Mobile Phase B (%) |
|---|---|---|
| Initial | 75 | 25 |
| 8.00 | 55 | 45 |
| 14.00 | 55 | 45 |
| 22.00 | 50 | 50 |
| 26.00 | 50 | 50 |
| 30.00 | 15 | 85 |
| 33.00 | 15 | 85 |
| 34.00 | 75 | 25 |
| 40.00 | 75 | 25 |

### Ultra-High Performance Liquid Chromatograph (UPLC)

Instrument Agilent 1290
Solubility Wave length: 220 nm
Column: Waters ACQuity UPLC BEH C18 2.1*150 mm, 1.7 μm
Detector: DAD
Column temperature: 40° C.
Flow rate: 0.3 mL/min
Mobile phase A: 0.037% TFA in water, v/v
Mobile phase B: 0.018% TFA in ACN
Diluent: ACN/H2O (1:1,v/v)
Injection volume: 5 μL
Needle Wash Solvent: ACN:H2O=1:1(v/v)
Gradient: Isocratic elution

| Time (min) | Mobile Phase A (%) | Mobile Phase B (%) |
|---|---|---|
| Initial | 95 | 5 |
| 5.00 | 5 | 95 |
| 6.00 | 95 | 5 |
| 8.00 | 95 | 5 |

| Acronyms | Full name |
|---|---|
| MeOH | Methanol |
| EtOH | Ethanol |
| ACN | Acetonitrile |
| TFA | Trifluoroacetic acid |
| DMSO | Dimethyl sulfoxide |
| IPAc | Isopropyl acetate |
| DCM | Dichloromethane |
| EA | Ethyl acetate |
| THF | Tetrahydrofuran |
| MTBE | Methyl tert-butyl ether |

### Example 1. Synthesis of Intermediates

### 1.1. Preparation of Compound 5

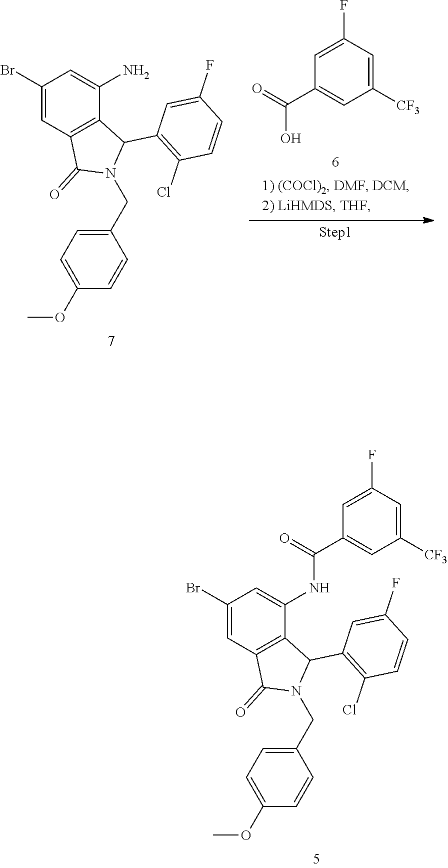 [SMILES: COc1ccc(CN2C(=O)c3cc(Br)cc(N)c3C2c2cc(F)ccc2Cl)cc1 ; O=C(O)c1cc(F)cc(C(F)(F)F)c1 ; COc1ccc(CN2C(=O)c3cc(Br)cc(NC(=O)c4cc(F)cc(C(F)(F)F)c4)c3C2c2cc(F)ccc2Cl)cc1]

### 1.1.1 Summary

Through screening, a mild EDCI/HOAt/DIPEA mediated amidation procedure was identified to replace the original cryogenic system employing (COCl)₂/DMF/LiHMDS; In addition, a simplified purification process was developed. The process was verified with a 100 g-scale reaction, which gave the amide product in 97.3% HPLC purity as a solution in 2-MeTHF. The solution could be used for next step without isolating the solid. The details were summarized as below.

### 1.1.2 Process Familiarization

The original TP condition with (COCl)₂/DMF/LiHMDS was repeated. Compound 7 was consumed, but 7.5% of Compound 6 was left as shown by HPLC.

### Repeat TP Condition:

| Starting Materials | | | | |
|---|---|---|---|---|
| Com- | Com- | | Reaction | |
| pound 7 | pound 6 | Reagents | Condition | IPC |
| 25.0 g | 14.2 g | DMF: 0.05 eq | −70~−60° | ~7.5% Compound 7 |
| | | (COCl)₂: 1.6 eq | C., 2 h | ~72.1% Compound |
| | | LiHMDS: 3.0 eq | | 5 left |

### 1.1.3 Screen Coupling Reagent

Five reactions were carried out to screen coupling reagent (HATU, PyBOP, EDCI/HOAt, EDCI/HOBt and EEDQ), DMF as solvent. Finally, EDCI/HOAt system gave the best IPC result.

### Results for Screen the Reaction Reagent:

| Starting Materials | | | | | IPC | | | | |
|---|---|---|---|---|---|---|---|---|---|
| Com- | Com- | | | Reaction | | | Com- | Com- | Com- |
| pound 7 | pound 6 | Reagent | DMF | Conditions | | | pound 7 | pound 6 | pound 5 |
| 2.0 g | 1.0 g | HATU(2.0 g, | 10 V | 25° C. | 2 | h | 83.4% | 10.9% | 4.1% |
| (1.0 eq) | (1.1 eq) | 1.2 eq) | | | 4 | h | 66.6% | 19.2% | 12.2% |
| | | DIPEA(0.65 g, | | | 20 | h | 61.9% | 7.5% | 25.5% |
| | | 1.2 eq) | | | | | | | |
| 2.0 g | 1.0 g | PyBOP(2.7 g, | 10 V | 25° C. | 2 | h | 85.9% | 7.8% | 1.0% |
| (1.0 eq) | (1.1 eq) | 1.2 eq) | | | 4 | h | 71.3% | 20.5% | 3.1% |
| | | DIPEA(0.65 g, | | | 20 | h | 75.7% | 8.3% | 7.1% |
| | | 1.2 eq) | | | | | | | |
| 2.0 g | 1.0 g | EDCI(1.6 g, | 10 V | 25° C. | 4 | h | 56.1% | 17.1% | 24.9% |
| (1.0 eq) | (1.1 eq) | 2.0 eq) | | | 20 | h | 44.0% | 3.0% | 47.1% |
| | | HOAt(0.63 g, | | | 68 | h | 21.8% | 2.0% | 71.2% |
| | | 1.1 eq) | | | | | | | |
| 2.0 g | 1.0 g | EDCI(1.6 g, | 10 V | 25° C. | 2 | h | 85.9% | 3.5% | 3.2% |
| (1.0 eq) | (1.1 eq) | 2.0 eq) | | | 4 | h | 71.7% | 17.3% | 7.5% |
| | | HOBt(0.63 g, | | | 20 | h | 68.1% | 2.9% | 19.5% |
| | | 1.1 eq) | | | | | | | |
| 2.0 g | 1.0 g | EEDQ(2.1 g, | 10 V | 25° C. | 2 | h | 41.8% | 0.7% | 0.4% |
| (1.0 eq) | (1.1 eq) | 2.0 eq) | | | 4 | h | 31.1% | 0.1% | 0.7% |
| | | | | | 20 | h | 32.0% | N/A | 0.5% |

### 1.1.4 Screen the Solvent System

When using DMF as solvent, a related imine impurity (RT19.52) derived from DMF & Compound 7 was detected. So DMAc was tried to replace DMF. The DMAc reaction moved slowly and stirring at 60° C. for 18 h, 71.4% Compound 5 was detected in IPC with only 16.3% Compound 7 left. The imine impurity formation was completely prevented.

### Results for Screen the Solvent System:

| Starting Materials | | | | | IPC | | | |
|---|---|---|---|---|---|---|---|---|
| Compound | Compound | | | Reaction | | Compound | Compound | Compound |
| 7 | 6 | Reagent | DMAC | Conditions | | 7 | 6 | 5 |
| 2.0 g | 1.3 g | EDCl(1.6 g, | 5 V | 60° C. | 18 h | 16.3% | 4.5% | 71.4% |
| (1.0 eq) | (1.5 eq) | 2.0 eq) | | | | | | |
| | | HOAt(0.86 g, | | | | | | |
| | | 1.5 eq) | | | | | | |

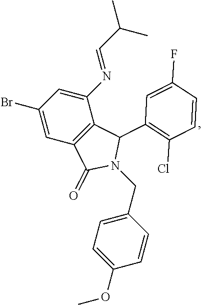 [SMILES: COc1ccc(CN2C(=O)c3cc(Br)cc(/N=C/C(C)C)c3C2c2cc(F)ccc2Cl)cc1 ; ","]

RT 19.52 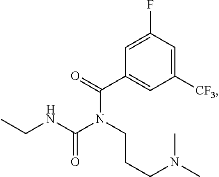 [SMILES: CCNC(=O)N(CCCN(C)C)C(=O)c1cc(F)cc(C(F)(F)F)c1]
RT 8.32 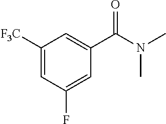 [SMILES: CN(C)C(=O)c1cc(F)cc(C(F)(F)F)c1]
RT 10.49

1.1.5 Screen the Temperature with DIPEA/DMAC

DIPEA/DMAC condition was evaluated on 60° C., 40° C. and 25° C. IPC results showed that reaction can move faster as the temperature increased. In the meanwhile, impurities RT19.52 and RT10.49 can be prevented. Reaction at 40° C. produced the best result, with 93.0% Compound 5 and 0.1% Compound 7 in IPC.

Results for Screen Temperature with DIPEA/DMAC:

| Starting Materials | | | | | IPC | | | |
|---|---|---|---|---|---|---|---|---|
| Com- | Com- | | | Reaction | | Com- | Com- | Com- |
| pound 7 | pound 6 | Reagent | DMAc | Conditions | | pound 7 | pound 6 | pound 5 |
| 2.0 g | 1.3 g | EDCI(1.6 g, | 5 V | 60° C. | 18 h | 3.5% | 4.7% | 89.9% |
| (1.0 eq) | (1.5 eq) | 2.0 eq) | | | | | | |
| | | HOAt(0.86 g, | | | | | | |
| | | 1.5 eq) | | | | | | |
| | | DIPEA(1.1 g, | | | | | | |
| | | 2.0 eq) | | | | | | |
| 2.0 g | 1.3 g | EDCI(1.6 g, | 5 V | 25° C. | 26 h | 1.6% | 3.3% | 91.8% |
| (1.0 eq) | (1.5 eq) | 2.0 eq) | | | | | | |
| | | HOAt(0.86 g, | | | | | | |
| | | 1.5 eq) | | | | | | |
| | | DIPEA(1.1 g, | | | | | | |
| | | 2.0 eq) | | | | | | |
| 20..0 g | 13.1 g | EDCI(16.2 g, | 5 V | 40° C. | 16 h | 0.1% | 3.8% | 93.0% |
| (1.0 eq) | (1.5 eq) | 2.0 eq) | | | | | | |
| | | HOAt(8.6 g, | | | | | | |
| | | 1.5 eq) | | | | | | |
| | | DIPEA(11.0 g, | | | | | | |
| | | 2.0 eq) | | | | | | |

### 1.1.6 Evaluation of the New Procedure

A scale-up reaction using 80 g Compound 7 was carried out to verify the process with DMAc in 40° C. IPC showed a typical result. After work up, 275.2 g 2-MeTHF solution was obtained with 97.7% HPLC purity. The 2-MeTHF solution was telescoped to next step directly.

### Evaluation of New Procedure:

| Starting Materials | | | | | | | |
|---|---|---|---|---|---|---|---|
| Com- | Com- | | | Reaction | | Note | |
| pound 7 | pound 6 | Reagent | DMAc | Conditions | IPC | Weight | Purity |
| 80.0 g | 52.4 g | EDCI(2.0 eq) | 5 V | 40° C. | 0.1% | 275.2 g | 97.7% |
| (1.0 eq) | (1.5 eq) | HOAt(1.5 eq) | | 17 h | Compound 7, | 2-MeTHF | HPLC |
| | | DIPEA(2.0 eq) | | | 2.8% | solution | purity |
| | | | | | Compound 6, | | |
| | | | | | 93.0% | | |
| | | | | | Compound 5 | | |

### 1.1.7 Verification of Process

A verification batch using 100 g Compound 7 was carried out. After stirring at 40° C. for 16 h, the reaction IPC showed 93.0% Compound 5 and only 0.1% Compound 7. After a typical work-up and purification, 350.6 g of 2-MeTHF solution was obtained with 97.3% o HPLC purity. The 2-MeTHF solution was telescoped to next step directly.

### Result of Verification:

| Starting Materials | | | | | | | |
|---|---|---|---|---|---|---|---|
| Com- | Com- | | | Reaction | | Note | |
| pound 7 | pound 6 | Reagent | DMAc | Conditions | IPC | Weight | Purity |
| 100 g | 65.6 g | EDCI(2.0 eq) | 5 V | 40° C. | 0.1% | 350.6 g | 97.3% |
| (1.0 eq) | (1.5 eq) | HOAt(1.5 eq) | | 16 h | Compound 7, | 2-MeTHF | HPLC |
| | | DIPEA(2.0 eq) | | | 3.2% | solution | purity |
| | | | | | Compound 6, | | |
| | | | | | 93.0% | | |
| | | | | | Compound 5 | | |

### 1.1.8 Process

1. Charge Compound 7 (100.0 g, 1.00±0.01×) into R1 under N2
2. Charge Compound 6 (65.6 g, 0.66±0.01×) into R1 under N2
3. Charge DMAc(470.0 g, 4.5-5.0×) into R1
4. Charge HOAt (42.9 g, 0.43±0.01×) into R1 under N2
5. Adjust R1 to 20˜30° C.
6. Charge DIPEA (55.0 g, 0.55±0.02×) into R1 under N2 at 20˜30° C.
7. Stir R1 for 0.5-1 h at 20˜30° C.
8. Charge EDCI (80.6 g, 0.81±0.01×) into R1 under N2 at 20˜30° C.
9. Adjust R1 to 35˜40° C.
10. Stir R1 for 16-20 h at 35˜40° C.
11. IPC: Compound 7/Compound 5=Report
12. Stir R1 for 4-6 h at 35˜40° C.
13. IPC: Compound 7/Compound 5=Report
14. Charge H2O (900 g, 9.0±0.2×) into R2
15. Charge Na2CO3 (100 g, 1.00±0.02×) into R2
16. Adjust R2 to 20˜30° C.
17. Stir R2 for 0.5-1 h at 20˜30° C.
18. Drum 10% Na2CO3 aqueous solution
19. Charge H2O (900 g, 9.0±0.2×) into R2
2θ. Charge NH4Cl (100 g, 1.00±0.02×) into R2
21. Adjust R2 to 20˜30° C.
22. Stir R2 for 0.5-1 h at 20˜30° C.
23. Drum 10% NH4Cl aqueous solution
24. Charge H2O (450 g, 4.5±0.1×) into R2
25. Charge NaCl (50 g, 0.50±0.01×) into R2
26. Adjust R2 to 20˜30° C.
27. Stir R2 for 0.5-1 h at 20˜30° C.
28. Drum 10% NaCl aqueous solution
29. Adjust R1 to 20˜30° C.
30. Charge EA(900 g, 9.0-10.0×) into R1
31. Charge Process water (1500 g, 15.0±0.3×) into R1 under N2 at 20˜30° C.
32. Stir R1 for 0.5-1 h at 20˜30° C.
33. Stand R1 for 0.5-1 h at 20˜30° C.
34. Separate: Charge the aqueous layer into T1, Charge the organic layer into T2
35. Charge the aqueous layer in T1 into R1
36. Charge EA(450 g, 4.5-5.0×) into R1
37. Adjust R1 to 20˜30° C.
38. Stir R1 for 0.5-1 h at 20˜30° C.
39. Stand R1 for 0.5-1 h at 20˜30° C.
40. Separate: Charge the aqueous layer into T1, Charge the organic layer into T2
41. IPC: Residual Compound 5 in aqueous layer of T1:Report
42. Drum the aqueous layer in T1
43. Charge the organic layer in T2 into R1
44. Charge 10% Na2CO3 aqueous solution (500 g, 5.0±0.1×) into R1
45. Adjust R1 to 20˜30° C.
46. Stir R1 for 0.5-1 h at 20˜30° C.
47. Stand R1 for 0.5-1 h at 20˜30° C.
48. Separate: Charge the aqueous layer into T3
49. Charge 10% Na2CO3 aqueous solution (500 g, 5.0±0.1×) into R1
50. Adjust R1 to 20˜30° C.
51. Stir R1 for 0.5-1 h at 20˜30° C.
52. Stand R1 for 0.5-1 h at 20˜30° C.
53. Separate: Charge the aqueous layer into T3, drum the aqueous layer in T3
54. Charge 10% NH4Cl aqueous solution (500 g, 5.0±0.1×) into R1
55. Adjust R1 to 20˜30° C.
56. Stir R1 for 0.5-1 h at 20˜30° C.
57. Stand R1 for 0.5-1 h at 20˜30° C.
58. Separate: Charge the aqueous layer into T4
59. Charge 10% NH4Cl aqueous solution (500 g, 5.0±0.1×) into R1
60. Adjust R1 to 20˜30° C.
61. Stir R1 for 0.5-1 h at 20˜30° C.
62. Stand R1 for 0.5-1 h at 20˜30° C.
63. Separate: Charge the aqueous layer into T4, drum the aqueous layer in T4
64. Charge 10% NaCl aqueous solution (500 g, 5.0±0.1×) into R1
65. Adjust R1 to 20˜30° C.
66. Stir R1 for 0.5-1 h at 20˜30° C.
67. Stand R1 for 0.5-1 h at 20˜30° C.
68. Separate: Charge the aqueous layer into T5, drum the aqueous layer in T5. Charge organic layer into T6
69. Clear R1
70. Charge organic layer in T6 into R1
71. Concentrate the organic layer in R1 to 3-4V below 45° C.
72. Charge 2-MeTHF(430.0 g, 4.3-5.0×) into R1
73. Concentrate the organic layer in R1 to 3-4V below 45° C.
74. Charge 2-MeTHF(430.0 g, 4.3-5.0×) into R1
75. Concentrate the organic layer in R1 to 3-4V below 45° C.
76. Charge 2-MeTHF(430.0 g, 4.3-5.0×) into R1
77. Concentrate the organic layer in R1 to 3-4V below 45° C.
78. IPC: Residual EA of Compound 5 in RI:Report, KF of Compound 5 in 2-MeTHF solution≤0.5%
79. Charge 2-MeTHF(430.0 g, 4.3-5.0×) into R1
80. Concentrate the organic layer in R1 to 3-4V below 45° C.
81. IPC: Residual EA of Compound 5 in RI:Report, KF of Compound 5 in 2-MeTHF solution≤0.5%
82. Drum 2-MeTHF solution and rinse R1 with 2-MeTHF(86.0 g, 0.9±0.4×)
83. IPC: Purity of Compound 5 in 2-MeTHF solution

### 1.2. Preparation of Compound 4

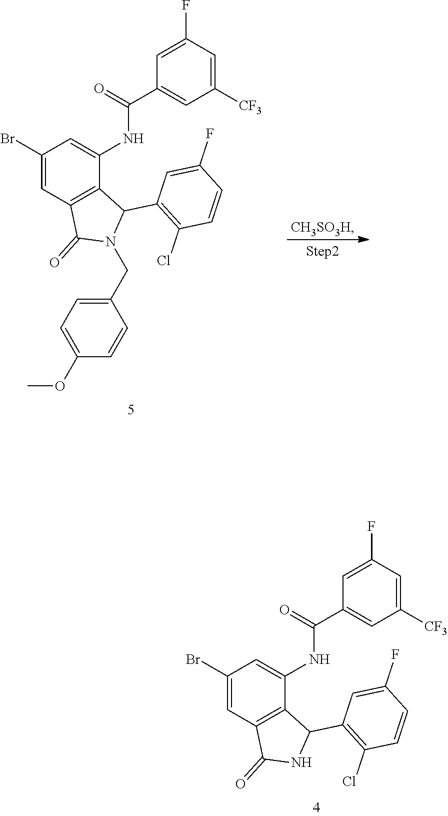 [SMILES: COc1ccc(CN2C(=O)c3cc(Br)cc(NC(=O)c4cc(F)cc(C(F)(F)F)c4)c3C2c2cc(F)ccc2Cl)cc1 ; "CH3SO3H," ; O=C(Nc1cc(Br)cc2c1C(c1cc(F)ccc1Cl)NC2=O)c1cc(F)cc(C(F)(F)F)c1]

### 1.2.1 Summary

A telescoped process was developed from Compound 7 to Compound 4. The original process went smoothly but ˜3% impurity (RT11.1) was generated. After temperature screening, 50° C. is deemed suitable to minimize RRT 11.1 impurity formation. A crystallization process in MTBE/Heptane was developed to isolate and purify product. The typical process was verified at 100 g scale reaction, and after typical work up, purification and isolation, product with 99.7% purity was obtained in ˜70% yield (two steps). The details were summarized as below.

### 1.2.2 Process Familiarization

The original TP condition with 70eq MsOH was repeated. 0.1% Compound 5 left and 89.2% Compound 4 were detected in IPC, ˜3% impurity (RT11.1) generated, and work up process will be further developed.

### Repeat TP Condition:

| Starting | | | | | |
|---|---|---|---|---|---|
| Materials | | Reaction | IPC | | |
| Compound 5 | Reagent | Conditions | | Compound 5 | Compound 4 |
| 2.0 g | MSOH | 60° C. | 21 h | 0.1% | 89.2% |
| (1.0 eq) | (7.5 V) | | | | |

### 1.2.3 Screen the Temperature

Reaction temperature was evaluated at 80° C., 40° C., 50° C. and 55° C. Reaction at 80° C. produced 39% of RRT 11.1 impurity, whose structure was shown below. Reaction at 40° C. was too slow. Reaction at 50° C. moved fast and clean, in which RRT 11.1 can be prevented effectively.

### Results for Screen the Temperature:

| | | | IPC | | | |
|---|---|---|---|---|---|---|
| Starting Materials | | Reaction | | | | RT 11.1 min |
| Compound 5 | Reagent | Conditions | | Compound 5 | Compound 4 | impurity |
| 20 g 2- | MsOH | 80° C. | 22 h | 0.1% | 48.4% | 39.1% |
| MeTHF | (70 eq) | | | | | |
| solution | | | | | | |
| (1.0 eq) | | | | | | |
| 20 g 2- | MsOH | 40° C. | 18 h | 58.5% | 31.6% | 0.2% |
| MeTHF | (70 eq) | | | | | |
| solution | | | | | | |
| (1.0 eq) | | | | | | |
| 20 g 2- | MsOH | 50° C. | 24 h | 4.4% | 89.8% | 0.8% |
| MeTHF | (70 eq) | | 40 h | 0.7% | 95.7% | 1.2% |
| solution | | | 48 h | 0.3% | 92.8% | 1.6% |
| (1.0 eq) | | | | | | |
| 21 g 2- | MsOH | 55° C. | 43 h | 0.1% | 92.1% | 4.0% |
| MeTHF | (70 eq) | | 49 h | 0.1% | 90.9% | 4.7% |
| solution | | | | | | |
| (1.0 eq) | | | | | | |

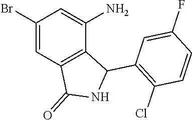 [SMILES: Nc1cc(Br)cc2c1C(c1cc(F)ccc1Cl)NC2=O]

RT 11.1

### 1.2.4 Verification of Process

A scale-up reaction using 350 g of amide Compound 5 as a solution in 2-MeTHF (1˜2V) was carried out to verify the new process. IPC is normal. After typical work up and purification, 104 g solid was obtained with 99.7% HPLC purity in ˜70% yield (two steps).

### Result of Verification:

Starting

| Materials | | | IPC | | | | |
|---|---|---|---|---|---|---|---|
| Com- | | Reaction | Com- | Com- | RT 11.1 min | Note | |
| pound 5 | Reagent | Conditions | pound 5 | pound 4 | impurity | Weight | Purity |
| 350 g | MsOH | 50~55° C. | 0.7% | 94.5% | 0.8% | 104 g | 99.7% |
| 2-MeTHF | (70 eq) | 27 h | | | | solid | HPLC |
| solution | | | | | | | purity |
| (1.0 eq) | | | | | | | |

1. Charge Compound 5 in 2-MeTHF solution into R1 under N2
2. Concentrate the organic layer in R1 to 1.5-2.5V below 45° C.
3. Adjust R1 to 15˜30° C.
4. Charge CH3SO3H (1414 g, 14±0.2×) into R1 at 15˜30° C.
5. Adjust R1 to 50˜55° C.
6. Stir R1 for 24-30 h at 50˜55° C.
7. IPC: Compound 5/Compound 4=Report
8. Stir R1 for 2-8 h at 50˜55° C.
9. IPC: Compound 5/Compound 4=Report
10. Charge H2O (630 g, 6.3±0.2×) into R2
11. Charge NH4Cl (70 g, 0.7˜0.8×) into R2
12. Adjust R2 to 20˜30° C.
13. Stir R2 for 0.5-1 h at 20˜30° C.
14. Drum 10% NH4Cl aqueous solution
15. Charge H2O (560 g, 5.6±0.2×) into R2
16. Charge NaCl (140 g, 1.4˜1.6×) into R2
17. Adjust R2 to 20˜30° C.
18. Stir R2 for 0.5-1 h at 20˜30° C.
19. Drum 20% NaCl aqueous solution
20. Adjust R1 to 15˜30° C.
21. Charge 2-MeTHF(600 g, 5.0˜7.0×) into R1 at 15˜30° C.
22. Charge H2O (420 g, 4.0˜5.0×) into R1 at 15˜30° C.
23. Adjust pH=10-11 by 30% NaOH(2000 g, 18-22×) at 15˜30° C.
24. Adjust R1 to 15˜25° C.
25. Stir R1 at 15˜25° C. for 0.5-1 h
26. Stand R1 for 0.5-1 h at 15˜25° C.
27. Separate: Charge aqueous layer into T1, Charge the organic layer into T2
28. Charge the aqueous layer in T1 into Ri
29. Charge 2-MeTHF(600 g, 5.0˜7.0×) into R1
30. Adjust R1 to 15˜25° C.
31. Stir R1 at 15˜25° C. for 0.5-1 h
32. Stand R1 for 0.5-1 h at 15˜25° C.
33. Separate: Charge aqueous layer into T1
34. IPC: Residual Compound 4 in aqueous layer:Report
35. Drum the aqueous layer into T1
36. Charge the organic layer in T2 into R1
37. Charge 10% NH4Cl aqueous solution(700 g, 7.0±0.5×) into R1
38. Adjust R1 to 15˜25° C.
39. Stir R1 at 15˜25° C. for 0.5-1 h
40. Stand R1 for 0.5-1 h at 15˜25° C.
41. Separate: charge aqueous layer into T3
42. Charge 20% NaCl aqueous solution(700 g, 7.0±0.5×) into R1
43. Adjust R1 to 15˜25° C.
44. Stir R1 at 15˜25° C. for 0.5-1 h
45. Stand R1 for 0.5-1 h at 15˜25° C.
46. Separate: Charge aqueous layer into T4, Charge organic layer into T5
47. Clean R1
48. Charge the organic layer in T5 into R1
49. Concentrate the organic layer in R1 to 5-7V below 45° C.
50. Charge MTBE(450 g, 4.0˜5.0×) into R1
51. Concentrate the organic layer in R1 to 5-7V below 45° C.
52. Charge MTBE(450 g, 4.0˜5.0×) into R1
53. Concentrate the organic layer in R1 to 5-7V below 45° C.
54. IPC: Residual 2-MeTHF in organic layer:Report
55. Adjust R1 to 40˜45° C.
56. Stir R1 at 40˜45° C. for 0.5-1 h
57. Adjust R1 to 20˜25° C. for 2-4 h
58. Stir R1 at 20˜25° C. for 2-4 h
59. Charge MTBE(180 g, 1.5˜2.5×) into R1
60. Charge n-Heptane(550 g, 5.0˜7.0×) into R1 at 20˜25° C.
61. Stir R1 for 6-1Oh at 20˜25° C.
62. IPC: Purity of Compound 4 wet cake:Report, Residual Compound 4 in mother layer:Report
63. Centrifuge
64. Charge MTBE/n-Heptane(1/1, v/v, 1.0˜3.0×) into R1 to rinse the cake
65. Centrifuge
66. IPC: Purity of Compound 4 wet cake:Report
67. Dry the wet cake at 70˜80° C. for 16-24 h.
68. IPC: KF of Compound 4: <0.5%, Residual MTBE, 2-MeTHF and n-Heptane of Compound 4:Report
69. Dry the wet cake at 70˜80° C. for 8-12 h.
70. IPC: KF of Compound 4: ≤0.5%, Residual MTBE, 2-MeTHF and n-Heptane of Compound 4:Report
71. Package the product

### 1.3. Pilot Plant Production Results

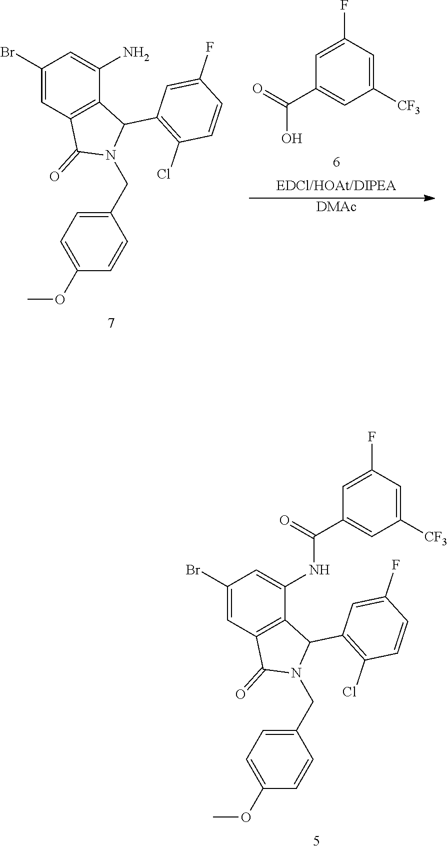

### Material Dispensing Summary Table

| | | Actual Amounts | | | |
|---|---|---|---|---|---|
| | | | | Assay for | |
| Materials | | Equiv. | Mass | reactants | Moles |
| Class | Name | eq | kg | w/w % | mol. |
| Reactants | 4-amino-6-bromo-3-(2-chloro-5- | 1.0 | 12.7 | N/A | 26.7 |
| | fluorophenyl)-2-(4- | | | | |
| | methoxybenzyl)isoindolin-1-one | | | | |
| | Compound 7 | | | | |
| | 3-fluoro-5-(trifluoromethyl)benzoic acid | 1.5 | 8.4 | N/A | 40.4 |
| | Compound 6 | | | | |
| | 1-Hydroxy-7-azabenzotriazole | 1.5 | 5.5 | N/A | 40.4 |
| | HOAT | | | | |
| | 1-Ethyl-3-(3- | 2.0 | 10.3 | 97%~103% | 53.7 |
| | dimethyllaminopropyl)carbodiimide•HCl | | | | |
| | EDC•HCl | | | | |
| | N,N-Diisopropylethylamine | 2.0 | 6.8 | N/A | 52.6 |
| | DIPEA | | | | |
| Chemicals | N,N-Dimethylacetamide DMAc | N/A | 123 | N/A | N/A |
| | Sodium carbonate | N/A | 12.8 | N/A | N/A |
| | Ammonium chloride | N/A | 12.9 | N/A | N/A |
| | 25% NaCl solution | N/A | 28 | N/A | N/A |
| | 2-Methyltetrahydrofuran | N/A | 193 | N/A | N/A |
| | 2-MeTHF | | | | |
| | Ethyl acetate | N/A | 189 | N/A | N/A |
| | EtOAc | | | | |
| | Process water | N/A | 463 | N/A | N/A |

### Process Description

| | | Comments/Observations/Results |
|---|---|---|
| Operation | | (X = 12.6 kg) |
| 1. | Charge process water (116 kg, 9.2 X) into X1 | |
| 2. | Charge sodium carbonate (12.8 kg, 1.02 X) into X1 in | |
| | portions | |
| 3. | Stir X1 for 0.5-1 h until the material were dissolved and | |
| | the solution is clear | |
| 4. | Load the material from the X1 into new steel-plastic | Total net of 10% Na₂CO₃ aqueous |
| | composite drums that purged with nitrogen and label | solution: 127.8 kg |
| | material tag | |
| 5. | Rinse inner wall of R5608 to visual clean, rinse water | |
| | into waste water pipes | |
| 6. | Charge process water (115 kg, 9.1 X) into X1 | |
| 7. | Charge Ammonium chloride (12.9 kg, 1.02 X) into X1 | |
| | in portions | |
| 8. | Stir X1 for 0.5-1 h until the material were dissolved and | |
| | the solution is clear | |
| 9. | Load the material from the X1 into new steel-plastic | Total net of 10% Ammonium |
| | composite drums that purged with nitrogen and label | chloride aqueous solution: 126.6 kg |
| | material tag | |
| 10. | Rinse inner wall of R5608 to visual clean, rinse water | |
| | into waste water pipes | |
| 11. | Charge process water (42 kg, 3.3 X) into new steel- | |
| | plastic composite drum of STEP 14 | |
| 12. | Charge 25% NaCl solution (28 kg, 2.2 X) into STEP | |
| | 14 drum via peristaltic pump | |
| 13. | Tympanic bulla by nitrogen at 1-2 m³/h for 10-20 min | |
| 14. | Weight the material in steel-plastic composite drum and | Total net of 10% NaCl aqueous |
| | label material tag | solution: 69.5 kg |
| 15. | Dry R5608 for 1-2 h at 70-80° C. under vacuum, then | |
| | adjust R5608 to 0-40° C. | |
| 16. | Charge DMAc (62 kg, 4.9 X) into R5608 via spray ball | |
| 17. | Adjust R5608 at 60-70° C. and stir R5608 at 60-70° C. | |
| | for 30-60 min, then adjust R5608 to 20-30° C. | |
| 18. | Load the solvent from the reactor into the galvanized | |
| | steel drums that purged with nitrogen and label waste | |
| | tag | |
| 19. | Charge compound 7 (12.7 kg, 1.01 X) into R5608 in | |
| | portions via flexible isolator | |
| 20. | Charge compound 6 (8.4 kg, 0.67 X) into R5608 in | |
| | portions via flexible isolator | |
| 21. | Stand R5608 for 5-10 min | |
| 22. | Charge DMAc (59 kg, 4.7 X) into R5608 via spray ball | |
| 23. | Charge HOAT (5.5 kg, 0.44 X) into R5608 in portions | |
| | via flexible isolator | |
| 24. | Adjust R5608 to 20-30° C. | |
| 25. | Adding DIPEA (6.8 kg, 0.54 X) into R5608 slowly with | |
| | the batch temperature controlled at 20-30° C. via | |
| | peristaltic pump | |
| 26. | Rinse the charging pipe with DMAc (2 kg, 0.2 X) and | |
| | transfer the rinse into R5608 via peristaltic pump | |
| 27. | Adjust R5608 to 20-30° C. | |
| 28. | Stir R5608 at 20-30° C. for 0.5-1 h | |
| 29. | Charge EDC•HCl (10.3 kg, 0.82 X) into R5608 in | |
| | portions with batch temperature controlled at 20-30° C. | |
| | via flexible isolator | |
| 30. | Adjust R5608 to 35-40° C. | |
| 31. | Stir R5608 at 35-40° C. for 16-20 h | |
| 32. | IPC: compound 7/compound 5 (%): report | 0.08% |
| 33. | Adjust R5608 to 20-30° C. | |
| 34. | Charge EtOAc (123 kg, 9.8 X) into R5608 | |
| 35. | Adjust R5608 to 20-30° C. | |
| 36. | Charge process water (190 kg, 15.1 X) into R5608 at | |
| | 20-30° C. via peristaltic pump | |
| 37. | Stir R5608 at 20-30° C. for 30-60 min | |
| 38. | Allow R5608 to stand for 30-60 min at 20-30° C. | |
| 39. | Transfer R5608 aqueous layer into T1 and label | |
| | material tag | |
| 40. | Load the organic phase in R5608 into new steel-plastic | |
| | composite drums flowed by nitrogen and label IPC | |
| | material tag | |
| 41. | Charge compound 5 DMAc aqueous layer in T1 of | |
| | STEP 39 into R5608 | |
| 42. | Charge EtOAc (60 kg, 4.8 X) into R5608 | |
| 43. | Adjust R5608 to 20-30° C. | |
| 44. | Stir R5608 at 20-30° C. for 30-60 min | |
| 45. | Allow R5608 to stand for 30-60 min at 20-30° C. | |
| 46. | Transfer R5608 aqueous layer into T1 and label | Estimate the volume of T1 aqueous |
| | material tag | layer: 293 L |
| 47. | IPC: Residual compound 5 (%, w/w): report | 0.1% |
| 48. | Charge compound 5 organic layer of STEP 40 into | |
| | R5608 | |
| 49. | Charge 10% Na₂CO₃ aqueous solution (63 kg, 5.0 X) of | |
| | STEP 4 into R5608 | |
| 50. | Adjust R5608 to 20-30° C. | |
| 51. | Stir R5608 at 20-30° C. for 30-60 min | |
| 52. | Allow R5608 to stand for 30-60 min at 20-30° C. | |
| 53. | Transfer R5608 aqueous layer into T2 and label | |
| | material tag | |
| 54. | Charge 10% Na₂CO₃ aqueous solution (63 kg, 5.0 X) of | |
| | STEP 4 into R5608 | |
| 55. | Adjust R5608 to 20-30° C. | |
| 56. | Stir R5608 at 20-30° C. for 30-60 min | |
| 57. | Allow R5608 to stand for 30-60 min at 20-30° C. | |
| 58. | Transfer R5608 aqueous layer into T2 and label | |
| | material tag | |
| 59. | Charge 10% Ammonium chloride aqueous (64 kg, 5.1 | |
| | X) of STEP 9 into R5608 | |
| 60. | Adjust R5608 to 20-30° C. | |
| 61. | Stir R5608 at 20-30° C. for 30-60 min | |
| 62. | Allow R5608 to stand for 30-60 min at 20-30° C. | |
| 63. | Transfer R5608 aqueous layer into T3 and label | |
| | material tag | |
| 64. | Charge 10% Ammonium chloride aqueous (63 kg, 5.0 | |
| | X) of STEP 9 into R5608 | |
| 65. | Adjust R5608 to 20-30° C. | |
| 66. | Stir R5608 at 20-30° C. for 30-60 min | |
| 67. | Allow R5608 to stand for 30-60 min at 20-30° C. | |
| 68. | Transfer R5608 aqueous layer into T3 and label | |
| | material tag | |
| 69. | Charge 10% NaCl aqueous (69 kg, 5.5 X) of STEP 14 | |
| | into R5608 | |
| 70. | Adjust R5608 to 20-30° C. | |
| 71. | Stir R5608 at 20-30° C. for 30-60 min | |
| 72. | Allow R5608 to stand for 30-60 min at 20-30° C. | |
| 73. | Transfer R5608 aqueous layer into T3 and label | |
| | material tag | |
| 74. | Load the organic phase in R5608 into new steel-plastic | |
| | composite drums flowed by nitrogen and label IPC | |
| | material tag | |
| 75. | Rinse R5608 with process water to visual clean, then | |
| | dry R5608 for 1-2 h at 70-90° C. under vacuum and | |
| | adjust R5608 to 0-40° C. | |
| 76. | Charge compound 5 organic layer of STEP 74 into | |
| | R5608 via charging device and diaphragm pump and | |
| | Ft-1 | |
| 77. | Rinse the charging pipe with EtOAc (6 kg, 0.5 X) and | |
| | transfer the rinse into R5608 via charging device, | |
| | diaphragm pump and Ft-1 | |
| 78. | Concentrate R5608 mixture at ≤45° C. inner | 40 L |
| | temperature under reduced pressure to 38-50 L (3.0-4.0 X) | |
| 79. | Charge 2-MeTHF (4.3-5.0 X) into R5608 via spray | 61 + 61 + 62 kg |
| | ball, and then concentrate R5608 mixture at ≤45° C. | Total: 184 kg |
| | inner temperature under reduced pressure to 38-50 L | Concentrate terminal volume: 40 L |
| | (3.0-4.0 X) for three times | |
| 80. | Adjust R5608 to 20-30° C. | |
| 81. | IPC: Water content (%, w/w): ≤0.5%, residual EtOAc | Water content: 0.0% |
| | (%, w/w): report | Residual EtOAc: 0.3% |
| 82. | Charge the material of R5608 into STEP 85 of new | |
| | steel-plastic composite drum flowed by nitrogen | |
| 83. | Charge 2-MeTHF (9 kg, 0.7 X) into R5608 to rinse | |
| | charging device, diaphragm and spray ball | |
| 84. | Charge the material of R5608 into STEP 85 of new | |
| | steel-plastic composite drum flowed by nitrogen | |
| 85. | Load the material in R5608 into new steel-plastic | Total net of compound 5 2-MeTHF |
| | composite drums flowed by nitrogen and label IPC | solution: 43.1 kg |
| | material tag | |
| 86. | IPC: Purity of compound 5 (%): report | 95.1% |

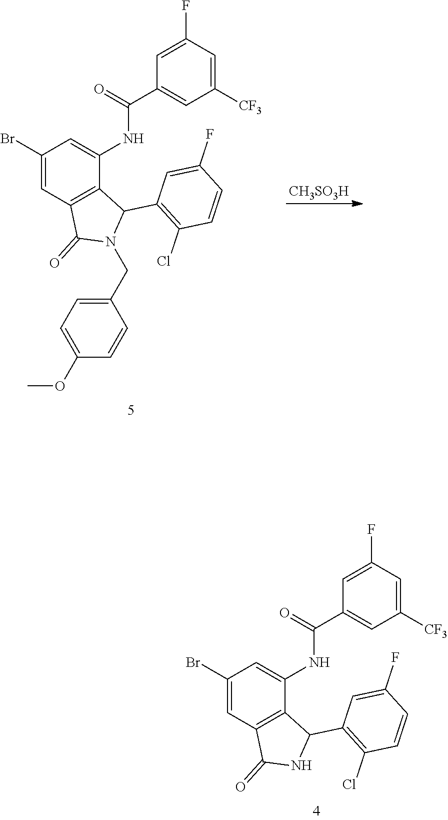 [SMILES: COc1ccc(CN2C(=O)c3cc(Br)cc(NC(=O)c4cc(F)cc(C(F)(F)F)c4)c3C2c2cc(F)ccc2Cl)cc1 ; "CH3SO3H" ; O=C(Nc1cc(Br)cc2c1C(c1cc(F)ccc1Cl)NC2=O)c1cc(F)cc(C(F)(F)F)c1]

### Material Dispensing Summary Table

| | | Actual Amounts | | | |
|---|---|---|---|---|---|
| | | | | Assay for | |
| Materials | | Equiv. | Mass | reactants | Moles |
| Class | Name | eq. | kg | w/w % | mol. |
| Reactants | Compound 5 | 1.0 | 42.8 | N/A | 64.3 |
| | Methanesulfonic acid | N/A | 175 | ≥99.0% | N/A |
| Chemicals | 2- | N/A | 367 | N/A | N/A |
| | Methyltetrahydrofuran | | | | |
| | 2-MeTHF | | | | |
| | Ammonium chloride | N/A | 10 | N/A | N/A |
| | 25% NaCl solution | N/A | 75 | N/A | N/A |
| | Methyl-tert-butylether | N/A | 153 | N/A | N/A |
| | MTBE | | | | |
| | n-heptane | N/A | 71 | N/A | N/A |
| | Process water | N/A | 245 | N/A | N/A |

### Process Description

| | | Comments/Observations/Results |
|---|---|---|
| Operation | | (X = 12.6 kg) |
| 1. | Charge process water (81 kg, 6.4 X) into T3 of STEP 4 | |
| 2. | Charge Ammonium chloride (10 kg, 0.8 X) into T3 of | |
| | STEP 4 in portions | |
| 3. | Bubble by nitrogen in T3 of STEP 4 for 20-60 min | |
| 4. | Load the material in T3 and label material tag | 10% Ammonium chloride aqueous |
| | | solution |
| 5. | Charge process water (18 kg, 1.4 X) into new steel-plastic | |
| | composite drum of STEP 8 | |
| 6. | Charge 25% NaCl solution (75 kg, 6.0 X) into STEP 8 | |
| | drum via peristaltic pump | |
| 7. | Tympanic bulla by nitrogen at 1-2 m³/h for 10-20 min | |
| 8. | Load the material in steel-plastic composite drum and | 20% NaCl aqueous solution |
| | label material tag | |
| 9. | Wash R5201 with 2-MeTHF (82 kg, 6.5 X) via spray ball, | |
| | then load the solvent into the galvanized steel drums that | |
| | purged with nitrogen and label waste tag | |
| 10. | Charge compound 5 2-MeTHE solution (42.8 kg) into | |
| | R5201 in portions | |
| 11. | Rinse the charging pipe with 2-MeTHF (6 kg, 0.5 X) and | |
| | transfer the rinse into R5201 | |
| 12. | Concentrate R5201 mixture at ≤45° C. inner temperature | |
| | under reduced pressure to 19-21 L | |
| 13. | Adjust R5201 to 15-30° C. | |
| 14. | Adding Methanesulfonic acid (175 kg, 13.9 X) into R5201 | |
| | slowly with batch temperature controlled at 15-30° C. via | |
| | peristaltic pump | |
| 15. | Rinse the charging pipe with 2-MeTHF (3 kg, 0.2 X) and | |
| | transfer the rinse into R5201 via peristaltic pump | |
| 16. | Adjust R5201 to 50-55° C. | |
| 17. | Stir R5201 at 50-55° C. for 24-30 h | |
| 18. | IPC: compound 5/compound 4 (%): report | 0.7% |
| 19. | Adjust R5201 to 15-30° C. | |
| 20. | Adding 2-MeTHF (78 kg, 6.2 X) into R5201 slowly with | |
| | the batch temperature controlled at 15-30° C. via peristaltic | |
| | pump | |
| 21. | Charge process water (56 kg, 4.4 X) into R5201 at 15- | |
| | 30° C. via peristaltic pump | |
| 22. | Charge Liquid sodium hydroxide (247 kg, 19.6 X) into | pH = 11 |
| | R5201 slowly at 15-30° C. via peristaltic pump and adjust | |
| | the pH to 10-11 | |
| 23. | Adjust R5201 to 15-25° C. | |
| 24. | Stir R5201 at 15-25° C. for 30-60 min | |
| 25. | Allow R5201 to stand for 30-60 min at 15-25° C. | |
| 26. | Transfer R5201 aqueous layer into T1 and label material | |
| | tag | |
| 27. | Load the organic phase in R5201 into new steel-plastic | |
| | composite drums flowed by nitrogen and label IPC | |
| | material tag | |
| 28. | Charge compound 4 aqueous layer in T1 of STEP 26 into | |
| | R5201 | |
| 29. | Charge 2-MeTHF (77 kg, 6.1 X) into R5201 | |
| 30. | Adjust R5201 to 15-25° C. | |
| 31. | Stir R5201 at 15-25° C. for 30-60 min | |
| 32. | Allow R5201 to stand for 30-60 min at 15-25° C. | |
| 33. | Transfer R5201 aqueous layer into T1 and label material | |
| | tag | |
| 34. | IPC: Residual compound 4 (%, w/w): report | 0.02% |
| 35. | Charge compound 4 organic layer of STEP 27 into | |
| | R5201 | |
| 36. | Charge 10% Ammonium chloride aqueous (90 kg, 7.1 X) | |
| | of STEP 4 into R5201 | |
| 37. | Adjust R5201 to 15-25° C. | |
| 38. | Stir R5201 at 15-25° C. for 30-60 min | |
| 39. | Allow R5201 to stand for 30-60 min at 15-25° C. | |
| 40. | Transfer R5201 aqueous layer into T2 and label material | |
| | tag | |
| 41. | Charge 20% NaCl aqueous (92 kg, 7.3 X) of STEP 8 into | |
| | R5201 | |
| 42. | Adjust R5201 to 15-25° C. | |
| 43. | Stir R5201 at 15-25° C. for 30-60 min | |
| 44. | Allow R5201 to stand for 30-60 min at 15-25° C. | |
| 45. | Transfer R5201 aqueous layer into T2 and label material | |
| | tag | |
| 46. | Load the organic phase in R5201 into new steel-plastic | |
| | composite drums flowed by nitrogen and label IPC | |
| | material tag | |
| 47. | Charge process water (90 kg) into R5201 to rinse charging | |
| | device, diaphragm pump and spray ball and drain off the | |
| | washing water in R5201 to sewer | |
| 48. | Rinse R5201 with process water to visual clean and | |
| | discharge the washing water | |
| 49. | Dry R5201 for 1-2 h at 70-90° C., then adjust R5201 to 0- | |
| | 40° C. | |
| 50. | Charge 2-MeTHF (121 kg, 9.6 X) into R5201 via charging | |
| | device, diaphragm pump, Ft-1 and spray ball | |
| 51. | Heat R5201 to reflux (75-85° C.), distill (75-85° C.) R5201 | |
| | for 15-30 min and reflux (75-85° C.) R5201 for 15-30 min, | |
| | then adjust R5201 to 20-30° C. and load the solvent into | |
| | the galvanized steel drums that purged with nitrogen and | |
| | label waste tag | |
| 52. | Charge compound 4 organic layer of STEP 46 into | |
| | R5201 via charging device, diaphragm pump and Ft-1 | |
| 53. | Concentrate R5201 mixture at ≤45° C. inner temperature | |
| | under reduced pressure to 63-88 L | |
| 54. | Charge MTBE (62 kg, 4.9 X) into R5201 via charging | |
| | device, diaphragm pump and Ft-1 | |
| 55. | Concentrate R5201 mixture at ≤45° C. inner temperature | |
| | under reduced pressure to 63-88 L | |
| 56. | Charge MTBE (61 kg, 4.8 X) into R5201 via charging | |
| | device, diaphragm pump and Ft-1 | |
| 57. | Concentrate R5201 mixture at ≤45° C. inner temperature | |
| | under reduced pressure to 63-88 L | |
| 58. | Adjust R5201 to 15-25° C. | |
| 59. | IPC: Residual 2-MeTHF (%, w/w): report | 23.1% |
| 60. | Adjust R5201 to 40-50° C. | |
| 61. | Stir R5201 at 40-50° C. for 2-4 h | |
| 62. | Adjust R5201 to 20-25° C. slowly | |
| 63. | Stir R5201 at 20-25° C. for 2-4 h | |
| 64. | Charge MTBE (30 kg, 2.4 X) into R5201 via peristaltic | |
| | pump and Ft-1 | |
| 65. | Adjust R5201 to 20-25° C. | |
| 66. | Adding n-heptane (71 kg, 5.6 X) into R5201 slowly with | |
| | the batch temperature controlled at 20-25° C. via peristaltic | |
| | pump and Ft-1 | |
| 67. | Stir R5201 at 20-25° C. for 6-10 h | |
| 68. | IPC: Residual compound 4 in supernatant (%, w/w): | Residual compound 4 in supernatant: 1.1% |
| | report, purity of compound 4 in solid (%): report | Purity of compound 4 in solid: 98.8% |
| 69. | Spread centrifuge bag in M1 | |
| 70. | Transfer R5201 material in portions into M1 for | |
| | centrifuging. During the centrifuging, maintain the reactor | |
| | temperature at 20-25° C. and agitation | |
| 71. | Charge MTBE/n-heptane = 1/1 (v/v) solution (38 kg, 3.0 | |
| | X) to rinse the wet cake (the rinse is used to rinse the | |
| | reactor first if it is the last centrifuge run) | |
| 72. | Load the filtrate into the galvanized steel drums purged by | |
| | nitrogen and label material tag | |
| 73. | Load solid into plastic drum lined with double LDPE bags | Total net of compound 4 wet cake: |
| | and label IPC material tag | 18.4 kg |
| 74. | IPC: Purity of compound 4 (%): report | 99.4% |
| 75. | Put compound 4 wet cake of STEP 73 into drying bag, | |
| | then put to D1, heat D1 to 70-80° C. | |
| 76. | Dry the material in D1 at 70-80° C. under vacuum for 16- | |
| | 24 h | |
| 77. | Adjust D1 to ≤40° C. | |
| 78. | IPC: Water content (%, w/w) ≤0.5%, residual MTBE (%, | Water content: 0.7% |
| | w/w): report, residual 2-MeTHF (%, w/w): report, | Residual MTBE: 2.9% |
| | residual n-heptane (%, w/w): report | Residual 2-MeTHF: 3.4% |
| | | Residual n-heptane <0.01% |
| 79. | Dry the material in D1 at 70-80° C. under vacuum for | |
| | 8-12 h | |
| 80. | Adjust D1 to ≤40° C. | |
| 81. | IPC: Water content (%, w/w) ≤0.5%, residual MTBE (%, | Water content: 0.3% |
| | w/w): report, residual 2-MeTHF (%, w/w): report, | Residual MTBE: 2.4% |
| | residual n-heptane (%, w/w): report | Residual 2-MeTHF: 1.3% |
| | | Residual n-heptane <0.01% |
| 82. | Adjust D1 to 20-40° C. | |
| 83. | Weight and load the material of D1 into the drum and label | |
| | IPC material tag | |
| 84. | Sieve the material of STEP 83 in isolator, then load the | Total net of compound 4: 12.3 kg |
| | sieved product to the fiber drums with iron lid with double | |
| | LDPE bags and label IPC material tag | |
| 85. | IPC: | |

| | |
|---|---|
| Testing Item/Specification Limit | Testing Result |
| Appearance/white to gray solid | Gray solid |
| Identification by HPLC/Retention time | Retention time corresponds to |
| corresponds to that of the reference standard | that of the reference standard |
| Purity, HPLC/≥99% | 99% |
| Water content, KF/≤0.5% | 0.3% |
| Assay, HPLC (%, w/w)/report | 88.8% |
| Residual solvents, HSGC (%, w/w) | |
| Methyl tert-butyl ether/report | 2.2% |
| 2-Methyltetrahydrofuran/report | 1.5% |
| n-heptane/report | <0.0100% |

### Example 2. Synthesis of Compounds I-1, IT-1, and ITT-1

### Main Chain:

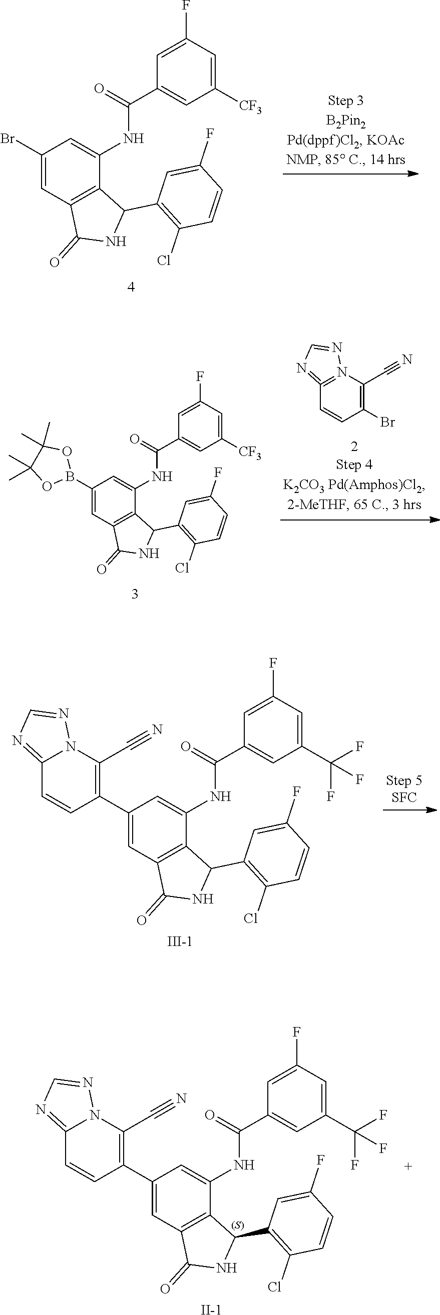

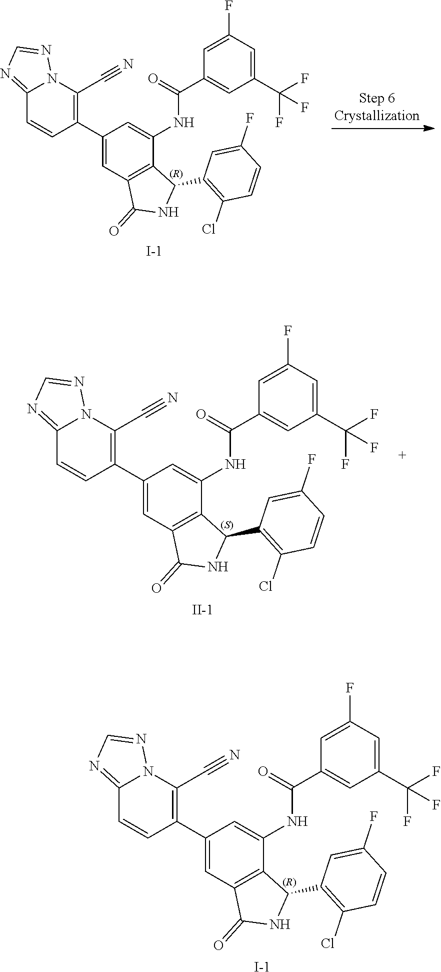

### Recovery Chain:

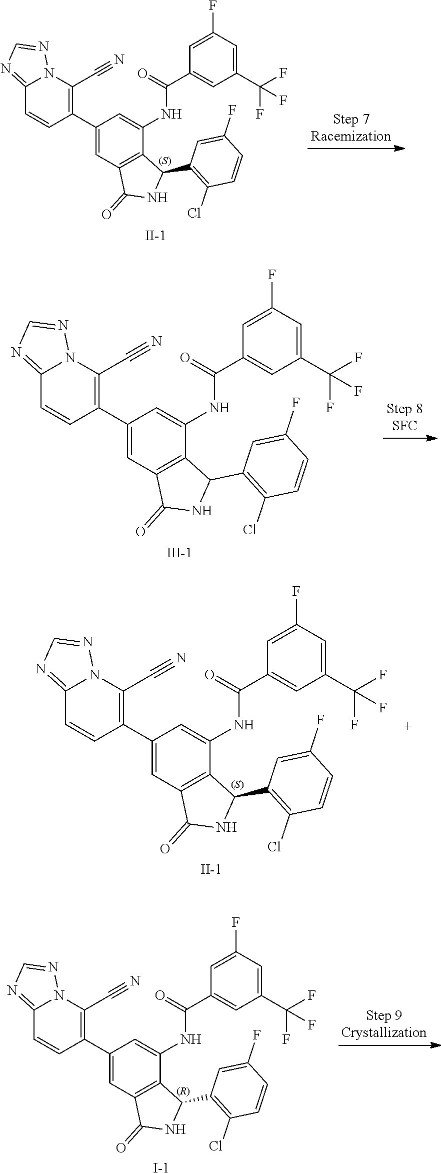

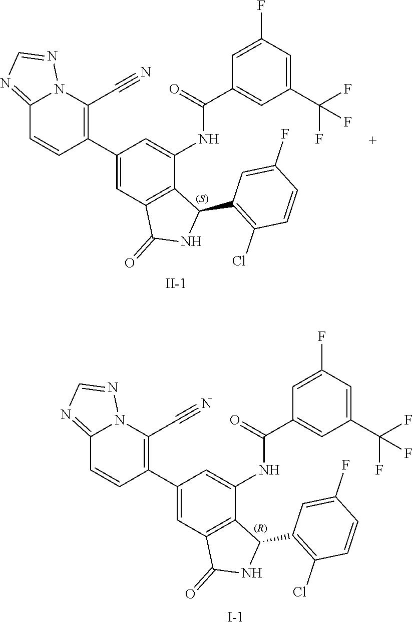

### Preparation of Compound 3

| Starting Materials | | Reaction Conditions | | | | |
|---|---|---|---|---|---|---|
| Com- | | | | | Temp | |
| pound 4 | B2Pin2 | KOAc | Pd(dppf)Cl₂ | NMP | time | IPC |
| 3 g | 1.68 g | 1.62 g | 0.141 g | 24 mL | 85-90° C. | Compound 3: |
| 1.0 eq | 1.2 eq | 3 eq | 0.035 eq | 8 V | 16 h | 92.8% |
| | | | | | | Homo- |
| | | | | | | coupling: 1.5% |
| 5 g(assay | 2.79 g | 2.70 g | 0.235 g | 40 mL | 85-90° C. | Compound 3: |
| corrected) | 1.2 eq | 3 eq | 0.035 eq | 8 V | 18 h | 95.0% |
| 1.0 eq | | | | | | Homo- |
| | | | | | | coupling: 0.54% |

### Preparation of Compound III-1

| Compound 3 | | | Compound III-1 | | | |
|---|---|---|---|---|---|---|
| Starting Materials & | | | Reaction | | | |
| Reaction Condition | | | Conditions | | | |
| | Reagents | | | Reagents | | |
| Material | Conditions | IPC1 | Material | Conditions | IPC2 | |
| Compound 4 | Programmed | Stir for 11 h | Compound 2 | Programmed | Stir for 3 h | Stir for 16 h |
| 3 g (assay | warm to | Compound 3: | (1.1 eq) | warm to | Compound 2: | Compound 2: |
| corrected) | 85-90° C. | 95.0% | PdCl₂(Amphos) | 60-65° C. | N.D | N.D |
| B2Pin2 | over 2 h | Homo- | (0.05 eq) | over 1.5 h | Compound 3: | Compound 3: |
| (1.2 eq) | | coupling: 0.9% | K₂CO₃ | | 0.02% | N.D. |
| KOAc(3 eq) | | | (20%, 3 eq) | | III-1: 79.4% | III-1: 76.0% |
| Pd(dppf)Cl2 | | | | | Homo- | Homo- |
| (0.035 eq) | | | | | coupling: 7.4% | coupling: 7.4% |

### Demo Batch

400 g (assay corrected) Compound 4 was carried out for demo batch. For step 3, consistent IPC result was seen, and the resulting 2-MeTHF solution with 500 ppm of NMP residual. For step 4, IPC was typical, but no solid precipitated out after cooling down and adding water as usual. After extraction and solvent switching, the product was crystallized from DMF/acetone/water=6.25V/6.25V/4.75V. After filtration and drying, 318 g product with 97.8% % purity in 71.3% crude yield was obtained.

Discussion: Similar NMP residual of Compound 3 solution (around 500 ppm) were detected, but the product can't be precipitated from reaction solution directly as before, this isolated process is not reproducible. So DMF was tested as the reaction solvent, and after reaction, acetone and water were added to precipitate product directly.

### Preparation of III-1

| STEP 3 | | | STEP 4 | | |
|---|---|---|---|---|---|
| Starting Materials & | | | Reaction | | |
| Reaction Condition | | | Conditions | | |
| | Reagents | | | Reagents | |
| Material | Conditions | IPC1 | Material | Conditions | IPC2 |
| Compound | NMP | Compound | Compound | 2-MeTHF | Compound 2: |
| 4 400 g | KOAc(3 eq) | 3: 93.4% | 2 | PdCl₂(Amphos) | 3.6% |
| (assay | Pd(dppf)Cl2 | Homo- | (1.1 eq) | (0.05 eq) | Compound 3: |
| corrected) | (0.035 eq) | coupling: | | K₂CO₃ | 0.02% |
| B2Pin2 | 85-90° C. | 2.0% | | (20%, 3 eq) | III-1: 76.9% |
| (1.2 eq) | 12 h | | | 60-65° C. 3 h | Homo- |
| | | | | | coupling: 7.4% |

### III-1:

| Weight | Purity | Appearance | Yield | QNMR | Residual Pd |
|---|---|---|---|---|---|
| 318 g | 97.8% | Gray solid | 71.3% crude | 87.7% | 740 ppm |

### Work-Up Study

| Work up | Results & observation | |
|---|---|---|
| Crystallization | 1) | Cooling down, no solid precipitated out |
| Separate | 1) | Separate |
| | 2) | Wash the organic layer with water |
| Concentrate | Concentrated to 2 V below 45° C. | |
| Crystallization | 1) | Add DMF/Acetone = 6.25 V/6.25 V |
| | 2) | Add 3 V water dropwise over 2 h, solid |
| | | precipitated, stir at 20-25° C. over |
| | | 3 h, more solid precipitated out |
| | 3) | Add 1 V water, stir at 20-25° C. over 1 h |
| | | Mother liquor: |
| | 4) | Add 0.75 V water, stir at 20-25° C. over 1 h |
| | | Mother liquor: wetcake: 97.6% |
| | 5) | Filter |

| | |
|---|---|
| Dry | Dry at 45° C., obtain 318 g gray solid |

### Solvent Screening

20 g Compound 4 was carried out to try DMF process. For step 3, consistent IPC result was seen. For step 4, use DMF as solvent, but only 67.7% IPC purity, lower than before (77%). DMF can't be used for reaction solvent, 2-MeTHF will continue to be used.

### Preparation of III-1

| STEP 3 | | | STEP 4 | | |
|---|---|---|---|---|---|
| Starting Materials & | | | Reaction | | |
| Reaction Condition | | | Conditions | | |
| | Reagents | | | Reagents | |
| Material | Conditions | IPC1 | Material | Conditions | IPC2 |
| Compound 4 | NMP | Compound 3: | Compound | DMF | Compound 2: |
| 20 g (assay | KOAc(3 eq) | 93.2% | 2 | PdCl₂(Amphos) | 2.2% |
| corrected) | Pd(dppf)Cl2 | Homo- | (1.1 eq) | (0.05 eq) | Compound 3: |
| B2Pin2 | (0.035 eq) | coupling: | | K₂CO₃ | 0.2% |
| (1.2 eq) | 85-90° C. | 2.7% | | (20%, 3 eq) | III-1: 67.7% |
| | 2 h | | | 60-65° C. 3 h | Homo- |
| | | | | | coupling: 10.0% |

### Process Optimization (2-MeTHF Process)

20 g Compound 4 was carried out for work-up optimization. For step 3, consistent IPC result was seen. For step 4, still using 2-MeTHF as solvent, IPC still typical, no solid precipitated out after cooling down and adding water. After extraction and water washing, 2-MeTHF was exchanged to DMF, and crystallized from DMF/acetone/water=6V:6V:6V. After crystallization, 20.0 g product with 94.3% purity and 89.8% assay in 80.5% yield (assay corrected) was obtained.

### Preparation of III-1

| STEP 3 | | | STEP 4 | | |
|---|---|---|---|---|---|
| Starting Materials & | | | Reaction | | |
| Reaction Condition | | | Conditions | | |
| | Reagents | | | Reagents | |
| Material | Conditions | IPC1 | Material | Conditions | IPC2 |
| Compound 4 20 g | NMP | Compound | Compound | 2-MeTHF | Compound 2: |
| (assay corrected) | KOAc(3 eq) | 3: 93.8% | 2 | PdCl₂(Amphos) | 2.8% |
| B2Pin2 (1.2 eq) | Pd(dppf)Cl2 | Homo- | (1.1 eq) | (0.05 eq) | Compound 3: |
| | (0.035 eq) | coupling: | | K₂CO₃ | 0.3% |
| | 85-90° C. | 1.5% | | (20%, 3 eq) | III-1: 79.3% |
| | 2 h | | | 60-65° C. 3 h | Homo- |
| | | | | | coupling: 7.1% |

### Step 2 Work-Up Study:

| Work up | Results & observation | |
|---|---|---|
| Separate and Wash | 1) | Separate the organic layer |
| | 2) | Reverse extraction of aqueous layer with 2 V EtOAc |
| | 3) | Wash the organic layer with 3 V 1N HCl solution |
| | 4) | Wash the organic layer with 3 V 10% NaCl solution |
| Concentrate | 1) | Concentrated to 2 V below 45° C. |
| Crystallization | 1) | Add 2 V DMF, concentrate to 2-3 V |
| | 2) | Add 4 V DMF and 6 V Acetone |
| | 3) | Add 3 V water dropwise over 2 h, solid precipitated, stir |
| | | at 20-25° C. over 3 h, more solid precipitated out |
| | 4) | Add 2 V water, stir at 20-25° C. over 1 h |
| | | Mother liquor: wet cake: 98.3% purity |
| | 5) | Add 1 V water, stir at 20-25° C. |
| Drying | 1) | Dry at 50-60° C. |

### III-1:

| | | | | | Residual |
|---|---|---|---|---|---|
| Weight | Purity | Appearance | Yield | Assay | Pd |
| 20.0 g | 94.3% | Gray solid | 80.5% | 89.8% | 4962 ppm |

### Typical PROCEDURE

1. Charge 226 g (5.09-6.22×) NMP into R1
2. Charge 40 g (1.0×) Compound 4 into R1
3. Stir R1 for 0.5-1.0 h at 20-25° C. to form a clear solution
4. Charge 22.34 g B2Pin2 (0.53-0.59×) into R1 under N₂ protection
5. Charge 21.58 g KOAc (0.51-0.57×) into R1 under N₂ protection
6. Charge 100 g (2.25-2.75×) NMP into R1
7. Bubble with N₂ over 1 h at 20-30° C.
8. Charge 1.877 g (0.045-0.057×) Pd(dppf)Cl2 into R1
9. Replace R1 with N₂ gas for 3 times
10. Adjust R1 to 85-90° C.
11. Stir R1 for 2-17 h at 85-90° C.
12. IPC: Compound 4/Compound 3≤1.0%
13. Adjust R1 to 20-25° C.
14. Charge 288 g (6.48-7.92×) EtOAc into R1
15. Charge 320 g (7.20-8.80×) process water into R1
16. Stir R1 for 0.5-1 h at 20-30° C.
17. Stand R1 for 0.5-1 h
18. Separate the upper layer into T1 and remove the bottom layer into T2
19. Charge aqueous layer from T2 into R1
20. Charge 180 g (4.05-4.95×) EtOAc into R1
21. Stir R1 for 0.5-1 h at 20-30° C.
22. Stand R1 for 0.5-1 h
23. Separate the upper layer into T1 and remove the bottom layer into T2
24. Charge aqueous layer from T2 into R1
25. Charge 180 g (4.05-4.95×) EtOAc into R1
26. Stir R1 for 0.5-1 h at 20-30° C.
27. Stand R1 for 0.5-1 h
28. Remove the bottom layer into T2
29. Charge organic layer from T1 into R1
30. Filter through 10 g (0.2-0.3×) diatomite into T2 at 20-30° C.
31. Wash the cake with 36 g (0.9-1.0×) EtOAc
32. Charge the organic layer from T2 into R1
33. Charge 200 g (4.50-5.50×) 5% NaCl solution into R1
34. Stir R1 for 0.5-1 h at 20-30° C.
35. Stand R1 for 0.5-1 h
36. Separate and remove the bottom layer into T1
37. Charge 200 g (4.50-5.50×) 5% NaCl solution into R1
38. Stir R1 for 0.5-1 h at 20-30° C.
39. Stand R1 for 0.5-1 h
40. Separate and remove the bottom layer into T1
41. Charge 200 g (4.50-5.50×) 5% NaCl solution into R1
42. Stir R1 for 0.5-1 h at 20-30° C.
43. Stand R1 for 0.5-1 h
44. Separate and remove the bottom layer into T1
45. Filter through 40 g (0.9-1.1×) silica gel and wash the cake with 450 g (11-14×) EtOAc
46. Concentrate R2 to 2-3V below 40° C.
47. Charge 172 g (6.18-7.56×) 2-MeTHF into R2
48. Concentrate R2 to 2-3V below 40° C.
49. Charge 275 g (6.18-7.56×) 2-MeTHF into R2
50. Concentrate R2 to 3-4V below 40° C.
51. Charge 155 g (3.00-5.50×) 2-MeTHF into R2
52. Charge 18.0 g (0.40-0.50×) Compound 2 into R2
53. Charge 152 g (3.4-4.2×) 20% K2CO3 solution into R2
54. Bubble with N₂ over 1 h at 20-30° C.
55. Charge 2.6 g (0.062-0.078×) Pd(Amphos)Cl2 into R2
56. Replace R2 with N₂ gas for 3 times
57. Adjust R2 to 60-65° C.
58. IPC: Compound 3/Compound III-1≤1.0%
59. Adjust R2 to 20-30° C.
60. Stir R2 for 1-2 h at 20-30° C.
61. Stand R2 for 0.5-1 h
62. Separate and remove the bottom layer into T1, charge the upper layer into T2
63. Charge the solution from T1 into R2
64. Charge 108 g (2.5-4.0×) EtOAc into R2
65. Stir R2 for 0.5-1 h at 20-30° C.
66. Stand R2 for 0.5-1 h
67. Separate and remove the bottom layer into T1
68. Charge the solution from T2 into R2
69. Charge 120 g (2.8-3.3×) 10% NaCl solution into R2
70. Stir R2 for 0.5-1 h at 20-30° C.
71. Stand R2 for 0.5-1 h
72. Separate and remove the bottom layer into T1
73. Pull sample to analysis
74. Residual III-1 in aqueous layer: report
75. Filter through 1.2 g (0.25-0.35×) silica thiol and wash the cake with 320 g (5-12×) EtOAc
76. Concentrate R2 to 2-3V below 40° C.
77. Charge 76 g (1.7-2.1×) DMF into R2
78. Concentrate R2 to 3-4V below 40° C.
79. Charge 162 g (3.6-4.5×) DMF into R2
80. Charge 198 g (4.4-5.5×) Acetone into R2
81. Add drop-wise 200 g (4-7×) water into R2 over 4 h at 20-30° C.
82. Stir R2 for 2-3 h at 20-30° C.
83. Check Compound I-1 wet cake purity: Report
84. Check Compound I-1 mother liquid purity: Report
85. Filter
86. Wash the wet cake with 60 g (1-2×) water
87. Wash the cake with 120 g (1-2×) Acetone.
88. Dried under vacuum at 50˜60° C. for 12˜18 h.
89. Load in drums.

### Preparation and Crystallization of Compound I-1

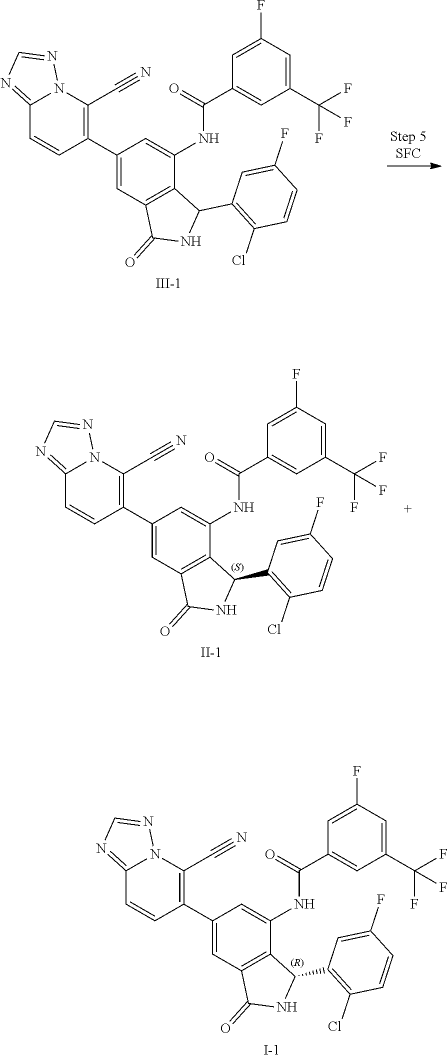

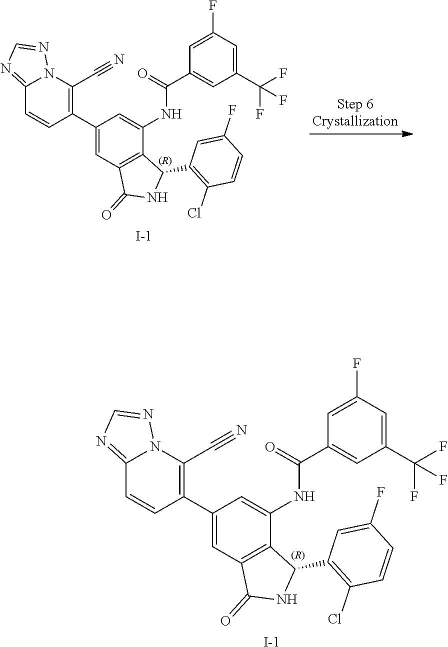

### Summary

Demo batch was purified by SFC preparation to give 102 g working standard with 98.0% purity and 99.5% chiral purity after slurry. For manufacture batch, 7.65 kg I-1 was obtained after PreHPLC, then 7.34 kg product with 99.2% purity and 98.3% chiral purity (GLP batch) was produced after slurry.

Stability study of compound I-1 solution was done, and it was stable when pH at 4˜5.

### PreHPLC Separation

300 g racemate was separated by PreHPLC. 1) 141 g wet cake (I-1) was obtained with 98.7% purity and 99.5% chiral purity. 2) 165.8 g wet cake (II-1) was obtained. 7.65 kg I-1 after PreHPLC preparation with 99.3% purity, 98.8% chiral purity and 125 ppm Pd residual was obtained.

### Result of SFC Separation:

| | Weight | Purity | Chiral purity | Residual Pd |
|---|---|---|---|---|
| I-1 | 141 g wet cake | 98.7% | 99.5% | 587 ppm |

### Working Standard Preparation

Slurry 141 g wet cake (I-1) with 10V EA/Heptane (1:5) in 25° C. for 14h, and after filtration and drying, 102.7 g off-white solid with 98.0% purity and 99.5% chiral purity was obtained. Solvent residual: MeOH, Acetone, DCM, 2-Me-THF, NMP<100 ppm, EA=2370 ppm, n-Heptane=212 ppm, DMF=732 ppm.

### Result of Working Standard

| | Appear- | | Chiral | Residual | Residual | |
|---|---|---|---|---|---|---|
| Weight | ance | Purity | purity | Pd | Cl- | ROI |
| 102.7 g | Off White | 99.2% | 99.5% | 464 ppm | 4250 ppm | 0.16% |
| | solid | | | | | |

### Slurry Process of 1-1

20 g I-1 after SFC preparation was carried out to evaluate slurry process (EtOAc/Heptane: 2V/10V). 19.0 g product with 99.4% purity, 98.5% chiral purity and 100% assay was obtained.

7.65 kg I-1 was carried out for slurry. 7.34 kg product with 99.2% purity and 98.3% chiral purity was obtained. Solvent: ACN=216 ppm, EtOAc-41291 ppm, n-Heptane=669 ppm, DMF=182 ppm, NMP=134 ppm, MeOH, Acetone, DCM, 2-Me-THF <100 ppm

### Result of Slurry:

| | Slurry | | | | | Chiral | | Residual | |
|---|---|---|---|---|---|---|---|---|---|
| HPLC | solvent | Weight | | Appearance | Purity | purity | Assay | Pd | |
| 99.4% | EtOAc/Heptane | 19.0 | g | Off-White | 99.4% | 98.5% | 100% | 94 | ppm |
| | 2 V/10 V | | | solid | | | | | |
| 99.4% | EtOAc/Heptane | 7.34 | kg | Off-White | 99.2% | 98.3% | 96.2% | 149 | ppm |
| | 2 V/10 V | | | solid | | | | | |

### Slurry Process:

1. Charge I-1 (7.65 kg) into R1.
2. Charge (EA 15.3 L) into R1.
3. Charge (N-HEPTANE 76.5 L) into R1.
4. Stir R1 for 4 hr at 20-30° C.
5. Filter reaction mixture.
6. Dry the wet cake at 40-50° C. for 17 hr.
7. Obtain I-1 (7.35 kg).

### Stability of 1-1 During Concentration

To study the chiral stability of product I-1 during concentration, three batches I-1 from Prep-HPLC was tried under different conditions: 1) The ee % of the product changes from 98.20% to 78.95% after stirring over 18 h at 45-50° C. with pH 6-7. 2) The ee % of the product changes from 98.20% to 97.64% after stirring over 18 h at 20-25° C. with pH 6-7. 3) The ee % of the product changes from 98.20% to 98.15% after stirring over 18 h at 45-50° C. with pH 5-6.

Two reactions at 0.5 g I-1 (after SFC preparation) were carried out for stability study. 1) Compound is stable after stirring over 20 h at 10-20° C. with pH 4. 2) The ee % of this compound change from 98.43% to 98.16% after stirring over 20 h at 40-50° C. with pH 4.

### Result of I-1:

| ee % before | | | | | |
|---|---|---|---|---|---|
| concentration | Tem. | pH | Time | ee % | Purity |
| ee % 98.20% | 45-50° C. MeOH | 6-7 | Stir for 18 h | 78.95% | / |
| | 20-25° C. MeOH | 6-7 | Stir for 18 h | 97.64% | / |
| | 45-50° C. | 5-6 | Stir for 18 h | 98.15% | / |
| | MeOH, 1N HCl | | | | |
| ee % 98.43% | 10-20° C. | 4 | Stir for 4 h | 98.33% | 99.53% |
| | MeOH, 1N HCl | | Stir for 20 h | 98.39% | 99.54% |
| | | | Stir for 68 h | 98.27% | 99.53% |
| | 40-50° C. | 4 | Stir for 4 h | 98.38% | 99.51% |
| | MeOH, 1N HCl | | Stir for 20 h | 98.16% | 99.37% |
| | | | Stir for 68 h | 97.92% | 99.29% |

Result: 1) the compound is stable under 10˜20° C. within 68 h, 2) the purity and chiral purity only have slight degrade under 40˜50° C. stirring for 68 h.

### Light Impact (Chiral Purity)

Four reactions were carried out to study the impact of light on chiral purity under neutral and basic condition. After stirring for 96 h, the ee % of product was almost unchanged in avoid-light condition while decreased to 81.68% in under-light condition in neutral system. In base system, ee % decreased more obviously than in neutral system. ee % of product in avoid-light condition decrease to 43.30% while under-light condition decrease to 30.84%.

### Result of Light Impact

| Material | | Time | | | | |
|---|---|---|---|---|---|---|
| ee % | Condition | 0 h | 1 h | 6 h | 24 h | 96 h |
| 98.96% | Avoid light | 98.96% | / | 98.94% | 98.96% | 98.92% |
| | neutral | | | | | |
| | Under light | | | 98.87% | 97.15% | 81.68% |
| | neutral | | | | | |
| | Avoid light | / | 97.91% | 94.79% | 80.92% | 43.30% |
| | Basic(KOAc) | | | | | |
| | Under light | | | 94.35% | 76.17% | 30.84% |
| | basic(KOAc) | | | | | |

Two reactions were carried out to study fluorescence under neutral and basic conditions. No fluorescence was visible by eyes under UV lamp.

### Impurity Preparation

7.9 g Compound 4 (assay corrected) was carried out to do Miyarau and Suzuki reaction for homo-coupling preparation. 92.2% IPC purity was observed, but ˜10% BHT detected. After washing with water and crystallized by DMF/acetone/water=9:6:4, 11.4g homo-coupling with 95.7% purity was obtained.

Two reactions on 90 g Compound 4 were carried out for homo-coupling preparation. IPCs showed good result. After work-up, 127 g homo-coupling with 93.9% purity and 92.2% QNMR was obtained.

### Reaction of Homo-Coupling:

| Boric acid preparation | | | Homo-coupling preparation | | |
|---|---|---|---|---|---|
| Starting Materials & | | | Starting Materials & | | |
| Reaction Condition | | | Reaction Condition | | |
| | Reagents | | | Reagents | |
| Material | Conditions | IPC1 | Material | Conditions | IPC2 |
| Compound 4 | NMP(8 V) | Compound 3: | Compound 4 | 2-MeTHF | Homo- |
| 7.9 g (assay | KOAc(3 eq) | 83.8% | (0.8 eq) | PdCl₂(Amphos) | coupling: |
| corrected) | Pd(dppf)Cl2 | Homo- | | (0.05 eq) | 92.2% |
| B2Pin2 | (0.035 eq) | coupling: | | K₂CO₃ | |
| (1.2 eq) | 85-90° C. | 7.3% | | (20%, 3 eq) | |
| | 4 h | | | 60-65° C., 19 h | |
| Compound 4 | NMP(8 V) | Compound 3: | Compound 4 | 2-MeTHF | Homo- |
| 20 g (assay | KOAc(3 eq) | 97.9% | (0.86 eq) | PdCl₂(Amphos) | coupling: |
| corrected) | Pd(dppf)Cl2 | Homo- | | (0.05 eq) | 90.9% |
| B2Pin2 | (0.035 eq) | coupling: | | K₂CO₃ | |
| (1.2 eq) | 85-90° C. | 0.98% | | (20%, 3 eq) | |
| | 3 h | | | 60-65° C., 1 h | |
| Compound 4 | NMP(8 V) | Compound 3: | Compound 4 | 2-MeTHF | Homo- |
| 72 g (assay | KOAc(3 eq) | 98.5% | (0.88 eq) | PdCl₂(Amphos) | coupling: |
| corrected) | Pd(dppf)Cl2 | Homo- | | (0.05 eq) | 91.0% |
| B2Pin2 | (0.035 eq) | coupling: | | K₂CO₃ | |
| (1.2 eq) | 85-90° C. | 0.45% | | (20%, 3 eq) | |
| | 4 h | | | 60-65° C., 1 h | |

Result of homo-coupling:

| | | | |
|---|---|---|---|
| Weight | Appearance | Purity | QNMR |

| | | | | |
|---|---|---|---|---|
| 11.4 | g | grey solid | 95.7% purity | 75.6% |
| 127 | g | grey solid | 93.9% purity | 92.2% |

Impurity@14.9 min is BHT which comes from THF. Due to the poor solubility of homo-coupling impurity, THF was used as a co-solvent.

### Spiking Reaction

20 g product was carried out for spiking impurity. 98.5% purity with 0.73% homo-coupling and 97.6% chiral purity was obtained after spiking 0.175 g homo-coupling and 0.173 g II-1.

Another 20 g batch was carried out for spiking impurity, 98.4% purity with 0.90% homo-coupling and 97.1% chiral purity was obtained after spiking 0.227 g homo-coupling and 0.252 g II-1.

### Result of Spiking:

| Before spike | | | | | | |
|---|---|---|---|---|---|---|
| | Chiral | | Homo- | | | Chiral |
| Purity | purity | Amount | coupling | II-1 | Purity | purity |
| Purity: | 98.3% | 20 g | 0.175 g | 0.173 g | Purity: | 97.6% |
| 99.2% | | | 0.88% | 0.87% | 98.5% | |
| Homo: | | | | | Homo: | |
| 0.07% | | | | | 0.73% | |
| | | | 0.195 g | 0.221 | Purity: | 97.3% |
| | | | | | 98.5% | |
| | | 20 g | 0.98% | 1.11% | Homo: | |
| | | | | | 0.77% | |
| | | | 0.227 g | 0.252 g | Purity: | 97.1% |
| | | | | | 98.4% | |
| | | | 1.14% | 1.26% | Homo: | |
| | | | | | 0.90% | |

### Racemization Process

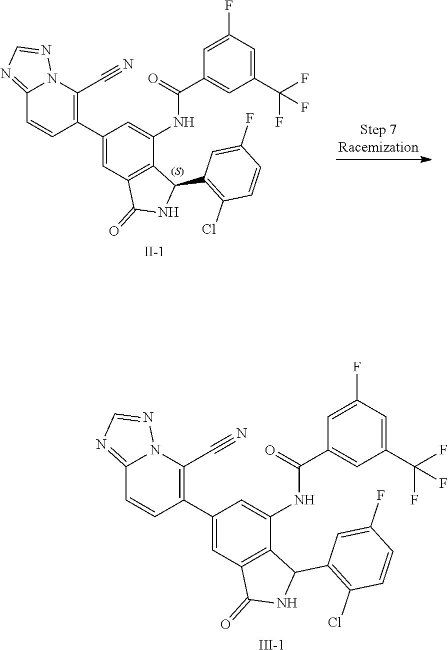

### Summary

To recover compound I-1 from compound II-1, different base and solvent for racemization were screened. KOAc in THF/ACN give the better result. Small-test and verification were successful. Three batches of II-1 were carried out for scale-up in kilo lab.

### Condition Optimization

Six reactions were carried out for racemization process screening. Base (KOAc/NaHCO₃/TEA) and solvent (THF+ACN/THF/THF+ACN+H2O) were screened at 20˜30° C. KOAc in THF/ACN give the better result.

### Reaction of Condition Screening

| | | IPC | | | | | |
|---|---|---|---|---|---|---|---|
| | | HPLC | ee % | HPLC | ee % | HPLC | ee % |
| Solvent | base | 6 h | | 20 h | | 32 h | |
| THF/ACN | NaHCO3 | / | | 98.8% | II-1:I-1 = | 98.3% | II-1:I-1 = |
| 10 V/5 V | | | | | 97:3 | | 94:6 |
| THF 10 V | KOAc | | | 98.6% | II-1:I-1 = | 97.9% | II-1:I-1 = |
| | | | | | 84:16 | | 70:30 |
| THF/ACN/water | KOAc | | | 98.5% | II-1:I-1 = | 91.3% | II-1:I-1 = |
| 10 V/5 V/0.5 V | | | | | 63:37 | | 57:43 |
| THF/ACN | KOAc | 98.3% | II-1:I-1 = | 98.1% | II-1:I-1 = | / | |
| 10 V/5 V | | | 71:29 | | 56:44 | | |
| THF 10 V | TEA | 98.4% | II-1:I-1 = | | | | |
| | | | 98:2 | | | | |
| THF/ACN | TEA | 98.5% | II-1:I-1 = | | | | |
| 10 V/5 V | | | 97:3 | | | | |

### Small-Test and Verification

20 g II-1 was carried out for racemization process. The reaction goes well (I-1: II-1=51.4%: 48.6%). After acid wash, water wash and slurry in EA/MeOH, 16.6 g product with 99.0% purity and 49.6% chiral purity was obtained.
65 g II-1 was carried out for verification. The reaction still goes well (I-1: II-1=51.5%: 48.5%). After typical work-up, 55.8 g product with 98.5% purity and 49.2% chiral purity was obtained.

### Reaction of Racemization

| | | | | IPC | |
|---|---|---|---|---|---|
| II-1 | Solvent | KOAc | | HPLC | Chiral purity |
| 20 g | THF/ACN = 10 V/5 V | 3.22 | g | 97.1% | II-1:I-1 = |
| | | | | | 51:49 |
| 65 g | THF/ACN = 5 V/2.5 V | 10.48 | g | 96.9% | II-1:I-1 = |
| | | | | | 51.5:48.5 |

### Results of Racemization

| Amount | Appearance | Purity | Chiral purity |
|---|---|---|---|
| 16.6 g | Off-white solid | 99.0% | 49.6% |
| 55.8 g | Off-white solid | 98.5% | 49.2% |

### Scale Up

3.12 kg II-1 (assay corrected) was carried out for scale-up, IPC after 19 h shows II-1/1−1=52.4%/47.6%. 2.55 kg crude II-1 (assay corrected) was carried out for scale-up, IPC shows II-1/1−1=52.0%/48.0%. 2.24 kg crude II-1 (assay corrected) was carried out for scale-up, IPC shows II-1/1−1=50.6%/49.4%. Combine product of three batches above. After work-up, 7.114 kg crude product with 97.8% purity, 97.5% assay and 49.0% chiral purity was obtained.

### Reaction of Racemization

| | | | | | IPC | |
|---|---|---|---|---|---|---|
| | | | | | | Chiral |
| II-1 | Solvent | KOAc | Temp | Time | HPLC | purity |
| 3.12 kg | THF/ACN = | 505 g | 25-35° C. | 15 h | 95.4% | II-1/I-1 = |
| | 5 V/2.5 V | 1.0 eq | | | | 53.5/46.5 |
| | | | | 17 h | 96.4% | II-1/I-1 = |
| | | | | | | 52.8/47.2 |
| | | | | 19 h | / | II-1/I-1 = |
| | | | | | | 52.4/47.6 |
| 2.55 kg | THF/ACN = | 420 g | 25-35° C. | 14 h | 97.0% | II-1/I-1 = |
| | 5 V/2.5 V | 1.0 eq | | | | 54.3/45.7 |
| | | | | 18 h | / | II-1/I-1 = |
| | | | | | | 52.5/47.5 |
| | | | | 19 h | / | II-1/I-1 = |
| | | | | | | 52.0/48.0 |
| 2.24 kg | THF/ACN = | 370 g | 25-35° C. | 22 h | 95.6% | II-1/I-1 = |
| | 5 V/2.5 V | 1.0 eq | | | | 50.6/49.4 |

### Work-Up Study

| Work up | Results & observation | |
|---|---|---|
| Quench | 2) | Add 10 V 7% NaHCO3 solution dropwise, part of |
| | | product precipitate out |
| | 3) | Filter and collect solid |
| Extraction | 3) | Extract filtrate with 2-MeTHF(5 V) + EtOAc(5 V) |
| | 4) | Separate and discard aqueous layer |
| Wash with | 1) | Combine solid and organic layer |
| HCl | 2) | Add 5 V 2-MeTHF, still part solid cannot dissolve |
| | 3) | Add 5 V 1N HCl solution dropwise to adjust pH = 2-3 |
| | 4) | Filter and collect solid |
| | 5) | Separate and discard aqueous layer |
| Wash with | 6) | Wash organic layer with 5 V water for 2 times |
| water | 7) | Wash organic layer with 5 V brine |
| Concentrate | 1) | Concentrate organic layer below 35-45° C. under |
| | | vacuum |
| Slurry | 1) | Combine concentrated solution and previous solid, |
| | | Slurry with EA/MeOH = 0.2 V/2 V at room |
| | | temperature |
| | 2) | Filter |

### Results of Racemization

| Amount | Appearance | Purity | Assay | KF | Chiral purity |
|---|---|---|---|---|---|
| 7.114 kg | Off-white solid | 97.8% | 97.5% | 0.16% | 48.8% |

### Racemization Process

1. Charge II-1 (3.3 kg) into R1.
2. Charge (THF 20 L) into R1.
3. Charge (ACN 10 L) into R1.
4. Stir R1 for 1 h at 25-35° C.
5. Charge (KOAc 505 g, 1.004 eq) into R1.
6. Stir R1 for 19 hr at 25-35° C.
7. Add 3750 mL 7% NaHCO₃ solution dropwise.
8. Stir R1 for 0.5 h at 20-30° C.
9. Filter reaction mixture.
10. Charge (EtOAc 16.5 L) into R1.
11. Charge (2-MeTHF 16.5 L) into R1.
12. Stir R1 for 0.5 h at 20-30° C.
13. Separate the upper layer and remove the bottom layer.
14. Charge (2-MeTHF 23.1 L) into R1.
15. Charge 16.5 L 1N HCl into R1 to adjust pH=2-3
16. Stir R1 for 0.5 h at 20-30° C.
17. Filter reaction mixture.
18. Separate the upper layer and remove the bottom layer.
19. Charge (Water 16.5 L) into R1.
20. Stir R1 for 0.5 h at 20-30° C.
21. Separate the upper layer and remove the bottom layer.
22. Charge (Water 16.5 L) into R1.
23. Stir R1 for 20 min at 20-30° C.
24. Separate the upper layer and remove the bottom layer.
25. Charge 16.5 L brine into R1
26. Stir R1 for 0.5 h at 20-30° C.
27. Separate the upper layer and remove the bottom layer.
28. Concentrate R1 below 40-50° C. under vacuum.
29. Dry the wet cake at 40-50° C. for 14 hr.
30. Obtain III-1 (3.1 kg).

### Preparation and Crystallization of Compound I-1

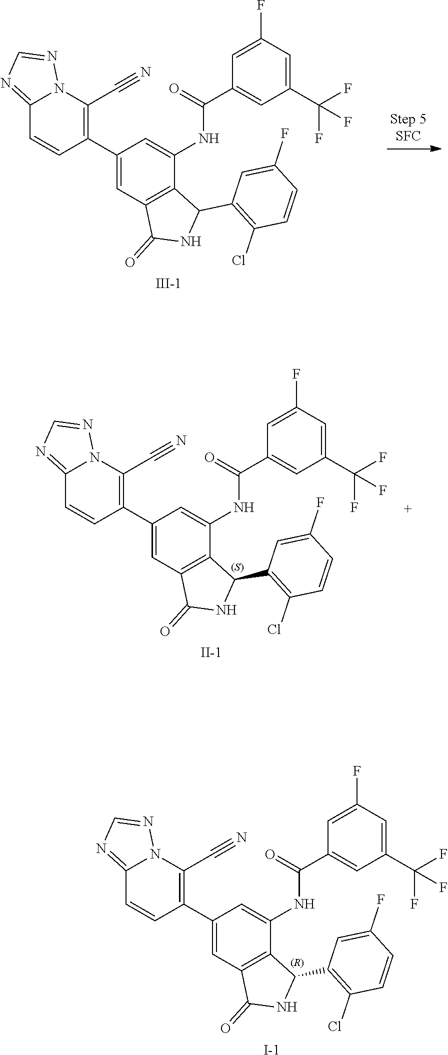

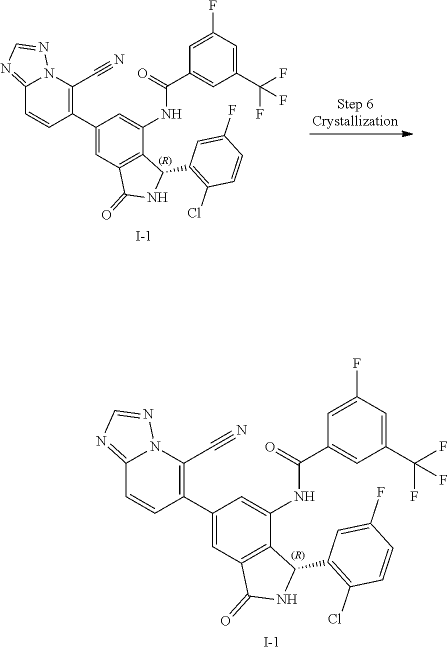

7.11 kg III-1 was separated by Pre-HPLC, to give 3.1 kg I-1, finally 2.42 kg product was obtained after slurry with Heptane/EA, ˜10% EA residual. The results is in table below.

### Results:

| Amount | Slurry solvent | Purity | KF | Chiral purity | Residual Pd |
|---|---|---|---|---|---|
| 2.42 kg | EtOAc/Heptane | 99.7% | 0.32% | 99.6% | 9 ppm |
| | 2 V/10 V | | | | |

### Pilot Plant Production Results

### Preparation of Compound III-1

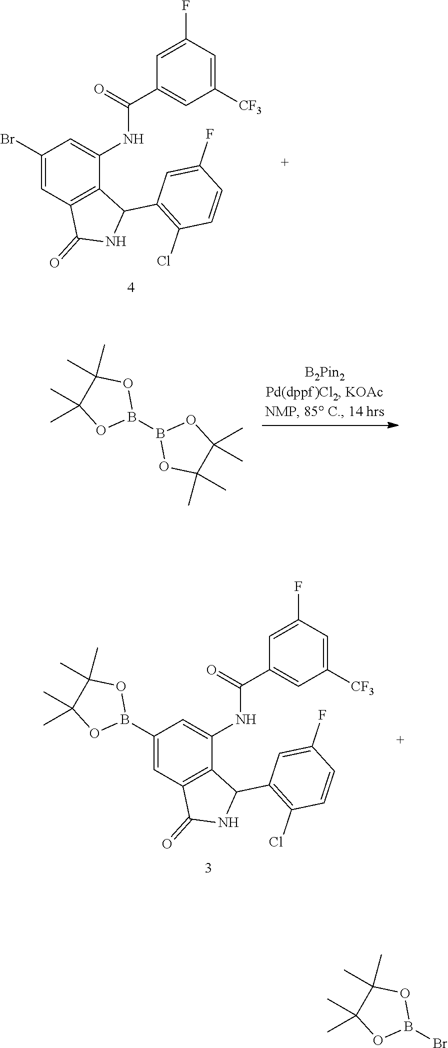

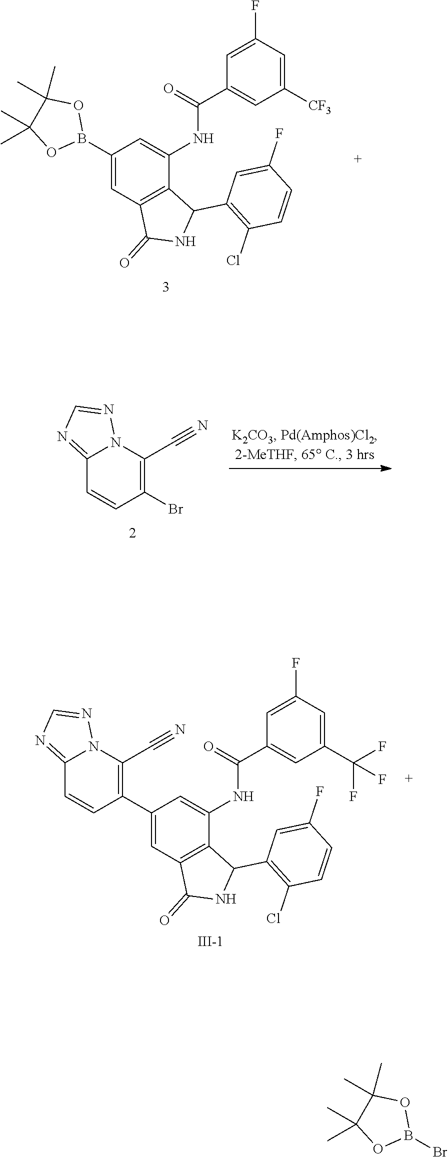

### Results for the Preparation of III-1

| Starting Material | | | | |
|---|---|---|---|---|
| (based on | Product | | | |
| Compound 4, | (crude | | | |
| assay corrected) | amount) | Purity | Assay | Yield |
| 20.7 kg | 18.80 kg | 97.9% | 88.6% | 72% |

### Material Dispensing Summary Table

| | | Actual Amounts | | | |
|---|---|---|---|---|---|
| | | | | Assay for | |
| Materials | | Equiv. | Mass | reactants | Moles |
| Class | Name | eq. | kg | w/w % | mol. |
| Reactants | Compound 4 | 1.00 | 11.20 | 89.9% | 37.9 |
| | | | 12.00 (20.7^{a}) | 88.8% | |
| | Bis(pinacolato)diboron | 1.20 | 11.6 | ≥97% | 45.7 |
| | 6-bromo- | 1.10 | 9.3 | / | 41.7 |
| | [1,2,4]triazolo[1,5,a]pyridine-5- | | | | |
| | carbonitrile | | | | |
| | (Compound 3) | | | | |
| Chemicals | Potassium acetate | / | 11.2 | / | / |
| | [1,1,- | / | 1.2 | / | / |
| | Bis(diphenylphosphino)ferrocene] | | | | |
| | dichloropalladium(II), complex with | | | | |
| | dichloromethane | | | | |
| | Potassium carbonate | / | 17 | / | / |
| | Diatomite | / | 16 | / | / |
| | Silica gel | / | 23 | / | / |
| | Bis(di-tert-butyl (4- | / | 1.3 | / | / |
| | dimethylaminophenyl) phosphine) | | | | |
| | dichloropalladium(II) | | | | |
| | silica thiol | / | 8 | / | / |
| | NMP | / | 167 | / | / |
| | EtOAc | / | 1219 | / | / |
| | 2-MeTHF | / | 487 | / | / |
| | DMF | / | 123 | / | / |
| | Acetone | / | 127 | / | / |
| | Process water | / | 360 | / | / |
| | 5% NaCl aq. solution | / | 304 | / | / |
| | 10% NaCl aq. solution | / | 60 | / | / |

Note:
^{a}corrected by assay

| | | Comments/Observations/Results | |
|---|---|---|---|
| Operation | | (X = 20.5 kg) | |
| 1. | Charge NMP (124 kg) into R1 | | |
| 2. | Adjust R1 to 60-70° C., stir for 10-60 min | | |
| 3. | Adjust R1 to 20-40° C. | | |
| 4. | Take sample for R&D for testing | | |
| 5. | Use test: Compound 4/Compound 3 (%) = N.D. | Use test pass | |
| 6. | Load R1 material into galvanized steel drums | Washing NMP | |
| 7. | Charge NMP (116 kg 5.66 X) into R1 | | |
| 8. | Adjust R1 to 20-25° C. | | |
| 9. | Charge Compound 4 (assay corrected 20.7 kg 1.01 X) into | | |
| | R1 | | |
| 10. | Adjust R1 to 20-25° C., stir R1 at 20-25° C. for 0.5-1.0 h until | | |
| | the solid is dissolved | | |
| 11. | Charge Bis(pinacolato)diboron (11.6 kg 0.57 X) | | |
| 12. | Charge Potassium acetate (11.2 kg 0.55 X) into R1 | | |
| 13. | Charge NMP (51 kg 2.49 X) into R1 | | |
| 14. | Adjust R1 to 20-30° C., stir R1 and bubble with N₂ at 20- | | |
| | 30° C. over 1 h | | |
| 15. | Charge [1,1,- | | |
| | Bis(diphenylphosphino)ferrocene]dichloropalladium(II), | | |
| | complex with dichloromethane (1.2 kg 0.059 X) into R1 | | |
| 16. | Adjust R1 to 85-90° C., stir R1 at 85-90° C. for 2-17 h | | |
| 17. | Adjust R1 to 30-40° C. | | |
| 18. | IPC: Compound 4/Compound 3 (%) ≤1.0% | N.D. | |
| 19. | Adjust R1 to 20-25° C. | | |
| The 1^{st} stratification and filter | | | |
| 20. | Charge EtOAc (161 kg 7.85 X) into R1 | | |
| 21. | Charge process water (170 kg 8.29 X) into R1 | | |
| 22. | Adjust R1 to 20-30° C., stir R1 at 20-30° C. for 0.5-1 h | | |
| 23. | Allow R1 to stand for 0.5-1 h | | |
| 24. | Transfer R1 aqueous layer into new steel-plastic composite | Compound 3 aqueous | |
| | drums | layer: 375.8 kg | |
| 25. | Load the organic layer into STEP 29 drums | Compound 3 NMP/EtOAc | |
| | | solution | |
| 26. | Charge STEP 24 Compound 3 aqueous layer into R1 | | |
| 27. | Extract the aqueous layer with EtOAc (100 kg 4.88 X) | | |
| 28. | Transfer R1 aqueous layer into new steel-plastic composite | | |
| | drums | | |
| 29. | Load R1 organic layer into new steel-plastic composite | Compound 3 NMP/EtOAc | |
| | drums | solution: 305.0 kg | |
| 30. | Charge STEP28 Compound 3 aqueous layer into R1 | | |
| 31. | Extract the aqueous layer with EtOAc (100 kg 4.88 X) | | |
| 32. | Transfer R1 aqueous layer into new steel-plastic composite | Compound 3 aqueous | |
| | drums | layer: 300.6 kg | |
| 33. | IPC: Residual Compound 3 (% w/w): Report | 0.04% | |
| 34. | Charge STEP 29 Compound 3 NMP/EtOAc solution into | | |
| | R1 | | |
| 35. | Adjust R1 to 20-30° C. | | |
| 36. | Spread two filter cloths into F1 | | |
| 37. | Charge diatomite (6 kg 0.3 X) into F1 | | |
| 38. | Charge EtOAc (40 kg) to F1 to moisten the diatomite | | |
| 39. | Press solvent of F1 out to galvanized steel drums | Washing EtOAc | |
| 40. | Press filter R1 material in portions via F1, filter to dryness | | |
| | after loading, transfer the filtrate to the new steel-plastic | | |
| | composite drum of STEP 44 | | |
| 41. | Rinse R1 with EtOAc (50 kg 2.44 X) | | |
| 42. | Press filter R1 rinse EtOAc via F1, filter to dryness after | | |
| | loading, transfer the filtrate to the new steel-plastic | | |
| | composite drum of STEP 44 | | |
| 43. | Protect flexible isolator with N₂. Load F1 wet cake into fiber | Compound 3 Diatomite | |
| | drum lined with double LDPE bag | | |
| 44. | Load the filtrate into the new galvanized steel drums or | Compound 3 NMP/EtOAc | |
| | steel-plastic composite drums | solution: 485.6 kg | |
| The 2^{nd} stratification and filter | | | |
| 45. | Charge STEP 44 Compound 3 NMP/EtOAc solution into | | |
| | R1 | | |
| 46. | Wash the organic phase in R1 with 5% NaCl aq. solution | | |
| | three times (103 kg 5.02 X; 100 kg, 4.88 X; 101 kg; 4.93 | | |
| | X;) | | |
| 47. | Transfer R2 aqueous layer into new steel-plastic composite | Compound 3 aqueous | |
| | drums | layer: 407.0 kg | |
| 48. | Spread two filter cloths into F2 | | |
| 49. | Charge silica gel (23 kg 1.12 X) into F2 | | |
| 50. | Charge EtOAc (438 kg) to F2 to moisten the silica gel | | |
| 51. | Press solvent of F2 out to galvanized steel drums | Washing EtOAc | |
| 52. | Press filter R1 material in portions via F2, filter to dryness | | |
| | after loading, transfer the filtrate to the new steel-plastic | | |
| | composite drum of STEP 56 | | |
| 53. | Rinse R1 with EtOAc (286 kg 14 X) | | |
| 54. | Press filter R1 rinse EtOAc via F2, filter to dryness after | | |
| | loading, transfer the filtrate to the new steel-plastic | | |
| | composite drum of STEP 56 | | |
| 55. | Protect flexible isolator with N₂. Load F2 wet cake into fiber | Compound 3 silica gel | |
| | drum lined with double LDPE bag | | |
| 56. | Load the filtrate into the new galvanized steel drums or | Compound 3 NMP/EtOAc | |
| | steel-plastic composite drums | solution: 656.0 kg | |
| 57. | Wash R2 with 2-MeTHF (119 kg 5.8 X) | | |
| 58. | Take the washing 2-MeTHF for R&D for testing | | |
| 59. | Use test: N.D. | | |
| 60. | Load R2 material into galvanized steel drums | Washing 2-MeTHF | |
| 61. | Charge process water (68 kg 3.32 X) into R2 | | |
| 62. | Charge K₂CO₃ (17 kg 0.83 X) into R2 | | |
| 63. | Adjust R2 to 20-30° C. | | |
| 64. | Stir R2 at 20-30° C. no less than 0.5-1.0 h until the solid is | | |
| | dissolved | | |
| 65. | Load the material in R2 into new steel-plastic composite | 20% K₂CO₃ aq. solution | |
| | drums | | |
| 66. | Wash R2 with process water | | |
| 67. | Charge STEP 56 Compound 3 NMP/EtOAc solution into | 655.2 | kg |
| | R2 in portions | | |
| 68. | Concentrate R2 mixture at ≤40° C. inner temperature under | | |
| | reduced pressure to 2-3 X | | |
| 69. | Charge 2-MeTHF (142 kg 6.9 X) into R2 | | |
| 70. | Concentrate R2 mixture at ≤40° C. inner temperature under | | |
| | reduced pressure to 2-3 X | | |
| 71. | Charge 2-MeTHF (134 kg 6.5 X) into R2 | | |
| 72. | Concentrate R2 mixture at ≤40° C. inner temperature under | | |
| | reduced pressure to 3-4 X | | |
| 73. | Charge 2-MeTHF (92 kg 4.5 X) into R2 | | |
| 74. | Adjust R2 to 20-30° C. | | |
| 75. | Charge Compound 2 (9.3 kg 0.45 X) | | |
| 76. | Charge 20% K₂CO₃ aq. solution (79 kg 3.85 X) into R1 | | |
| 77. | Adjust R2 to 20-30° C., stir R2 and bubble with N₂ at 20- | | |
| | 30° C. over 1 h | | |
| 78. | Charge Bis(di-tert-butyl (4-dimethylaminophenyl) | | |
| | phosphine) dichloropalladium(II) (1.3 kg 0.063 X) into R2 | | |
| 79. | Adjust R2 to 60-65° C., stir R2 at 60-65° C. for 3-4 h | | |
| 80. | Adjust R2 to 30-40° C. | | |
| 81. | IPC: Compound 3/III-1 (%) ≤1.0% | 0.2% | |
| 82. | Adjust R2 to 20-30° C., allow R2 to stand for 0.5-1 h | | |
| 83. | Transfer R2 aqueous layer into the drums of STEP 90 | III-1 aqueous layer | |
| 84. | Load R2 organic layer into new steel-plastic composite | III-1 2-MeTHF | |
| | drums | solution: 169.4 kg | |
| 85. | Charge III-1 aqueous layer (73.2 kg) of STEP 83 into R2 | | |
| 86. | Extract the aqueous layer in R2 with EtOAc (67 kg 3.27 X) | | |
| 87. | Transfer R2 aqueous layer into the drums of STEP 90 | III-1 aqueous layer | |
| 88. | Charge STEP 84 III-1 2-MeTHF solution (169.2 kg) into | | |
| | R2 | | |
| 89. | Wash the organic layer in R2 with 10% NaCl aq. solution | | |
| | (60 kg 2.9 X) | | |
| 90. | Transfer R2 aqueous layer into the new steel-plastic | III-1 aqueous | |
| | composite drums | layer: 139.6 kg | |
| 91. | IPC: Residual of III-1 (% w/w): Report | 0.007% | |
| 92. | Charge silica thiol (8 kg) into R2 | | |
| 93. | Adjust R2 to 25-35° C., stir R2 for 5-10 h at 25-35° C. | | |
| 94. | Spread two filter cloths into F3 | | |
| 95. | Charge diatomite (10 kg 0.49 X) into F3 | | |
| 96. | Charge EtOAc (31 kg) to F3 to moisten the diatomite | | |
| 97. | Press solvent of F3 out to galvanized steel drums | Washing EtOAc | |
| 98. | Press filter R2 material in portions via F3, filter to dryness | | |
| | after loading, transfer the filtrate to the new steel-plastic | | |
| | composite drum of STEP 102 | | |
| 99. | Rinse R2 with EtOAc (346 kg 17 X) | | |
| 100. | Press filter R2 rinse EtOAc via F3, filter to dryness after | | |
| | loading, transfer the filtrate to the new steel-plastic | | |
| | composite drum of STEP 102 | | |
| 101. | Protect flexible isolator with N₂. Load F3 wet cake into fiber | III-1 silica thiol | |
| | drum lined with double LDPE bag | contain Pd | |
| 102. | Load the filtrate into the new galvanized steel drums or | III-1 2-MeTHF/EtOAc | |
| | steel-plastic composite drums | solution: 566.8 kg | |
| 103. | Charge STEP 102 III-1 2-MeTHF/EtOAc solution into R2 | 566.4 | kg |
| | in portions | | |
| 104. | Concentrate R2 mixture at ≤40° C. inner temperature under | | |
| | reduced pressure to 2-3 X | | |
| 105. | Charge DMF (35 kg 1.7 X) into R2 | | |
| 106. | Concentrate R2 mixture at ≤40° C. inner temperature under | | |
| | reduced pressure to 3-4 X | | |
| 107. | Charge DMF (88 kg 4.3 X) into R2 | | |
| 108. | Charge Acetone (104 kg 5.1 X) into R2 | | |
| 109. | Adjust R2 to 20-30° C. | | |
| 110. | Charge process water (82 kg 4 X) into R2 in portions at 20- | | |
| | 30° C. over 4 h | | |
| 111. | Stir R2 at 20-30° C. for 0.5-1 h | | |
| 112. | IPC: | | |

| Test item | Expected value | Result |
|---|---|---|
| Residual of III-1 in filtration (%, w/w) | Report | 0.6% |
| Purity of III-1 (%) | Report | 98.2% |

| 113. | Spread centrifuge bag in M1 | | |
|---|---|---|---|
| 114. | Isolated the wet cake by centrifuge for 1 load and rinse the | | |
| | wet cake with process water (40 kg 2 X) and acetone (23 X | | |
| | 1.1 X), respectively for each load | | |
| 115. | Load solid into plastic drum lined with double PE bags | III-1 wet cake: 25.20 kg | |
| 116. | IPC: Residual of Pd (ppm): Report | 281 | ppm |
| 117. | Load the filtrate into the new steel-plastic drum | III-1 acetone/water mother liquor | |
| 118. | IPC: Residual of III-1 in filtration (%, w/w): Report | 0.6% | |
| 119. | Put the STEP 115 III-1 wet cake into D1 | | |
| 120. | Heat D1 to 50-60° C. | | |
| 121. | Dry D1 under reduced pressure at 50-60° C. for 6-12 h | | |
| 122. | Dry D1 under reduced pressure at 50-60° C. for 12-24 h | | |
| 123. | IPC: | | |

| Test item | Expected value | Result |
|---|---|---|
| Water Content (%, w/w) | Report | 0.4% |
| Residual of 2-MeTHF (%, w/w) | Report | 0.1% |
| Residual of DMF (%, w/w) | Report | <0.10 |
| Residual of n-heptane (%, w/w) | Report | <0.10 |
| Residual of EtOAc (%, w/w) | Report | 0.8% |
| Residual of NMP (%, w/w) | Report | <0.25 (N.D.) |
| Residual of acetone (%, w/w) | Report | 10.6% |

| 124. | Weigh the tare of empty fiber drum lined with double LDPE | III-1: 18.80 kg |
|---|---|---|
| | bags, label IPC material tag, and then load the material of | |
| | dryer into the drum | |
| 125. | IPC: | |

| Test item | Expected value | Result |
|---|---|---|
| Purity of III-1 (%) | Report | 97.9% |
| Assay of III-1 (%, w/w) | Report | 88.6% |

### Example 3. Synthesis of Compounds I-1, II-1, and III-1

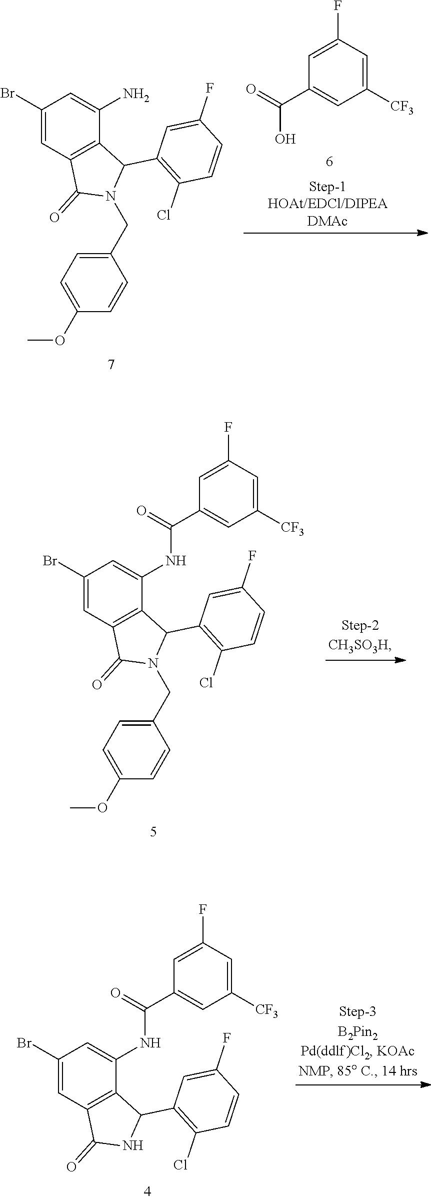

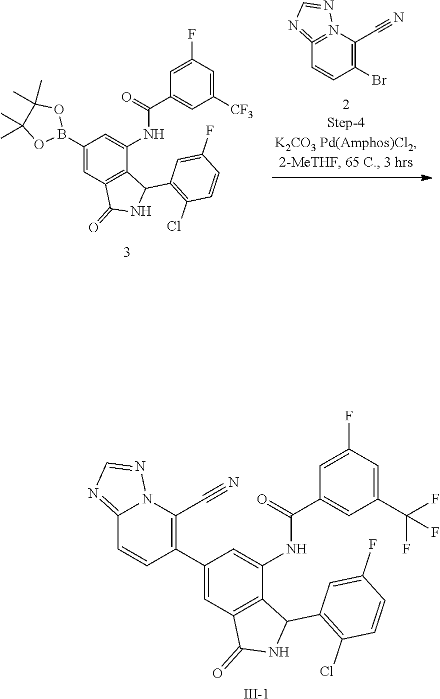

### 3.1 Preparation of Compound 5

1. Charge Compound 7 (100.0 g, 1.00±0.01×) into R1 under N2
2. Charge Compound 6 (65.6 g, 0.66±0.01×) into R1 under N2
3. Charge DMAc (470.0 g, 4.5-5.0×) into R1
4. Charge HOAt (42.9 g, 0.43±0.01×) into R1 under N2
5. Adjust R1 to 20˜30° C.
6. Charge DIPEA (55.0 g, 0.55±0.02×) into R1 under N₂ at 20˜30° C.
7. Stir R1 for 0.5-1 h at 20˜30° C.
8. Charge EDCI (80.6 g, 0.81±0.01×) into R1 under N₂ at 20˜30° C.
9. Adjust R1 to 35˜40° C.
10. Stir R1 for 16-20 h at 35˜40° C.
11. IPC: Compound 7/Compound 5=Report
12. Stir R1 for 4-6 h at 35˜40° C.
13. IPC: Compound 7/Compound 5=Report
14. Charge H2O (900 g, 9.0±0.2×) into R2
15. Charge Na2CO₃ (100 g, 1.00±0.02×) into R2
16. Adjust R2 to 20˜30° C.
17. Stir R2 for 0.5-1 h at 20˜30° C.
18. Drum 10% Na2CO3 aqueous solution
19. Charge H2O (900 g, 9.0±0.2×) into R2
20. Charge NH4Cl (100 g, 1.00±0.02×) into R2
21. Adjust R2 to 20˜30° C.
22. Stir R2 for 0.5-1 h at 20˜30° C.
23. Drum 10% NH4Cl aqueous solution
24. Charge H2O (450 g, 4.5±0.1×) into R2
25. Charge NaCl (50 g, 0.50±0.01×) into R2
26. Adjust R2 to 20˜30° C.
27. Stir R2 for 0.5-1 h at 20˜30° C.
28. Drum 10% NaCl aqueous solution
29. Adjust R1 to 20˜30° C.
30. Charge EA (900 g, 9.0-10.0×) into R1
31. Charge Process water (1500 g, 15.0±0.3×) into R1 under N₂ at 20˜30° C.
32. Stir R1 for 0.5-1 h at 20˜30° C.
33. Stand R1 for 0.5-1 h at 20˜30° C.
34. Separate: Charge the aqueous layer into T1, Charge the organic layer into T2
35. Charge the aqueous layer in T1 into R1
36. Charge EA (450 g, 4.5-5.0×) into R1
37. Adjust R1 to 20˜30° C.
38. Stir R1 for 0.5-1 h at 20˜30° C.
39. Stand R1 for 0.5-1 h at 20˜30° C.
40. Separate: Charge the aqueous layer into T1, Charge the organic layer into T2
41. IPC: Residual Compound 5 in aqueous layer of T1: Report
42. Drum the aqueous layer in T1
43. Charge the organic layer in T2 into R1
44. Charge 10% Na2CO3 aqueous solution (500 g, 5.0±0.1×) into R1
45. Adjust R1 to 20˜30° C.
46. Stir R1 for 0.5-1 h at 20˜30° C.
47. Stand R1 for 0.5-1 h at 20˜30° C.
48. Separate: Charge the aqueous layer into T3
49. Charge 10% Na2CO3 aqueous solution (500 g, 5.0±0.1×) into R1
50. Adjust R1 to 20˜30° C.
51. Stir R1 for 0.5-1 h at 20˜30° C.
52. Stand R1 for 0.5-1 h at 20˜30° C.
53. Separate: Charge the aqueous layer into T3, drum the aqueous layer in T3
54. Charge 10% NH4Cl aqueous solution (500 g, 5.0±0.1×) into R1
55. Adjust R1 to 20˜30° C.
56. Stir R1 for 0.5-1 h at 20˜30° C.
57. Stand R1 for 0.5-1 h at 20˜30° C.
58. Separate: Charge the aqueous layer into T4
59. Charge 10% NH4Cl aqueous solution (500 g, 5.0±0.1×) into R1
60. Adjust R1 to 20˜30° C.
61. Stir R1 for 0.5-1 h at 20˜30° C.
62. Stand R1 for 0.5-1 h at 20˜30° C.
63. Separate: Charge the aqueous layer into T4, drum the aqueous layer in T4
64. Charge 10% NaCl aqueous solution (500 g, 5.0±0.1×) into R1
65. Adjust R1 to 20˜30° C.
66. Stir R1 for 0.5-1 h at 20˜30° C.
67. Stand R1 for 0.5-1 h at 20˜30° C.
68. Separate: Charge the aqueous layer into T5, drum the aqueous layer in T5. Charge organic layer into T6
69. Clear R1
70. Charge organic layer in T6 into R1
71. Concentrate the organic layer in R1 to 3-4V below 45° C.
72. Charge 2-MeTHF (430.0 g, 4.3-5.0×) into R1
73. Concentrate the organic layer in R1 to 3-4V below 45° C.
74. Charge 2-MeTHF (430.0 g, 4.3-5.0×) into R1
75. Concentrate the organic layer in R1 to 3-4V below 45° C.
76. Charge 2-MeTHF (430.0 g, 4.3-5.0×) into R1
77. Concentrate the organic layer in R1 to 3-4V below 45° C.
78. IPC: Residual EA of Compound 5 in RI: Report, KF of Compound 5 in 2-MeTHF solution≤0.5%
79. Charge 2-MeTHF (430.0 g, 4.3-5.0×) into R1
80. Concentrate the organic layer in R1 to 3-4V below 45° C.
81. IPC: Residual EA of Compound 5 in RI: Report, KF of Compound 5 in 2-MeTHF solution≤0.5%
82. Drum 2-MeTHF solution and rinse R1 with 2-MeTHF (86.0 g, 0.9±0.4×)
83. IPC: Purity of Compound 5 in 2-MeTHF solution

### 3.2 Preparation of Compound 4

1. Charge Compound 5 in 2-MeTHF solution into R1 under N2
2. Concentrate the organic layer in R1 to 1.5-2.5V below 45° C.
3. Adjust R1 to 15˜30° C.
4. Charge CH3SO3H (1414 g, 14±0.2×) into R1 at 15˜30° C.
5. Adjust R1 to 50˜55° C.
6. Stir R1 for 24-30 h at 50˜55° C.
7. IPC: Compound 5/Compound 4=Report
8. Stir R1 for 2-8 h at 50˜55° C.
9. IPC: Compound 5/Compound 4=Report
10. Charge H2O (630 g, 6.3±0.2×) into R2
11. Charge NH4Cl (70 g, 0.7˜0.8×) into R2
12. Adjust R2 to 20˜30° C.
13. Stir R2 for 0.5-1 h at 20˜30° C.
14. Drum 10% NH4Cl aqueous solution
15. Charge H2O (560 g, 5.6±0.2×) into R2
16. Charge NaCl (140 g, 1.4˜1.6×) into R2
17. Adjust R2 to 20˜30° C.
18. Stir R2 for 0.5-1 h at 20˜30° C.
19. Drum 20% NaCl aqueous solution
20. Adjust R1 to 15˜30° C.
21. Charge 2-MeTHF (600 g, 5.0˜7.0×) into R1 at 15˜30° C.
22. Charge H2O (420 g, 4.0˜5.0×) into R1 at 15˜30° C.
23. Adjust pH=10˜11 by 30% NaOH (2000 g, 18˜22×) at 15˜30° C.
24. Adjust R1 to 15˜25° C.
25. Stir R1 at 15˜25° C. for 0.5-1 h
26. Stand R1 for 0.5-1 h at 15˜25° C.
27. Separate: Charge aqueous layer into T1, Charge the organic layer into T2
28. Charge the aqueous layer in T1 into R1
29. Charge 2-MeTHF (600 g, 5.0˜7.0×) into R1
30. Adjust R1 to 15˜25° C.
31. Stir R1 at 15˜25° C. for 0.5-1 h
32. Stand R1 for 0.5-1 h at 15˜25° C.
33. Separate: Charge aqueous layer into T1
34. IPC: Residual Compound 4 in aqueous layer: Report
35. Drum the aqueous layer into T1
36. Charge the organic layer in T2 into R1
37. Charge 10% NH4Cl aqueous solution (700 g, 7.0±0.5×) into R1
38. Adjust R1 to 15˜25° C.
39. Stir R1 at 15˜25° C. for 0.5-1 h
40. Stand R1 for 0.5-1 h at 15˜25° C.
41. Separate: charge aqueous layer into T3
42. Charge 20% NaCl aqueous solution (700 g, 7.0±0.5×) into R1
43. Adjust R1 to 15˜25° C.
44. Stir R1 at 15˜25° C. for 0.5-1 h
45. Stand R1 for 0.5-1 h at 15˜25° C.
46. Separate: Charge aqueous layer into T4, Charge organic layer into T5
47. Clean R1
48. Charge the organic layer in T5 into R1
49. Concentrate the organic layer in R1 to 5-7V below 45° C.
50. Charge MTBE (450 g, 4.0˜5.0×) into R1
51. Concentrate the organic layer in R1 to 5-7V below 45° C.
52. Charge MTBE (450 g, 4.0˜5.0×) into R1
53. Concentrate the organic layer in R1 to 5-7V below 45° C.
54. IPC: Residual 2-MeTHF in organic layer: Report
55. Adjust R1 to 40˜45° C.
56. Stir R1 at 40˜45° C. for 0.5-1 h
57. Adjust R1 to 20˜25° C. for 2-4 h
58. Stir R1 at 20˜25° C. for 2-4 h
59. Charge MTBE (180 g, 1.5˜2.5×) into R1
60. Charge n-Heptane (550 g, 5.0˜7.0×) into R1 at 20˜25° C.
61. Stir R1 for 6-10 h at 20˜25° C.
62. IPC: Purity of Compound 4 wet cake: Report, Residual Compound 4 in mother layer: Report
63. Centrifuge
64. Charge MTBE/n-Heptane (1/1, v/v, 1.0˜3.0×) into R1 to rinse the cake
65. Centrifuge
66. IPC: Purity of Compound 4 wet cake: Report
67. Dry the wet cake at 70˜80° C. for 16-24 h.
68. IPC: KF of Compound 4: ≤0.5%, Residual MTBE, 2-MeTHF and n-Heptane of Compound 4: Report
69. Dry the wet cake at 70˜80° C. for 8-12 h.
70. IPC: KF of Compound 4: ≤0.5%, Residual MTBE, 2-MeTHF and n-Heptane of Compound 4: Report
71. Package the product

### 3.3 Preparation of Compound III-1

### 3.3.1 Study of Purging Residual Pd

### Small-Test (DMF)

6 g crude product with 95.8% purity was carried out to do recrystallization to upgrade purity and remove residual Pd. Crude product was dissolved in DMF and stirred with 0.3× silica thiol at room temperature over 17 h. After filtration, the product was recrystallized by DMF/Acetone/water=6.25V/6.25V/4.75V. 4.97 g product with 99.1% purity and 88.3% assay was obtained. Residual Pd decrease from 2106 ppm to 76 ppm.

### Result of III-1

| | | | | | | Residual | | Solvent |
|---|---|---|---|---|---|---|---|---|
| Condition | Weight | | Purity | Appearance | Assay | Pd | | residual |
| / | 6 | g | 95.8% | Grey | 84.9% | 2106 | ppm | / |
| 0.3X silica thiol | 4.97 | g | 99.1% | Off-white | 88.3% | 76 | ppm | Acetone: 8.56% |
| DMF/acetone/water | | | | | | | | DMF: 0.85% |
| 6.25 V/6.25 V/4.75 V | | | | | | | | KF: 0.21% |

### Verification (DMF)

40 g Compound 3 was carried out for verification and use-test. The IPCs show the reaction (two steps) goes normal. Use-test of B2Pin2 and Pd(dppf) 2Cl2 passed. After work-up and first crystallization from DMF/Acetone/Water, wet cake with 97.7% purity and 1539 ppm Pd residual was obtained. And then after further purification by DMF/Acetone/Water, 30.58 g product with 99.2% purity and 89.2% assay in 61.1% yield was obtained. Residual Pd decrease from 1539 ppm to 79 ppm.

### Reaction of III-1

| STEP 3 | | | STEP 4 | | |
|---|---|---|---|---|---|
| Starting Materials & | | | Starting Materials & | | |
| Reaction Condition | | | Reaction Condition | | |
| | Reagents | | | Reagents | |
| Material | Conditions | IPC1 | Material | Conditions | IPC2 |
| Compound 4 | NMP | Compound 3: | Compound 2 | 2-MeTHF | III-1: 84.7%, |
| 40 g (assay | KOAc(3 eq) | 96.6% | (1.0 eq) | PdCl₂(Amphos) | Homo- |
| corrected) | Pd(dppf)Cl2 | | | (0.05 eq) | coupling: |
| B2Pin2 (1.2 eq) | (0.035 eq) | | | K₂CO₃ | 6.0% |
| | 85-90° C. | | | (20%, 3 eq) | |
| | 3 h | | | 60-65° C. 3 h | |

### Result of III-1

| Lot/Batch | | | | | | Residual | | Solvent |
|---|---|---|---|---|---|---|---|---|
| # | Crystallization | Weight | Purity | Appearance | Assay | Pd | | residual |
| First | DMF/acetone/water | Wet | 97.7% | Off-white | / | 1539 | ppm | / |
| crystallization | 6.25 V/6.25 V/4.75 V | cake | | | | | | |
| Purification | 0.3X silica thiol | 30.58 g | 99.2% | Off-white | 89.2% | 79 | ppm | KF: 0.5% |
| Recrystallization | DMF/acetone/water | | | | | | | |
| | 6.25 V/6.25 V/4.75 V | | | | | | | |

### Impurity Profile

| | | 0.61 | | | | | 1.00 | | 1.30 | |
|---|---|---|---|---|---|---|---|---|---|---|
| | | (compound 2) | 0.74 | 0.86 | 0.94 | 0.98 | (III-1) | 1.01 | HomCoup | 1.30 |
| batch | IPC | 0.70 | | 0.09 | 0.92 | 0.57 | 80.99 | 0.84 | 5.13 | 0.43 |
| | Product | | | | 0.18 | 0.52 | 98.19 | | 0.52 | 0.11 |
| | IPC | 5.66 | 1.08 | 1.24 | 0.66 | 0.10 | 73.78 | 1.27 | 3.67 | 6.04 |
| | Cake 1 | 0.23 | 0.10 | 0.21 | | 0.09 | 95.79 | 0.37 | 0.25 | 2.17 |
| | Cake 2 | | | 0.03 | | 0.10 | 99.11 | | 0.02 | 0.54 |
| | IPC | | 0.12 | 0.15 | 1.28 | 0.40 | 84.70 | | 6.00 | 1.16 |
| | Cake 1 | | | 0.02 | 0.49 | 0.38 | 97.71 | | 0.53 | 0.32 |
| | Cake 2 | | | | 0.32 | 0.35 | 99.17 | | 0.04 | 0.12 |
| | IPC | 1.49 | 0.37 | 0.12 | 1.03 | 0.54 | 78.80 | 0.13 | 5.69 | 0.67 |
| | Cake 1 | 0.03 | | 0.04 | 0.55 | 0.05 | 97.89 | 0.09 | 0.68 | 0.18 |

### Proposed Structure:

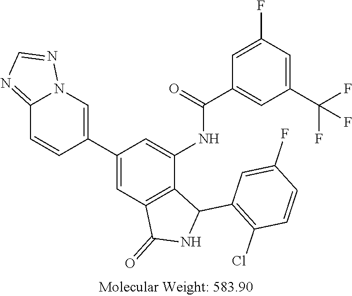 [SMILES: O=C(Nc1cc(-c2ccc3ncnn3c2)cc2c1C(c1cc(F)ccc1Cl)NC2=O)c1cc(F)cc(C(F)(F)F)c1]

### Accumulation

79.4 g (assay corrected) was carried out for accumulation to screen different conditions. For step 3, IPC shows 100% conversion and 95.7% purity in HPLC. For step 4, IPC shows 78.8% purity and 5.69% homo-coupling impurity. After work-up and first crystallization, 66 g crude product with 97.9% purity and 89.0% assay in 66.3% yield was obtained. Residual Pd is 221 ppm.

### Reaction of III-1

| STEP 3 | | | STEP 4 | | |
|---|---|---|---|---|---|
| Starting Materials & | | | Starting Materials & | | |
| Reaction Condition | | | Reaction Condition | | |
| | Reagents | | | Reagents | |
| Material | Conditions | IPC1 | Material | Conditions | IPC2 |
| Compound 4 | NMP | Compound 3: | Compound 2 | 2-MeTHF | Compound 2: |
| 79.4 g (assay | KOAc(3 eq) | 95.7% | (1.1 eq) | PdCl₂(Amphos) | 1.49% |
| corrected) | Pd(dppf)Cl2 | | | (0.05 eq) | III-1: |
| B2Pin2 (1.2 eq) | (0.035 eq) | | | K₂CO₃ | 78.80% |
| | 85-90° C. | | | (20%, 3 eq) | Homo- |
| | 5 h | | | 60-65° C. 3 h | coupling: |
| | | | | | 5.69% |

### Results of III-1

| | | | | | Residual | Solvent |
|---|---|---|---|---|---|---|
| Crystallization | Weight | Purity | Appearance | Assay | Pd | residual |
| DMF/acetone/water | 66 g | 97.9% | Off-white | 89.0% | 221 ppm | Acetone: 8.64%, |
| 6.25 V/6.25 V/5 V | | | | | | EtOAC: 0.08%, |
| | | | | | | 2MeTHF: 0.01%, |
| | | | | | | DMF: 1.04%, |
| | | | | | | NMP: 0.23% |

### Screen the Way of Adsorption

### Filter Through 0.6× Silica Thiol in Column:

15 g crude III-1 were carried out to remove residual Pd. III-1 was dissolved in 4V DMF and filter through 0.6× silica thiol in column. After recrystallized by DMF/Acetone/water=5.45V/5.45V/4.54V, 13.95 g product with 99.2% purity and 91.5% assay was obtained. Residual Pd is 30 ppm.

Stir with 0.6× Silica Thiol:

15 g crude III-1 were carried out to remove residual Pd. III-1 was dissolved in 4V DMF, then stir with 0.6× silica thiol over 20h, After filtration and recrystallized by DMF/Acetone/water-5.45V/5.45V/4.54V, 13.43 g product with 99.3% purity and 92.3% assay was obtained. Residual Pd is 13 ppm.

Stir with 0.3× Silica Thiol for Twice:

15 g crude III-1 were carried out to remove residual Pd. III-1 was dissolved in 4V DMF, then stir with 0.3× silica thiol over 20 h. After filtration, the organic layer stirred with 0.3× silica thiol over 8 h. After filtration and recrystallized by DMF/Acetone/water=5.45V/5.45V/4.54V, 13.59 g product with 99.1% purity and 90.5% assay was obtained. Residual Pd is 10 ppm.

### Result of III-1

| | | | | Residual | Purity | | | |
|---|---|---|---|---|---|---|---|---|
| Material | Process | Amount | Assay | Pd | HPLC | 0.94 | 1.49 | 1.50 |
| 15 g, | Filter | 13.95 g | 91.5% | 30 ppm | 99.2% | 0.43 | 0.19 | 0.12 |
| purity | through | | | | | | | |
| 97.9%, | 0.6X | | | | | | | |
| assay | silica | | | | | | | |
| 89.0%, | thiol in | | | | | | | |
| 221 ppm | column | | | | | | | |
| | slurry | 13.43 g | 92.3% | 13 ppm | 99.3% | 0.47 | 0.07 | 0.07 |
| | with 0.6X | | | | | | | |
| | silica | | | | | | | |
| | thiol one | | | | | | | |
| | time | | | | | | | |
| | slurry | 13.59 g | 90.5% | 10 ppm | 99.1% | 0.48 | 0.23 | 0.11 |
| | with 0.3X | | | | | | | |
| | silica | | | | | | | |
| | thiol for | | | | | | | |
| | two times | | | | | | | |

### Result of III-1

| | Residual solvent |
|---|---|
| batch# | Dry at 50° C. for 20 h |
| | Acetone: 4.81%, EtOAC: 0.01%, 2-Me-THF: <LOQ(0.01%), |
| | DMF: 4.42%, NMP: 0.25% |
| | Acetone: 5.44%, EtOAC: <LOQ(0.01%), 2-Me- |
| | THF: <LOQ(0.01%), DMF: 4.21%, NMP: 0.22% |
| | Acetone: 4.47%, EtOAC: <LOQ(0.01%), 2-Me- |
| | THF: <LOQ(0.01%), DMF: 5.58%, NMP: 0.13% |

### Screen the Types of Sorbents

30 g crude III-1 (414 ppm Pd) from plant was dissolved into DMF, dealt with 0.5× of silica thiol. After filtration, the filtrate was divided into 4 portions:
1) Crystallization directly: After crystallized by DMF/Acetone/water=6.25V/6.25V/4.75V. 6.36 g product with 99.0% purity in 84.8% crude yield was obtained, residual Pd is 36 ppm.
2) Dealt with 0.5× silica thiol and then crystallization: After crystallized by DMF/Acetone/water=6.25V/6.25V/4.75V. 6.48 g product with 98.7% purity in 86.4% crude yield was obtained, residual Pd is 17 ppm.
3) Dealt with 0.2× activated carbon and then crystallization: 5.32 g product with 98.8% purity in 70.9% crude yield was obtained, residual Pd is 5 ppm.
4) Dealt with 0.5× regular silica gel and then crystallization: 6.02 g product with 98.4% purity in 80.3% crude yield was obtained. Residual Pd is 29 ppm.

### Reaction of III-1

| | | | Crystallization Result | | | | |
|---|---|---|---|---|---|---|---|
| Starting | First | Second | | | Residual | | |
| Materials | treatment | treatment | Amount | Purity | Pd | | Note |
| 30 g III-1 | 15 g silica | / | 6.36 g | 99.0% | 36 | ppm | |
| (96.3% | thiol | | | | | | |
| purity | 120 mL | 0.5X silica | 6.48 g | 98.7% | 17 | ppm | |
| 414 ppm | DMF | thiol | | | | | |
| Pd) | 30-40° C. | 0.2X activated | 5.32 g | 98.8% | 5 | ppm | Extra 3 V DMF |
| | 24 h | carbon | | | | | added lead to low |
| | | | | | | | yield and low Pd |
| | | | | | | | residual |
| | | 0.5X regular | 6.02 g | 98.4% | 29 | ppm | |
| | | silica gel | | | | | |

### Screen the Solvent of Crude III-1

### DCM+MeOH:

As it's hard to filtrate when dissolving the crude III-1 with DMF, 15 g crude III-1 was dissolved into DCM/MeOH=16.7V/4V, dealt with 0.5× of silica thiol twice. After concentration and solvent swap, the crude product was crystallized by DMF/Acetone/water=6.25V/6.25V/4.75V. 12.73 g product with 98.6% purity and in 84.9% crude yield was obtained. Residual Pd is 20 ppm. After solvent swapping, ˜10% MeOH residual in DMF solution.

### DCM+MeOH+DMF:

30 g crude III-1 was dissolved into DCM/MeOH/DMF=16.7V/4V/IV, dealt with 0.5× of silica thiol twice. After concentration and solvent swap, the crude product was crystallized by DMF/Acetone/water=6.25V/6.25V/4.75V. 27.5 g product with 98.6% purity was obtained. Residual Pd is 23 ppm.

### Verification (DCM+MeOH+DMF):

50 g crude III-1 was carried out for verification. The product was dissolved into DCM/MeOH/DMF=16.7V/4V/IV, dealt with 0.5× of silica thiol twice. After typical work up, 44.3 g product with 98.3% purity was obtained. Residual Pd is 15 ppm. 20 g III-1 from verification batch was carried out for recrystallization. After recrystallized by DMF/Acetone/water=6.25V/6.25V/4.75V, 18.5 g product with 99.1% purity was obtained. Residual Pd was decreased from 15 ppm to 12 ppm. The purity of DMF solution after concentration and solvent swapping, have slight degradation.

### Recrystallization for Twice:

30 g crude III-1 was dissolved into DCM/MeOH/DMF=16.7V/4V/IV, dealt with 0.5× of silica thiol. After crystalized by DMF/Acetone/water-6.25V/6.25V/4.75V, 27.1 g product with 98.1% purity was obtained. Residual Pd is 23 ppm. The product was carried out for recrystallization. 24.49 g product with 99.3% purity was obtained. Residual Pd is 22 ppm.

### Reaction of III-1

| Starting | First | Second | Crystallization Result | | | | |
|---|---|---|---|---|---|---|---|
| Materials | treatment | treatment | Amount | | Purity | Residual Pd | Residual solvent |
| 15 g III-1 | 0.5X silica | 0.5X silica | 12.73 | g | 99.0% | 20 ppm | MeOH < LOQ(1000 ppm); |
| (96.3% | thiol | thiol | | | | | Acetone102249 ppm; |
| purity | DCM/MeOH | | | | | | DCM < LOQ(1000 ppm); |
| 414 ppm | 16.7 V/4 V | | | | | | EtOAC < LOQ(1000 ppm); |
| Pd) | 20-30° C. | | | | | | 2MeTHF < LOQ(1000 ppm); |
| | 20 h | | | | | | DMF 8291 ppm; |
| | | | | | | | NMP < LOQ(2460 ppm); |
| 30 g III-1 | 0.5X silica | 0.5X silica | 27.5 | g | 98.7% | 23 ppm | MeOH < LOQ(1000 ppm); |
| (96.3% | thiol | thiol | | | | | Acetone102249 ppm; |
| purity | DCM/MeOH | | | | | | DCM < LOQ(1000 ppm); |
| 414 ppm | 16.7 V/4 V | | | | | | EtOAC < LOQ(1000 ppm); |
| Pd) | 20-30° C. | | | | | | 2MeTHF < LOQ(1000 ppm); |
| | 20 h | | | | | | DMF 8291 ppm; |
| | | | | | | | NMP < LOQ(2460 ppm); |
| 50 g III-1 | 0.5X silica | 0.5X silica | 44.3 | g | 98.3% | 15 ppm | MeOH: <1000 ppm; |
| (96.3% | thiol | thiol | | | | | Acetone: 92419 ppm; |
| purity | DCM/MeOH | | | | | | DCM: ND; |
| 414 ppm | 16.7 V/4 V | | | | | | EA: <1000 ppm; |
| Pd) | 20-30° C. | | | | | | 2-MeTHF: ND; |
| | 19 h | | | | | | DMF: 14024 ppm; |
| | | | | | | | NMP: <2460 ppm |
| 20 g III-1 | Recrystallized | | 18.5 | g | 99.1% | 12 ppm | MeOH: <1000 ppm; |
| (98.3% | DMF/Acetone/water | | | | | | Acetone: 90904 ppm; |
| purity | 6.25 V/6.25 V/4.75 V | | | | | | DCM: ND; |
| 15 ppm | | | | | | | EA: <1000 ppm; |
| Pd) | | | | | | | 2-Me-THF: ND; |
| | | | | | | | DMF: 16057 ppm; |
| | | | | | | | NMP: <2460 ppm |
| 30 g III-1 | 0.5X silica | Crystallization | 27.1 | g | 98.1% | 23 ppm | / |
| (96.3% | thiol | DMF/Acetone/water | | | | | |
| purity | DCM/MeOH | 6.25 V/6.25 V/4.75 V | | | | | |
| 414 ppm | 16.7 V/4 V | Recrystallization | 24.49 | g | 99.3% | 22 ppm | MeOH: ND; |
| Pd) | 20-30° C. | DMF/Acetone/water | | | | | Acetone: 109949 ppm; |
| | 19 h | 6.25 V/6.25 V/4.75 V | | | | | DCM: 7509; |
| | | | | | | | EA: ND; |
| | | | | | | | 2-Me-THF: ND; |
| | | | | | | | DMF: 20572 ppm; |
| | | | | | | | NMP: ND |

### Work-Up Study

| Work up | Batch 1 | Batch 2 |
|---|---|---|
| First | stir at 20-30° C. over 20 h | stir at 20-30° C. over 20 h |
| treatment | purity: 96.2% | purity: 95.8% |
| 0.5X silica | | |
| thiol | | |
| Second | stir at 20-30° C. over 20 h | stir at 20-30° C. over 20 h |
| treatment | purity: 96.0% | purity: 95.5% |
| 0.5X silica | | |
| thiol | | |
| Concentra- | Concentrated to 2 V below | Concentrated to 2 V below |
| tion | 40° C. and swap solvent | 40° C. and swap solvent |
| | to DMF, over 3 h | to DMF, over 5 h |
| | purity: 96.2% | purity: 95.3% |
| Crystalliza- | Crystallized by | Crystallized by |
| tion | DMF/Acetone/water = | DMF/Acetone/water = |
| | 6.25 V/6.25 V/4.75 V | 6.25 V/6.25 V/4.75 V |
| | Mother liquor: | Mother liquor: |
| | Wet cake: 98.7% | Wet cake: 98.3% |
| Dry | Dry at 45° C., obtain 27.53 g | Dry at 45° C., obtain 44.3 g |
| | off-white solid | off-white solid |

### Purification of 10 g I-1 for Genotoxicity Study

20 g I-1 was carried out for further purification to get 10 g purified product. After dealt with silica thiol and crystallization, 19.51 g product with 99.3% purity, 98.47% chiral purity was obtained. The residual Pd is 67 ppm. After second purification, 19.77 g crude product with 99.4% purity and 99.08% chiral purity. Residual Pd is 7 ppm, ˜10% EA residual.

### Reaction of I-1

| Crude | | | | Residual | | | Chiral |
|---|---|---|---|---|---|---|---|
| amount | | Condition | Product | Pd | | Purity | purity |
| 20 | g | 20 V | 19.51 g | 67 | ppm | 99.3% | 98.47% |
| | | DCM:MeOH = 5:1 | | | | | |
| | | 1.0X silica thiol | | | | | |
| | | EtOAc/Heptane | | | | | |
| | | 2.5 V:9 V | | | | | |
| 19.51 | g | 19 V | 19.77 g | 7 | ppm | 99.4% | 99.08% |
| | | DCM:MeOH = 8:1 | | | | | |
| | | 1.0X silica thiol | | | | | |
| | | EtOAc/Heptane | | | | | |
| | | 2.5 V:9 V | | | | | |

### Stability of the Process

3 g product was dissolved in MeOH, after stirring over 18 h at 34° C., the purity of the III-1 is 96.0%, 0.15% impurity @RRT 0.74 generated. After stirring over 60 h at 50° C., this impurity increased to 3.44%, other two impurities slight increased.

3 g product was dissolved in DCM/MeOH/DMF, then concentrated to 3V under 40° C., stirred for 17 h at 40° C., the purity of the III-1 is 94.57%, 0.15% impurity @RRT 1.26 generated.

| | HPLC/RRT Purity | | | | | | | |
|---|---|---|---|---|---|---|---|---|
| Tem/time | 0.41 | 0.74 | 0.94 | 0.96 | 1.00 | 1.26 | 1.37 | 1.38 |
| Before stirring | 0.10 | N.D. | 0.62 | N.D. | 96.23 | N.D. | 1.98 | 0.31 |
| 18 h, 34° C. | 0.10 | 0.15 | 0.59 | N.D. | 95.98 | N.D. | 2.33 | 0.29 |
| 34 h, 50° C. | 0.15 | 2.49 | 0.67 | 0.20 | 93.53 | 0.19 | 2.13 | 0.26 |
| 42 h, 50° C. | 0.17 | 2.98 | 0.67 | 0.25 | 92.67 | 0.38 | 2.15 | 0.20 |
| 60 h, 50° C. | 0.19 | 3.45 | 0.67 | 0.40 | 91.45 | 0.58 | 2.26 | 0.23 |
| 16 h, 40° C. | 0.11 | 0.03 | 0.68 | N.D. | 94.57 | 0.33 | 2.51 | 0.46 |

### 3.3.2 Stability of III-1 & I-1 Solid

Purity of I-1 (99.2%) is almost unchanged (99.2%) after 55 days.

Purity of III-1 (99.0%) is almost unchanged (99.1%) after 38 days.

### Pilot Plant Production Results

### Preparation of Compound 5

### Results for the Preparation of Compound 5

| Starting Material | Product | |
|---|---|---|
| (based on Compound 7) | (2-MeTHF solution) | Purity |
| 47.0 kg | 163.4 kg | 94.5% |

### Material Dispensing Summary Table

| | | Actual Amounts | | | |
|---|---|---|---|---|---|
| | | | | Assay for | |
| Materials | | Equiv. | Mass | reactants | Moles |
| Class | Name | eq. | kg | w/w % | mol. |
| Reactants | 4-amino-6-bromo-3-(2-chloro-5- | 1.00 | 47.0 | / | 98.8 |
| | fluorophenyl)-2-(4- | | | | |
| | methoxybenzyl)isoindolin-1-one | | | | |
| | (Compound 7) | | | | |
| | 3-fluoro-5-(trifluoromethyl)benzoic acid | 1.51 | 31.0 | / | 149.0 |
| | (Compound 6) | | | | |
| | 1-Hydroxy-7-azabenzotriazole | 1.51 | 20.3 | / | 149.1 |
| | (HOAT) | | | | |
| | 1-Ethyl-3-(3- | 2.03 | 38.4 | 97%-103% | 200.3 |
| | dimethyllaminopropyl)carbodiimide•HCl | | | | |
| | (EDCI) | | | | |
| | N,N-Diisopropylethylamine | 2.08 | 26.6 | / | 205.8 |
| | (DIPEA) | | | | |
| Chemicals | EA | / | 674 | / | / |
| | Process water | / | 1558 | / | / |
| | 2-Me THF | / | 726 | / | / |
| | DMAc | / | 240 | / | / |
| | NH₄Cl | / | 47.9 | / | / |
| | NaCl | / | 24 | / | / |
| | Na₂CO₃ | / | 47.9 | / | / |

| | | Comments/Observations/Results |
|---|---|---|
| Operation | | (X = 47.0 kg) |
| 1. | Prepare 10% Na₂CO₃ aqueous solution | Na₂CO₃: 47.9 kg; |
| | with Na₂CO₃ and process water | Process water: 424 kg |
| 2. | Prepare 10% NH₄Cl aqueous solution | NH₄Cl: 47.9 kg |
| | with NH₄Cl and process water | Process water: 430 kg |
| 3. | Prepare 10% NaCl aqueous solution | NaCl: 24.0 kg |
| | with NaCl and process water | Process water: 212 kg |
| 4. | Charge Compound 7 (0.99-1.01 X) into R1 | 47.0 kg |
| | | 1.00 X |
| 5. | Charge Compound 6 (0.65-0.67 X) into R1 | 31.0 kg |
| | | 0.66 X |
| 6. | Charge DMAc (4.5-5.0 X) into R1 | 220 kg |
| | | 4.7 X |
| 7. | Start agitator of R1 and set | |
| | agitation speed at 75 rpm | |
| 8. | Charge HOAT (0.42-0.44 X) into R1 | 20.3 kg |
| | | 0.43 X |
| 9. | Adjust R1 to 20-30° C. inner temperature | |
| 10. | Charge DIPEA (0.53-0.57 X) into R1 slowly | 26.6 kg |
| | with inner temperature 20-30° C. | 0.57 X |
| 11. | Stir R1 at 20-30° C. inner temperature | |
| | for 0.5-1.0 h | |
| 12. | Charge EDCI (0.80-0.82 X) into R1 with | 38.4 kg |
| | inner temperature 20-30° C. | 0.82 X |
| 13. | Charge DMAc (10-30 kg) into R1 | 20 kg |
| 14. | Adjust R1 to 35-45° C. inner temperature | |
| 15. | Stir R1 at 35-45° C. inner temperature | |
| | for 16-20 h | |
| 16. | IPC: Compound 7/Compound 5 (% area): Report | 0.05 |
| 17. | Adjust R1 to 20-30° C. inner temperature | |
| 18. | Charge EA (9.0-10.0 X) into R1 | 442 kg |
| | | 9.4 X |
| 19. | Charge process water (14.7-15.3 X) into | 704 kg |
| | R1 with inner temperature 20-30° C. | 15.0 X |
| 20. | Stir R1 at 20-30° C. inner temperature | |
| | for 0.5-1.0 h | |
| 21. | Allow R1 to stand for 0.5-1.0 h at | |
| | 20-30° C. inner temperature | |
| 22. | Load the bottom aqueous layer from | <Compound 5 |
| | reactor into T1 | aqueous solution> |
| 23. | Transfer the organic layer of | <Compound 5 |
| | reactor to T2 | EA solution> |
| 24. | Charge Compound 5 aqueous solution | |
| | of STEP 22 into R1 | |
| 25. | Extract the aqueous layer with | 232 kg |
| | EA (4.5-5.0 X) | 4.9 X |
| 26. | Load the bottom aqueous layer from | <Compound 5 |
| | reactor into T1 | aqueous solution> |
| 27. | IPC: Residual Compound 5 (LC, % | 0.0008 |
| | w/w): Report | |
| 28. | Charge Compound 5 EA solution of | < Compound 5 |
| | STEP 23 into R1 | EA solution> |
| 29. | Charge 10% Na₂CO₃ aqueous solution | 233 kg |
| | (4.9-5.1 X) from STEP 1 into R1 | 5.0 X |
| 30. | Adjust R1 to 20-30° C. inner temperature | |
| 31. | Stir R1 at 20-30° C. inner temperature | |
| | for 0.5-1.0 h | |
| 32. | Allow R1 to stand for 0.5-1.0 h at | |
| | 20-30° C. inner temperature | |
| 33. | Load the bottom aqueous layer from | <Compound 5 Na₂CO₃ |
| | reactor into plastic drum | aqueous solution> |
| 34. | Wash the organic layer in R1 with | 237 kg |
| | 10% Na₂CO₃ aqueous solution (4.9- | 5.0 X |
| | 5.1 X) from STEP 1 | |
| 35. | Load the bottom aqueous layer from | <Compound 5 Na₂CO₃ |
| | reactor into plastic drum | aqueous solution> |
| 36. | Wash the organic layer in R1 with | 236 kg |
| | 10% NH₄Cl aqueous solution (4.9- | 5.0 X |
| | 5.1 X) from STEP 2 | |
| 37. | Load the bottom aqueous layer from | <Compound 5 NH₄Cl |
| | reactor into plastic drum | aqueous solution> |
| 38. | Wash the organic layer in R1 with | 239 kg |
| | 10% NH₄Cl aqueous solution (4.9- | 5.1 X |
| | 5.1 X) from STEP 2 | |
| 39. | Load the bottom aqueous layer from | <Compound 5 NH₄Cl |
| | reactor into plastic drum | aqueous solution> |
| 40. | Wash the organic layer in R1 with | 236 kg |
| | 10% NaCl aqueous solution (4.9- | 5.0 X |
| | 5.1 X) from STEP 3 | |
| 41. | Load the bottom aqueous layer from | <Compound 5 NaCl |
| | reactor into plastic drum | aqueous solution> |
| 42. | Transfer the organic layer of | <Compound 5 |
| | reactor to T2 | EA solution> |
| 43. | Clean R1 with process water | |
| | to pH to 7-8 | |
| 44. | Charge Compound 5 EA solution of | |
| | from STEP 42 into R1 | |
| 45. | Start agitator of R1 and set | |
| | agitation speed at 75 rpm | |
| 46. | Concentrate R1 mixture at <45° | |
| | C. inner temperature under reduced | |
| | pressure to a volume of 3-4 X | |
| 47. | Charge 2-MeTHF (4.3-5.0 X) | 234 kg |
| | into R1 via Ft1 | 5.0 X |
| 48. | Concentrate R1 mixture at <45° | |
| | C. inner temperature under reduced | |
| | pressure to a volume of 3-4 X | |
| 49. | Charge 2-MeTHF (4.3-5.0 X) | 234 kg |
| | into R1 via Ft1 | 5.0 X |
| 50. | Concentrate R1 mixture at <45° | |
| | C. inner temperature under reduced | |
| | pressure to a volume of 3-4 X | |
| 51. | Charge 2-MeTHF (4.3-5.0 X) | 234 kg |
| | into R1 via Ft1 | 5.0 X |
| 52. | Concentrate R1 mixture at <45° | |
| | C. inner temperature under reduced | |
| | pressure to a volume of 3-4 X | |
| 53. | IPC: Residual EtOAc (GC, % w/w): Report | 0.41 |
| | Water content (% w/w) ≤0.5 | 0.0 |
| | Purity of Compound 5 (LG, % area): Report | 94.5 |
| 54. | Adjust R1 to 20-30° C. inner temperature | |
| 55. | Load the R1 material into from | |
| | STEP 57 drum | |
| 56. | Charge 2-MeTHF (0.5-1.3 X) | 24 kg |
| | into R1 via Ft1 | 0.5 X |
| 57. | Load the R1 material into new | <Compound 5 |
| | steel-plastic composite drum | 2-MeTHF solution> |
| | | 163.4 kg |

### Preparation of Compound 4

### Results:

| Starting Material | | | | |
|---|---|---|---|---|
| (based on Compound 7) | Product | Purity | Assay | Yield |
| 47.0 kg | 48.05 kg | 98.9% | 89.2% | 80% |

### Material Dispensing Summary Table

| | | Actual Amounts | | |
|---|---|---|---|---|
| | | | Assay for | |
| Materials | | Mass | reactants | Moles |
| Class | Name | kg | w/w % | mol. |
| Reactants | Compound 5 2-MeTHF | 163.0 | / | / |
| | solution | | | |
| | Methanesulfonic acid | 660 | ≥99.0% | 6867.4 |
| Chemicals | MTBE | 621 | / | / |
| | n-Heptane | 328 | / | / |
| | Process water | 814 | / | / |
| | 2-MeTHF | 700 | / | / |
| | NH₄Cl | 34.9 | / | / |
| | NaCl | 70.4 | / | / |
| | Liquid Sodium | 898 | / | / |
| | hydroxide | | | |

| | | | Comments/Observations/Results |
|---|---|---|---|
| Operation | | | (X = 47.0 kg) |
| 1. | Prepare MTBE/n-heptane solution with MTBE | | MTBE: 71 kg; |
| | (0.52-1.56 X) and n-heptane (0.48-1.44 X) | | n-heptane: 68 kg |
| 2. | Prepare 10% NH₄Cl aqueous solution with | | NH₄Cl: 34.9 kg |
| | NH₄Cl and process water | | Process water: 309 L |
| 3. | Prepare 20% NaCl aqueous solution with | | NaCl: 70.4 kg |
| | NaCl and process water | | Process water: 275 L |
| 4. | Charge all Compound 5 2-MeTHF solution | | 163.0 kg |
| | into R1 | | |
| 5. | Charge 2-MeTHF (10-50 kg) into R1 | | 30 kg |
| 6. | Start agitator of R1 and set agitation | | |
| | speed at 75 rpm | | |
| 7. | Concentrate R1 mixture at <45° C. inner | | |
| | temperature under reduced pressure to | | |
| | a volume of 1.5-2.5 X | | |
| 8. | Adjust R1 to 15-30° C. inner temperature | | |
| 9. | Charge Methanesulfonic acid (13.8-14.2 X) | | 660 kg |
| | into R1 slowly with inner temperature 15-30° C. | | 14.0 X |
| 10. | Charge 2-MeTHF (10-50 kg) into R1 | | 30 kg |
| 11. | Adjust R1 to 45-55° C. inner temperature | | |
| 12. | Stir R1 at 45-55° C. inner temperature | | |
| | for 24-30 h | | |
| 13. | IPC: Compound 5/Compound 4 (LC, % area): Report | | 2.2 |
| 14. | Stir R1 at 45-55° C. inner temperature | | |
| | for 2-8 h | | |
| 15. | IPC: Compound 5/Compound 4 (LC, % area): Report | | 1.0 |
| 16. | Adjust R1 to 15-30° C. inner temperature | | |
| 17. | Charge 2-MeTHF (5.0-7.0 X) into R1 with | | 320 kg |
| | inner temperature 15-30° C. | | 6.8 X |
| 18. | Charge process water (4.0-5.0 X) into R1 | | 230 kg |
| | slowly with inner temperature 15-30° C. | | 4.9 X |
| 19. | Feed Liquid Sodium hydroxide (18-22 X) into | | 898 kg 19 X; |
| | R1 slowly with inner temperature 15-30° | | pH = 11 |
| | C., till pH = 10-11 | | |
| 20. | Adjust R1 to 15-25° C. inner temperature | | |
| 21. | Stir R1 at 15-25° C. inner temperature | | |
| | for 30-60 min | | |
| 22. | Allow R1 to stand for 30-60 min at 15-25° | | |
| | C. inner temperature | | |
| 23. | Load the bottom aqueous layer from | | <Compound 4 |
| | reactor into T1/T2 | | aqueous solution> |
| 24. | Transfer the organic layer of | | <Compound 4 |
| | reactor to T3 | | 2-MeTHF solution> |
| 25. | Charge Compound 4 aqueous solution | | |
| | from STEP 23 into R1 | | |
| 26. | Extract the aqueous layer with 2-MeTHF | | 320 kg |
| | (5.0-7.0 X) | | 6.8 X |
| 27. | Load the bottom aqueous layer from | | <Compound 4 |
| | reactor into T1/T2 | | aqueous solution> |
| 28. | IPC: Residual of Compound 4 (LC, % w/w): Report | | N.D. |
| 29. | Charge Compound 4 2-MeTHF solution | | |
| | STEP 24 into R1 | | |
| 30. | Wash the organic layer in R1 with 10% NH₄Cl | | 345 kg |
| | aqueous solution (6.7-7.5 X) from STEP 2 | | 7.3 X |
| 31. | Load the bottom aqueous layer from | | <Compound 4 NH₄Cl |
| | R1 into T4 | | aqueous solution> |
| 32. | Wash the organic layer in R1 with 20% NaCl | | 344 kg |
| | aqueous solution (6.5-7.5 X) from STEP 3 | | 7.3 X |
| 33. | Load the bottom aqueous layer from | | <Compound 4 NaCl |
| | reactor into plastic drum | | aqueous solution> |
| 34. | Transfer the organic layer of reactor to T3 | | <Compound 4 |
| | | | 2-MeTHF solution> |
| 35. | Wash R1 with process water to clean | | |
| 36. | Charge Compound 4 2-MeTHF solution of from | | |
| | STEP 34 into R1 | | |
| 37. | Start agitator of R1 and set agitation | | |
| | speed at 75 rpm | | |
| 38. | Concentrate R1 mixture at <45° C. inner | | |
| | temperature under reduced pressure to | | |
| | a volume of 5-7 X | | |
| 39. | Charge MTBE (4.0-5.0 X) into R1 via Ft1 | | 220 kg |
| | | | 4.7 X |
| 40. | Concentrate R1 mixture at <45° C. inner | | |
| | temperature under reduced pressure to | | |
| | a volume of 5-7 X | | |
| 41. | Charge MTBE (4.0-5.0 X) into R1 via Ft1 | | 220 kg |
| | | | 4.7 X |
| 42. | Concentrate R1 mixture at <45° C. inner | | |
| | temperature under reduced pressure to | | |
| | a volume of 5-7 X | | |
| 43. | IPC: Residual 2-MeTHF (GC, % w/w): Report | | 25 |
| 44. | Adjust R1 to 35-45° C. inner temperature | | |
| 45. | Stir R1 at 35-45° C. inner temperature | | |
| | for 2-4 h | | |
| 46. | Adjust R1 to 15-25° C. inner temperature | | |
| | with 2-4 h | | |
| 47. | Stir R1 at 15-25° C. inner temperature | | |
| | for 2-4 h | | |
| 48. | Charge MTBE (1.5-2.5 X) into R1 via Ft1 | | 110 kg |
| | | | 2.3 X |
| 49. | Charge n-heptane (5.0-7.0 X) into R1 with | | 260 kg |
| | inner temperature 15-25° C. via Ft1 | | 5.5 X |
| 50. | Stir R1 at 15-25° C. inner temperature | | |
| | for 6-10 h | | |
| 51. | IPC: Compound 4 in mother liquor (GC, % w/w): Report; | | 1.1 |
| | Purity of Compound 4 in wet cake (LC, % area) | | 98.0 |
| 52. | Spread a centrifuge bag on the centrifuge | | |
| 53. | Isolate the wet cake by centrifuge for 2 | | 1^{st} load: 69 |
| | loads and rinse the wet cake with STEP 1 | | kg 1.5 X; |
| | MTBE/n-heptane solution (0.5-1.5 X) | | 2^{nd} load: 64 |
| | for each load | | kg 1.4 X |
| 54. | Load the solid into fiber drum lined with | | <Compound 4 |
| | double antistatic PE bags | | wet cake> |
| | | | 64.90 kg |
| 55. | IPC: Purity of Compound 4 in wet cake (LC, % area) | | 98.4 |
| 56. | Load the mother liquor into plastic- | | <Compound 4 MTBE/ |
| | steel composite drums | | n-heptane |
| | | | mother liquor> |
| 57. | Put the STEP 54 Compound 4 wet cake into | | |
| | antistatic drying bags, then put the | | |
| | bag into D1 | | |
| 58. | Adjust the pressure of D1 to ≤−0.085 MPa | | |
| 59. | Set the jacket temperature of the dryer | | |
| | to 70-80° C. | | |
| 60. | Dry D1 under reduced pressure at | | |
| | 70-80° C. for 16-24 h | | |
| 61. | IPC: | | |
| | Test item | Expected value | Result |
| | Water Content (%, w/w) | Report | 0.10 |
| | Residual of MTBE (GC, %, w/w) | Report | 2.7 |
| | Residual of 2-MeTHF (GC, %, w/w) | Report | 5.0 |
| | Residual of n-heptane (GC, %, w/w) | Report | <0.0100 |
| 62. | Dry D1 under reduced pressure at | | |
| | 70-80° C. for 8-12 h | | |
| 63. | IPC: | | |
| | Test item | Expected value | Result |
| | Water Content (%, w/w) | Report | 0.2 |
| | Residual of MTBE (GC, %, w/w) | Report | 2.5 |
| | Residual of 2-MeTHF (GC, %, w/w) | Report | 5.3 |
| | Residual of n-heptane (GC, %, w/w) | Report | <0.0100 |
| 64. | Load the dryer into empty fiber | | Compound 4 |
| | drum lined with double LDPE bags | | 48.05 kg |
| 65. | IPC: | | |
| | Test item | Expected value | Result |
| | Purity of Compound 4 (LC, % area) | Report | 98.9 |
| | Assay of Compound 4 (%, % w/w) | Report | 89.2 |

### Preparation of Compound III-1

### Results:

| Starting Material | Product | | | |
|---|---|---|---|---|
| (based on Compound 4) | (crude amount) | Purity | Assay | Yield |
| 43.50 kg | 34.40 kg | 95.4% | 85.2% | 68% |

### Material Dispensing Summary Table

| | | Actual Amounts | | | |
|---|---|---|---|---|---|
| | | | | Assay for | |
| Materials | | Equiv. | Mass | reactants | Moles |
| Class | Name | eq. | kg | w/w % | mol. |
| Reactants | Compound 4 | 1.00 | 43.5 (38.8) | 89.2% | 71.1 |
| | Bis(pinacolato)diboron | 1.22 | 22.0 | ≥97% | 86.6 |
| | B₂Pin2 | | | | |
| | 6-bromo- | 1.10 | 17.5 | / | 78.5 |
| | [1,2,4]triazolo[1,5,a]pyridine-5- | | | | |
| | carbonitrile | | | | |
| | (Compound 2) | | | | |
| | Potassium acetate | 3.01 | 21.0 | ≥99.0% | 214.0 |
| | (KOAc) | | | | |
| | [1,1,- | 0.036 | 1.85 | Pd ≥ 14.0% | 2.53 |
| | Bis(diphenylphosphino)ferrocene] | | | | |
| | dichloropalladium(II), complex | | | | |
| | with dichloromethane | | | | |
| | (Pd(dppf)Cl₂) | | | | |
| | Bis(di-tert-butyl (4- | 0.05 | 2.55 | / | 3.60 |
| | dimethylaminophenyl) | | | | |
| | phosphine) | | | | |
| | dichloropalladium(II) | | | | |
| | (Pd(aMPHOS)Cl₂) | | | | |
| Chemicals | Potassium carbonate | / | 34.9 | / | / |
| | (K₂CO₃) | | | | |
| | Diatomite | / | 11 | / | / |
| | Silica gel | / | 44.5 | / | / |
| | NMP | / | 520 | / | / |
| | EtOAc | / | 1925 | / | / |
| | 2-MeTHF | / | 998 | / | / |
| | DMF | / | 234 | / | / |
| | Acetone | / | 350 | / | / |
| | Process water | / | 1370 | / | / |
| | NaCl | / | 50.5 | / | / |

| | | | Comments/Observations/Results |
|---|---|---|---|
| Operation | | | (X = 39.0 kg) |
| 1. | Prepare 10% NaCl aqueous solution with | | NaCl: 15.6 kg |
| | NaCl and process water | | Process water: 120 kg |
| 2. | Prepare 5% NaCl aqueous solution with | | NaCl: 34.9 kg |
| | NaCl and process water | | Process water: 620 kg |
| 3. | Prepare 20% K₂CO₃ aqueous solution with | | K₂CO₃: 34.9 kg |
| | K₂CO₃ and process water | | Process water: 132 kg |
| 4. | Clean R1 with NMP | | 199 kg |
| 5. | Charge NMP (5.09-6.22 X) into R1 | | 220 kg |
| | | | 5.64 X |
| 6. | Start agitator of R1 and set agitation | | |
| | speed at 75 rpm | | |
| 7. | Charge Compound 4 (assay corrected | | 38.8 kg |
| | 0.99-1.01 X) into R1 | | 0.99 X |
| 8. | Stir R1 for 0.5-1.0 h until the | | |
| | solid is dissolved | | |
| 9. | Charge Bis(pinacolato)diboron (0.53- | | 22.0 kg |
| | 0.59 X) into R1 | | 0.56 X |
| 10. | Charge Potassium acetate (0.51- | | 21.0 kg |
| | 0.57 X) into R1 | | 0.54 X |
| 11. | Charge NMP (2.25-2.75 X) into R1 | | 101 kg |
| | | | 2.59 X |
| 12. | Bubble with N₂ over 1 h | | |
| 13. | Charge [1,1,- | | 1.85 kg |
| | Bis(diphenylphosphino)ferrocene]di- | | 0.047 X |
| | chloropalladium(II), complex with | | |
| | dichloromethane (0.045-0.060 X) into R1 | | |
| 14. | Adjust R1 to 85-95° C. with inner temperature | | |
| 15. | Stir R1 at 85-95° C. with inner | | |
| | temperature for 3-17 h | | |
| 16. | Adjust R1 to 40-60° C. with inner temperature | | |
| 17. | IPC: Compound 4/Compound 3 | | N.D. |
| | (LC, % area) ≤1.0 | | |
| 18. | Adjust R1 to 20-30° C. with inner temperature | | |
| 19. | Charge EtOAc (7.20-8.80 X) into R1 | | 317 L |
| | | | 8.13 X |
| 20. | Charge process water (7.20-8.80 X) into R1 | | 314 kg |
| | | | 8.05 X |
| 21. | Adjust R1 to 20-30° C. with inner temperature | | |
| 22. | Stir R1 at 20-30° C. with inner | | |
| | temperature for 0.5-1.0 h | | |
| 23. | Allow R1 to stand for 0.5-1.0 h | | |
| 24. | Transfer R1 aqueous layer into T1 | | <Compound 3 |
| | | | aqueous layer, |
| | | | contain Pd> |
| 25. | Load the organic layer into V1 | | |
| 26. | Charge STEP 24 Compound 3 aqueous | | |
| | layer into R1 | | |
| 27. | Extract the aqueous layer with | | 198 L 5.08 X; |
| | EtOAc (4.50-5.50 X) twice | | 197 L 5.05 X |
| 28. | Transfer R1 aqueous layer into T1 | | <Compound 3 |
| | | | aqueous layer, |
| | | | contain Pd> |
| 29. | IPC: Residual Compound 3 (LC, % w/w): Report | | 0.03 |
| 30. | Load R1 organic layer into V1 | | Compound 3 NMP/ |
| | | | EtOAc solution |
| 31. | Spread two filter cloths into F1 | | |
| 32. | Charge diatomite (0.2-0.3 X) into F1 | | 11.0 kg |
| | | | 0.3 X |
| 33. | Press filter V1 material in portions | | |
| | via F1 and Ft1, transfer the filtrate | | |
| | to R1 at 20-30° C. with inner temperature | | |
| 34. | Rinse V1 with EtOAc (0.9-4.0 X) | | 80 kg |
| | | | 2.1 X |
| 35. | Press filter V1 rinse EtOAc via F1 | | |
| | and Ft1, transfer the filtrate to | | |
| | R1 at 20-30° C. with inner temperature | | |
| 36. | Load the filter wet cake into fiber | | <Compound 3 |
| | drum lined with LDPE bag | | diatomite, |
| | | | contain Pd> |
| 37. | Wash the organic phase in R1 with | | 199 kg 5.10 X; |
| | 5% NaCl aq. solution (4.50- | | 201 kg 5.15 X; |
| | 5.50 X) from STEP 2 three times | | 200 kg 5.13 X |
| 38. | Transfer R1 aqueous layer into T2 | | <Compound 3 aqueous |
| | | | layer, contain Pd> |
| 39. | Spread two filter cloths into F2 | | |
| 40. | Charge silica gel (0.9-1.1 X) into F2 | | 39.5 kg |
| | | | 1.01 X |
| 41. | Charge EtOAc (50-100 kg) to F2 to | | 50 kg |
| | moisten the silica gel | | |
| 42. | Press solvent of F2 out to used | | <Washing EtOAc> |
| | iron drums | | |
| 43. | Press filter R1 material in portions | | |
| | via F2, filter not to dryness after | | |
| | loading, transfer the filtrate to V1 | | |
| 44. | Rinse R1 with EtOAc (11-14 X) | | 540 kg |
| | | | 14 X |
| 45. | Press filter R1 rinse EtOAc via | | |
| | F2, filter not to dryness after | | |
| | loading, transfer the filtrate to the V1 | | |
| 46. | Load F2 wet cake into fiber drum | | <Compound 3 |
| | lined with double LDPE bag | | silica gel> |
| 47. | Spread two filter cloths into F2 | | |
| 48. | Charge silica gel of STEP 46 into F2 | | |
| 49. | Charge silica gel (2-20 kg) into F2 | | 5 kg |
| 50. | Press filter V1 material via F2, | | |
| | filter not to dryness after loading, | | |
| | transfer the filtrate to R1 | | |
| 51. | Rinse V1 with EtOAc (429-546 kg) | | 488 kg |
| | | | 12.5 X |
| 52. | Press filter V1 rinse EtOAc via F2, | | |
| | filter to dryness after loading, | | |
| | transfer the filtrate to R1 | | |
| 53. | Concentrate R1 mixture at <40° C. | | |
| | inner temperature under reduced | | |
| | pressure to 2-3 X | | |
| 54. | Charge 2-MeTHF (7.2-8.8 X) into R1 | | 309 L |
| | | | 7.9 X |
| 55. | Concentrate R1 mixture at <40° C. | | |
| | inner temperature under reduced | | |
| | pressure to 2-3 X | | |
| 56. | Charge 2-MeTHF (195-273 kg) into R1 | | 234 kg |
| | | | 6.0 X |
| 57. | Concentrate R1 mixture at <40° C. | | |
| | inner temperature under reduced | | |
| | pressure to 3-4 X | | |
| 58. | Charge 2-MeTHF (3.00-5.50 X) into R1 | | 198 kg |
| | | | 5.08 X |
| 59. | Adjust R1 to 20-30° C. with inner temperature | | |
| 60. | Load the R1 material into new steel-plastic | | <Compound 3 |
| | composite drum, record the weight in STEP 62 | | 2-MeTHF solution> |
| 61. | Charge 2-MeTHF (10-30 kg) into R1 | | 26 kg |
| 62. | Load the R1 material into new steel- | | <Compound 3 |
| | plastic composite drum | | 2-MeTHF solution> |
| | | | 378.2 kg |
| 63. | Clean R1 with 2-Me THF | | 194 kg |
| 64. | Charge all Compound 3 2-MeTHF solution | | |
| | from STEP 62 into R1 | | |
| 65. | Charge Compound 2 (0.40-0.50 | | 17.5 kg |
| | X) into R1 | | 0.45 X |
| 66. | Charge 2-MeTHF (50-120 kg) into R1 | | 80 kg |
| 67. | Charge 20% K₂CO₃ aq. solution | | 147 kg |
| | (3.4-4.2 X) from STEP 3 into R1 | | 3.8 X |
| 68. | Bubble with N₂ over 1 h at 20-30° | | |
| | C. with inner temperature | | |
| 69. | Charge Bis(di-tert-butyl (4-dimethylaminophenyl) | | 2.55 kg |
| | phosphine) dichloropalladium(II) (0.062- | | 0.065 X |
| | 0.078 X) into R1 | | |
| 70. | Adjust R1 to 60-70° C. with inner temperature | | |
| 71. | Stir R1 at 60-70° C. with inner | | |
| | temperature for 3-4 h | | |
| 72. | Adjust R1 to 40-50° C. with inner temperature | | |
| 73. | IPC: Compound 3/III-1 (LC, % area) ≤1.0 | | 0.0 |
| 74. | Adjust R1 to 20-30° C. with inner temperature | | |
| 75. | Allow R1 to stand for 0.5-1 h | | |
| 76. | Transfer R1 aqueous layer into the steel- | | <III-1 |
| | plastic composite drums | | aqueous layer> |
| 77. | Load R1 organic layer intoT3 | | <III-1 |
| | | | 2-MeTHF solution> |
| 78. | Charge III-1 aqueous layer of | | |
| | STEP 83 into R1 | | |
| 79. | Extract the aqueous layer in R1 | | 126 kg |
| | with EtOAc (2.5-4.0 X) | | 3.2 X |
| 80. | Transfer R1 aqueous layer into the | | <III-1 aqueous |
| | steel-plastic composite drums | | layer, contain Pd> |
| 81. | Charge STEP 84 III-1 2-MeTHF | | |
| | solution into R1 | | |
| 82. | Wash the organic layer in R1 with 10% | | 128 kg |
| | NaCl aq. solution (2.8-3.3 X) from STEP 1 | | 3.3 X |
| 83. | Transfer R1 aqueous layer into the | | <III-1 aqueous |
| | new steel-plastic composite drums | | layer, contain Pd> |
| 84. | IPC: Residual of III-1 (% w/w): Report | | 0.001 |
| 85. | Concentrate R1 mixture at <40° C. inner | | |
| | temperature under reduced pressure to 2-3 X | | |
| 86. | Charge DMF (1.7-2.1 X) into R1 | | 72 kg |
| | | | 1.8 X |
| 87. | Concentrate R1 mixture at <40° C. inner | | |
| | temperature under reduced pressure to 3-4 X | | |
| 88. | Charge DMF (3.6-4.5 X) into R1 | | 162 kg |
| | | | 4.2 X |
| 89. | Charge Acetone (4.4-5.5 X) into R1 | | 194 kg |
| | | | 5.0 X |
| 90. | Charge process water (4-8 X) into R1 in | | 184 kg |
| | portions at 20-30° C. with inner | | 5 X |
| | temperature over 4 h | | |
| 91. | Stir R1 at 20-30° C. with inner | | |
| | temperature for 2-3 h | | |
| 92. | IPC: Residual of III-1 in filtration | | 0.7 |
| | (LC, %, w/w): Report | | |
| 93. | Spread centrifuge bag in M1 | | |
| 94. | Isolated the wet cake by centrifuge and | | 156 kg |
| | rinse the wet cake with acetone (1-4 X) | | 4 X |
| 95. | Load solid into fiber drum lined | | <III-1 |
| | with double PE bags | | wet cake> |
| | | | 46.25 kg |
| 96. | IPC: Purity of III-1 in wet | | 96.3 |
| | cake (% area): Report | | |
| 97. | Load the filtrate into the new | | <III-1 acetone/ |
| | steel-plastic drum | | water mother liquor> |
| 98. | IPC: Residual of III-1 in filtration | | 0.5 |
| | (LC, %, w/w): Report | | |
| 99. | Put the STEP 115 III-1 wet cake into | | |
| | drying bag, then put the bag into D1 | | |
| 100. | Adjust the pressure of D1 to ≤−0.085 MPa | | |
| 101. | Set the jacket temperature of the | | |
| | dryer to 50-60° C. | | |
| 102. | Dry D1 under reduced pressure at | | |
| | 50-60° C. for 12-18 h | | |
| 103. | IPC: | | |
| | Test item | Expected value | Result |
| | Water Content (%, w/w) | Report | 0.3 |
| | Residual of 2-MeTHF (GC, ppm) | Report | <998 |
| | Residual of DMF (GC, ppm) | Report | 5397 |
| | Residual of acetone (GC, ppm) | Report | 84332 |
| | Residual of NMP (GC, ppm) | Report | <2465 |
| | Residual of EtOAc (GC, ppm) | Report | <992 |
| 104. | Load the dryer into empty fiber drum | | <III-1> |
| | lined with double LDPE bags | | 34.40 kg |
| 105. | IPC: | | |
| | Test item | Expected value | Result |
| | Purity of III-1 (LC, % area) | Report | 95.4 |
| | Assay of III-1 (LC, %, w/w) | Report | 85.2 |
| | Trace Metals-Pd (ICP-OES, ppm) | Report | 430 |

### Purification of Compound III-1

### Results:

| Starting | Product | | | |
|---|---|---|---|---|
| Material | (crude amount) | Purity | Assay | Yield |
| 34.1 kg | 27.80 kg | 99.2% | 89.6% | 86% |

### Material Dispensing Summary Table

| | | Actual Amounts | | | |
|---|---|---|---|---|---|
| | | | | Assay for | |
| Materials | | Equiv. | Mass | reactants | Moles |
| Class | Name | eq. | kg | w/w % | mol. |
| Reactants | III-1 | 1.00 | 34.1 | 85.2% | 55.0 |
| Chemicals | DMF | / | 210 | / | / |
| | DCM | / | 1636 | / | / |
| | MeOH | / | 236 | / | / |
| | Silica thiol | / | 34.3 | / | / |
| | Diatomite | / | 9.1 | / | / |
| | Acetone | / | 564 | / | / |
| | Process water | / | 296 | / | / |

| | | | Comments/Observations/Results |
|---|---|---|---|
| Operation | | | (X = 34.0 kg) |
| 1. | Charge DCM (8.5-12.8 X) into R1 | | 364 kg |
| | | | 10.7 X |
| 2. | Charge III-1 into R1 (0.99-1.01 X) | | 34.1 kg |
| | | | 1.00 X |
| 3. | Charge DCM (9.2-13.7 X) into R1 | | 410 kg |
| | | | 12.1 X |
| 4. | Charge MeOH (2.5-3.8 X) into R1 | | 110 kg |
| | | | 3.2 X |
| 5. | Charge DMF (0.76-1.14 X) into R1 | | 36 kg |
| | | | 1.06 X |
| 6. | Adjust R1 to 20-30° C. with inner temperature | | |
| 7. | Stir R1 at 20-30° C. with inner temperature | | |
| | for 0.3-1.0 h | | |
| 8. | Concentrate R1 mixture at <35° C. inner | | |
| | temperature under reduced pressure to 68-170 L | | |
| 9. | Charge DMF (1-6 X) into R1 | | 68 kg |
| | | | 2 X |
| 10. | Concentrate R1 mixture at <40° C. inner | | |
| | temperature under reduced pressure to 3-6 X | | |
| 11. | Charge DMF (1-6 X) into R1 | | 80 kg |
| | | | 2 X |
| 12. | Charge MeOH (2.5-3.8 X) into R1 | | 110 kg |
| | | | 3.2 X |
| 13. | Charge DCM (18-26 X) into R1 | | 754 kg |
| | | | 22.2 X |
| 14. | Adjust R1 to 20-30° C. with inner temperature | | |
| 15. | Stir R1 at 20-30° C. with inner temperature | | |
| | for 0.1-1.0 h till clear | | |
| 16. | Charge silica thiol (0.3-0.9 X) into R1 | | 17.1 kg |
| | | | 0.5 X |
| 17. | Stir R1 at 20-30° C. with inner | | |
| | temperature for 15-20 h | | |
| 18. | Charge diatomite (0.05-0.30 X) into F1 | | 2.0 kg |
| | | | 0.06 X |
| 19. | Transfer mixture from R1 into T1 via | | <III-1 organic |
| | filter F1 and cartridge filter | | layer> 76 kg |
| 20. | Load cartridge filter wet cake into | | <III-1 diatomite/ |
| | fiber drum lined with double LDPE bag | | silica thiol> |
| 21. | Transfer mixture from R1 into T1 via | | <III-1 |
| | filter F1 and cartridge filter | | organic layer> |
| | and bag filter | | |
| 22. | Load F1 and bag filter wet cake | | <III-1 diatomite/ |
| | into STEP 20 bag | | silica thiol> |
| 23. | Spread two filter clothes into F1 | | |
| 24. | Charge diatomite (0.05-0.30 X) into F1 | | 2.1 kg |
| | | | 0.06 X |
| 25. | Charge STEP 22 III-1 diatomite/ | | 7.55 kg |
| | silica thiol into F1 | | |
| 26. | Transfer mixture from R1 into T1 | | <III-1 organic |
| | via filter F1 and cartridge filter | | layer> 1010 kg |
| 27. | Charge all III-1 organic layer | | |
| | from STEP 26 into R1 | | |
| 28. | Charge silica thiol (0.3-0.9 X) into R1 | | 17.5 kg |
| | | | 0.5 X |
| 29. | Adjust R1 to 20-30° C. with inner temperature | | |
| 30. | Stir R1 at 20-30° C. with inner | | |
| | temperature for 15-20 h | | |
| 31. | Load F1 wet cake into fiber drum | | <III-1 diatomite/ |
| | lined with double LDPE bag | | silica thiol, |
| | | | contain Pd> |
| 32. | Spread two filter clothes into F2 | | |
| 33. | Charge diatomite (1.7-10.2 kg) into F2 | | 5.0 kg |
| 34. | Transfer mixture from R1 into T2 via | | <III-1 organic |
| | filter F2 and cartridge filter | | layer> 1056 kg |
| 35. | Charge MeOH (0.4-0.8 X) into R1 | | 16 kg |
| | | | 0.5 X |
| 36. | Charge DCM (1.6-3.2 X) into R1 | | 108 kg |
| | | | 3.2 X |
| 37. | Transfer mixture from R1 into T2/T1 | | <III-1 organic |
| | via filter F2 and cartridge filter | | layer> 124 kg |
| 38. | Clean R2 with Acetone | | 156 kg |
| 39. | Charge the half of III-1 organic | | |
| | layer into R2 | | |
| 40. | Start agitator of R2 and set | | |
| | agitation speed at 85 rpm | | |
| 41. | Concentrate R2 mixture at <35° C. | | |
| | inner temperature under reduced | | |
| | pressure to 200-300 L | | |
| 42. | Charge the Residual of III-1 | | |
| | organic layer into R2 | | |
| 43. | Concentrate R2 mixture at <35° C. | | |
| | inner temperature under reduced | | |
| | pressure to 68-290 L | | |
| 44. | Charge DMF (12-25 kg) into R2 | | 12 kg |
| | via cartridge filter | | |
| 45. | Concentrate R2 mixture at <40° C. | | |
| | inner temperature under reduced | | |
| | pressure to 3-6 X | | |
| 46. | Charge DMF (12-25 kg) into R2 | | 14 kg |
| | via cartridge filter | | |
| 47. | Charge Acetone (4-6 X) into R2 | | 168 kg |
| | via cartridge filter | | 5 X |
| 48. | Adjust R2 to 20-30° C. with | | |
| | inner temperature | | |
| 49. | Charge process water (0.5-1.7 X) into | | 40 kg |
| | R2 with inner temperature 20-30° C. | | 1.2 X |
| | via cartridge filter NLT 2 h | | |
| 50. | Stir R2 at 20-30° C. with inner | | |
| | temperature for 1-3 h | | |
| 51. | Charge process water (2.8-4.3 X) into | | 106 kg |
| | R2 with inner temperature 20-30° C. | | 3.1 X |
| | via cartridge filter NLT 2 h | | |
| 52. | Stir R2 at 20-30° C. with inner | | |
| | temperature for 1-5 h | | |
| 53. | Spread centrifuge bag on M1 | | |
| 54. | Isolate the wet cake by centrifuge | | 120 kg 3.5 X; |
| | and rinse the wet cake with acetone | | 150 kg 4.4 X; |
| | (1-5 X), process water (2-6 X), | | 120 kg 3.5 X |
| | acetone (1-5 X), respectively | | |
| 55. | Load wet cake into fiber drum | | <III-1 wet |
| | lined with double PE bag | | cake> 35.25 kg |
| 56. | IPC: Purity of III-1 in wet cake | | 99.1 |
| | (LC, % area): Report | | |
| 57. | Load the mother liquor into plastic- | | <III-1 DMF/acetone |
| | steel composite drums | | mother liquor> |
| 58. | Put the STEP 55 III-1 wet cake into | | |
| | drying bag, then put the bag into D1 | | |
| 59. | Adjust the pressure of D1 | | |
| | to ≤−0.085 MPa | | |
| 60. | Set the jacket temperature of | | |
| | the dryer to 50-60° C. | | |
| 61. | Dry D1 under reduced pressure | | |
| | at 50-60° C. for 12-18 h | | |
| 62. | IPC: | | |
| | Test item | Expected value | Result |
| | Water Content (%, w/w) | Report | 0.1 |
| | Residual of 2-MeTHF (GC, ppm) | Report | <998 |
| | Residual of DMF (GC, ppm) | Report | <4420 |
| | Residual of acetone (GC, ppm) | Report | 88099 |
| | Residual of NMP (GC, ppm) | Report | <2465 |
| | Residual of MeOH (GC, ppm) | Report | <997 |
| | Residual of EtOAc (GC, ppm) | Report | <992 |
| | Residual of DCM (GC, ppm) | Report | <1007 |

| | | |
|---|---|---|
| 63. | Load the dryer into empty fiber drum lined | |
| | with double LDPE bags | |
| 64. | Sieve the product (10 mesh) | |
| 65. | Load the sieved product into empty fiber | <III-1> |
| | drum lined with double LDPE bags | 27.85 kg |
| 66. | Release testing and sample retention | |
| 67. | Take the material from STEP 65 drum, divide | <III-1> |
| | the material equally into 2 fiber drum lined | 27.80 kg |
| | with double LDPE bags | |

### Example 4. Preparation of Free Base Crystal Forms

### 4.1 Polymorphs of I-1 and III-1

This polymorph screening program of compound I-1 free form was performed with two batches of the pure enantiomer. Both batches are crystalline and is assigned as Form A. The polymorphic behaviors of the enantiomer were investigated by equilibration at 25° C. and 50° C., equilibration under a temperature cycle, crystallization from hot saturated solutions by slow cooling, slow evaporation, precipitation by addition of anti-solvent, and DSC heating-cooling cycle experiments. Relative stability of identified polymorphs was investigated by competitive slurry experiments.

During this study, in total 8 crystalline patterns were identified, including 3 enantiopure polymorphs, named I-1 Form A, Form B, and Form C: 2 racemate forms, named III-1 Form E and Form F: 3 partially racemized mixtures, namely, III-1 Form A, Form C, and Form D; and I pattern with unknown identity, named III-1 Form B. (Table 4.1). The 3 enantiopure polymorphs are all non-solvated/anhydrous forms. There is no sufficient material to confirm the identity of Form B. In addition, amorphous form was obtained by equilibration in THF at 50° C. and slow evaporation in acetone, ACN, THF and 1,4-dioxane.

I-1 Form A is an enantiopure P2 polymorph. It is an anhydrate. The ee % is 100% by SFC, and of high crystallinity. DSC shows a melting peak at Tₒₙₛₑₜ of 264.8° C. with an enthalpy of about 94 J/g. TGA shows about 0.4% weight loss at about 260° C. No residual solvent was detected by ¹H-NMR. KF shows this sample contains about 0.6% water by weight. I-1 Form A could be obtained from multiple solvent systems.

I-1 Form B is also an enantiopure P2 polymorph. It is an anhydrate. I-1 Form B was obtained by equilibration in 1,4-dioxane at 50° C. for 10 days. The ee % of I-1 Form B is 100% by SFC, and of medium crystallinity. DSC shows multiple thermal events. TGA shows about 1.0% weight loss at about 245° C. ¹H-NMR shows about 0.3% 1,4-dioxane by weight (0.02 equivalent by molar ratio).

I-1 Form C is an enantiopure P2 polymorph. It is an anhydrate. I-1 Form C was obtained from THF/heptane (2:3, v/v) and THF/MTBE (1:4, v/v) by addition of anti-solvent experiments. The ee % of I-1 Form C is 100% by SFC, and of high crystallinity. DSC shows multiple thermal events. TGA shows about 0.9% weight loss at about 250° C.

III-1 Form A is a partially racemized mixture due to racemization during equilibration. It was obtained by equilibration in MeOH, EtOH/water (50:50, v/v) and ACN/water (80): 20, v/v) at 50° C. for 10 days. The ee % of III-1 Form A is 76.9%, and of medium crystallinity. DSC shows an endothermic peak at Tₒₙₛₑₜ of 285.7° C. with an enthalpy of about 109 J/g. TGA shows about 1.1% weight loss at about 267° C. No residual solvent was detected by ¹H-NMR.

III-1 Form B was only obtained by equilibration in THF/water (85:15, v/v) at 25° C. for 2 weeks. III-1 Form B is of low crystallinity. DSC shows multiple thermal events. No sufficient material for SFC test to confirm whether III-1 Form B is a racemic mixture or enantiopure polymorph of I-1. III-1 Form B was not reproduced.

III-1 Form C is a partially racemized mixture. It was obtained by equilibration in acetone/water (60:40, v/v) at 50° C. for 10 days. The ee % of III-1 Form C is 56.9%, and of medium crystallinity. DSC shows multiple thermal events. TGA shows about 6.7% weight loss at about 187° C. ¹H-NMR shows about 3.8% acetone by weight (0.4 equivalent by molar ratio). KF shows this batch contains about 1.0% water by weight.

III-1 Form D is a partially racemized mixture. It was obtained from MeOH by slow evaporation. The ee % of III-1 Form D is 68.6%, and of medium crystallinity. DSC shows an endothermic peak at Tₒₙₛₑₜ of 279.4° C. with an enthalpy of about 106 J/g. TGA shows about 3.4% weight loss at about 251° C. No residual solvent was detected by ¹H-NMR. KF shows this sample contains about 2.0% water by weight.

III-1 Form E is a racemic mixture. It was obtained from MeOH/MTBE (1:4, v/v) by addition of anti-solvent. The ee % of III-1 Form E is 2.7%, and of medium crystallinity.

III-1 Form F is a racemic mixture. The ee % of III-1 Form F is-0.3%, and of high crystallinity. DSC shows multiple thermal events.

### 4.2 Polymorphs of II-1

The ee % of II-1 is-100% by SFC. This batch is amorphous form. It was used for equilibration experiments attempting to crystallize II-1 enantiomer polymorphs and provide reference for I-1 enantiomer polymorphs determination. Three polymorphs of enantiomer II-1 were identified, including II-1 Form A, Form B and Form C.

II-1 Form A was obtained by equilibration in methanol, EtOH/water (50:50, v/v), ACN/water (80:20, v/v) at 50° C. for 6 days. The ee % of II-1 Form A is-99.66% by SFC, and of medium crystallinity.

II-1 Form B was obtained by equilibration in 1,4-dioxane at 50° C. for 6 days. The ee % of II-1 Form B is-99.48% by SFC, and of medium crystallinity.

II-1 Form C was obtained by equilibration in THF/water (85:15, v/v) at 25° C. for 6 days. The ee % of II-1 Form C is-99.34% by SFC, and of medium crystallinity.

### 4.3 Investigation of Factors Impacting Racemization

It is found that racemization easily occurs for I-1 in different solvents, resulting in the difficulty to understand polymorphic behaviors. Therefore, the factors impacting racemization were investigated including pH, temperature, molecular sieve and experimental duration. It was found that basic pH, high temperature and longer time and exposure to light can speed up racemization. It is worth noted that racemization can be accelerated when solvents are pretreated by molecular sieve, since the pH value is shifted to basic after pretreatment.

During DSC test for I-1 Form A, different heating rates result in different thermal events. To investigate whether racemization occurs during heating, I-1 Form A was heated to different temperatures then obtained solids were tested by SFC. Based on the results, racemization indeed occurred during heating at certain high temperatures. The results indicate that melting onset in DSC may not be used to distinguish different enantiopure P2 polymorphs.

### 4.4 Relationships of I-1 Polymorphs

Relative stability of the I-1 anhydrates (Form A and Form C) was investigated by competitive equilibration experiments at 25° C. Form A is the only product in different solvents at 25° C., suggesting that Form A is the more stable anhydrate. However, no more relationship or polymorph landscape can be explored due to the racemization.

### 4.5 Evaluation of I-1 Form A

Feasibility of formulation processes was evaluated for I-1 Form A by compression experiments, grinding and granulation simulation experiments. I-1 Form A shows good tolerance to these processes with no form change. After compression, the peaks of I-1 Form A became slightly broader. After dry grinding, peak intensity of Form A became slightly decreased.

The desired I-1 easily racemized, resulting in difficulty to proceed polymorph screening study. Process control is recommended to minimize racemization in downstream manufacture, formulation processes and storage. Also, salt screening is recommended to identify a suitable salt form that may solve the racemization issue and is suitable for downstream development.

TABLE 4.1
Summary of identified patterns

| Compound | Polymorph | Preparation | Comments |
|---|---|---|---|
| I-1 | Form A | 1) Equilibration experiments | Batch #1: |
| | (Anhydrate) | at 25° C. in majority of | XRPD: high crystallinity |
| | | solvents | DSC: RT-300° C.: 10° C./min: |
| | | 2) Equilibration experiments | Melting onset 264.8° C. (94 J/g) |
| | | at 50° C. in majority of | DSC: RT-300° C.: 2° C./min: |
| | | solvents | Endothermic: 261.4° C. (68 J/g), |
| | | 3) Anti-solvent in | 274.7° C. (48 J/g); exothermic: |
| | | acetone/heptane (1:2, | 265.8° C. (10 J/g) |
| | | v/v), acetone/MTBE (1:4, | TGA: 0.4%@260° C. ¹H- |
| | | v/v); | NMR(DMSO-d₆): no solvent residue. |
| | | 4) Temperature cycling | KF: 0.6% water by weight |
| | | experiments in majority | PLM: columnar crystals(16-25 μm) |
| | | of solvents | As received: |
| | | | ee % (SFC): 100.0%* |
| | | 5) Competitive equilibration | ee % (SFC): 97.7%#; |
| | | experiments from | After heating to 260° C. |
| | | THF/heptane(2:3, v/v), | at 2° C./min by |
| | | Ethyl acetate/ | DSC: |
| | | heptane(1:1, v/v), | XRPD: low crystallinity |
| | | 1,4-dioxane, | ee % (SFC): 88.1%* |
| | | THF/MTBE(1:4, v/v), | After heating to 270° C. |
| | | THF/ACN(2:1, v/v). | at 2° C./min by |
| | | | DSC: |
| | | | ee % (SFC): 10.9%* |
| | | | After heating to 300° C. |
| | | | at 2° C./min by |
| | | | DSC: |
| | | | DSC: Endothermic: 261.4° C. (68 J/g), |
| | | | 274.7° C. (48 J/g); exothermic: |
| | | | 265.8° C. (10 J/g) |
| | | | XRPD: amorphous form |
| | | | ee % (SFC): 2.2%* |
| | | | Batch #2: |
| | | | XRPD: high crystallinity |
| | | | DSC: 2° C./min: Endothermic: |
| | | | 251.0° C. (82 J/g), |
| | | | 267.1° C. (24 J/g) |
| III-1 | Form A | Equilibration in MeOH, | Equilibration in EtOH/water: |
| | | EtOH/water(50:50, v/v), | XRPD: medium crystallinity |
| | | ACN/water(80:20, v/v) at | DSC: Melting onset 285.7° |
| | | 50° C. for 10 days | C. (109 J/g) |
| | | | TGA: 1.1%@267° C. |
| | | | ¹H-NMR (DMSO-d₆): no solvent |
| | | | residue. |
| | | | ee % (SFC): 76.9%*, |
| | | | ee % (SFC): 75.0%#; |
| | | | Equilibration in MeOH: |
| | | | DSC: thermal event: |
| | | | melting onset |
| | | | 285.6° C. (117 J/g) |
| | | | TGA: weight loss: 1.2%@260° C. |
| | | | ¹H-NMR (DMSO-d₆): 0.1% MeOH |
| | | | by weight (0.02 equiv.) |
| | | | ee % (SFC): 48.4%* |
| | | | Equilibration in ACN/water: |
| | | | DSC: Endothermic: |
| | | | 264.7° C. (2 J/g), |
| | | | 284.5° C. (113 J/g); exothermic: |
| | | | 268.4° C. (2 J/g) |
| | | | ee % (SFC): 50.7%*. |
| I-1 | Form B | Equilibration in | XRPD: medium crystallinity |
| | (Anhydrate) | 1,4-dioxane at | DSC: Endothermic: 250.3° C. (52 J/g), |
| | | 50° C. for 10 days | 262.6° C. (26 J/g), 277.9° C. (26 J/g); |
| | | | exothermic: 257.2° C. (3 J/g) |
| | | | TGA: 1.0%@245° C. |
| | | | ¹H-NMR (DMSO-d₆): 0.3% 1,4- |
| | | | dioxane by weight (0.02 equiv.) |
| | | | ee % (SFC): 100.0%* |
| N/A | Form B | Equilibration in | XRPD: low crystallinity |
| | | THF/water | DSC: Endothermic: 70.4° C. (2 J/g), |
| | | (85:15, v/v) at 25° | 97.9° C. (10 J/g), 185.2° C. (19 J/g), |
| | | C. for 2 weeks | 231.2° C. (2 J/g), 275.3° C. (62 J/g), |
| | | | 284.7° C. (6 J/g) |
| | | | No sufficient material for further |
| | | | characterization. |
| III-1 | Form C | Equilibration in | XRPD: medium crystallinity |
| | | acetone/water | DSC: Endothermic: 157.4° C. (41 J/g), |
| | | (60:40, v/v) at 50° | 264.6° C. (10 J/g), 280.8° C. (92 J/g); |
| | | C. for 10 days | exothermic: 181.7° C. (32 J/g), |
| | | | 268.7° C. (8 J/g) |
| | | | TGA: 1.4% @ 83° C. 5.3%@188° C. |
| | | | ¹H-NMR (DMSO-d₆): 3.7% acetone |
| | | | by weight (0.4 equiv.) |
| | | | KF: 1.0% water by weight |
| | | | (0.3 equiv.) |
| | | | ee % (SFC): 56.9%* |
| III-1 | Form D | Slow evaporation | XRPD: medium crystallinity |
| | | from MeOH | DSC: melting onset 279.4° |
| | | | C. (106 J/g) |
| | | | TGA: 3.4%@251° C. |
| | | | ¹H-NMR(DMSO-d₆): no solvent |
| | | | residue |
| | | | KF: 2.0% water by weight |
| | | | (0.6 equiv.) |
| | | | ee % (SFC): 68.6%* |
| I-1 | Form C | Anti-solvent in | THF/MTBE: |
| | (Anhydrate) | THF/heptane (2:3, v/v), | XRPD: high crystallinity |
| | | THF/MTBE (1:4, v/v) | DSC: Endothermic: 257.9° C. (54 |
| | | | J/g), 276.2° C. (44 J/g); |
| | | | exothermic: 267.3° C. (3 J/g) |
| | | | TGA: 0.9%@250° C. |
| | | | ¹H-NMR (DMSO-d₆): 0.08% MTBE |
| | | | by weight |
| | | | PLM: |
| | | | ee % (SFC): 100.0%* |
| III-1 | Form E | Anti-solvent in MeOH/MTBE | XRPD: medium crystallinity |
| | | (1:4, v/v) | ee % (SFC): 2.7%* |
| III-1 | Form F | crystallization from | XRPD: high crystallinity |
| | | DMF/Acetone water | DSC: Endothermic: 147.5° C. (127 J/g), |
| | | | 266.9° C. (3 J/g), 280.9° C. (78 J/g); |
| | | | exothermic: 218.8° C. (70 J/g) |
| | | | ee % (SFC): −0.3%* |

*SFC test was immediately performed after preparation.
#SFC test was performed after preparation for 12 h.

### 4.6 Starting Material Used for Polymorph Screening

| Identity | I-1 | I-1 | II-1 | III-1 |
|---|---|---|---|---|
| Polymorph | Form A | Form A + minor | Amorphous | Form F |
| | | peaks at 2theta | form | (Acetone solvate |
| | | 23.4°, 14.4°, 20.7° | | form) |
| | | and 27.3° | | |
| | | (characteristic | | |
| | | peaks of II-1) | | |

### Properties of the Starting Material

| | | Result | Result | Result | Result |
|---|---|---|---|---|---|
| Parameter | Method | I-1 Batch#1 | I-1 Batch#2 | II-1 | III-1 |
| Chiral purity | SFC | 100% | 95.5%\hat{ } | −100%\hat{ } | −0.3%\hat{ } |
| Chemical purity | HPLC | 99.2% | // | // | // |
| | (220 nm) | | | | |
| X-ray diffraction | 3-40° (2 | Form A, high | Form A + | Amorphous | Form F |
| | theta) | crystallinity | minor peaks | form | |
| | | | at 2theta | | |
| | | | 13.3°, 14.4°, | | |
| | | | 20.6° and 27.3° | | |
| | | | (characteristic | | |
| | | | peaks of II-1) | | |
| DSC melting | DSC | RT-300° C.: | 2° C./min: | 2° C./min: | 2° C./min: |
| onset and enthalpy | | 10° C./min: | endothermic: | no obvious | endothermic: |
| | | Melting onset | 251.0° C. | melting | 147.5° C. |
| | | 264.8° C. (94 J/g) | (82 J/g), | | (127 J/g), |
| | | RT-300° C.: | 267.1° C. | | 266.9° C. |
| | | 2° C./min: | (24 J/g) | | (3 J/g), |
| | | Endothermic: | | | 280.9° C. |
| | | 261.4° C. (68 J/g), | | | (78 J/g); |
| | | 274.7° C. (48 J/g); | | | exothermic: |
| | | exothermic: | | | 218.8° C. |
| | | 265.8° C. (10 J/g) | | | (70 J/g) |
| Thermogravimetry | TGA, | 0.4%@260° C. | // | // | // |
| | 10° C./min | | | | |
| Residual solvent | ¹H-NMR | No residual | // | // | // |
| | (DMSO-d₆) | solvent | | | |
| Morphology | PLM | Columnar | // | // | // |
| | | crystals(16- | | | |
| | | 25 μm) | | | |
| FT-IR | ATR | | // | // | // |
| KF | Coulometric | 0.6% water by | // | // | // |
| | | weight (0.16 | | | |
| | | equiv.) | | | |

Explanation:
“//” Not carried out.
\hat{ }SFC test was immediately performed after preparation.

### 4.7 Test Conditions

### Approximate Solubility at 25° C.

About 5 mg of I-1 Form A was weighed into a 2 mL glass vial and aliquots of 20 μL of each solvent (pretreated by molecular sieves) was added to determine solubility at 25° C. Max. volume of each solvent added was 1 mL. Approximate solubility was determined by visual observation.

| Solvent | Solubility (mg/mL) |
|---|---|
| Water | <5 |
| Methanol | 15-17 |
| Ethanol | 6-7 |
| Isopropanol | <5 |
| Acetone | 43-52 |
| Methyl ethyl ketone | 26-28 |
| Ethyl acetate | 6-7 |
| Isopropyl acetate | <5 |
| Acetonitrile | 10-11 |
| Dichloromethane | <5 |
| Heptane | <5 |
| t-Butyl methyl ether | <5 |
| Tetrahydrofuran | 52-65 |
| 1,4-Dioxane | 29-33 |
| Toluene | <5 |
| Benzyl alcohol | 16-18 |
| 2-MeTHF | 5-6 |
| Anisole | <5 |
| Chloroform | <5 |
| Nitromethane (NM) | 7-8 |

Equilibration with Solvents at 25° C. For 2 Weeks

| Solvent | XRPD | Comments |
|---|---|---|
| Water | I-1 + I-2 mixture | // |
| Methanol | I-1 + I-2 mixture | Endothermic: 264.6° C. (5 J/g), |
| | | 282.1° C. (102 J/g); |
| | | Exothermic: 177.8° C. (3 J/g), 268.1° C. |
| | | (4 J/g) |
| | | Weight loss: 2.0%@230° C. |
| | | ¹H-NMR(DMSO-d₆): 0.28% MeOH |
| | | by weight(0.053 equiv.) |
| Ethanol | I-1 Form A | // |
| Acetone | I-1 + I-2 mixture | // |
| Methyl ethyl ketone | I-1 Form A | // |
| Ethyl acetate | I-1 Form A | // |
| Isopropyl acetate | I-1 Form A | // |
| Acetonitrile | I-1 Form A | // |
| t-Butyl methyl ether | I-1 Form A | // |
| Dichloromethane | I-1 Form A | // |
| Tetrahydrofuran | I-1 Form A | // |
| 1,4-Dioxane | I-1 Form A | // |
| Benzyl alcohol | I-1 Form A | // |
| 2-MeTHF | I-1 Form A | // |
| MeOH/water(55:45, | I-1 Form A | // |
| v/v) a.w. 0.7 | | |
| EtOH/water(50:50, v/v) | I-1 Form A + one | // |
| a.w. 0.9 | small peak at 2 theta | |
| | 3.4° | |
| Acetone/water(60:40, | I-1 + I-2 mixture | // |
| v/v) a.w. 0.8 | | |
| ACN/water(90:10, v/v) | I-1 + I-2 mixture | Endothermic: 265.1° C. (24 J/g), |
| a.w. 0.9 | | 281.0° C. (88 J/g); |
| | | Exothermic: 180.4° C. (3 J/g), 269.9° C. |
| | | (9 J/g) |
| | | Weight loss: 1.2%@240° C. |
| | | ¹H-NMR(DMSO-d₆): 0.38% ACN by |
| | | weight (0.057 equiv.) |
| | | ee % (SFC): 89.6%* |
| THF/water(85:15, v/v) | III-1 Form B | Target concentration: 510 mg/mL |
| a.w. 0.9 | | Endothermic: 70.4° C. (2 J/g), 97.9° C. |
| | | (10 J/g), 185.2° C. (19 J/g), 231.2° C. |
| | | (2 J/g), 275.3° C. (62 J/g), 284.7° C. |
| | | (6 J/g) |
| | | No sufficient material for |
| | | characterization |

*SFC test was immediately performed after preparation.
The water activity of a binary solvent system is calculated based on UNIFAC method UNIQUAC Functional-group Activity Coefficients).

About 50 mg of I-1 Form A was weighed into a 2 mL glass vial and equilibrated in suitable amount of solvents (pretreated by molecular sieves) at 25° C. for 2 weeks with a stirring plate and with protection from light. Obtained suspensions were filtered with 0.45 μm nylon membrane and the solid parts (wet cake) were investigated by XRPD. When differences were observed, additional investigations were performed (e.g. DSC, TGA, ¹H-NMR, SFC).

Equilibration with Solvents at 50° C. For 10 Days

| Solvent | XRPD | Comments |
|---|---|---|
| Water | I-1 Form A | // |
| Methanol | III-1 Form A | Thermal event: melting onset 285.6° C. |
| | | (117 J/g) |
| | | Weight loss: 1.2%@260° C. |
| | | ¹H-NMR (DMSO-d₆): 0.1% MeOH by weight |
| | | (0.02 equiv.) |
| | | ee % (SFC): 48.4%* |
| Ethanol | I-1 Form A | // |
| Acetone | I-1 Form A | // |
| Methyl ethyl ketone | I-1 Form A | // |
| Ethyl acetate | I-1 Form A | // |
| Isopropyl acetate | I-1 Form A | // |
| Acetonitrile | I-1 Form A | // |
| t-Butyl methyl ether | I-1 Form A | // |
| Anisole | I-1 Form A | // |
| Tetrahydrofuran | Practically | // |
| | amorphous form | |
| 1,4-Dioxane | I-1 Form B | Endothermic: 250.3° C. (52 J/g), 262.6° C. |
| | | (26 J/g), 277.9° C. (26 J/g); |
| | | Exothermic: 257.2° C. (3 J/g) |
| | | Weight loss: 1.0%@245° C. |
| | | ¹H-NMR (DMSO-d₆): 0.3% 1,4-dioxane by |
| | | weight (0.02 equiv.) |
| | | ee % (SFC): 100.0%* |
| Benzyl alcohol | I-1 Form A + one | // |
| | small peak at 2 | |
| | theta 3.5° | |
| 2-MeTHF | I-1 Form A + I-1 | Thermal event: melting onset 264.4° C.(85 J/g) |
| | Form B | Weight loss: 2.1%@255° C. |
| | | ¹H-NMR (DMSO-d₆): no residual solvent |
| MeOH/water(55:45, | I-1 Form A | // |
| v/v) a.w. 0.7 | | |
| EtOH/water(50:50, | III-1 Form A | Thermal event: melting onset 285.9° C. |
| v/v) a.w. 0.9 | | (118 J/g) |
| | | Weight loss: 1.1%@267° C. |
| | | ¹H-NMR (DMSO-d₆): no residual solvent |
| | | ee % (SFC): 76.9%*, |
| | | ee % (SFC): 75.0%#; |
| Acetone/water(60:40, | III-1 Form C | Endothermic: 157.4° C. (41 J/g), 264.6° C. |
| v/v) a.w. 0.8 | | (10 J/g), 280.8° C. (92 J/g); |
| | | Exothermic: 181.7° C. (32 J/g), 268.7° C. (8 J/g) |
| | | Weight loss: 1.4% @ 83° C., 5.3%@187° C. |
| | | ¹H-NMR (DMSO-d₆): 3.8% acetone by weight |
| | | (0.4 equiv.) |
| | | KF: 1.0% water by weight (0.3 equiv.) |
| | | ee % (SFC): 56.9%* |
| ACN/water(80:20, | III-1 Form A | Endothermic: 264.7° C. (2 J/g), 284.5° C. |
| v/v) a.w. 0.9 | | (113 J/g); |
| | | Exothermic: 268.4° C. (2 J/g) |
| | | ee % (SFC): 50.7%* |
| THF/water(85:15, | I-1 Form A | // |
| v/v) a.w. 0.9 | | |

*SFC test was immediately performed after preparation.
#SFC test was performed after preparation for about 12 h.
The water activity of a binary solvent system is calculated based on UNIFAC method (UNIQUAC Functional-group Activity Coefficients).

About 50 mg of I-1 Form A was weighed into 2 mL glass vials and equilibrated in suitable amount of solvents (pretreated by molecular sieves) at 50° C. for 10 days with a stirring plate and with protection from light. Obtained suspensions were filtered with 0.45 μm nylon membrane and the solid parts (wet cake) were investigated by XRPD. When differences were observed, additional investigation was performed (e.g. DSC, TGA, ¹H-NMR, SFC).

### Precipitation by Addition of Anti-Solvent

| Solvents | Anti-solvent | XRPD | Comments |
|---|---|---|---|
| MeOH | MTBE(4V) | I-1 Form E | stirred at 25° C. for 23 days |
| | | | ee % (SFC): 2.7%* |
| MeOH | Toluene(4V) | // | Clear solution |
| Acetone | Heptane(2V) | I-1 Form A | No comments |
| Acetone | MTBE(4V) | I-1 Form A | No comments |
| THF | Heptane(1.5V) | I-1 Form C | No comments |
| THF | MTBE(4V) | I-1 Form C | Endothermic: 257.9° C. (54 |
| | | | J/g), 276.2° C.(44 J/g); |
| | | | exothermic: 267.3° C. (3 J/g) |
| | | | Weight loss: 0.9%@250° C. |
| | | | ¹H-NMR (DMSO-d₆): 0.08% |
| | | | MTBE by weight |
| | | | ee % (SFC): 100.0%* |

“//” Not carried out.
“V” The volume ratio of anti-solvent to good solvent
*SFC test was immediately performed after preparation.

I-1 Form A was dissolved in a good solvent. Obtained solution was filtered by 0.45 μm nylon filter to get a clear solution. Then anti-solvents were added into the clear solution slowly at 25° C. on a stirring plate with protection from light. The solvents used were pretreated by molecular sieve. Precipitates were collected by filtration. The solid part (wet cake) was investigated by XRPD. And clear solutions were placed at 25° C. with stirring for one week, when solids were observed, the solid part (wet cake) was collected and investigated by XRPD. When differences were observed, additional investigations were performed (e.g. DSC, TGA, ¹H-NMR, SFC).

### Crystallization at Room Temperature by Slow Evaporation

| Solvents | XRPD | Comments |
|---|---|---|
| MeOH | III-1D | Thermal event: melting onset 279.4° C. |
| | | (106 J/g) |
| | | Weight loss: 3.4%@251° C. |
| | | ¹H-NMR(DMSO-d₆): no residual |
| | | solvent |
| | | KF: 2.0% water by weight (0.6 equiv.) |
| | | ee % (SFC): 68.6%* |
| Acetone | Amorphous form | // |
| ACN | Amorphous form | // |
| THF | Amorphous form | // |
| 1,4-Dioxane | Amorphous form | // |

Explanation
“//”: No comments.
*SFC test was immediately performed after preparation.

Based on approximate solubility results, I-1 Form A was dissolved in suitable amount of solvents (pretreated by molecular sieves). Obtained solutions were filtered by 0.45 μm nylon filter. Obtained clear solutions were slowly evaporated upon protecting from light. Solid residues were examined for their polymorphic form. When differences were observed, additional investigations were performed (e.g. DSC, TGA, ¹H-NMR, SFC).

Crystallization from Hot Saturated Solutions by Slow Cooling

| Solvents | XRPD | Comments |
|---|---|---|
| MeOH | // | clear solution |
| Acetone | // | clear solution |
| MEK | // | clear solution |
| ACN | // | clear solution |
| THF | // | clear solution |
| 1,4-Dioxane | low crystallinity, | ee %(SFC): −0.2%* |
| | racemic mixture | |
| Benzyl alcohol | // | clear solution |

“//”: Not carried out.
*SFC test was immediately performed after preparation.

Approximate 50 mg of batch I-1 Form A were dissolved in the minimal amount of selected solvents (without pretreatment by molecular sieves) at 50° C. Obtained solutions were filtered by 0.45 μm nylon filter. Obtained clear solutions were cooled to 5° C. at 0.1° C./min and with protection from light. Precipitates were collected by filtration. The solid parts (wet cake) were investigated by XRPD. When differences were observed, additional investigations were performed (e.g. DSC, TGA, ¹H-NMR, SFC). When no precipitation was obtained or only a few solids were obtained, the solutions were placed at 5° C. for crystallization.

### Temperature Cycling Experiments

| Solvents | XRPD | Comments |
|---|---|---|
| Methanol | I-1 + I-2 | Endothermic: 107.4° C. (3 J/g), 155.7° C. (1 J/g), 263.9° C. |
| | mixture | (5 J/g), 282.5° C. (106 J/g); exothermic: 268.3° C. (5 J/g) |
| | | Weight loss: 1.1%@150° C. |
| | | ¹H-NMR (DMSO-d₆): no residual solvent |
| | | ee %(SFC): 89.4%* |
| Ethanol | I-1 Form A | Endothermic: 263.6° C. (9 J/g), 282.0° C. (108 J/g); |
| | | exothermic: 268.6° C. (6 J/g) |
| Acetone | I-1 Form A | Endothermic: 264.5° C. (55 J/g), 279.1° C. (55 J/g); |
| | | exothermic: 269.4° C. (9 J/g) |
| Methyl ethyl ketone | I-1 Form A | No comments |
| Ethyl acetate | I-1 Form A | No comments |
| Acetonitrile | I-1 Form A | No comments |
| Tetrahydrofuran | // | Clear solution, target concentration: 250 mg/mL |
| 1,4-Dioxane | // | Clear solution, target concentration: 250 mg/mL |
| Benzyl alcohol | I-1 Form A | No comments |
| 2-MeTHF | I-1 Form A | No comments |
| MeOH/water(55:45, | I-1 Form A | Thermal events: melting onset 281° C. (96 J/g) |
| v/v) a.w. 0.7 | | Weight loss: 0.4%@247° C. |
| | | ¹H-NMR (DMSO-d₆): no residual solvent |
| | | ee %(SFC): 100.0%* |
| EtOH/water(50:50, v/v) | I-1 Form A | No comments |
| a.w. 0.9 | | |
| Acetone/water(60:40, | I-1 Form A | Endothermic: 263.7° C. (1 J/g), 281.6° C. (90 J/g); |
| v/v) a.w. 0.8 | | exothermic: 267.9° C. (0.4 J/g) |
| ACN/water(80:20, v/v) | I-1 Form A | Endothermic: 263.9° C. (9 J/g), 281.9° C. (106 J/g); |
| a.w. 0.9 | | exothermic: 267.8° C. (9 J/g) |
| THF/water(85:15, v/v) | // | Clear solution, target concentration: 250 mg/mL |
| a.w. 0.9 | | |

About 50 mg of I-1 Form A was equilibrated in different solvents (pretreated by molecular sieves) under a temperature cycle between 5° C. to 50° C. at a heating/cooling rate of 0.2° C./min for 10 cycles and with protection from light. After 10 cycles, precipitates were collected at 5° C. by filtration. The solid parts (wet cake) were investigated by XRPD. When differences were observed, additional investigations were performed (e.g. DSC, TGA, 1H-NMR, SFC).

### Behavior Under Heating and Cooling

| Heating rate | Thermal events |
|---|---|
| Cycle 1: 30° C. to 280° C. at | Step 1: melting peak: |
| 10° C./min; 280° C. to −20° C. | 265.0° C.(100 J/g); |
| at 20° C./min; reheat to 300° C. | Step 3: glass transition: midpoint: |
| at 10° C./min. | 147.8° C., delta Cp: 0.4 J/(g · ° C.) |
| Cycle 2: 30° C. to 280° C. at | Step 1: melting peak: |
| 10° C./min; 280° C. to −20° C. | 263.9° C.(99 J/g); |
| at 2° C./min; reheat to 300° C. | Step 3:glass transition: midpoint: |
| at 10° C./min. | 157.5° C., delta Cp: 0.3 J/(g · ° C.) |

Polymorphic behavior of I-1 Form A was investigated by two different heating-cooling cycles of DSC. Cycle 1:30° C. to 280° C. at 10° C./min; 280° C. to −20° C. at 20° C./min; reheat to 300° C. at 10° C./min. Cycle 2:30° C. to 280° C. at 10° C./min; 280° C. to-20° C. at 2° C./min; reheat to 300° C. at 10° C./min.

### 4.8 Preparation of Polymorphs

### 4.8.1 Preparation of III-1 Form A

### Equilibration Experiments

I-1 was weighed into 2 mL glass vials and equilibrated in suitable amount of solvents (without pretreatment by molecular sieves) at different temperature on stirring plates and with protection from light. Obtained suspension was filtered with 0.45 μm nylon membrane and the solid part (wet cake) was investigated by XRPD and SFC to determine the form and chiral purity.

| | | | XRPD (3 | |
|---|---|---|---|---|
| Solvent | Temperature | pH | days) | Comments |
| Methanol | 50° C. | 6.21 | I-1 + II-1 | ee %(SFC): 87.9%* |
| | | | mixture | |
| EtOH/water | 50° C. | 6.82 | I-1 + II-1 | ee %(SFC): 91.6%* |
| (50:50, v/v) | | | mixture | |
| a.w. 0.9 | | | | |
| ACN/water | 50° C. | 6.79 | I-1 + II-1 | ee %(SFC): 67.4%* |
| (80:20, v/v) | | | mixture | |
| a.w. 0.9 | | | | |
| ACN/water | 50° C. | 4.48 | 3 days: I-1 + | 3 days: |
| (80:20, v/v) | | | II-1 mixture | ee %(SFC): 89.6%* |
| a.w. 0.9 | | | 5 days: I-1 + | 5 days: |
| | | | II-1 mixture | ee %(SFC): 88.9%* |
| ACN/water | 25° C. | 7.27 | 3 days: I-1 + | Added III-1 |
| (80:20, v/v) | | | II-1 mixture | Form A seeds |
| a.w. 0.9 | | | 8 days: I-1 + | 3 days: |
| | | | II-1 mixture | ee %(SFC): 90.9%* |
| | | | | 8 days: |
| | | | | ee %(SFC): 89.3%* |

*SFC test was immediately performed after preparation.
The water activity of a binary solvent system is calculated based on UNIFAC method (UNIQUAC Functional-group Activity Coefficients).

### Precipitation by Addition of Anti-Solvent

I-1 was dissolved in a good solvent. Obtained solutions were filtered by 0.45 μm nylon filter. Anti-solvents were added into the obtained solutions slowly. The solvents used were not pretreated by molecular sieves. The III-1 Form A seeds were added into the solutions properly with a stirring plate and with protection from light. Precipitates were collected by filtration. The solid parts (wet cake) were investigated by XRPD and SFC to determine the form and chiral purity.

| | Anti- | | |
|---|---|---|---|
| Solvents | solvent | XRPD | Comments |
| MeOH | MTBE(4V) | // | Stirred at 25° C. for 1 |
| | | | week, and no sufficient |
| | | | material for tests |
| MeOH | Toluene(4V) | // | Stirred at 25° C. for 1 |
| | | | week, and no sufficient |
| | | | material for tests |
| MeOH | Water(0.1V) | stirred for 2 h: | Stirred for 2 h |
| | | low crystallinity | ee %(SFC): 98.8%* |
| | | I-1 Form A | Stirred for 2 days |
| | | stirred for 2 days: | ee %(SFC): 99.2%* |
| | | I-1 Form A | |

“V” The volume ratio of anti-solvent to good solvent.
*SFC test was immediately performed after preparation.

### Slow Cooling

Approximate 50 mg of I-1 were dissolved in the minimal amount of selected solvents (without pretreatment by molecular sieves) at 50° C. Obtained solutions were filtered by 0.45 μm nylon filter. Obtained clear solutions were cooled to 5° C. at 0.1° C./min. III-1 Form A seeds were added into the solutions properly. Precipitates were collected by filtration. The solid parts (wet cake) were investigated by XRPD and SFC to determine the form and chiral purity.

| Solvents | Comments (added III-1 Form A seeds) |
|---|---|
| MeOH | Hold at 5° C. for 1 week, and no sufficient material for tests |
| ACN | Hold at 5° C. for 1 week, and no sufficient material for tests |
| Acetone | Hold at 5° C. for 1 week, and no sufficient material for tests |
| 1,4- | Hold at 5° C. for 1 week, and no sufficient material for tests |
| Dioxane | |

### 4.8.2 Preparation of I-1 Form B

### Equilibration Experiments

I-1 was weighed into 2 mL glass vials and equilibrated in suitable amount of solvents (without pretreatment by molecular sieves) at 50° C. on a stirring plate with protection from light and added I-1 Form B seeds properly. Obtained suspension was filtered with 0.45 μm nylon membrane and the solid part (wet cake) was investigated by XRPD and SFC to determine the form and chiral purity.

| Solvents | XRPD | Comments |
|---|---|---|
| THF | I-1 + II-1 | Added I-1 Form B seeds and stirred for 4 days |
| | mixture | ee %(SFC): 82.5%* |
| 1,4-Dioxane | I-1 + II-1 | Added I-1 Form B seeds and stirred for 4 days |
| | mixture | ee %(SFC): 80.1%* |
| 1,4-Dioxane | I-1 + II-1 | With molecular sieve and stirred for 6 days |
| | mixture | ee %(SFC): 88.26%* |
| 1,4-Dioxane | I-1 + II-1 | Without molecular sieve and stirred for 6 days |
| | mixture | ee %(SFC): 81.26%* |

*SFC test was immediately performed after preparation.

### 4.8.3 Preparation of III-1 Form B

### Equilibration Experiments

I-1 was weighed into 2 mL glass vials and equilibrated in suitable amount of THF/water (85:15, v/v) (without pretreatment by molecular sieves) at 25° C. on a stirring plate and added III-1 Form B seeds properly. Obtained suspension was filtered with 0.45 μm nylon membrane and the solid part (wet cake) was investigated by XRPD and SFC to determine the form and chiral purity.

| | | Comments(added III-1 |
|---|---|---|
| Solvents | XRPD (4 days) | Form B seeds) |
| THF/water(85:15, v/v) | I-1 + II-1 mixture | ee %(SFC): 93.8%* |
| a.w. 0.9 | | |

*SFC test was immediately performed after preparation.
The water activity of a binary solvent system is calculated based on UNIFAC method (UNIQUAC Functional-group Activity Coefficients).

### 4.8.4 Preparation of III-1 Form D

### Slow Evaporation

Based on approximate solubility results, about 50 mg of I-1 was dissolved in a suitable amount of solvents (without pretreatment by molecular sieves). Obtained solutions were filtered by 0.45 μm nylon filter. Obtained clear solutions were slowly evaporated upon protecting from light. Solid residues were examined for their polymorphic form by XRPD and SFC to determine chiral purity.

| Solvents | XRPD | Comments |
|---|---|---|
| MeOH | I-1 + II-1 | At 25° C. for 12 days |
| | mixture | ee %(SFC): 98.0%* |
| MeOH/DCM(1:1, v/v) | I-1 + II-1 | Added I-1 Form D seeds |
| | mixture | ee %(SFC): 88.9%* |
| MeOH/DCM(1:1, v/v) | // | Added SEF2 seeds targeting to single |
| | | crystal: fine particles |

*SFC test was immediately performed after preparation.
“//” Not carried out.

### Precipitation by Addition of Anti-Solvent

Batch I-1 was dissolved in a good solvent. Obtained solutions were filtered by 0.45 μm nylon filter. Anti-solvents were added into the obtained solutions slowly. The solvents used were not pretreated by molecular sieves. The III-1 Form D seeds were added into the solutions properly on a stirring plate with protection from light. Precipitates were collected by filtration. The solid parts (wet cake) were investigated by XRPD and SFC to determine the form and chiral purity.

| | | | Comments(added III-1 |
|---|---|---|---|
| Solvent | Anti-solvent | XRPD | Form D seeds) |
| Acetone | Heptane(2V) | I-1 Form A | stirred for 1 h |
| | | | ee %(SFC): 94.9%* |

“V” The volume ratio of anti-solvent to good solvent
*SFC test was immediately performed after preparation.

### 4.8.5 Preparation of I-1 Form C

### Precipitation by Addition of Anti-Solvent

I-1 Form A was dissolved in a good solvent. Obtained solutions were filtered by 0.45 μm nylon filter. Anti-solvents were added into the obtained solutions slowly. The solvents used were not pretreated by molecular sieves. I-1 Form C seeds were added into the solutions properly on a stirring plate with protection from light. Precipitates were collected by filtration. The solid parts (wet cake) were investigated by XRPD and SFC to determine the form and chiral purity.

| | | | Comments(added |
|---|---|---|---|
| Solvent | Anti-solvent | XRPD | I-1 Form C seeds) |
| THF | MTBE(1.5V) | I-1 Form C | Stirred for 2 h |
| | | | ee %(SFC): 99.5%* |

*SFC test was immediately performed after preparation
VThe volume ratio of anti-solvent to good solvent

### 4.9 Crystallization of II-1

### Equilibration Experiments

II-1 was weighed into 2 mL glass vials and equilibrated in suitable amount of solvents (without pretreatment by molecular sieve) at different temperature (e.g. 25° C., 50° C.) on stirring plates with protection from light. Obtained suspensions were filtered with 0.45 μm nylon membrane and the solid parts (wet cakes) were investigated by XRPD and SFC to determine the form and chiral purity.

| Solvent | Temperature | XRPD (6 days) | Comments |
|---|---|---|---|
| Methanol | 50° C. | II-1 Form A (include the | ee %(SFC): −99.66%* |
| | | characteristic peak of 2Theta | |
| | | 16.7°, 20.7°) | |
| EtOH/water(50:50, | 50° C. | II-1 Form A (include the | ee %(SFC): −99.78%* |
| v/v) a.w. 0.9 | | characteristic peak of 2Theta | |
| | | 16.7°, 20.7°) | |
| ACN/water(80:20, | 50° C. | II-1 Form A (include the | ee %(SFC): −99.24%* |
| v/v) a.w. 0.9 | | characteristic peak of 2Theta | |
| | | 16.7°, 20.7°) | |
| 1,4-dioxane | 50° C. | II-1 Form B | ee %(SFC): −99.48%* |
| THF/water(85:15, | 25° C. | II-1 Form C (include the | ee %(SFC): −99.34%* |
| v/v) a.w. 0.9 | | characteristic peak of 2Theta | |
| | | 10.9°, 17.5°, 20.9°, 25.4°) | |

*SFC test was immediately performed after preparation.
The water activity of a binary solvent system is calculated based on UNIFAC method (UNIQUAC Functional-group Activity Coefficients).

### Competitive Equilibration Experiment

Competitive slurry were conducted to determine thermodynamic relationships of the two anhydrates I-1 Form A and Form C. Form A was used to prepare saturated solutions in THF/heptane (2:3, v/v), THF/MTBE (1:4, v/v), MeOH/DCM (1:2, v/v), ethyl acetate/heptane (1:1, v/v), 1,4-dioxane and THF/ACN (2:1, v/v) at 25° C. All the used solvents were not pretreated by molecular sieves. Then 5 mg of Form A and 5 mg of Form C were respectively added to the saturated solutions. Obtained suspensions were stirred at 25° C. for 3-5 days. Wet solids were isolated by centrifugal filtration and investigated by XRPD and SFC.

| | 25° C. | |
|---|---|---|
| Solvent | XRPD | Comments |

Initial I-1 Form A + Form C

| | | |
|---|---|---|
| THF/heptane(2:3, v/v) | Form A | stirred for 3-4 days: |
| | | ee %(SFC): 96.9%* |
| THF/MTBE(1:4, v/v) | Similar to Form A, | stirred for 3-4 days: |
| | low crystallinity | ee %(SFC): 96.8%* |
| MeOH/DCM(1:2, v/v) | Low crystallinity, | stirred for 3-4 days: |
| | I-1 + II-1 mixture | ee %(SFC): 57.4%* |
| Ethyl acetate/ | Form A | stirred for 3-4 days: |
| heptane(1:1, v/v) | | ee %(SFC): 96.4%* |

Initial Form A

| | | |
|---|---|---|
| MeOH/DCM(1:2, v/v) | Low crystallinity, | stirred for 5 days: |
| | I-1 + II-1 mixture | Solids: |
| | | ee %(SFC): −23.12%* |
| | | Supernatant: |
| | | ee %(SFC): 97.78%* |

Initial I-1 Form A + Form C

| | | |
|---|---|---|
| 1,4-dioxane | Form A | // |
| THF/MTBE(1:4, v/v) | Form A, low | // |
| | crystallinity | |
| THF/ACN(2:1, v/v) | Form A, low | // |
| | crystallinity | |

*SFC test was immediately performed after preparation.
“//”: No comments

### Behavior Under Compression

About 20 mg of I-1 Form A was compressed for 5 minutes at 2 MPa, 5 MPa and 10 MPa with a hydraulic press. XRPD characterization was performed to investigate the polymorphic behavior under compression.

| Pressure | XRPD |
|---|---|
| 2 Mpa | No form change, but peaks became slightly broader |
| 5 Mpa | No form change, but peaks became slightly broader |
| 10 Mpa | No form change, but peaks became slightly broader |

### Grinding Simulation Experiments

About 50 mg of I-1 Form A was ground manually with a mortar and pestle for 1, 3 and 5 min. Form transformation and degree of crystallinity was evaluated by XRPD.

| | XRPD |
|---|---|
| Dry grinding for 1 min | No form and crystallinity change, |
| Dry grinding for 3 min | No form change, but crystallinity |
| | slightly decreased |
| Dry grinding for 5 min | No form change, but crystallinity |
| | slightly decreased |

### Granulation Simulation Experiments

Ethanol (without pretreatment by molecular sieve) and pure water were added drop wise to about 20 mg of I-1 Form A separately until solid is wetted sufficiently. Manually grind the samples with a mortar and pestle for about 5 min. Samples were dried under ambient condition for 10 min. Form transformation and degree of crystallinity were evaluated by XRPD.

| Solvent | XRPD |
|---|---|
| Ethanol | No form and crystallinity change |
| water | No form and crystallinity change |

II-1 was weighed into 2 mL glass vials and equilibrated in suitable amount of solvents (without pretreatment by molecular sieve) at different temperature (e.g. 25° C., 50° C.) on stirring plates with protection from light. Obtained suspensions were filtered with 0.45 μm nylon membrane and the solid parts (wet cakes) were investigated by XRPD and SFC to determine the form and chiral purity.

| Solvent | Temperature | XRPD (6 days) | Comments |
|---|---|---|---|
| Methanol | 50° C. | II-1 Form A (include the | ee %(SFC): −99.66%* |
| | | characteristic peak of 2Theta | |
| | | 16.7°, 20.7°) | |
| EtOH/water(50:50, | 50° C. | II-1 Form A (include the | ee %(SFC): −99.78%* |
| v/v) a.w. 0.9 | | characteristic peak of 2Theta | |
| | | 16.7°, 20.7°) | |
| ACN/water(80:20, | 50° C. | II-1 Form A (include the | ee %(SFC): −99.24%* |
| v/v) a.w. 0.9 | | characteristic peak of 2Theta | |
| | | 16.7°, 20.7°) | |
| 1,4-dioxane | 50° C. | II-1 Form B | ee %(SFC): −99.48%* |
| THF/water(85:15, | 25° C. | II-1 Form C (include the | ee %(SFC): −99.34%* |
| v/v) a.w. 0.9 | | characteristic peak of 2Theta | |
| | | 10.9°, 17.5°, 20.9°, 25.4°) | |

*SFC test was immediately performed after preparation.
The water activity of a binary solvent system is calculated based on UNIFAC method (UNIQUAC Functional-group Activity Coefficients).

### 4.10 Competitive Equilibration Experiment

Competitive slurry were conducted to determine thermodynamic relationships of the two anhydrates I-1 Form A and Form C. Form A was used to prepare saturated solutions in THF/heptane (2:3, v/v), THF/MTBE (1:4, v/v), MeOH/DCM (1:2, v/v), ethyl acetate/heptane (1:1, v/v), 1,4-dioxane and THF/ACN (2:1, v/v) at 25° C. All the used solvents were not pretreated by molecular sieves. Then 5 mg of Form A and 5 mg of Form C were respectively added to the saturated solutions. Obtained suspensions were stirred at 25° C. for 3-5 days. Wet solids were isolated by centrifugal filtration and investigated by XRPD and SFC.

| | 25° C. | |
|---|---|---|
| Solvent | XRPD | Comments |

Initial Form A + Form C

| | | |
|---|---|---|
| THF/heptane(2:3, v/v) | Form A | stirred for 3-4 days: |
| | | ee %(SFC): 96.9%* |
| THF/MTBE(1:4, v/v) | Similar to Form A, | stirred for 3-4 days: |
| | low crystallinity | ee %(SFC): 96.8%* |
| MeOH/DCM(1:2, v/v) | Low crystallinity, | stirred for 3-4 days: |
| | I-1 + II-1 mixture | ee %(SFC): 57.4%* |
| Ethyl acetate/ | Form A | stirred for 3-4 days: |
| heptane(1:1, v/v) | | ee %(SFC): 96.4%* |

Initial Form A

| | | |
|---|---|---|
| MeOH/DCM(1:2, v/v) | Low crystallinity, | stirred for 5 days: |
| | I-1 + II-1 mixture | Solids: |
| | | ee %(SFC): −23.12%* |
| | | Supernatant: |
| | | ee %(SFC): 97.78%* |

Initial Form A + Form C

| | | |
|---|---|---|
| 1,4-dioxane | Form A | // |
| THF/MTBE(1:4, v/v) | Form A, low | // |
| | crystallinity | |
| THF/ACN(2:1, v/v) | Form A, low | // |
| | crystallinity | |

*SFC test was immediately performed after preparation.
“//”: No comments

### 4.11 Behavior Under Compression

About 20 mg of Form A was compressed for 5 minutes at 2 MPa, 5 MPa and 10 MPa with a hydraulic press. XRPD characterization was performed to investigate the polymorphic behavior under compression.

### 4.12 Grinding Simulation Experiments

About 50 mg of Form A was ground manually with a mortar and pestle for 1, 3 and 5 min. Form transformation and degree of crystallinity was evaluated by XRPD.

### 4.13 Granulation Simulation Experiments

Ethanol (without pretreatment by molecular sieve) and pure water were added drop wise to about 20 mg of Form A separately until solid is wetted sufficiently. Manually grind the samples with a mortar and pestle for about 5 min. Samples were dried under ambient condition for 10 min. Form transformation and degree of crystallinity were evaluated by XRPD.

### Example 5. Preparation of Salts of Compounds of Formulae I, II, and III, and Crystal Forms Thereof

### 5.1 Summary

### Salt Screening

I-1 is a small molecule with pKa(s) of 2.69 and 9.64 according to calculation by Marvin Sketch. I-1 Form A was used in this salt screening study. Considering the racemization behavior of the desired free form of I-1 in different solvent systems and operational conditions, the major goal of the salt screening study is to identify potential salts that provide better chiral stability.

Based on the pKa of I-1 free form, 23 counter ions were selected as salt/cocrystal forming agents. Methanol, acetone and isopropyl acetate were used as screening solvents. 1.0 equivalent of selected counter ions/coformers was applied for salt screening. Slurry equilibration, slow evaporation and anti-solvent addition were used as crystallization methods. In total, about 70 screening experiments were conducted.

From the screening, 8 salts and their polymorphs were identified, including III-2 Form A, I-2 Form A, III-2 Form B, I-3 Form A, I-4 Form A, III-6 Form A, I-5 Form A and I-5 Form B (Table 5.2, and Table 5.3). Among these salts, I-3 Form A, I-4 Form A and I-5 Form A show high chiral purity, suggesting that they are I-1 enantiomer salts. However, I-5 Form A is a methanol solvate thus excluded from further evaluation. Therefore, I-3 Form A and I-4 Form A were selected as salt candidates and scaled up for full evaluation.

### Salt Candidates Evaluation

I-3 Form A and I-4 Form A were scaled up successfully. The scale up batches are the same polymorphs as those of the screening samples. The two salt candidates were evaluated in comparison with I-1 Form A in terms of chemical and physicochemical properties, stability, solubility, suspension stability, solution stability and hygroscopicity.

### Crystallinity and Thermal Properties:

I-1 Form A is an anhydrate. It is of high crystallinity. DSC shows multiple thermal events. TGA shows about 1.6% weight loss at about 250° C. No residual solvent is detected by ¹H-NMR. I-3 Form A is an anhydrate. It is of high crystallinity. Stoichiometric ratio of free form to p-toluene sulfonic acid is 1:1 by ¹H-NMR. DSC shows a melting peak at Tₒₙₛₑₜ of 251.2° C. Decomposition occurred upon melting. TGA shows about 0.8% weight loss at about 214° C. ¹H-NMR shows about 0.2% acetone by weight (0.03 equivalent by molar ratio). I-4 Form A is a hydrate. It is of medium crystallinity. KF shows about 2.5% water by weight (0.86 equivalent by molar ratio). Stoichiometric ratio of free form to methanesulfonic acid is 1:1.1 based on ¹H-NMR. DSC shows multiple thermal events. TGA shows about 1.5% weight loss at about 100° C. No residual solvent was detected.

### Initial Chemical Purity and Chiral Purity:

I-1 Form A, I-3 Form A and I-4 Form A have chemical purity of 99.0%, 99.5% and 99.6%, respectively and chiral purity of 99.0%, 98.6% and 99.5%, respectively.

### Bulk Stability:

Bulk stability of I-1 Form A and the 2 salt candidates was investigated at 25° C./92% RH in an open container, at 40° C./75% RH in an open container and at 60° C. in a tight container over 1 week. I-3 Form A is chemically and physically stable under these conditions. I-3 Form A is chemically stable and shows no XRPD change during bulk stability study. However, I-3 Form A was found to convert to a hydrate in the hygroscopicity study, suggesting its physical stability is sensitive to environmental humidity. I-4 Form A is chemically stable but physically unstable at 25° C./92% RH and 40° C./75% RH. All the 3 salts show no obvious racemization based on chiral purity results.

### Solubility:

Solubility of I-1 Form A and the 2 salt candidates were measured in pH 2.0 HCl buffer solution, water, and 3 bio-relevant media (SGF, FaSSIF-V1 and FeSSIF-V1) and current lyophilization solvent mixture of MeOH/DCM (1:2, v/v) at 37° C. for 2 h and 24 h. I-1 Form A and the two salts show very low solubility (<LOQ. LOQ=0.5 μg/mL) in pH 2 HCl buffer solution and pure water. In SGF, all the free form and the 2 salts show comparable solubility. In FeSSIF-V1 and FaSSIF-V1, the 2 salts show higher kinetic solubility than that of I-1 Form A at 2h, but decreased to similar solubility due to dissociation of the salts at 24 h. when the residual solids was fully dissociated to the free form. In addition, residual solids were checked by chiral HPLC to confirm whether racemization occurs. No obvious racemization was observed for the residual solids isolated from the media except for FeSSIF-V1, where significant racemization occurred possibly due to the components of FeSSIF-V1 (such as high level of sodium and acetate ions, which were known to accelerate racemization). In MeOH/DCM (1:2, v/v), the solubility of I-1 Form A, I-3 Form A and I-4 Form A is >145 mg/mL, >75 mg/mL and >111 mg/mL, respectively.

Suspension stability: Suspension stability of I-1 Form A and the 2 salt candidates was investigated in pure water, 0.5% (w/w) Tween 80 aqueous solution and 0.5% (w/w) MC (400 cP)+2% (w/w) Vitamin E TPGS aqueous solution at 25° C. for 2 h and 24 h. Target concentration was 2 mg/mL. I-1 Form A and the 2 salts are chemically stable in the 3 vehicles. I-1 Form A is physically stable, but the 2 salts convert to I-1 Form A or amorphous form, suggesting dissociation occurred. For I-1 Form A, there is no racemization in these vehicles. For the 2 salts, no racemization is observed in water, but slight decrease of chiral purity is found in 0.5% (w/w) Tween 80. Due to extremely high solubility of the salts in 0.5% (w/w) MC (400 cP)+2% (w/w) Vitamin E TPGS vehicle (>2 mg/mL), no sufficient residual solids of the 2 salts was collected for chiral purity test.

To confirm whether racemization occurs for the 2 salts in 0.5% (w/w) MC (400 cP)+2% (w/w) Vitamin E TPGS aqueous solution, the suspension stability in this vehicle was repeated at target concentration of 10 mg/mL. Both salts are chemically stable and show comparable solubility. Residual solids show no racemization. After the test, the mesylate salt was found to have converted to amorphous form. For the tosylate salt, according to XRPD results, the 2 h sample of the residual solids showed a form change, but the 24 h sample was remained unchanged. Based on variable humidity XRPD, I-3 Form A sample collected at 2 h had actually converted to a hydrate (I-3 Form B) due to increased environmental humidity. The hygroscopicity of these salts were further investigated.

### Solution Stability:

Solution stability of I-1 Form A and I-3 Form A was investigated in methanol and THF/ACN (2:1, v/v) at 25° C. Target concentration is 4 mg/mL. All the physical forms are chemically stable at 25° C. for at least 24 h and no racemization was observed in all the solutions.

### Hygroscopicity:

I-1 Form A is hygroscopic. It absorbs about 5.5% water by weight from 40% RH to 95% RH. After the DVS test, there is no form and crystallinity change. I-4 Form A is very hygroscopic. It absorbs about 40% water by weight from 40% RH to 95% RH. Form changed after the DVS test. I-3 Form A is stable when relative humidity is below: 40%. It starts to absorb water when RH>40% and potential hydrate form generates at high humidity based on DVS data. The sorption and desorption are reversible. After a DVS cycle, obtained sample is still I-3 Form A by XRPD.

### Investigation of I-3 Form A in Different Humidity

### Variable Humidity XRPD:

According to the DVS result of I-3 Form A, two hydrate forms (a mono-hydrate, and a bis-hydrate) may exist when relative humidity is above 50% RH. Variable humidity XRPD (VH-XRPD) was investigated attempting to capture the potential hydrate(s). The VH-XRPD results show that at 70% RH, I-3 Form A has fully converted to a new form, I-3 Form B, whose pattern started to show up in the XRPD at 50% RH, suggesting that the starting point of hydrate formation could be even lower, but kinetics is too slow to be captured by variable humidity XRPD.

### Humidity Chamber:

To further evaluate form conversion of I-3 Form A, this salt was placed in different humidity (30% RH, 40% RH and 50% RH) chambers for 1 week. Based on XRPD results, I-3 Form A converted to I-3 Form B at 40% RH and 50% RH. Form conversion is even observed at 30% RH since the XRPD shows additional peaks belongs to Form B. The results suggest that I-3 Form A is sensitive to humidity and needs to be well protected from moisture during manufacturing and storage.

### Conclusion

Above all, I-3 Form A has high crystallinity, high melting point, reasonable stoichiometry, good chemical and physical bulk stability, and good solution stability. According to free form polymorph study, I-1 Form A is found to be easily racemized in certain organic solvents, including methanol and IPAc, which were used for salt preparation. I-3 Form A show good chiral stability in salt screening solvents (MeOH, acetone and IPAc) and scale up solvent (acetone), showing advantage in chiral stability. Therefore, the tosylate salt is a promising candidate and recommended for further development. However, I-3 Form A is sensitive to moisture and hydrate form formation is found to be readily occurred at relatively low humidity condition (30-50% RH). Polymorph screening work of the tosylate salt is warranted to identify the most suitable physical form.

### 5.2. General Information

### Compound:

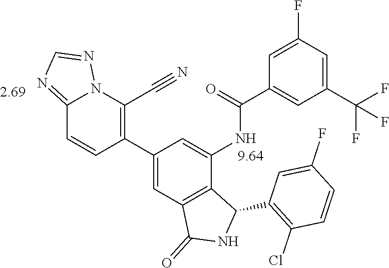 [SMILES: N#Cc1c(-c2cc(NC(=O)c3cc(F)cc(C(F)(F)F)c3)c3c(c2)C(=O)N[C@H]3c2cc(F)ccc2Cl)ccc2ncnn12]

pKa(s): 2.69 and 9.64 calculated by Marvin Sketch.

### 5.3 Starting Material Used for Salt Screening

### Compound I-1 Form A

| Parameter | Method | Result |
|---|---|---|
| Chemical purity | HPLC | 99.0% |
| Chiral purity | HPLC | 99.2% |
| X-ray diffraction | 3-40° (2 theta) | Form A, high crystallinity |
| DSC melting onset | DSC, 10° C./min | Exothermic: 255.9° C. (7 J/g), 267.7° C. |
| and enthalpy | | (3 J/g); endothermic: 279.4° C. (89 J/g) |
| Thermogravimetry | TGA, 10° C./min | 1.6%@ 250° C. |
| Residual solvent | ¹H-NMR (DMSO-d₆) | No residual solvent |

### 5.4 Salt Screening

About 5 mg of I-1 Form A was weighed into a 2 mL glass vial and aliquots of 20 μL of each solvent (pretreated by molecular sieves) was added to determine solubility at 25° C. Max. volume of each solvent added was 1 mL. Approximate solubility was determined by visual observation.

### Approximate Solubility of I-1 Form A

| | Solubility |
|---|---|
| Solvent | (mg/mL, 25° C.) |
| Water | <5 |
| Methanol | 12-17 |
| Ethanol | <5 |
| Isopropanol | <5 |
| Acetone | 12-17 |
| Methyl ethyl ketone | 12-17 |
| Isopropyl acetate | <5 |
| Acetonitrile | 10-17 |
| Tetrahydrofuran | 33-64 |
| Trifluoroethanol | 12-17 |
| Dichloromethane | <5 |

### Screening Experiments

Based on calculated pKa of 2.69 and 9.64, 10 Class I acids, 6 Class II acids, 1 Class III acid, 1 Class I base and 5 coformers were selected to pursue potential salt and cocrystal opportunities. About 50 mg of I-1 Form A were added to a suitable amount of solvent and 1.0 equivalent of counter ions were added under stirring at 50° C. for 2 hours and then at 25° C. for at least 12 hours. MeOH, acetone and IPAc were used as screening solvents.

For those clear solutions obtained, half volume were evaporated in a fume hood; the rest were treated by anti-solvents addition.

Obtained suspensions were taken out and centrifuged. Solids obtained were analyzed by XRPD and salt screening results were summarized in Table 5.1, Table 5.2, and Table 5.3. Hits with high or medium crystallinity were further characterized according to Table 5.4.

### Counter Ions Used for Salt Screening

| Counter ions | pKa(s) | M.W. | Class |
|---|---|---|---|
| HCl | −6.1 | 36.46 | I |
| Sulfuric acid | −3, 1.92 | 98.08 | I |
| Phosphoric acid | 1.96, 7.12, 12.32 | 98 | I |
| L-malic acid | 3.4, 5.09 | 134.09 | I |
| L-tartaric acid | 3.02, 4.36 | 150.09 | I |
| Citric acid | 3.12, 4.76, 6.39 | 192.13 | I |
| Fumaric acid | 3.03, 4.38 | 116.08 | I |
| L-aspartic acid | 1.88, 3.65, 9.60(B) | 133.11 | I |
| L-glutamic acid | 2.19, 4.25, 9.67(B) | 147.13 | I |
| Maleic acid | 1.92, 6.23 | 116.08 | I |
| Gentisic acid | 2.93 | 154.12 | II |
| Malonic acid | 2.82 | 104.06 | II |
| Methanesulfonic acid | −1.2 | 96.10 | II |
| p-Toluenesulfonic acid | −1.34 | 172.21 | II |
| Naphthalene-2-sulfonic acid | 0.17 | 208.24 | II |
| Naphthalene-1,5-disulfonic acid | −3.37, −2.64 | 332.26 | II |
| Hydrobromic acid | <−6 | 80.92 | III |
| Sodium hydroxide | 14 | 40 | I |
| Theophylline | N/A | 180.16 | N/A |
| Theobromine | N/A | 180.16 | N/A |
| Nicotinic acid | 4.85 | 123.11 | II |
| Nicotinamide | N/A | 122.12 | N/A |
| Isonicotinamide | N/A | 122.12 | N/A |

TABLE 5.1
Salt screening results (slurry crystallization)

| | A | B | C |
|---|---|---|---|
| Counter ions | MeOH | Acetone | Isopropyl acetate |
| Free form | − (I-1 Form A) | − (I-1 Form A) | − (I-1 Form A) |
| 1.0 equiv. HCl | − (I-1 Form A) | + (III-2 Form A) | + (I-2 Form A, low |
| | | Chiral purity: 30.8% | crystallinity) |
| | | | Chiral purity: 89.7% |
| 1.0 equiv. | − (I-1 Form A) | thin suspension | thin suspension |
| sulfuric acid | | | |
| 1.0 equiv. | − (I-1 Form A) | thin suspension | − (I-1 Form A) |
| phosphoric acid | | | |
| 1.0 equiv. L- | − (I-1 Form A) | thin suspension | − (I-1 Form A) |
| malic acid | | | |
| 1.0 equiv. L- | − (I-1 Form A) | thin suspension | − (I-1 Form A) |
| tartaric acid | | | |
| 1.0 equiv. citric | − (I-1 Form A) | thin suspension | − (I-1 Form A) |
| acid | | | |
| 1.0 equiv. | − (I-1 Form A) | − (physical mixtures | − (physical mixtures |
| fumaric acid | | of I-1 Form A and | of I-1 Form A and |
| | | fumaric acid) | fumaric acid) |
| 1.0 equiv. L- | − (physical mixtures | − (physical mixtures | − (physical mixtures |
| aspartic acid | of I-1 Form A and | of I-1 Form A and | of I-1 Form A and |
| | L-aspartic acid) | L-aspartic acid) | L-aspartic acid) |
| 1.0 equiv. L- | − (physical mixtures | − (physical mixtures | − (physical mixtures |
| glutamic acid | of I-1 Form A and | of I-1 Form A and | of I-1 Form A and |
| | L-glutamic acid) | L-glutamic acid) | L-glutamic acid) |
| 1.0 equiv. maleic | − (I-1 Form A) | thin suspension | − (I-1 Form A) |
| acid | | | |
| 1.0 equiv. | − (I-1 Form A) | thin suspension | − (I-1 Form A) |
| gentisic acid | | | |
| 1.0 equiv. | − (I-1 Form A) | thin suspension | − (I-1 Form A) |
| malonic acid | | | |
| 1.0 equiv. | I-1 Form A + | thin suspension | thin suspension |
| methanesulfonic | additional one peak | | |
| acid | 2theta 9.2° | | |
| 1.0 equiv. p- | + (I-3 Form A) | + (I-3 Form A) | + (I-3 Form A) |
| toluenesulfonic | | Chiral purity: 99.8% | |
| acid | | | |
| 1.0 equiv. | I-1 Form A + one peak | + (III-6 Form A, low | thin suspension |
| naphthalene-2- | 9.3° | crystallinity) | |
| sulfonic acid | | Chiral purity: 43.2% | |
| 1.0 equiv. | + (I-1 Form A ) | thin suspension | 3 days: AF; |
| naphthalene-1,5- | Chiral purity: 100% | | for another |
| disulfonic acid | dried at 50° C. for 21 h: | | 3 days: AF |
| | low crystallinity | | |
| | Chiral purity: 100% | | |
| 1.0 equiv. | − (I-1 Form A) | thin suspension | clear solution |
| Hydrobromic | | | |
| acid | | | |
| 1.0 equiv. | clear solution | thin suspension | 3 days: practically |
| sodium | | | amorphous; for |
| hydroxide | | | another 3 days: AF |
| 1.0 equiv. | − (physical mixtures | − (physical mixtures | − (physical mixtures |
| theophylline | of I-1 Form A and | of I-1 Form A and | of I-1 Form A and |
| | theophylline) | theophylline) | theophylline) |
| 1.0 equiv. | − (physical mixtures | − (physical mixtures | − (physical mixtures |
| theobromine | of I-1 Form A and | of I-1 Form A and | of I-1 Form A and |
| | theobromine) | theobromine) | theobromine) |
| 1.0 equiv. | − (physical mixtures | − (physical mixtures | − (physical mixtures |
| nicotinic acid | of I-1 Form A and | of I-1 Form A and | of I-1 Form A and |
| | nicotinic acid) | nicotinic acid) | nicotinic acid) |
| 1.0 equiv. | − (I-1 Form A | thin suspension | − (physical mixtures |
| nicotinamide | additional of 2theta | | of I-1 Form A and |
| | 17.1°, 17.5°) | | nicotinamide) |
| 1.0 equiv. | − (I-1 Form A) | thin suspension | − (physical mixtures |
| isonicotinamide | | | of I-1 Form A and |
| | | | isonicotinamide) |
| Counter ions | ACN | Acetone | Isopropyl acetate |
| 1.0 equiv. HCl | + (III-2 Form B) | + (III-2 Form A) | clear solution |
| | Chiral purity: 20.2% | Chiral purity: 45.4% | |
| Counter ions | MeOH/water(9:1, v/v) | | |
| 1.0 equiv. | − (physical mixtures of | // | // |
| naphthalene-1,5- | I-1 Form A, I-5 Form | | |
| disulfonic acid | B and aphthalene-1,5- | | |
| | disulfonic acid) | | |
| | Chiral purity: 99.3% | | |

“+”: Salt hits
“−”: Free form, counter ions, or physical mixtures
“AF”: Amorphous form
“//”: Not carried out

TABLE 5.2
Salt screening results (slow evaporation)

| | A | B | C |
|---|---|---|---|
| Counter ions | MeOH | Acetone | IPAc |
| 1.0 equiv. sulfuric acid | // | AF | AF |
| 1.0 equiv. phosphoric acid | // | AF | // |
| 1.0 equiv. L-malic acid | // | AF | // |
| 1.0 equiv. L-tartaric acid | // | AF | // |
| 1.0 equiv. citric acid | // | AF | // |
| 1.0 equiv. maleic acid | // | AF | // |
| 1.0 equiv. gentisic acid | // | AF | // |
| 1.0 equiv. malonic acid | // | AF | // |
| 1.0 equiv. methanesulfonic | // | AF | AF |
| acid | | | |
| 1.0 equiv. naphthalene-2- | // | // | AF |
| sulfonic acid | | | |
| 1.0 equiv. naphthalene-1,5- | // | AF | // |
| disulfonic acid | | | |
| 1.0 equiv. Hydrobromic | // | AF | AF |
| acid | | | |
| 1.0 equiv. sodium | AF | AF | // |
| hydroxide | | | |
| 1.0 equiv. nicotinamide | // | AF | // |
| 1.0 equiv. isonicotinamide | // | Practically | // |
| | | amorphous | |

“//”: Not carried out
“AF”: Amorphous form

### 5.3 Salt Screening Results (Anti-Solvent)

5.3 Salt screening results (anti-solvent)

| | A | B | C |
|---|---|---|---|
| Counter ions | MeOH/MTBE | Acetone/MTBE | IPAc/MTBE |
| 1.0 equiv. sulfuric acid | // | thin suspension | AF |
| 1.0 equiv. phosphoric acid | // | −(I-1 Form B) | // |
| 1.0 equiv. L-malic acid | // | −(I-1 Form B) | // |
| 1.0 equiv. L-tartaric acid | // | −(I-1 Form B) | // |
| 1.0 equiv. citric acid | // | −(I-1 Form B) | // |
| 1.0 equiv. maleic acid | // | −(I-1 Form B) | // |
| 1.0 equiv. gentisic acid | // | −(I-1 Form B) | // |
| 1.0 equiv. malonic acid | // | −(I-1 Form B) | // |
| 1.0 equiv. methanesulfonic | // | + (I-4 Form A) | thin suspension |
| acid | | Chiral purity: 98.3% | |
| 1.0 equiv. naphthalene-2- | // | // | AF |
| sulfonic acid | | | |
| 1.0 equiv. naphthalene-1,5- | // | + (I-5 Form B) | // |
| disulfonic acid | | Chiral purity: 88.3% | |
| 1.0 equiv. Hydrobromic acid | // | thin suspension | thin suspension |
| 1.0 equiv. sodium hydroxide | Practically | AF | // |
| | amorphous | | |
| 1.0 equiv. nicotinamide | // | −(I-1 Form B) | // |
| 1.0 equiv. isonicotinamide | // | −(I-1 Form B) | // |
| 1.0 equiv. HCl | // | // | AF |

“+”: Salt hits
−Free form, counter ions, or physical mixtures
“AF”: Amorphous form
“//”: Not carried out

TABLE 5.4
Characterization of crystalline hits

| | | | | I-3 Form A | |
|---|---|---|---|---|---|
| | I-1 Form A | III-2 Form A | I-2 Form A | (anhydrate) | III-6 Form A |
| Counter ion | // | I | I | II | II |
| Class | | | | | |
| Solvent | MeOH, | Acetone | IPAc | MeOH, acetone, | Acetone |
| | acetone, IPAc | | | IPAc | |
| Chiral purity | 99.2% | 30.8% | 89.7% | 99.8% | 43.2% |
| (by HPLC) | | | | | |
| Crystallinity | High | Medium | Low | High | low |
| (By XRPD) | crystallinity | crystallinity | crystallinity | crystallinity | crystallinity |
| Melting onset | Exothermic: | Endothermic: | Endothermic: | 251.1° C. | Endothermic: |
| (DSC, ° C.) | 255.9° C., | 151.8° C., | 121.3° C., | | 47.0° C. |
| | 267.7° C.; | 275.5° C.; | 276.5° C., | | 145.8° C. |
| | endothermic: | exothermic: | 284.3° C.; | | |
| | 279.4° C. | 225.2° C.; | exothermic: | | |
| | | | 237.1° C. | | |
| Enthalpy | Exothermic: | Endothermic: | Endothermic: | 97 | Endothermic: |
| (DSC, J/g) | 7, 3; | 75, 81; | 97, 45, 14; | | 3, 52 |
| | endothermic: | exothermic: 67 | exothermic: 50 | | |
| | 89 | | | | |
| Weight loss | 1.6%@250° C. | 8.0%@194° C. | 12.6%@168° C. | 1.2%@214° C. | 6.8%@190° C. |
| (TGA, %) | | | | | |
| Stoichiometric | // | // | // | free form:counter- | free form:counter- |
| ratio | | | | ion = 1:1 | ion = 1:0.36 |
| (by ¹H-NMR) | | | | | |
| Residual solvent | No residual | 6.3% acetone | 3.7% 2- | 0.3% acetone | 4.8% acetone |
| (by ¹H-NMR) | solvent | by weight | propanol | by weight | by weight |
| | | (0.7 equiv.) | by weight | (0.04 equiv) | (0.6 equiv.) |
| | | | (0.4 equiv); | | |
| | | | 1.4% IPAc | | |
| | | | by weight | | |
| | | | (0.09 equiv.) | | |

| | I-5 Form A | | | |
|---|---|---|---|---|
| | (methanol solvate) | I-4 Form A | I-5 Form B | III-2 Form B |
| Counter | II | II | II | I |
| ion Class | | | | |
| Solvent | MeOH | Acetone/MTBE(1:4, v/v) | Acetone/MTBE | ACN |
| | | | (1:4, v/v) | |
| Chiral purity | 100% dried at 50° | 98.3% | 88.3% | 20.3% |
| (by HPLC) | C. for 21 h: 100% | | | |
| Crystallinity | Medium crystallinity | Low | Medium | Low |
| (By XRPD) | dried at 50° C. for 21 h: | crystallinity | crystallinity | crystallinity |
| | low crystallinity | | | |
| Melting onset | Endothermic: 32.7° C., | Endothermic: 39.0° C., | // | // |
| (DSC, ° C.) | 128.0° C., 213.1° C. | 192.1° C., 224.6° C. | | |
| | dried at 50° C. for 21 h: | | | |
| | endothermic: 32.5° C., | | | |
| | 112.2° C. | | | |
| | 217.8° C. | | | |
| Enthalpy | Endothermic: 63, 10, 26 | Endothermic: 55, 72, 41 | // | // |
| (DSC, J/g) | dried at 50° C. for 21 h: | | | |
| | endothermic: 33, 7, 7 | | | |
| Weight loss | 2.4%@85.5° C., | 1.4%@100° C. | // | // |
| (TGA, %) | 1.4%@180° C. | | | |
| Stoichiometric | free form:counter- | free form:counter- | free form:counter- | // |
| ratio | ion = 1:0.6 | ion = 1:1.2 | ion = 1:0.94 | |
| (by ¹H-NMR) | dried at 50° C. for 21 h: | | | |
| | free form:counter- | | | |
| | ion = 1:0.6 | | | |
| Residual solvent | 3.2% MeOH by weight | 0.7% MTBE | 3.0% acetone | 2.1% ACN |
| (by ¹H-NMR) | (0.8 equiv.) | by weight | by weight | by weight |
| | dried at 50° C. for 21 h: | (0.06 equiv.) | (0.5 equiv.) | (0.32 equiv.) |
| | no solvent residue | | | |

### 5.5 Preparation of Salt Candidates

I-3 Form A and I-4 Form A were scaled up for further evaluation.

| Salts | Procedure and observations |
|---|---|
| I-3 Form A | 1. 1000 mg of I-1 Form A was weighed into a 40 mL glass vial |
| | and 3 mL acetone was added into the vial under stirring at |
| | 50° C. for about 5 min with protection from light by covering |
| | aluminum foil. (suspension); |
| | 2. About 10 mg seeds were added to the solution and kept |
| | stirring at 50° C. for about 2 min with protection from light by |
| | covering aluminum foil. (suspension); |
| | 3. About 1.0 equiv. p-toluene sulfonic acid (about 334 mg) was |
| | dissolved in acetone first and then added the counter-ion in |
| | the suspension solution slowly with protection from light by |
| | covering aluminum foil. (suspension). |
| | 4. The mixtures were kept stirring at 50° C. for about 2 hours |
| | with protection from light by covering aluminum foil. |
| | (suspension); |
| | 5. Then cooled naturally to 25° C. and kept stirring for about 5 |
| | days with protection from light by covering aluminum foil. |
| | (suspension); |
| | 6. The solids were collected by centrifugal filtration and dried |
| | at ambient condition for about 12 h; |
| | 7. 1014.53 mg I-3 Form A was obtained as an off-white solid in |
| | a yield of 76%. |
| I-4 Form A | 1. 300 mg of I-1 Form A was weighed into a 20 mL glass vial |
| | and 1.4604 mL acetone was added into the vial under stirring |
| | at 50° C. for about 5 min with protection from light by |
| | covering aluminum foil. (suspension); |
| | 2. About 0.3396 mL methanesulfonic acid (1.0 equiv. by molar |
| | ratio which was diluted with acetone first) was added in the |
| | suspension solution slowly with protection from light by |
| | covering aluminum foil. (suspension); |
| | 3. The mixtures were kept stirring at 50° C. for about 2 hours |
| | with protection from light by covering aluminum foil |
| | (suspension); |
| | 4. Then cooled naturally to 25° C. and kept stirring for about |
| | 0.5 hour with protection from light by covering aluminum |
| | foil (suspension); |
| | 5. Then filtered the solution by 0.45 μm nylon filter to obtain |
| | clear solution; |
| | 6. About 5 mg seeds were added to the solution; |
| | 7. Anti-solvent MTBE (anti-solvent: good solvent = 4:1, v/v) |
| | was added into the solution and kept stirring at 25° C. for |
| | about 6 days with protection from light by covering |
| | aluminum foil (suspension), monitor the samples by XRPD |
| | and chiral purity; |
| | 8. The solids were collected by centrifugal filtration and dried |
| | at ambient condition for about 12 h and then dried at 50° C. in |
| | a vacuum drier for about 12 h; |
| | 9. I-4 Form A was obtained as an off-white solid. |
| I-4 Form A (successful) | 1. 700 mg of I-1 Form A was weighed into a 40 mL glass vial |
| | and 2.409 mL acetone was added into the vial under stirring |
| | at 50° C. for about 5 min with protection from light by |
| | covering aluminum foil. (suspension); |
| | 2. About 0.79117 mL methanesulfonic acid (1.0 equiv. by molar |
| | ratio which was diluted with acetone first) was added in the |
| | suspension solution slowly with protection from light by |
| | covering aluminum foil.(suspension); |
| | 3. The mixtures were kept stirring at 50° C. for about 2 hours |
| | with protection from light by covering aluminum foil |
| | (suspension); |
| | 4. Then cooled naturally to 25° C. and kept stirring for about |
| | 0.5 hour with protection from light by covering aluminum |
| | foil (suspension); |
| | 5. Then filtered the solution by 0.45 μm nylon filter to obtain |
| | clear solution; |
| | 6. About 10 mg seeds were added to the clear solution; |
| | 7. Anti-solvent MTBE (anti-solvent: good solvent = 4:1, v/v) |
| | was added into the solution and kept stirring at 25° C. for |
| | about 3 days with protection from light by covering |
| | aluminum foil (suspension), monitor the samples by XRPD |
| | and Chiral purity; |
| | 8. The solids were collected by centrifugal filtration and dried |
| | at ambient condition for about 12 h and then dried at 50° C. in |
| | a vacuum drier for about 3 h; |
| | 9. 541.34 mg I-4 Form A was obtained as an off-white solid in |
| | a yield of 66%. |
| I-3 Form A | 1. 1000 mg of I-1 Form A was weighed into a 40 mL glass vial |
| | and 3 mL acetone was added into the vial under stirring at |
| | 50° C. for about 5 mins with protection from light by covering |
| | aluminum foil. (suspension); |
| | 2. About 10 mg seeds were added to the solution and kept |
| | stirring at 50° C. for about 2 mins with protection from light |
| | by covering aluminum foil. (suspension); |
| | 3. About 1.0 equiv. p-toluene sulfonic acid (about 334 mg) was |
| | dissolved in acetone first and then added the counter-ion in |
| | the suspension solution slowly with protection from light by |
| | covering aluminum foil. (suspension). |
| | 4. The mixtures(9 mL) were kept stirring at 50° C. for about |
| | 2 hours with protection from light by covering aluminum foil. |
| | (suspension); |
| | 5. Then cooled naturally to 25° C. and kept stirring for about |
| | 20 hs with protection from light by covering aluminum foil. |
| | (suspension); |
| | 6. The solids were collected by centrifugal filtration and dried |
| | at 50° C. by vacuum for about 2 h; |
| | 7. 1097.92 mg tosylate salt was obtained as an off-white solid |
| | in a yield of 83.6%. |
| I-3 Form A | 1. 12 g of I-1 Form A was weighed into a 100 mL glass vial of |
| | EasyMax and 70 mL acetone was added into the vial under |
| | stirring at 50° C. for about 5 mins with protection from light |
| | by covering aluminum foil. (suspension); |
| | 2. About 100 mg seeds were added to the solution and kept |
| | stirring at 50° C. for about 5 mins with protection from light |
| | by covering aluminum foil. (suspension); |
| | 3. About 1.0 equiv. p-toluene sulfonic acid monohydrate(about |
| | 3996.07 mg) was dissolved in 30 mL acetone first and then |
| | added the counter-ion in the suspension solution slowly with |
| | protection from light by covering aluminum foil. |
| | (suspension). |
| | 4. Then 25 mL acetone was added into the mixtures. The |
| | mixtures were stirred at 50° C. for about 1.5 h. Obtained I-3 |
| | Form A was added to the suspension. Additional 25 mL |
| | acetone was added to maintain suspension. |
| | 5. The mixtures (150 mL) were kept stirring at 50° C. for about |
| | 2 hours with protection from light by covering aluminum foil. |
| | (suspension); |
| | 6. Then cooled to 25° C. by 0.1° C./min and kept stirring for about |
| | 18 hs with protection from light by covering aluminum foil. |
| | (suspension); |
| | 7. The solids were collected by centrifugal filtration and dried |
| | at 50° C. by vacuum for at least 36 hs; |
| | 8. 14.7 g I-3 Form A was obtained as an off-white solid in a |
| | yield of 87.0%. |

### 5.6 Salt Candidates Evaluation

### Chemical and Physicochemical Properties

| | Physical form | | | |
|---|---|---|---|---|
| | | I-3 Form A | | I-4 Form A |
| Parameter | I-1 Form A | (anhydrate) | I-4 Form A | (hydrate) |

Initial chiral purity by HPLC

| | | | | |
|---|---|---|---|---|
| Chiral purity | 99% | 98.6% | stirred for 1.5 h: | 100% |
| [area %] | | | 99.7% | |
| | | | stirred for 24 h: | |
| | | | 99.5% | |
| | | | stirred for about | |
| | | | 3 d, 4 d, 5 d: 99.5% | |

Stoichiometry by ¹H-NMR

| | | | |
|---|---|---|---|
| // | free form:counter- | 5 d: free form: | free form:counter- |
| | ion = 1:1 | counter- | ion = 1:1.1 |
| | | ion = 1:1.3 | |
| | | 6 d: free form: | |
| | | counter- | |
| | | ion = 1:1.1 | |

Residual solvent(s) by ¹H-NMR

| | | | | |
|---|---|---|---|---|
| Weight (%) | No residual | 0.2% acetone by | 5 d: no residual | no residual solvent |
| | solvent | weight (0.03equiv) | solvent | |
| | | | 6 d: no residual | |
| | | | solvent | |

Water content by Karl Fisher

| | | | | |
|---|---|---|---|---|
| Weight (%) | // | // | // | 2.5% water by |
| | | | | weight (0.86 |
| | | | | equiv) |

Crystallinity by XRPD

| | | | | |
|---|---|---|---|---|
| High/medium/low | High | High crystallinity | 5 d: low | Medium |
| | crystallinity | | crystallinity | crystallinity |
| | | | 6 d: medium | |
| | | | crystallinity | |

DSC, heating rate [10° C./min]

| | | | | |
|---|---|---|---|---|
| Melting onset (° C.) | Exothermic: | 251.2° C. | 6 d: endothermic: | Endothermic: |
| | 255.9° C., | | 107.3° C., 193.8° C. | 108.0° C., 197.8° C. |
| | 267.7° C.; | | | |
| | endothermic: | | | |
| | 279.4° C. | | | |
| Melting enthalpy | Exothermic: | Decomposition | 6 d: endothermic: | Endothermic: 2, 30 |
| (J/g) | 7, 3; | occurred upon | 36, 51 | |
| | endothermic: | melting | | |
| | 89 | | | |

Themogravimetry, heating rate [10° C./min]

| | | | | |
|---|---|---|---|---|
| Weight loss in | 1.6% @ | 0.8% @214° C. | 6 d: 1.2%@ | 1.5% @ 100° C. |
| (%) @ (° C.) | 250° C. | | 172° C. | |

Morphology

| | | | | |
|---|---|---|---|---|
| PLM | Rod-like | Aggregated fine | // | Aggregated fine |
| | crystals | particles | | particles |

### Bulk Stability

I-1 Form A and the 2 salts were placed at 25° C./92% RH in an open container, at 40° C./75% RH in an open container and at 60° C. in a closed container for I week. Samples after I week were characterized by XRPD and HPLC and inspected for color change.

| | Physical form | | | | | |
|---|---|---|---|---|---|---|
| Parameter | I-1 Form A | | I-3 Form A | | I-4 Form A | |
| Initial purity | 99.0% | | 99.5% | | 99.6% | |
| by HPLC | | | | | | |
| Chiral purity | 99.0% | | 98.6% | | 99.5% | |
| | purity/chiral | CL | purity/chiral | CL | purity/chiral | CL |
| | purity | | purity | | purity | |

Solid state, 25° C./92% RH, open container, 1 week

| | | | | | | |
|---|---|---|---|---|---|---|
| Bulk (HPLC) | 99.0%/ | A | 99.4%/ | A | 99.4%/ | A |
| | 98.9% | | 98.3% | | 98.3% | |

| | | | |
|---|---|---|---|
| Bulk (XRPD) | No form change | No form change* | Form changed |

Solid state, 40° C./75% RH, open container, 1 week

| | | | | | | |
|---|---|---|---|---|---|---|
| Bulk (HPLC) | 99.0%/ | A | 99.3%/ | A | 99.0%/ | A |
| | 98.9% | | 98.5% | | 99.1% | |

| | | | |
|---|---|---|---|
| Bulk (XRPD) | No form change | No form change* | Form changed |

Solid state, 60° C., tight container, 1 week

| | | | | | | |
|---|---|---|---|---|---|---|
| Bulk (HPLC) | 99.0%/ | A | 99.5%/ | A | 99.5%/ | A |
| | 98.9% | | 98.3% | | 99.2% | |

| | | | |
|---|---|---|---|
| Bulk (XRPD) | No form change | No form change | No form change |

— Test not performed
A No change of color
B Slight discoloration
C Medium discoloration
D Strong discoloration
DC complete decomposition
“*”relative humidity was about 40% RH during XRPD test

### Solubility Study

Accurate 4 mg of I-1 Form A, 5.13 mg of I-3 Form A, 4.63 mg of I-4 Form A was weighed into a 8 mL glass vial, respectively. 2 mL of solubility medium was added. The salt amount used is equivalent to 4 mg anhydrous free form. Obtained suspensions/solutions were stirred at 37° C. at 400 rpm for 2 hours and 24 hours and then centrifuged at 37° C. at 14,000 rpm for 5 min. Supernatants were analyzed by UPLC and PH meter for solubility and pH value, respectively. Residual solids (wet cakes) were characterized by XRPD to determine physical form and then analyzed by HPLC for chiral purity.

Solubility study at 37° C., target concentration 2 mg/mL, equilibration for 2 hours and 24 hours, LOQ: 0.5 μg/mL

| | | I-1 Form A | | I-3 Form A | | I-4 Form A | |
|---|---|---|---|---|---|---|---|
| Exp. ID | Media | Solubility (pH) | XRPD | Solubility (pH) | XRPD | Solubility (pH) | XRPD |

| | |
|---|---|
| ES1 | MeOH/DCM (1:2, v/v) |

| 2 hours | >145 | mg/mL | clear | >75 | mg/mL | clear | >111 | mg/mL | clear |
|---|---|---|---|---|---|---|---|---|---|
| | | | solution | | | solution | | | solution |
| 24 hours | >145 | mg/mL | clear | >75 | mg/mL | clear | >111 | mg/mL | clear |

| | | |
|---|---|---|
| solution | solution | solution |

| | |
|---|---|
| ES2 | pH 2, HCl |

| | | | | | | |
|---|---|---|---|---|---|---|
| 2 hours | <LOQ | // | <LOQ | // | <LOQ | // |
| 24 hours | <LOQ (pH: 2.03) | I-1 | <LOQ (pH: 1.86) | I-1 | <LOQ (pH: 1.89) | I-1 |

| | | |
|---|---|---|
| Form A | Form A | Form A + |
| (solids - | (solids - | NaCl |
| chiral | chiral | (solids - |
| purity: | purity: | chiral |
| 98.9%) | 98.3%) | purity: |
| | | 99.3%) |

| | |
|---|---|
| ES3 | Water |

| | | | | | | |
|---|---|---|---|---|---|---|
| 2 hours | <LOQ | // | <LOQ | // | <LOQ | // |
| 24 hours | <LOQ (pH: 7.50) | I-1 | <LOQ (pH: 2.54) | I-1 | <LOQ (pH: 2.52) | I-1 |

| | | |
|---|---|---|
| Form A | Form A | Form A |
| (solids - | (solids - | (solids - |
| chiral | chiral | chiral |
| purity: | purity: | purity: |
| 98.9%) | 98.3%) | 99.2%) |

| | |
|---|---|
| ES4 | SGF, pH 2.0 |

| | | | | | | | | | |
|---|---|---|---|---|---|---|---|---|---|
| 2 hours | 11.2 | μg/mL | // | 4.2 | μg/mL | // | 3.2 | μg/mL | // |

| | | | | | | |
|---|---|---|---|---|---|---|
| 24 hours | 6.4 μg/mL (pH: 2.04) | I-1 | 1.3 μg/mL (pH: 1.92) | I-1 | 2.6 μg/mL (pH: 1.89) | I-1 |

| | | |
|---|---|---|
| Form A | Form A | Form A |
| (solids - | (solids - | (solids - |
| chiral | chiral | chiral |
| purity: | purity: | purity: |
| 98.9%) | 98.3%) | 99.2%) |

| | |
|---|---|
| ES5 | FeSSIF-V1, pH 5.0 |

| | | | | | | | |
|---|---|---|---|---|---|---|---|
| 2 hours | 20.5 | μg/mL | // | 176.1 μg/mL(chiral | // | 239.1 μg/mL(chiral | // |

| | | | purity: 97.1%) | | purity: 97.7%) | |
|---|---|---|---|---|---|---|
| 24 hours | 40.6 μg/mL (pH: 4.87) | I-1 | 19.0 μg/mL (pH: 4.86) | I-1 | 16.2 μg/mL (pH: 4.84) | I-1 |

| | | |
|---|---|---|
| Form A + | Form A + | Form A + |
| 2theta | NaCl | NaCl |
| 8.9° + | (solids - | (solids - |
| NaCl | chiral | chiral |
| (solids- | purity: | purity: |
| chiral | 66.4%) | 85.2%) |
| purity: | | |
| 85.7%) | | |

| | |
|---|---|
| ES6 | FaSSIF-V1, pH 6.5 |

| 2 hours | 4.5 μg/mL(chiral | // | 52.2 μg/mL (chiral | // | 44.1 μg/mL(chiral | // |
|---|---|---|---|---|---|---|
| | purity: 97.7%) | | purity: 96.1%) | | purity: 98.3%) | |
| 24 hours | 11.9 μg/mL (pH: 6.45) | I-1 | 0.7 μg/mL (pH: 6.17) | I-1 | 0.4 μg/mL (pH: 6.15) | I-1 |
| | (chiral purity: 97.7%) | Form A + | | Form A + | | Form A + |
| | | NaCl | | NaCl | | NaCl |
| | | (solids - | | (solids - | | (solids - |
| | | chiral | | chiral | | chiral |
| | | purity: | | purity: | | purity: |
| | | 98.9%) | | 98.4%) | | 99.3%) |

### Suspension Stability

Accurate 10 mg of I-1 Form A, 12.83 mg of I-3 Form A, 11.58 mg of I-4 Form A was weighed into a 8 mL glass vial, respectively. 5 mL of suspension vehicle was added, respectively. The salt amount used is equivalent to 10 mg anhydrous free form. These suspensions were stirred at 25° C. with 400 rpm. These suspensions were taken out at 2 hours and 24 hours, then centrifuged at 14,000 rpm for 5 min. The supernatants were analyzed by HPLC and pH meter. Solids obtained (wet cakes) were characterized by XRPD and HPLC to determine the chiral purity. Meanwhile, a part of suspension were withdrawn and dissolved by diluent (ACN:H₂O=1:1, v/v) to get a clear solution. Obtained clear solution was analyzed by HPLC to determine the chemical purity.

Accurate 49.2 mg of I-3 Form A, 46.3 mg of I-4 Form A was weighed into a 8 mL glass vial, respectively. 4 mL of solubility medium was added, respectively. The salt amount used is equivalent to 40 mg anhydrous free form. 4 mL of the aqueous vehicle (0.5% (w/w) MC (400 cP) and 2% (w/w) Vitamin E TPGS in water) was added, respectively. These suspensions were stirred at 25° C. with 400 rpm. These suspensions were taken out at 2 hours and 24 hours, then centrifuged at 14,000 rpm for 5 min. The supernatants were analyzed by HPLC for the solubility and chiral purity and analyzed by pH meter. Solids obtained (wet cakes) were characterized by XRPD and HPLC for chiral purity. Meanwhile, a part of suspension were withdrawn and dissolved by diluent (ACN:H₂O=1:1, v/v) to get a clear solution. Obtained clear solution was analyzed by HPLC to determine the chemical purity.

| | I-1 Form A | | | | | I-3 Form A | | | | | I-4 Form A | | | | |
|---|---|---|---|---|---|---|---|---|---|---|---|---|---|---|---|
| | Initial chiral purity: 98.9% | | | | | Initial chiral purity: 98.6% | | | | | Initial chiral purity: 99.5% | | | | |
| | | | Solu- | Chiral | | | | Solu- | Chiral | | | | Solu- | Chiral | |
| Exp. | | | bility | purity | | | | bility | purity | | | | bility | purity | |
| ID | Purity | pH | (μg/mL) | (solids) | XRPD | Purity | pH | (μg/mL) | (solids) | XRPD | Purity | pH | (μg/mL) | (solids) | XRPD |

Suspension stability at 25° C., target concentration 2 mg/mL, equilibration for 2 hours and 24 hours, LOQ: 0.25 μg/mL

| SUS1 | Pure water | | | | | | | | | | | | | | |
|---|---|---|---|---|---|---|---|---|---|---|---|---|---|---|---|
| 2 h | 99.1% | 6.79 | <LOQ | 98.8% | No form | 99.6% | 5.59 | <LOQ | 98.3% | No form | 99.5% | 2.81 | <LOQ | 99.7% | Amor- |
| | | | | | change | | | | | change | | | | | phous |
| | | | | | | | | | | | | | | | form |
| 24 h | 99.2% | 8.27 | <LOQ | 96.7% | No form | 99.5% | 2.85 | <LOQ | 98.3% | I-1 | 99.4% | 2.61 | <LOQ | 99.7% | I-1 |
| | | | | | change | | | | | Form A | | | | | Form A |
| SUS2 | 0.5%(w/w) Tween 80 in water | | | | | | | | | | | | | | |
| 2 h | 99.1% | 6.52 | 25.5 | 98.8% | No form | 99.4% | 3.11 | 244.6 | 98.0% | I-3 | 99.5% | 2.78 | 342.6 | 99.7% | Amor- |
| | chiral | | | | change | chiral | | | | Form A + | | | | | phous |
| | purity: | | | | | purity: | | | | 2 peaks | | | | | form |
| | 83.2% | | | | | 95.8% | | | | | | | | | |
| 24 h | 99.2% | 7.40 | 17.6 | 97.6% | No form | 99.4% | 2.63 | 17.9 | 94.2% | I-1 | 99.6% | 2.63 | 16.3 | 95.8% | I-1 |
| | chiral | | | | change | chiral | | | | Form A | | | | | Form A |
| | purity: | | | | | purity: | | | | | | | | | |
| | 93.0% | | | | | 98.5% | | | | | | | | | |
| SUS3 | 0.5%(w/w) MC(400 cP) and 2%(w/w) Vitamin E TPGS in water | | | | | | | | | | | | | | |
| 2 h | 99.5% | 5.02 | 47.9 | 98.7% | No form | 99.3% | 2.76 | 1220.9 | // | Amor- | 99.2% | 2.72 | 1210.7 | // | Amor- |
| | chiral | | | | change | chiral | | | | phous | | | | | phous |
| | purity: | | | | | purity: | | | | form | | | | | form |
| | 64.8% | | | | | 86.4% | | | | | | | | | |
| 24 h | 99.1% | 4.30 | 34.5 | 96.6% | No form | 99.3% | 2.62 | 1990.7 | // | Amor- | 99.6% | 2.61 | 2018.8 | // | Amor- |
| | chiral | | | | change | chiral | | | | phous | | | | | phous |
| | purity: | | | | | purity: | | | | form | | | | | form |
| | 97.7% | | | | | 98.3% | | | | | | | | | |

Suspension stability at 25° C., target concentration 10 mg/mL, equilibration for 2 hours and 24 hours, LOQ: 1 μg/mL

| SUS4 | 0.5(w/w) MC(400 cP) and 2%(w/w) Vitamin E TPGS in water | | | | | | | | | | | | | | |
|---|---|---|---|---|---|---|---|---|---|---|---|---|---|---|---|
| 2 h | // | // | // | // | // | 99.7% | 2.30 | 3433.2 | Liquid: | Form | 99.7% | 1.89 | 3947.2 | Liquid: | Amor- |
| | | | | | | | | | 99.4%; | changed | | | | 100%; | phous |
| | | | | | | | | | solid: | | | | | solid: | form |
| | | | | | | | | | 97.9% | | | | | 99.8% | |
| 24 h | // | // | // | // | // | 99.7% | 2.17 | 3083.9 | Liquid: | No form | 99.6% | 1.86 | 3813.0 | Liquid: | amor- |
| | | | | | | | | | 99.4%; | change | | | | 99.8%; | phous |
| | | | | | | | | | solid: | | | | | solid: | form |
| | | | | | | | | | 98.3% | | | | | 99.7% | |

“//”: Not carried out

### Solution Stability

Accurate 4 mg of I-1 Form A, 5.13 mg of I-3 Form A was weighed into a 8 mL glass vial, respectively. The salt amount used is equivalent to 4 mg anhydrous free form. 1 mL of different solvents was added respectively and clear solutions were obtained. The clear solutions were stirred at 25° C. with 400 rpm. The clear solutions were taken out at Oh, 6 h and 24 h, then diluent by ACN:H₂O=1:1, v/v and were analyzed by HPLC to determine the chemical purity and chiral purity.

| | I-1 Form A | | I-3 Form A | |
|---|---|---|---|---|
| | Initial chiral purity: 99.0% | | Initial chiral purity: 98.6% | |
| | Chemical | Chiral | Chemical | Chiral |
| Exp. ID | purity | purity | purity | purity |
| SOS1 | MeOH | | | |
| 0 h | 98.9% | 98.9% | 99.3% | 98.3% |
| 6 h | 99.0% | 98.9% | 99.3% | 98.3% |
| 24 h | 99.1% | 98.9% | 99.4% | 98.3% |
| SOS2 | THF/ACN(2:1, v/v) | | | |
| 0 h | 98.6% | 98.9% | 99.2% | 98.3% |
| 6 h | 98.7% | 98.9% | 99.3% | 98.3% |
| 24 h | 99.0% | 98.9% | 99.4% | 98.3% |

### Hygroscopicity

Water sorption and desorption behavior of I-1 Form A, I-3 Form A, and I-4 Form A was investigated by DVS at 25° C. with a cycle of 40-0-95-0-40% RH, dm/dt 0.002, min. equilibration time 60 min and max. equilibration time 360 min. XRPD was measured after the DVS test to determine form change.

| | I-1 Form A (anhydrate) | | | | I-3 Form A (anhydrate) | | | | I-4 Form A (hydrate) | | | |
|---|---|---|---|---|---|---|---|---|---|---|---|---|
| | 1^{st} | 1^{st} | 2^{nd} | 2^{nd} | 1^{st} | 1^{st} | 2^{nd} | 2^{nd} | 1^{st} | 1^{st} | 2^{nd} | 2^{nd} |
| | Sorp. | Desorp. | Sorp· | Desorp. | Sorp. | Desorp. | Sorp· | Desorp. | Sorp. | Desorp. | Sorp· | Desorp. |
| | (%) | (%) | (%) | (%) | (%) | (%) | (%) | (%) | (%) | (%) | (%) | (%) |
| 0% | 0.17 | 0.17 | 0.001 | 0.001 | 0.05 | 0.05 | 0.00 | 0.00 | // | 0.01 | 0.01 | // |
| 10% | 0.23 | 0.25 | 0.08 | 0.11 | 0.15 | 0.16 | 0.10 | 0.11 | // | 1.52 | 0.98 | // |
| 20% | 0.29 | 0.33 | 0.15 | 0.21 | 0.26 | 0.27 | 0.21 | 0.22 | // | 2.78 | 1.76 | // |
| 30% | 0.35 | 0.42 | 0.22 | 0.33 | 0.38 | 0.40 | 0.35 | 0.37 | // | 3.61 | 2.64 | // |
| 40% | 0.42 | 0.52 | 0.32 | 0.51 | 0.67 | 0.69 | 0.81 | 2.11 | 1.62 | 4.52 | 3.57 | 4.51 |
| 50% | 0.54 | // | // | 0.92 | 2.34 | // | // | 2.44 | 3.50 | 5.78 | 4.63 | 5.71 |
| 60% | 0.71 | // | // | 1.30 | 2.53 | // | // | 2.64 | 4.75 | 7.60 | 6.22 | 7.44 |
| 70% | 1.02 | // | // | 1.62 | 2.84 | // | // | 4.17 | 6.87 | 11.14 | 9.34 | 10.54 |
| 80% | 1.84 | // | // | 2.15 | 4.29 | // | // | 4.45 | 12.16 | 16.84 | 15.03 | 17.11 |
| 90% | 3.24 | // | // | 3.56 | 4.64 | // | // | 4.76 | 25.12 | 31.04 | 27.39 | 32.94 |
| 95% | 5.90 | // | // | 5.90 | 5.36 | // | // | 5.36 | 42.35 | 42.35 | 44.74 | 44.74 |

| XRPD | No form and | Obtained sample | Form changed |
|---|---|---|---|
| after | crystallinity change | is still Form A | |
| DVS | | | |
| test | | | |

“//”: Not carried out

### Investigation of I-3 Form A in Different Humidity

I-3 Form A was used as starting material. One RH cycle was applied at 25° C. XRPD analysis was carried out in each specific relative humidity. Cycle: 39% RH (initial)-10% RH (2h)-40% RH (2h)-50% RH (2h)-70% RH (2h). Meanwhile, I-3 Form A was exposed to 30% RH, 40% RH and 50% RH chamber for 1 week. Samples were analyzed by XRPD using air tight container.

| | Relative humidity at 25° C. | | |
|---|---|---|---|
| | (equilibration time) | XRPD | Comments |
| VH- | 39% (initial) | I-3 Form A | No comments |
| XRPD | 10% (2 h) | I-3 Form A | No comments |
| | 40% (2 h) | I-3 Form A | No comments |
| | 50% (2 h) | I-3 Form A + 2Theta 17.3° | No comments |
| | 70% (2 h) | I-3 Form B | No comments |
| Chamber | 30% (1 week) | I-3 Form A + 2Theta 17.5° | Air tight |
| | | | container was |
| | | | used for XRPD |
| | | | test. |
| | 40% (1 week) | I-3 Form B | Air tight |
| | | | container was |
| | | | used for XRPD |
| | | | test. |
| | 50% (1 week) | I-3 Form B | Air tight |
| | | | container was |
| | | | used for XRPD |
| | | | test. |

### Salt Candidates Risk Matrix

| | Counter | Chemical | Physical | | | |
|---|---|---|---|---|---|---|
| | ion | stability | stability | Hygroscopicity | Solubility | Polymorphism |
| I-1 Form | // | Stable | Stable | Hygroscopic; no | pH- | At least 3 |
| A | | | | form and | dependent | polymorphs, |
| | | | | crystallinity | solubility | and |
| | | | | change after the | profile | racemization |
| | | | | DVS test | | easily occurred. |
| I-3 Form | Class II | Stable | Stable | Sensitive to | pH- | At least two |
| A | | | | moisture and | dependent | polymorphs. |
| | | | | convert to | solubility | |
| | | | | potential hydrate | profile | |
| | | | | in high humidity. | | |
| I-3 Form | Class II | Stable | Stable | Very | pH- | One polymorph |
| A | | | | hygroscopic; | dependent | identified so far. |
| | | | | after the DVS | solubility | |
| | | | | test, form | profile | |
| | | | | changed. | | |

### Examples 1-A, 2-A, 3-A, and 4-A

The compounds described herein can be prepared in a number of ways based on the teachings contained herein and synthetic procedures known in the art. The following non-limiting examples illustrate the disclosures herein.

| Acronyms | Full name |
|---|---|
| MeOH | Methanol |
| EtOH | Ethanol |
| ACN | Acetonitrile |
| TFA | Trifluoroacetic acid |
| DMSO | Dimethyl sulfoxide |
| IPAc | Isopropyl acetate |
| DCM | Dichloromethane |
| EA | Ethyl acetate |
| THF | Tetrahydrofuran |
| MTBE | Methyl tert-butyl ether |

### Example 1-A. Preparation of Compounds I-1 and II-1 Separation of III-1 Mini Simulated Moving Bed (SMB) Demonstration

### Introduction

The separation of the enantiomers of III-1 was screened and the best separation conditions identified were Chiralpak IH with DCM/MeOH 90/10 v/v as mobile phase. The estimated productivity is 3.7 kg/day/kg of CSP. A 200 g sample of the racemic feed was separated on the mini SMB to demonstrate the separation and confirm the estimated productivity and to obtain the two enantiomers for additional R&D work.

### Process Preparation

### Process Description

The separation of racemic III-1 to obtain each enantiomer was demonstrated using Chiralpak IH, 20 μm, as the stationary phase and n-heptane/DCM 90/10 v/v as the mobile phase. The SMB unit is equipped with 8 columns of 10 cm in length and 1 cm in diameter. The enantiomers are separated into two process streams, the raffinate (II-1) and the extract (I-1).

The 10 mm unit was set up to operate in an 8-column mode using a 2-2-2-2 columns per zone configuration to mimic the larger units. The SMB was operated at ambient temperature (˜22° C.).

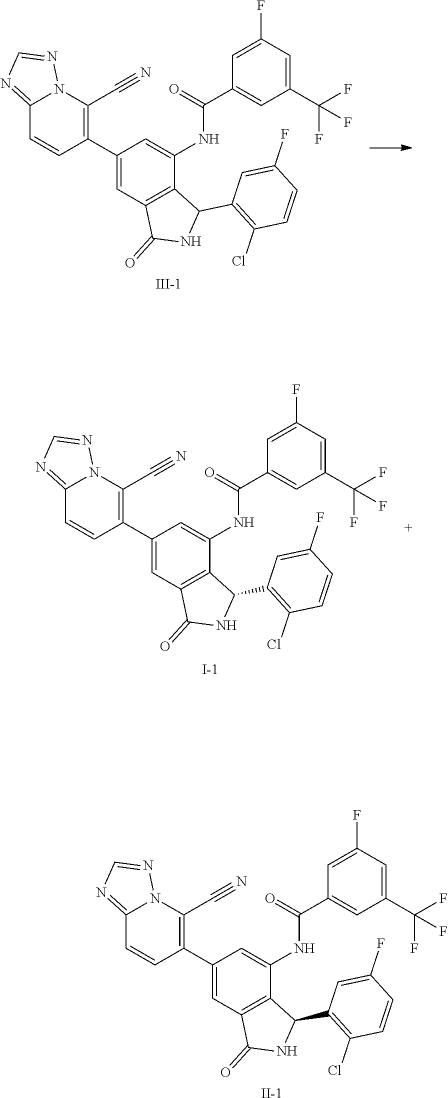

### Order of Elution

A dilute solution (1.75 g/l) of the reference standard was prepared and compared to a dilute solution of the feed to confirm the order of elution. I-1 elutes last and therefore will be recovered in the extract stream.

### Column Packing and Testing

An existing set of Chiralpak IH columns were tested with a 1.07 g/l feed sample at 1.9 ml/min. The columns were tested again at the completion of the run.

### Column Testing Before and After the Campaign

| Col | tR0 | tR1 | tR2 | N1 | N2 | A1 | A2 | a |
|---|---|---|---|---|---|---|---|---|
| Before | 2.320 | 2.750 | 5.080 | 426 | 158 | 1.71 | 1.06 | 6.42 |
| After | 2.440 | 2.745 | 5.229 | 411 | 155 | 1.65 | 1.04 | 9.14 |

With
t0: Retention time of the solvent indicative of the dead volume”
tRi: Retention time of enantiomer “i”
Ni: Number of plates (efficiency) for the peak “i”
Ai: Asymmetry factor for the peak “i”
Selectivity: Separation selectivity defined as a=(tR2−t0)/(tR1−t0)

The testing shows little difference between the columns before and after the campaign. The efficiency average remained good as well as the peaks asymmetries. A difference in retention time for the second peak (tR2) translates in a significant increase in selectivity. This could be the result of a small composition change in the mobile phase or the separation temperature between the two sets of experiment. Considering that the separation was stable during the demonstration run and that the peak shape is relatively unchanged, the difference in retention is not a major concern.

### Specifications

The target chiral purity for the desired enantiomer is greater than 99.0% e.p.

There is no specification for the undesired enantiomer, however, the undesired enantiomer should be recovered at the highest possible purity to maximize the recovery.

### Analytical Methods

### In Process Chiral Method

The chiral purity of the fraction collected from the SMB is measured using the following method. The samples are injected neat from the SMB fractions collected from the extract and raffinate streams.

### HPLC Method Used for Determination of Chiral Purity (In-Process Method)

| Column | Chiralpak IH 0.46 × 15.0 cm, 5 μm | |
|---|---|---|
| Mobile | phase 90/10 DCM/MeOH | |
| Injection | 10 | μL |
| Detection | 350 | nm |
| Temperature | 25° | C. |
| Total run time | 5 | min |

### Feed Preparation

A feed solution was prepared using the racemic feed provided.

### Solubility Comment

The feed solutions used for the loading study precipitated slightly while standing at room temperature at 25-26 g/l. Therefore, to eliminate the precipitation of the product the target concentration was reduced to 20 g/l and then again to 18 g/l as more solids precipitation was observed.

The solubility at 18 g/l was very sensitive to the temperature in the lab therefore the feed solution temperature should be controlled.

### Feed Lots for Separation

A total of ˜196.8 g of racemic feed material was processed during this run.

### Feed Solutions Prepared and Processed

| Feed | Net weight | Concentration (g/l) |
|---|---|---|
| 1 | 20.0 | 20.1 |
| 2 | 18.0 | 17.9 |
| 3 | 18.0 | 17.7 |
| 4 | 18.2 | 18.3 |
| 5 | 18.0 | 17.6 |
| 6 | 18.1 | 17.7 |
| 7 | 18.0 | 17.2 |
| 8 | 18.0 | 17.6 |
| 9 | 18.0 | 17.6 |
| 10 | 18.2 | 17.9 |
| 11 | 14.3 | 18 |
| Total | 196.8 | |

Approximately 3.8 grams was recovered as solution as unprocessed feeds.

### SMB Separation

### Eluent Run

The SMB is first started with eluent in lieu of feed to flush the equipment and condition the columns. This allows for verification that the operating pressure is within expected range as the equipment is limited to 18-20 bars operating pressure. Once the flow rates are stable, the system is stopped and put on feed. The cycle counter is then reset and a “B” designation is added as a suffix.

### Separation Optimization

The separation is started using a modified version of the parameters estimated from the loading study performed in the screening phase of the project. Typically, the feed rate is reduced from the optimum calculated by the modeling to start conservatively and ensure both purity and recovery.

### SMB Parameters

The separation purity was excellent right from the start and the pressure held steady at 15 bars. These parameters correspond to a productivity of 2.3 kgfeed/day/kg CSP which is below the optimum predicted by the model.

SMB Parameters. Flow Rates in ml/min and Time in Min.

| | | | | | | | Chiral | |
|---|---|---|---|---|---|---|---|---|
| | | | | | | | Purity (%) | |
| Run (Cycle#) | Zone 1 | Ex | F | Raf | El | Period | Ex | Raf |
| Start (6B) | 7.0 | 3.4 | 1.5 | 2.1 | 4.0 | 1.56 | 99.9 | 100.0 |
| Cycle 46B | 7.0 | 3.4 | 1.5 | 2.5 | 4.4 | 1.56 | 99.8 | 100.0 |
| End (169C) | 9.0 | 5.4 | 2.40 | 3.40 | 6.4 | 1.46 | 99.6 | 99.7 |

The feed rate was gradually increased, and the other parameters adjusted to maintain purity and recovery until the separation reached a maximum (no further increase of feed rate without loss of purity or significant loss of yield). The final set of parameters is reported in the table above.

Note that the feed flow rate was adjusted for the more dilute solution of the feed due to solubility issues (17.9 g/l vs 20.1 g/l). This accounts for the larger initial increase of the feed rate.

### Product Recovery and Shipping

### Product Isolation

The product streams were evaporated to dryness using the conditions in the table below. The products were collected in 4-liter HPLC solvent bottles. Once the contents are charged to the flask and solvent is almost completely evaporated, the vacuum is lowered to 100 mbar to finish drying.

### Rotary Evaporators Operating Conditions

| Evaporator | Vacuum (mbar) | Bath temperature (° C.) |
|---|---|---|
| Extract | 395-100 | 35 |
| Raffinate | 395-100 | 35 |

### Process Mass Balance

A mass balance is performed on the overall process based on the feed processed and the material recovered. This mass balance includes the feed for the campaign (196.8 g). A total of 179.3 g of material was recovered as extract, raffinate or unprocessed feed corresponding to an overall recovery of ˜91%. This is unusually low as an overall recovery of 97%-100% is more typical. This discrepancy could be due to the presence of residual solvent in the starting racemic feed not accounted for. An assay of the racemic feed received was not performed and therefore a fair evaluation of the process yield is not possible.

A total of 57.5 grams of within specification I-1 was recovered. An additional 25 grams of out of specification material was also recovered. If both lots were combined a total yield to 82.1 g at ˜99.4% chiral purity was recovered (i.e. 85%).

A total 93 g of raffinate at an average 96.8% e.p. was recovered corresponding to a raffinate yield of 93.3%.

The raffinate contains 2.94 g of product (extract) corresponding to a yield loss of ˜3%.

The yield calculation assumes that the product assay is 100%, therefore 50% of the mass is the desired enantiomer. However, the quantity of residual solvent in the racemic feed is unknown. In addition, two impurities are visible in the racemic feed by chiral chromatography. The response factor of these impurities is unknown and therefore the contribution of these impurities to the total racemic feed weight is unknown.

### Conclusion

The chiral separation of the enantiomers of III-1 was successfully performed on the 8×10 mm SMB using columns packed with Chiralpak IH and DCM/methanol 90/10 v/v as the mobile phase. A productivity of 3.5 kg/day/kg CSP was demonstrated with an in-process chiral purity greater than 99.5% which matches the estimations based on the modelling of the separation. The separation was stable, but a slow precipitation of the feed was observed that could be an issue at larger scale.

A net total of 57.5 g of the desired enantiomer was produced within specification. And an additional 25 g at 98.8% e.p. was produced.

### Process Development: Racemization and Isolation

Following the SMB process development, the process for the racemization of II-1 and the process for the isolation of the enantiomers as dry solids were also evaluated. Finally, a sample of the racemic feed to be processed on the kilo lab was submitted to a use-test.

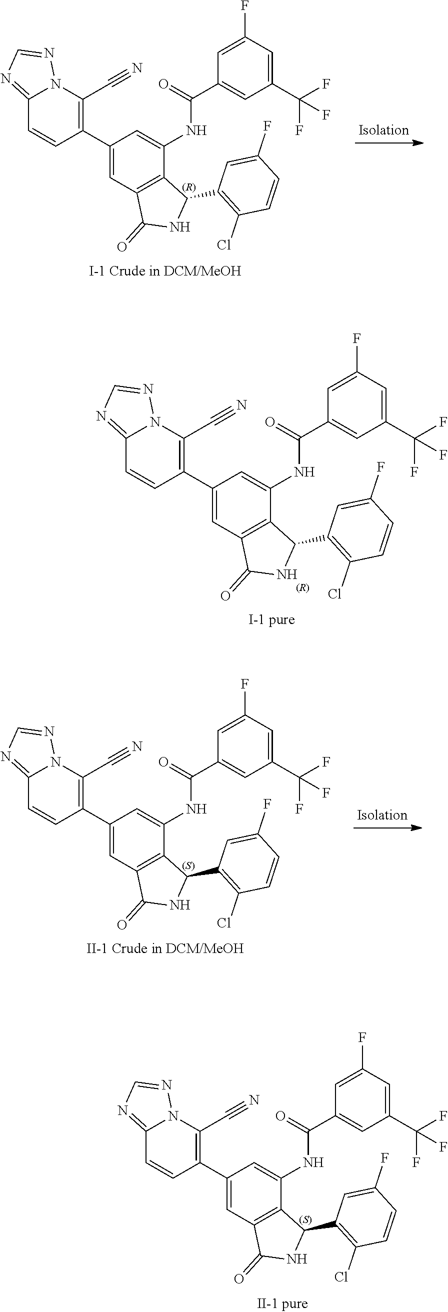

### Process Screening

### Isolation Conditions Screening

Several experiments were performed to develop an isolation process for obtaining the desired enantiomer as a dry solid. The table below summarizes the development effort.

### Experiment Summary for the Crystallizations Screening (Exp XXX)

| Exp# | | | Chiral purity, | Purity, |
|---|---|---|---|---|
| (XXX) | Material | Conditions | area % | area % |
| 005 | I-1 | DCM/MeOH + MTBE | 100 | 99.2 |
| 007 | I-1 | DCM/MeOH + n-heptan | 100 | 99.2 |
| 011 | II-1 | DCM/MeOH + n-heptane 35 → 15° C. | 98.7 | 98.5 |
| 014 | II-1 | Trituration in MeOH/EtOAc = 10:1 | 52.8 | 98.6 |
| 016 | II-1 | MeCN | 52.2 | 98.4 |

### Polymorph Analysis (XRPD)

The resulting crystals were analyzed using XRPD to assess their properties. The different crystallization methods result in different polymorphs.

### Residual Solvent Evaluation

The residual solvents in the crystalline dried materials were evaluated by 1H-NMR. Material of Experiment #-014 seems to be MeOH solvate.

### Residual Solvents in Crystallized Products

| | Wt % in Experiment # | | | | |
|---|---|---|---|---|---|
| Material | 005 | 007 | 011 | 014 | 016 |
| I-1 or II-1 | 99.95% | 99.94% | 99.71% | 95.61% | 99.56% |
| MeOH | 0.00% | 0.00% | 0.00% | 4.18% | 0.06% |
| MeCN | 0.00% | 0.00% | 0.00% | 0.00% | 0.29% |
| EtOAc | 0.00% | 0.00% | 0.00% | 0.21% | 0.09% |
| DCM | 0.02% | 0.06% | 0.24% | 0.00% | 0.00% |
| MTBE | 0.03% | 0.00% | 0.00% | 0.00% | 0.00% |
| n-heptane | 0.00% | 0.00% | 0.04% | 0.00% | 0.00% |

### Larger Scale Isolation Demonstration

A larger scale crystallization procedure with n-heptane as antisolvent was used to obtain I-1 from the SMB separation solution.

### Feed Material

A sample of compound I-1 crude in DCM/MeOH was used as starting material for the demonstration, the composition of the sample is below.

### I-1 Composition by 1H-NMR and HPLC:

Results by NMR

| Concentration | I-1 by HPLC | I-1 | MeOH | DCM |
|---|---|---|---|---|
| Wt % | 14.1 | 14.2 | 14.1 | 71.7 |

### Experimental Procedure

The procedure for the larger scale I-1 crystallization is outlined below

| Step # | Weight (g) | I-1, wt % | Procedure comments |
|---|---|---|---|
| 1 | 99.2 | 14.1 | compound I-1 crude in DCM/MeOH solution charged in 0.5 L |
| | | | reactor |
| | | | M(I-1) = 13.99 g |
| 2 | | | Distillation |
| | | | Tjacket = 50.0° C. |
| | | | Tcondenser = −10.0° C. |
| | | | Ti = 39.5-43.8° C. |
| | | | Stirring = 255 RPM |
| 3 | 42.4 | 33 | Stop distillation. |
| | | | m(distillate) = 56.8 g |
| | | | Tjacket = 40.0° C. |
| | | | Very thick white slurry obtained |
| 4 | 97.4 | 14.4 | n-heptane addition |
| | | | Addition rate = 2 mL/min |
| | | | m(n-heptane) = 55.0 g |
| 5 | | | Cooldown ramp from 40.0° C. to 20.0° C. over 2 hours |
| | | | Stirring = 300 RPM |
| 6 | | | Aging for 16 hours |
| 7 | 97.4 | 0.25 | Sample #2. Quantification of I-1 in solution. |
| | | | m(I-1 in solution)~(97.4 − 14)*0.0025 = 0.21 g |
| 8 | | | Filtration through 20 um filter |
| | | | Filter bed thickness: ~1 cm |
| | | | Filtration time: 2 minutes |
| | | | m(mother liquor) = 49.7 g |
| 9 | | | Slurry wash |
| | | | m(n-heptane) = 76.7 g |
| | | | Wash time - 2 minutes |
| | | | Filtration time - 1 minutes |
| 10 | | | Displacement wash |
| | | | m(n-heptane) = 65.0 g |
| | | | Filtration time - 2 minutes |
| | | | m(combined washes) = 142.2 g |
| | | | No I-1 detected in washes. |
| 11 | | | Drying under vacuum at 40° C. |
| | | | Purity: 99.09% |
| | | | RRT = 0.42 − 0.08% |
| | | | RRT = 0.80 − 0.15% |
| | | | RRT = 0.94 − 0.51% |
| | | | RRT = 1.54 − 0.13% |

### Results

The material produced was analyzed by RP chromatography. The material produced is very clean outside of 1 single impurity at RRT 0.94. This impurity was later identified as a impurity from the racemic feed.

The procedure developed above was tested on a sample of the material separated on the SMB unit.

### I-1 Isolation Line Procedure

1. Set reactor 1 jacket temperature to 25° C. and inert with nitrogen.
2. Charge I-1 solution to ˜⅓ of the reactor volume.
3. Set reactor 1 jacket temperature to 50° C. and start applying vacuum.
   Keep batch temperature≤45° C. during distillation.
   Continuous feed addition of I-1 solution while distilling.
4. Distillation is deemed complete when batch is concentrated to 3.7 S.
5. Set reactor 1 jacket temperature to 40° C. and stop distillation.
6. Charge n-heptane (RM-2049) over NLT 1 hour.
7. Cool the batch down to 20° C. over NLT 2 hours.
8. Age the batch at 20° C. for NLT 2 hours.
9. Transfer the batch on Aurora filter.
   The crystals are very fine and caution should be taken when applying vacuum.
10. Charge 3.5S of n-heptane (RM-2049) to the reactor as a rinse.
11. Transfer the rinse from the reactor to the Aurora filter for slurry wash.
12. Mix the slurry on the filter for NLT 5 min.
13. Apply gentle vacuum to the bottom of the Aurora to deliquor the cake.
14. Charge 3.5S of n-heptane (RM-2049) to the reactor as a rinse.
15. Transfer the rinse from the reactor to the Aurora filter for displacement wash.
16. Apply gentle vacuum to the bottom of the Aurora to deliquor the cake.
17. Heat Aurora jacket to 30° C.
   Dry the cake by passing nitrogen through the cake for NLT 12 hours.
   After 2 hours of drying, start stirring the cake regularly.
18. IPC #1 (drying completion).
19. Package when passing results for IPC #1 are obtained.

### Example 2-A. Racemization Process Development

### 2.1 Process Description

A concentrated solution of II-1 in DCM/Methanol is racemized to obtain III-1.

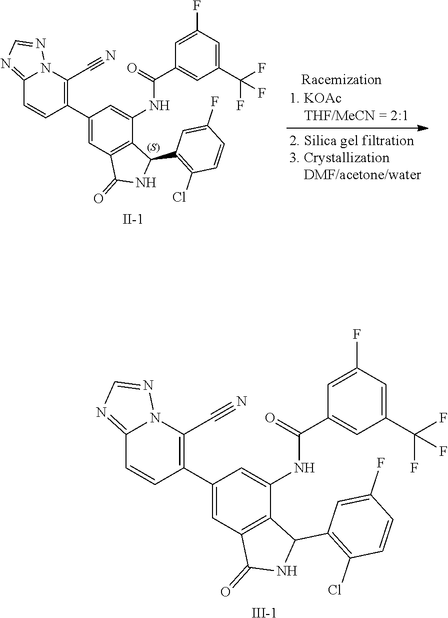

### Racemization Screening

### Racemization Using Basic Resins

The racemization was studied using a variety of basic resins to promote the conversion of the chiral center. Some resins failed to racemize II-1 to noticeable extent, while others generated significant quantities of side products. Potential resins to be used for racemization are:
Dowex 1×2-400 (exp #4).
Dowex marathon MSA in acetate (exp #6)
Amberlite FPC3500 (K) (exp #14)
Diaion WK100 (K) (exp #15).

### Base Induced Racemization

Additional experiments for the racemization of II-1 with KOAc were performed to eliminate the use of resins that cause the generation of impurities.

### Stability Profile

The compound solution was kept at ˜40° C. for 23 hours to evaluate the stability.

### Effect of Temperature on the Racemization

The racemization at 22° C. and 30° C. was performed to evaluate the effect of temperature on the racemization kinetics and impurity profile. The racemization reaction is significantly faster at 30° C. compared to 22° C. However, it also generates higher level of impurities, but this difference is insignificant when compared to the timepoint with similar reaction conversion degree.

### Light and Glassware Influence on Racemization

The effect of glassware surface pH and light on II-1 racemization was evaluated. There is no significant racemization observed over up to 13 days period in neutral and acidic glassware in both absence and presence of light. Noticeable racemization was observed, however, in basic glassware and this racemization was promoted by light.

### Solubility Evaluation

### Solubility in DCM/Methanol Mixtures

The solubility was evaluated on various ratio of DCM and Methanol mixtures. The maximum solubility is achieved with a ratio of 70/30 DCM/MeOH. The racemic material has limited solubility in DCM/MeOH=90:10 mixture (slightly less than desired for SMB separation).

### Solubility in Acetonitrile

Previously, crystallization from Acetonitrile was found as an attractive way for isolation of the racemic material. To select the best conditions for isolation, a small screening of crystallization conditions was performed. Crystallized product purity and residual in mother liquor were evaluated at 20, 10 and 0° C. with 0, 10 and 20 wt % of water.

Water during crystallization of the racemized material improves the impurity profile as the temperature increases and has a limited effect on the recovery (loss to mother liquor).

### Solubility in Acetone/Water Solution

Sequential wash of the II-1 wet cake with Acetone/water/acetone was previously evaluated. However, significant quantities of residual DMF were left on the wet cake and remained in the final dried product. Additionally, wet cake slurried in water was very viscous and difficult to deliquor. To decrease viscosity of the wet cake washes, maximize DMF solvent removal and minimize II-1 product loss, the solubility of II-1 in Acetone/Water mixtures was studied. Based on solubility data, Acetone/Water (60/40 wt/wt) was selected as the wash composition for the wet cake.

### Isolation Post Racemization

For the evaluation of the isolation method post racemization, the reaction mixture was split in two portions.

II-1 crystals were obtained in similar chiral and RP-HPLC purity from both isolation procedures.

### Proposed Racemization and Isolation Process

Since the racemization with KOAc was confirmed, generation of the racemized material on relatively larger scale was performed by utilizing solvent swap/racemization/filtration/crystallization approach. The procedure is described below.

| | Batch weight | II-1 | |
|---|---|---|---|
| Step # | g | wt % | Procedure comments |
| 1 | 219.6 | 20.8 | II-1 solution charged in 0.5 L reactor. |
| | | | m(II-1) = 45.90 g |
| 2 | 99.4 | | Distillation |
| | | | Tjacket = 50.0° C. Tcondenser = −10.0° C. Ti = |
| | | | 40.0-40.2° C. Stirring = 300 RPM m(distillate) = 120.2 g |
| 3 | 116.3 | | Solvent swap. Addition of MeCN in 40-50 g portions. |
| | | | P = 200 mbar Ti = 37.4° C. m(MeCN added) = 280.6 g |
| | | | m(distillate) = 263.7 g |
| 4 | 116.3 | | Sample #2 |
| | | | [MeOH] by 1H-NMR) <100 ppm |
| 5 | 408.4 | 11.2 | Added THF and MeCN. Total solvent in the batch: |
| | | | THF - 250 g (6.2 V) MeCN - 112.5 g (3.1 V) |
| 6 | 408.4 | 11.2 | Sample #3 (NMR): |
| | | | [MeCN] = 29.5 wt % [THF] = 70.5 wt % |
| 7 | 408.4 | | Sample #4 (HPLC assay): |
| | | | [II-1] = 11.2 wt % |
| 8 | 415.8 | | Added AcOK - 7.4 g |
| 9 | 415.8 | | Reaction stirring over 4 days. 50 g THF added |
| 10 | 465.8 | | Reaction mixture filtered through polish filter and then |
| | | | through SiliaFlash (5 g). |
| | | | Flowrate: 60 mL/min Backpressure: 5-10 psi |
| 11 | 595.8 | | Reactor was forward rinsed with 130 g THF |
| 12 | 55.8 | | Distillation |
| | | | Jacket T = 50.0° C. P = 300-400 mbar Ti = 35.7-41.7° C. |
| | | | m(distillate) = 540 g |
| 13 | 157.8 | | Added 102.0 g of MeCN. Solution become milky and |
| | | | crystallization started. |
| 14 | 145.8 | | 12.0 g of slurry removed |
| 15 | 169.9 | | 24.1 g of water added to achieve 20 wt % water |
| | | | Flow rate = 1 mL/min |
| 16 | | | Ramp: 20 → 0° C. over 1 hour |
| | | | Age overnight |
| 17 | | | Filtration time = 7 min |
| | | | Cake depth = 2.5 cm m(mother liquor) = 98.8 g |
| 18 | | | Wash with 50 g MeCN/water = 80:20 mixture. |
| | | | Wash time = 5 min |
| 19 | | | Yield: 27.34 g; 65% (after accounting for removed |
| | | | samples) Purity: 95.7% |

### Impurity Removal Evaluation

The racemization of II-1 with KOAc was previously performed. The formation of new impurity (RRT=1.05) during racemization reaction was observed. This impurity did not purge during crystallization from MeCN/water=80:20.

The purging of impurities during crystallization from DMF/Acetone/water (6.25V:6.25V:4.75V) was evaluated.

The crystallization from DMF/Acetone/water mixture is preferrable since a significantly better purging of the undesired impurities is observed.

### Effect of Air on the Generation of the RRT 1.05 Impurity

Since the impurity RRT 1.05 is identified as an oxidative product from the reaction, the presence of air during the racemization reaction was evaluated.

The reaction performed in the presence of air has higher levels of oxidation impurity (RRT=1.05). It is therefore, recommended to purge the reactor with N₂ prior to the racemization.

### Racemization Scale-Up

The racemization of II-1 with KOAc was previously performed on relatively larger scale. However, II-1 was crystallized from MeCN/water. The decision was made to perform the crystallization of II-1 from DMF/Acetone/water (6.25V:6.25V:4.75V). A large-scale demonstration of II-1 racemization with KOAc was performed followed by solvent swap from MeCN/THF to DMF for final crystallization.

### Procedure Description

| | Batch weight | II-1 | |
|---|---|---|---|
| Step # | g | wt % | Procedure comments |
| 1 | 89.25 | 20.7 | II-1 solution charged in 0.5 L reactor. |
| | | | m(II-1) = 18.4 g |
| 2 | 50.75 | | Distillation |
| | | | Tjacket = 50.0° C. Tcondenser = −10.0° C. Ti = 45.2° C. |
| | | | Stirring = 300 RPM m(distillate) = 38.5 g |
| 3 | 92.85 | | Solvent swap. Addition of MeCN in 40-50 g portions. |
| | | | P = 200-400 mbar Ti = 37.7-45.2° C. m(MeCN added) = |
| | | | 196.2 g m(distillate) = 154.1 g |
| 4 | 92.85 | | Sample #2 |
| | | | [MeOH] by 1H-NMR < 1000 ppm Jacket T = 20° C. |
| 5 | 222.85 | | Added m(THF) = 130 g |
| | | | Sample #3 (NMR):THF/MeCN = 2:1~8 V:4 V |
| 6 | 222.85 | | Sample #3 (NMR):THF/MeCN = 2:1~8 V:4 V |
| | | | Sample #4 (HPLC):[II-1] = 8.6 wt % |
| 7 | 222.85 | 8.6 | Sample #4 (HPLC):[II-1] = 8.6 wt % |
| 8 | 222.85 | | Added AcOK - 2.7 g |
| 9 | 222.85 | | Samples #5-7 (chiral HPLC) |
| | | | Reaction stirred for 46 hours |
| 10 | 222.85 | | Reaction mixture filtered through polish filter and then |
| | | | through SiliaFlash (5 g). |
| | | | Flowrate: 60 mL/min Backpressure: 0-5 psi |
| 11 | 272.85 | | Chased with 50 g THF |
| 12 | 29.55 | | Distillation |
| | | | Jacket T = 50.0° C. P = 300-380 mbar Ti = 34.9-40.3° C. |
| | | | m(distillate) = 243.3 g |
| 13 | 112.75 | | Added m(DMF) = 109.2 g in two portions |
| | | | P = 60 mbar Ti = 40.8° C. m(distillate) = 26.0 g |
| 14 | 112.75 | | Sample #8 (NMR): |
| | | | W(MeCN) = 5.4 wt % |
| | | | W(THF) = 2.4 wt % |
| 15 | 202.95 | | Added m(Acetone) = 90.2 g |
| 16 | 290.35 | | Added m(water) = 87.4 g |
| | | | Flow rate = 0.5 mL/min |
| 17 | | | Product filtered through 20 μm filter. |
| | | | M(Mother liquor) = 223.5 g [II-1] = 0.5 wt % |
| | | | Filtration time = 3 min |
| | | | Cake depth = 3 cm |
| 18 | | | Chased with 23.5 g of acetone. Slurry wash. M(wash) = |
| | | | 57.1 g. [II-1] = 0.4 wt % |
| 19 | | | Product dried under vacuum at 40° C. |
| 20 | | | m(II-1) = 16.46 g |
| | | | Yield: 86%. |
| | | | Purity: 98.05% |

### Solvate Observation

The analysis of the final material by 1H-NMR revealed that the product is not the expected acetone solvate, but a DMF solvate instead. This was likely due to vacuum oven drying process. Since acetone is much more volatile than DMF, it likely evaporated first. The oven headspace was then saturated with DMF vapors which replaced acetone forming the solvate.

This result suggests that larger quantity of acetone should be used in the final rinse.

This assumption was tested as follow.
A total of 1.17 g of the dried material (DMF solvate) was slurried in 5 g of acetone and allowed to stay for 1.5 hours at room temperature.
The slurry was filtered.
The resulting product was dried under vacuum to give 1.1 g of material (94%).
concentration of II-1 in the acetone wash (loss): [II-1]=0.2 wt %.
A 1H-NMR analysis shows mixed solvate produced: II-1/acetone/DMF (1/0.6/0.3).

This experiment supports the assumption that both DMF and acetone can fit in the II-1 crystal cavities because of their similar molecular size. Therefore, they can easily replace each other.

It is recommended to perform 2 or 3 acetone cake washes after the filtration to significantly reduce residual DMF in the wet cake and favor the acetone solvate formation during the drying step.

### Final Product Analysis

The final crystals obtained were tested for total purity by chromatography.

The total purity is 98.05% with the main impurities are RRT 0.94 and RRT 1.05.

The process to be implemented at kilo scale is described below.
1. Set reactor 1 jacket temperature to 25° C. and inert with nitrogen.
2. Charge II-1 solution to ˜⅓ of the reactor volume.
3. Set reactor 1 jacket temperature to 50° C. and start applying vacuum.
   Continuous feed addition (II-1 solution) while distilling.
4. Charge acetonitrile (RM-2004; 11S) while keeping the batch volume constant.
5. Distillation is deemed complete when batch is concentrated to ˜5 V (4 S).
6. Cool the batch to 25° C.
7. IPC #1 (specification NMT 1,000 ppm MeOH).
   Sample #2 ([II-1]=NLT 20 wt %.)
8. Calculate batch acetonitrile content.
   Calculate acetonitrile charge to bring total acetonitrile content to 5 V (3.93 S).
9. Charge Acetonitrile (RM-2004) calculated in step 8.
10. Charge 8.73 S (10 V) THF (RM-2126) to the batch.
11. Charge 0.16 S KOAc (RM-2014) to the batch.
   This is the start of the reaction.
12. Stir reaction mixture for NLT 20 hours.
13. IPC #3. Reaction completion
   specification: NMT 53% RTX1274075
14. Transfer reaction mixture through SiliaFlash silica cartridge to drum.
15. Chase with 1S THF (RM-2126).
16. Clean the reactor with water and Acetonitrile.
17. Transfer batch from the drum to the reactor.
   Fill ˜⅓ of the total reactor volume.
18. Set reactor 1 jacket temperature to 50° C. and start applying vacuum.
   Continuous Feed batch solution addition from the drum while distilling.
19. When the drum content is transferred to the reactor and the batch is concentrated to ˜3.5 S, add DMF (2.9 S) and continue distillation.
20. Distillation is deemed complete when distillation stalls at ≤50 mbar.
   R&D FIO for residual MeCN and THF
21. Add 3S DMF (RM-2148).
22. Cool batch to 20±5° C.
23. Add 4.9S Acetone (RM-4001).
24. Stir for NLT 15 min.
25. Add 4.75 S water (RM-3000) over NLT 4 hours.
26. Age the batch at 20±5° C. for NLT 2 hours.
27. Transfer the batch on Aurora filter.
   The crystals are very fine and caution should be taken when applying vacuum.
28. Charge Acetone (RM-4001)/water=60:40 to the reactor as a rinse.
29. Transfer the rinse from the reactor to the Aurora filter for slurry wash.
30. Mix the slurry on the filter for NLT 5 min.
31. Apply gentle vacuum to the bottom of the Aurora to deliquor the cake.
32. Charge Acetone (RM-4001)/water=60:40 to the reactor as a rinse.
33. Transfer the rinse from the reactor to the Aurora filter for slurry wash.
34. Apply gentle vacuum to the bottom of the Aurora to deliquor the cake.
35. Charge Acetone (RM-4001)/water=60:40 to the reactor as a rinse.
36. Transfer the rinse from the reactor to the Aurora filter for slurry wash.
37. Mix the slurry on the filter for NLT 5 min.
38. Apply gentle vacuum to the bottom of the Aurora to deliquor the cake.
39. Charge 3S Acetone (RM-4001) to the reactor as a rinse.
40. Transfer the rinse from the reactor to the Aurora filter for slurry wash.
41. Mix the slurry on the filter for NLT 5 min.
42. Apply gentle vacuum to the bottom of the Aurora to deliquor the cake.
43. Heat Aurora jacket to 30° C. Dry the cake by passing nitrogen through the cake for NLT 12 hours.
   After 2 hours of drying, start stirring the cake regularly.
44. IPC #4 (drying completion)
45. Package when passing results for IPC #4 are obtained.

### GMP Crude Feed Use Test

The crude feed material to be used for the kilo GMP campaign was tested on the mini-SMB to evaluate any issue prior to the campaign.

### Stability Testing

Prior to the SMB separation, a stability test was conducted to identify any potential problems with the racemized feed.

A column was packed with virgin Chiralpak IH, the same lot that is in the current mini SMB columns.

Stability injections
Solution 1, 1.9 g/l injected at 5 μl
Solution 2, 28 g/l injected at 250 μl×7
Injection sequence was repeated 10 times for a total of 70 overloaded injections on the column.

The retention times of the analytical injections are reproducible over the course of the experiment.

### SMB Separation

### Solubility

The solubility of the crude feed material was tested since the previous sample provided was containing insoluble material. Samples of feed at 20, 25 and 30 g./l were prepared in the SMB mobile phase. The samples were left at room temperature overnight and no precipitation was observed.

However, during the demonstration run (see below) solids precipitated in the feed solution upon standing at ambient temperatures which ranged between 18 and 22° C. This was the case with the 18-19 g/l feeds as well. The solids were filtered off before the feed was used. The feed vessel will have to be jacketed and held at 25° C. to keep the feed in solution.

### SMB Demonstration

Based on the observed solubility a feed solution at ˜28 g/l was prepared and was further filtered during processing to eliminate solids precipitation.

The separation was resumed using the parameters from the previous demonstration run.

The purity remained stable, and the only adjustment required was an increase to the switch time to maintain the I-1 purity above 99.5%. Under the current conditions the productivity is 3.2 kg/day/kg CSP.

### Separation Output

Bot products in the collection bottles were tested for chiral purity.
I-1 Extract 99.8%
II-1 Raffinate 95.5%

### Product Recovery

After the racemized feed was processed, the separation was stopped, and the products were isolated in the rotovaps.

### Mass Balance

A total of 120.2 g of crude racemic feed was processed.

### I-1 Material Isolation:

The procedure for the isolation of the desired enantiomer I-1 by concentrating the Extract stream from the SMB followed by the addition of n-heptane as an antisolvent was developed (see earlier in the document). The procedure was performed on a sample of the crude feed material separated on mini-SMB.

In this experiment, the extract was use-tested to evaluate the performance of the developed procedure.

The product is recovered as needles with high chiral purity (99.65%) and high total purity (99.4%) with only a main impurity at RRT 0.94. The overall yield for the isolation is >97%.

### II-1 Racemization

The procedure for the racemization of the undesired enantiomer II-1 followed by solvent swap to DMF/Acetone and addition of water as antisolvent have been developed (see earlier in the document). Based on the initial observations additional improvements were made as described above.

In this experiment, the raffinate (II-1) obtained from the SMB separation of the GMP material sample was use-tested to evaluate the performance of the modified and improved racemization procedure.

The product is recovered as needles with high total purity (99.3%) and only a main impurity at RRT 0.94. The overall yield for the racemization+isolation is just below 80%.

### SMB Separation of Racemized Feed

### SMB Parameters

A 25.5 g/l feed solution of racemized II-1 was prepared. The separation was resumed using the parameters from the previous run.

An adjustment was made to the switch time after 5 cycles to maintain the I-1 purity above 99.5%. The purity remained stable for the duration of the trial.

### Product Analysis

The product was collected in bottles and tested for chiral purity.
I-1 Extract 99.8%
II-1 Raffinate 100.0%

### Mass Balance

The solvent was evaporated from the extract and raffinate using a rotovap and the dry mass of each was obtained.
Extract 10.52 g
Raffinate 9.68 g

The extract and raffinate were then diluted to 10% w/w with methanol and was processed by chemist for final isolation.

### Second Pass Product Analysis

The material produced was isolated using the line procedure described in this document and gave final product with 99.30% achiral and 99.86% chiral purity.

### Use Test Conclusion

The use test of the racemic feed is successful. The SMB separation worked as previously demonstrated during the SMB process development. Insoluble in the feed material need to be closely monitored as this could potentially result in processing issued at the kilo scale (plugging of the filters). The procedure for the recovery of the desired I-1 as a solid worked well with good purity and yield. The procedure for the racemization of II-1 and the isolation of the resulting III-1 also worked well with high purity and good recovery.

### Conclusion

The process for the crystallization of the I-1 compound obtained after the SMB separation of the racemic I-1 was evaluated and a line procedure for the kilo scale lab process was provided. This procedure will also be used for the isolation of the undesired enantiomer.

The racemization process of the undesired enantiomer to was also evaluated and a line procedure was provided. The process preceded typically and gave crystalline III-1 in good yield and purity.

The GMP racemic feed to be used in the kilo lab campaign was evaluated and use test including SMB separation, isolation and racemization was performed successfully. However, the solubility of the acetone solvate must be monitored closely as solids precipitation has been observed and could result in processing issues.

The line procedures developed in this report are being converted to batch record procedures for the kilo campaign.

### Example 3-A. Synthesis of Compounds IV-1 and IV-2

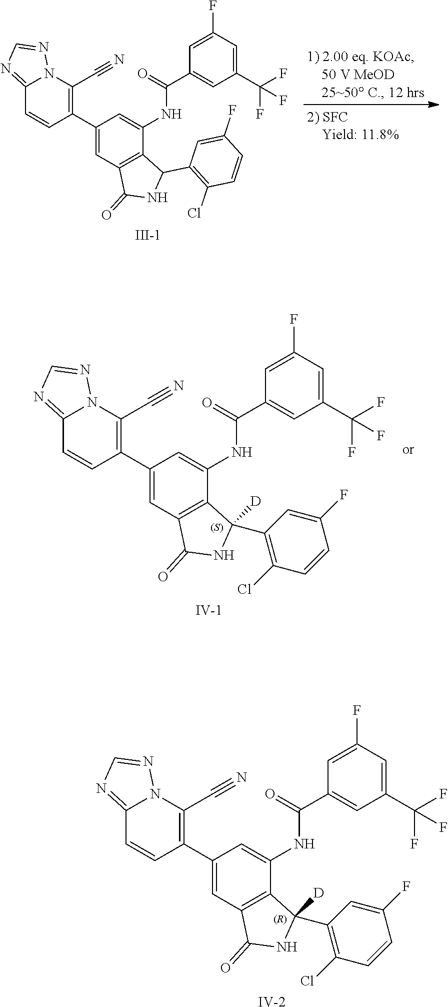

To a solution of III-1 (1.00 g, 1.64 mmol, 1.00 eq) in MeOD (100 mL) was added KOAc (322 mg, 3.28 mmol, 2.00 eq) at 25° C. Then the mixture was heated to 50° C. and stirred at 50° C. for 12 hrs. LCMS (EW31392-3-P1A) showed that III-1 was consumed and the desired MS (Rt=0.750 min) was detected. The mixture was concentrated and dilute with ethyl acetate (10.0 mL). The residue was poured into saturated NaHCO₃ solution (30.0 mL) and separated. The aqueous layer was extracted with ethyl acetate (10.0 mL*3). The combined organic layer was adjusted pH to 6 with acetic acid-D. Then the combined organic layer was dried over Na₂SO₄ and concentrated to give crude product (0.80 g). The crude product (0.80 g) was separated by SFC (column: DAICEL CHIRALCEL OD (250 mm*30 mm, 10 μm); mobile phase: [Neu-MeOH]; B %: 40%-40%, 3.3 mins) and concentrated. Two residues were dilute with Acetonitrile-d₃ (5.00 mL) and H₂O (10.0 mL), then lyophilized to give isomer 1 (120 mg, 193 μmol, 11.8% yield, 98.2% purity) as an off-white solid and isomer 2 (120 mg, 188 μmol, 11.4% yield, 95.4% purity) as a yellow solid.

Isomer 1:

LCMS: product: Rt=0.750 min, m/z=610.1 (M+H)⁺

MS: product: m/z=610.1 (M+H)⁺

HPLC: product: Rt=2.985 mins, 98.2% purity under 220 nm

SFC: product: Rt=1.893 mins, 100% ee under 220 nm

¹H NMR: 400 MHz, DMSO-d₆

δ 10.7 (s, 1H), 9.35 (s, 1H), 8.81 (s, 1H), 8.34 (d, J=9.6 Hz, 1H), 8.07 (s, 1H), 8.00-7.96 (m, 2H), 7.84 (s, 1H), 7.77-7.75 (m, 1H), 7.68 (s, 1H), 7.38-7.34 (m, 1H), 7.16-7.11 (m, 1H).

¹⁹F NMR: 400 MHz, DMSO-d₆

δ −61.34, −109.56

Special LCMS: (M+H+1D) purity: 96.8%

Isomer 2:

MS: product: m/z=610.0 (M+H)⁺

HPLC: product: Rt=2.986 mins, 95.4% purity under 220 nm

SFC: product: Rt=2.088 mins, 100% ee under 220 nm

¹H NMR: 400 MHz, DMSO-d₆

δ 10.7 (s, 1H), 9.35 (s, 1H), 8.81 (s, 1H), 8.34 (d, J=9.2 Hz, 1H), 8.07 (s, 1H), 8.00-7.95 (m, 2H), 7.84 (s, 1H), 7.77-7.75 (m, 1H), 7.68 (s, 1H), 7.38-7.34 (m, 1H), 7.16-7.11 (m, 1H).

¹⁹F NMR: 400 MHz, DMSO-d₆

δ −61.33, −109.59

Special LCMS: (M+H+1D) purity: 97.9%

### Example 4-A. Biological Assays

Selected compounds of the present disclosure were tested in an ADP-Glo Biochemical PIK3CA Kinase Assay. Compounds to be assayed were plated in 16 doses of 1:2 serial dilutions (20 nL volume each well) on a 1536-well plate, and the plate warmed to room temperature PIK3CA enzyme (e.g. H1047R, E542K, E545K, or wild-type) (1 μL of 2 nM solution in Enzyme Assay Buffer (comprising 50 mM HEPES pH 7.4, 50 mM NaCl, 6 mM MgCl₂, 5 mM DTT and 0.03% CHAPS)) was added and shaken for 10 seconds and preincubated for 30 minutes. To the well was added 1 μL of 200 μM ATP and 20 μM of diC8-PIP2 in Substrate Assay Buffer (50 mM HEPES pH7.4, 50 mM NaCl, 5 mM DTT and 0.03% CHAPS) to start the reaction, and the plate was shaken for 10 seconds, then spun briefly at 1500 rpm, and then incubated for 60 minutes at room temperature. The reaction was stopped by adding 2 μL of ADP-Glo reagent (Promega), and spinning briefly at 1500 rpm, and then incubating for 40 minutes. ADP-Glo Detection reagent (Promega) was added and the plate spun briefly at 1500 rpm, then incubated for 30 minutes. The plate was read on an Envision 2105 (Perkin Elmer), and the IC₅₀ values were calculated using Genedata software.

Results of the ADP-Glo Biochemical PIK3CA Kinase Assay using H₁₀₄₇R PIK3CA enzyme are presented in Table 1. Compounds having an IC₅₀ less than or equal to 100 nM are represented as “A”; compounds having an IC₅₀ greater than 100 nM but less than or equal to 500 nM are represented as “B”; compounds having an IC₅₀ greater than 500 nM but less than or equal to 1 μM are represented as “C”; compounds having an IC₅₀ greater than 1 μM but less than or equal to 10 UM are represented as “D”, and compounds having an IC₅₀ greater than 10 μM but less than or equal to 100 μM are represented as “E”.

Selected compounds of the present disclosure were tested in a MCF10A Cell-Based PIK3CA Kinase Assay, namely the CisBio Phospho-AKT (Ser473) HTRF assay, to measure the degree of PIK3CA-mediated AKT phosphorylation. MCF10A cells (immortalized non-transformed breast cell line) overexpressing hotspot PIK3CA mutations (including H1047R, E542K, and E545K mutations) were used. Cells were seeded at 5,000 cells per well in DMEM/F12 (Thermo Fisher Scientific) supplemented with 0.5 mg/ml hydrocortisone, 100 ng/ml Cholera Toxin, 10 μg/mL insulin, and 0.5% horse serum. Once plated, cells were placed in a 5% CO₂, 37° C. incubator to adhere overnight.

The following day, compounds were added to the cell plates in 12 doses of 1:3 serial dilutions. The dose response curves were run in duplicate. Compound addition was carried out utilizing an Echo 55 Liquid Handler acoustic dispenser (Labcyte). The cell plates were incubated for 2 hours in a 5% CO₂, 37° C. incubator. Following compound incubation, the cells were lysed for 60 min at room temperature. Finally, a 4-hour incubation with the HTRF antibodies was performed at room temperature. All reagents, both lysis buffer and antibodies, were used from the CisBio pAKT S473 HTRF assay kit, as per the manufacturers protocol. Plates were read on an Envision 2105 (Perkin Elmer), and the IC₅₀ values were calculated using Genedata software.

Results of the MCF10A Cell-Based PIK3CA Kinase Assay are presented in Table 1. Compounds having an IC₅₀ less than or equal to 1 μM are represented as “A”; compounds having an IC₅₀ greater than 1 μM but less than or equal to 5 μM are represented as “B”; compounds having an IC₅₀ greater than 5 μM but less than or equal to 10 μM are represented as “C”; compounds having an IC₅₀ greater than 10 μM but less than or equal to 36 μM are represented as “D”; and compounds having an IC₅₀ greater than 36 μM but less than or equal to 100 μM are represented as “E”.

| | Biochemical - PI3Ka His- | |
|---|---|---|
| | p110 H1047R FL 100 uM | |
| | ATP 10 uM diC8 PIP2 ADP- | |
| | Glo with 120 min pre- | Cell - PD - MCF10A |
| | incubation - 2181 | H1047R/−−2483 |
| Compound | (IC50, uM) | (IC50, uM) |
| IV-1 | D | |
| IV-2 | A | A |

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound in solid form, wherein the compound is compound I-1:
or a solvate thereof.

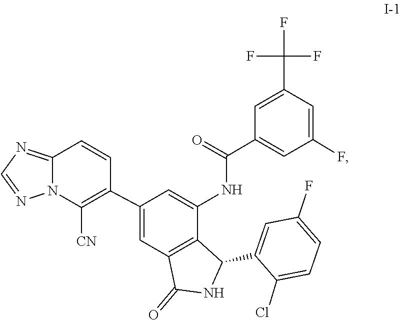 [SMILES: N#Cc1c(-c2cc(NC(=O)c3cc(F)cc(C(F)(F)F)c3)c3c(c2)C(=O)N[C@H]3c2cc(F)ccc2Cl)ccc2ncnn12]

2. The compound of claim 1, wherein the compound is amorphous.

3. The compound of claim 1, wherein the compound is crystalline.

4. The compound of claim 1, wherein the solid form is Form A.

5. The compound of claim 1, wherein the solid form is Form B.

6. The compound of claim 1, wherein the solid form is Form C.

7. A compound in solid form, wherein the compound is a compound of Formula (I): 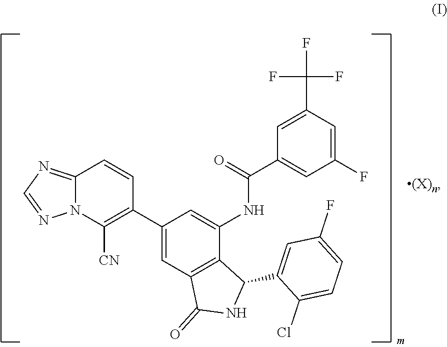
or a solvate thereof;
wherein:
m is 1, 2, 3, 4, 5, 6, 7, 8, or 9;
n is 0, 0.5, 1, 1.5, 2, 2.5, or 3; and
X is hydrochloric acid, p-toluene sulfonic acid, methane sulfonic acid, naphthalene-1,5-disulfonic acid, or 2-naphthalene sulfonic acid.

8. The compound of claim 1, wherein the compound is Compound I-2: 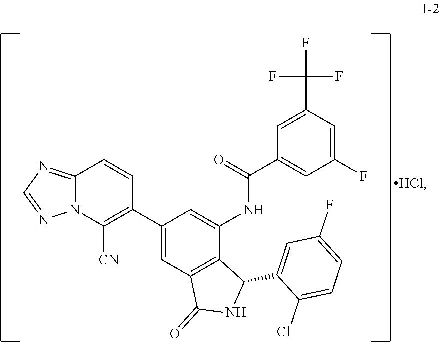
or a solvate thereof.

9. The compound of claim 8, wherein the solid form is Form A.

10. The compound of claim 1, wherein the compound is Compound I-3: 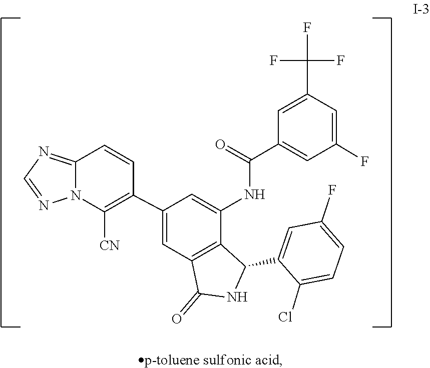
or a solvate thereof.

11. The compound of claim 10, wherein the solid form is Form A or Form B.

12. The compound of claim 1, wherein the compound is Compound I-4: 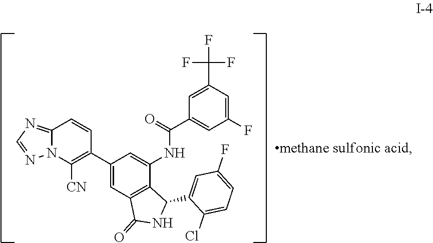
or a solvate thereof.

13. The compound of claim 12, wherein the solid form is Form A.

14. The compound of claim 1, wherein the compound is Compound I-5: 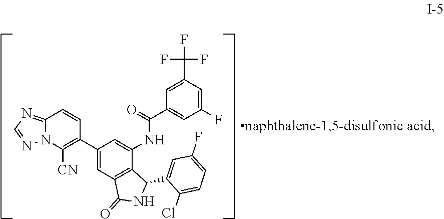
or a solvate thereof.

15. The compound of claim 14, wherein the solid form is Form A or Form B.

16. A compound in solid form, wherein the compound is of Formula (II) 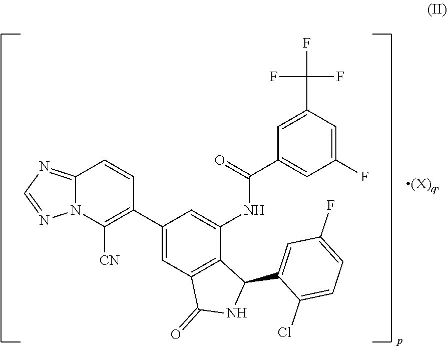
or a solvate thereof,
wherein:
p is 1, 2, 3, 4, 5, 6, 7, 8, or 9;
q is 0, 0.5, 1, 1.5, 2, 2.5, or 3; and
X is hydrochloric acid, p-toluene sulfonic acid, methane sulfonic acid, naphthalene-1,5-disulfonic acid, or 2-naphthalene sulfonic acid.

17. The compound of claim 16, wherein the compound is amorphous.

18. The compound of claim 16, wherein the compound is crystalline.

19. The compound of claim 16, wherein the compound is compound II-1: 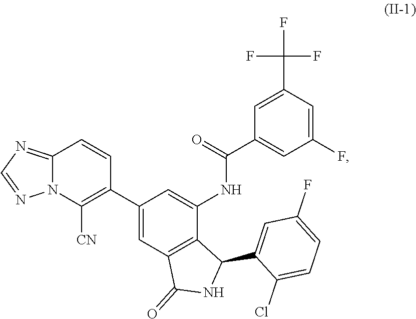 [SMILES: N#Cc1c(-c2cc(NC(=O)c3cc(F)cc(C(F)(F)F)c3)c3c(c2)C(=O)N[C@@H]3c2cc(F)ccc2Cl)ccc2ncnn12]
or a solvate thereof.

20. The compound of claim 19, wherein the solid form is Form A, Form B, or Form C.

21. A compound in solid form, wherein the compound is of Formula III: 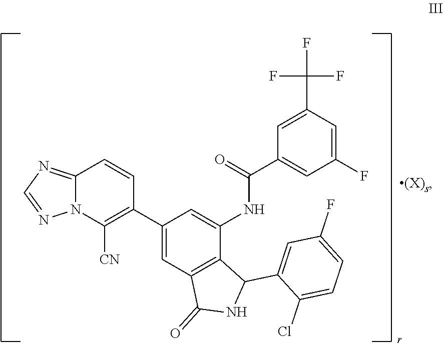
or a solvate thereof,
wherein:
r is 1, 2, 3, 4, 5, 6, 7, 8, or 9;
s is 0, 0.5, 1, 1.5, 2, 2.5, or 3; and
X is hydrochloric acid, p-toluene sulfonic acid, methane sulfonic acid, naphthalene-1,5-disulfonic acid, or 2-naphthalene sulfonic acid.

22. The compound of claim 21, wherein the compound is amorphous.

23. The compound of claim 21, wherein the compound is crystalline.

24. The compound of claim 21, wherein the compound is compound III-1 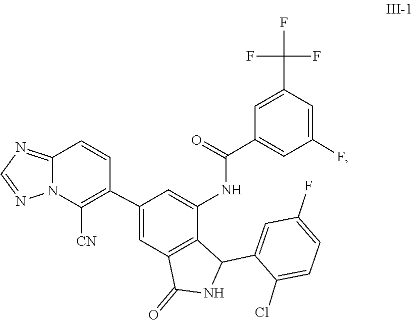 [SMILES: N#Cc1c(-c2cc(NC(=O)c3cc(F)cc(C(F)(F)F)c3)c3c(c2)C(=O)NC3c2cc(F)ccc2Cl)ccc2ncnn12]
or a solvate thereof.

25. The compound of claim 24, wherein the solid form is Form A, Form B, Form C, Form D, Form E, or Form F.

26. The compound of claim 21, wherein the compound is Compound III-2: 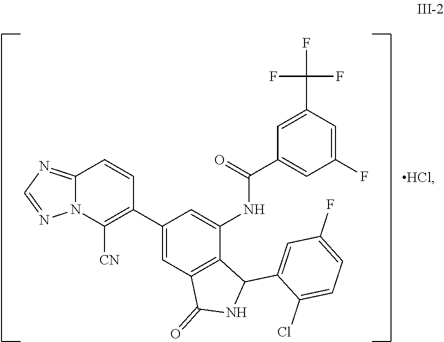
or a solvate thereof.

27. The compound of claim 26, wherein the solid form is Form A or Form B.

28. The compound of claim 21, wherein the compound is compound III-6 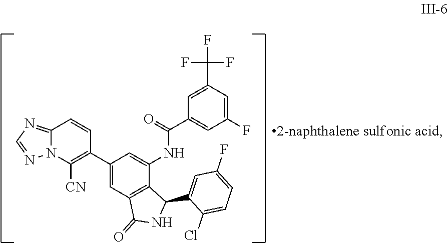
or a solvate thereof.

29. The compound of claim 28, wherein the solid form is Form A.

30. A pharmaceutical composition comprising a compound of any one of claims 1-29, and a pharmaceutically acceptable carrier.

31. A method of inhibiting PI3Kα activity in a subject in need thereof, comprising administering a therapeutically effective amount of a compound of any of claims 1-29, or a pharmaceutical composition of claim 30, to the subject.

32. A method of treating a cancer in a subject in need thereof, comprising administering a therapeutically effective amount of a compound of any one of claims 1-29, or a pharmaceutical composition of claim 30, to the subject.

33. The method of claim 31 or 32, further comprising administering a therapeutically effective amount of an antibody, an antibody-drug conjugate, a kinase inhibitor, an immunomodulator, or a histone deacetylase inhibitor.

34. A kit comprising a compound of any of claims 1-29.

35. The kit of claim 34, further comprising written instructions describing preparation of a pharmaceutical composition suitable for administration to a patient from the solid form or compound.

36. The kit of claim 34 or 35, further comprising written instructions describing how to administer the resulting composition to the patient.

37. The kit of any one of claims 34-36, further comprising a pharmaceutically acceptable excipient.

38. A process for preparing a crystalline form of a compound of Formula (I), comprising: a) preparing a solution of a compound of Formula (I): b) adjusting the temperature so that solid crystalline form of a compound of Formula (I) precipitates out of the solution; and c) isolating the solid crystalline form.

39. A process for preparing a crystalline form of a compound of Formula (II), comprising: a) preparing a solution of a compound of Formula (II): b) adjusting the temperature so that solid crystalline form of a compound of Formula (II) precipitates out of the solution; and c) isolating the solid crystalline form.

40. A process for preparing a crystalline form of a compound of Formula (III), comprising:
a) preparing a solution of a compound of Formula (III): b) adjusting the temperature so that solid crystalline form of a compound of Formula (III) precipitates out of the solution; and c) isolating the solid crystalline form.

41. A compound of Formula (IV-1) 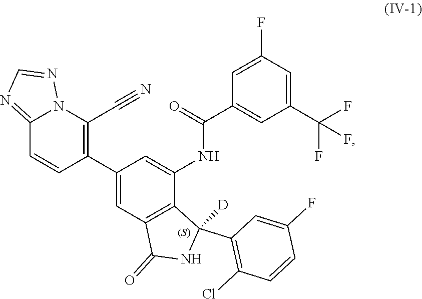
or a pharmaceutically acceptable salt thereof.

42. A compound of Formula (IV-2) 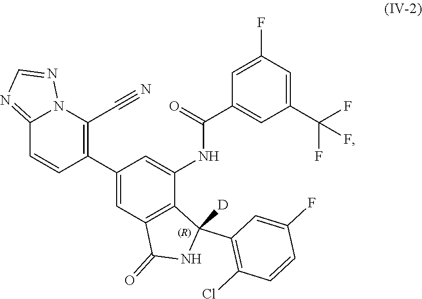
or a pharmaceutically acceptable salt thereof.

43. A pharmaceutical composition comprising a compound of claim 41 or 42, and a pharmaceutically acceptable carrier.

44. A method of inhibiting PI3Kα activity in a subject in need thereof, comprising administering a therapeutically effective amount of a compound of claim 41 or 42, or a pharmaceutical composition of claim 43, to the subject.

45. A method of treating a cancer in a subject in need thereof, comprising administering a therapeutically effective amount of a compound of claim 41 or 42, or a pharmaceutical composition of claim 43, to the subject.

46. The method of claim 44 or 45, further comprising administering a therapeutically effective amount of an antibody, an antibody-drug conjugate, a kinase inhibitor, an immunomodulator, or a histone deacetylase inhibitor.

47. A kit comprising a compound of claim 41 or 42.

48. The kit of claim 47, further comprising written instructions describing preparation of a pharmaceutical composition suitable for administration to a patient from the solid form or compound.

49. The kit of claim 47 or 48, further comprising written instructions describing how to administer the resulting composition to the patient.

50. The kit of any one of claims 47-49, further comprising a pharmaceutically acceptable excipient.

51. A process for preparing a compound of Formula IV-1 and a compound of Formula IV-2, comprising deuteration of compound III-1 followed by a purification step to separate the enantiomers, thereby forming compounds IV-1 and IV-2:

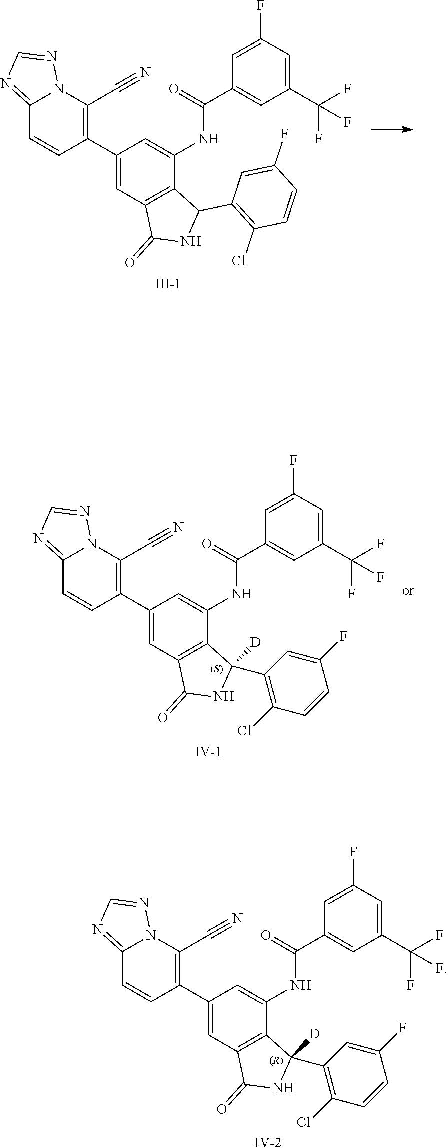

52. A process for preparing a compound of Formula I-1 and a compound of Formula I-2, comprising subjecting compound III-1 to a SMB separation, for example, as described in Example 1-A, thereby forming compounds I-1 and II-1:

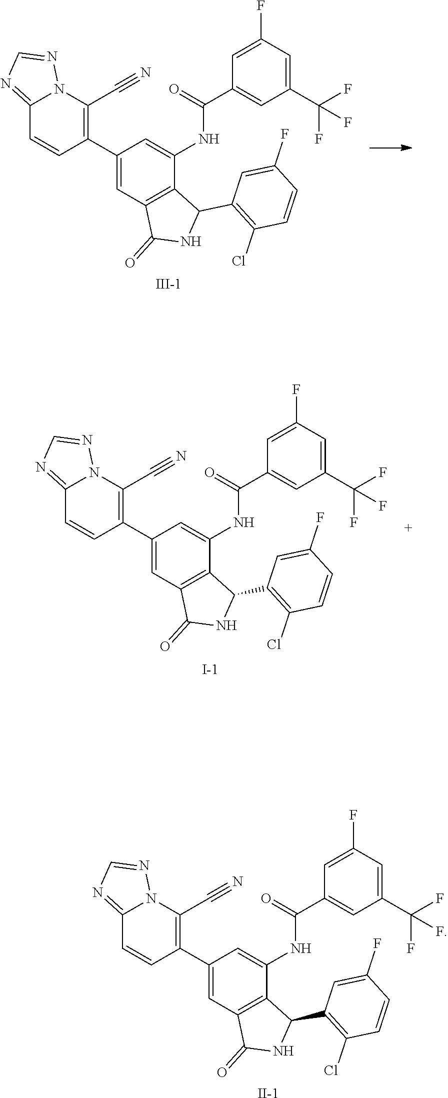

53. A process for preparing a compound of Formula III-1, comprising a racemization of compound II-1: [chemical structure]
